(19) United States
(12) Patent Application Publication
(10) Pub. No.: US 2024/0425500 A1
(43) Pub. Date: Dec. 26, 2024

(54) IMIDAZO[1,2-A]PYRIDINE DERIVATIVES

(71) Applicant: SYNGENTA CROP PROTECTION AG, Basel (CH)

(72) Inventors: Stephane André Marie JEANMART, Stein (CH); Mathias BLUM, Stein (CH); Alexandre Franco Jean Camille LUMBROSO, Basel (CH); Nicolas GERMAIN, Stein (CH); Martin POULIOT, Stein (CH)

(73) Assignee: SYNGENTA CROP PROTECTION AG, Basel (CH)

(21) Appl. No.: 18/700,358

(22) PCT Filed: Oct. 6, 2022
(86) PCT No.: PCT/EP2022/077777

§ 371 Date: Apr. 11, 2024

(30) Foreign Application Priority Data
Oct. 14, 2021 (EP) 21202562.1
Jul. 7, 2022 (EP) 22183563.0

(51) Int. Cl. C07D 471/04; A01N 43/90; A01N 47/18; A01N 47/36; A01P 3/00

(52) U.S. Cl. CPC C07D 471/04; A01N 43/90; A01N 47/18; A01N 47/36; A01P 3/00

(57) ABSTRACT

The current invention relates to compounds of the formula (I) wherein the substituents are as defined in claim 1, to processes and methods for preparing compounds of formula (I), to agrochemical compositions comprising compounds of formula (I) as defined in claim 1, to preparation of these compositions and to the use of the compounds or compositions in agriculture or horticulture for combating, preventing or controlling infestation of plants, harvested food crops, seeds or non-living materials by phytopathogenic microorganisms, in particular fungi.

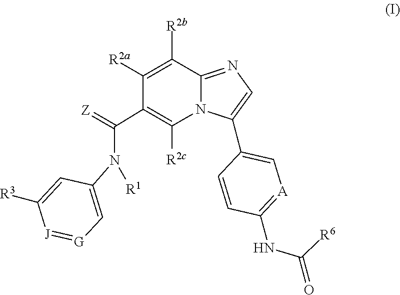

The present invention relates to microbiocidal imidazo[1,2-a]pyridine derivatives, e.g. as active ingredients, which have microbiocidal activity, in particular fungicidal activity, more particularly activity against oomycetes. The invention also relates to preparation of these imidazo[1,2-a]pyridine derivatives, to intermediates useful in the preparation of these imidazo[1,2-a]pyridine derivatives, to the preparation of these intermediates, to agrochemical compositions which comprise at least one of the imidazo[1,2-a]pyridine derivatives, to preparation of these compositions and to the use of the imidazo[1,2-a]pyridine derivatives or compositions in agriculture or horticulture for combating, controlling or preventing infestation of plants, harvested food crops, seeds or non-living materials by phytopathogenic microorganisms, in particular fungi, more particularly oomycetes.

It has now surprisingly been found that certain novel imidazo[1,2-a]pyridine derivatives have favourable fungicidal properties, in particular against oomycetes.

Therefore, in a first aspect, as embodiment 1, the present invention provides compounds of formula (I)

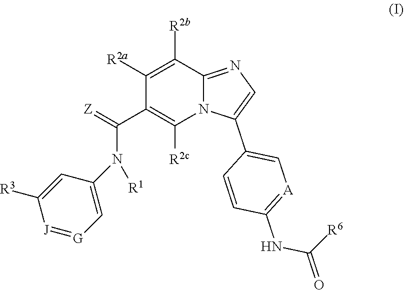

wherein Z is O or S, and preferably Z is O;
R¹ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen, hydroxy and CN;
R^{2a}, R^{2b} and R^{2c} independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;

### J is CR⁴ or N;

### G is CR⁵ or N;

wherein when J is N, then G is CR⁵; and when G is N, then J is CR⁴:
R³, R⁴ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl and C₁₋₆alkylcarbonyl groups is optionally substituted with one to three substituents independently selected from halogen and CN;

### A is CH or N; and

R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN;
or a salt or N-oxide thereof.

In a preferred embodiment 1 wherein Z is O, the present invention provides compounds of formula (I)

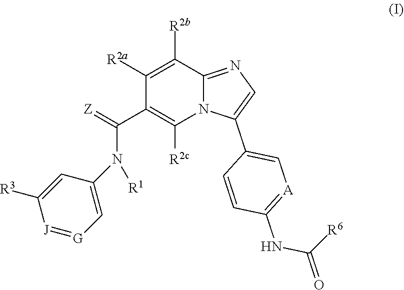

wherein R¹ is selected from C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, halogen, CN, C₁₋₄alkyl and C₁₋₄alkoxy;

### J is CR⁴ or N;

### G is CR⁵ or N;

wherein when J is N, then G is CR⁵; and when G is N, then J is CR⁴;
R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen;

### A is CH or N; and

R⁶ is selected from C₁₋₄alkyl, C₁₋₄alkoxy, C₃₋₆cycloalkyl and C₃₋₆cycloalkyl-C₁₋₃alkyl, wherein each of said groups is optionally substituted with one to three substituents independently selected from halogen;
or a salt or N-oxide thereof.

In a preferred embodiment 1 wherein Z is S, the present invention provides compounds of formula (I)

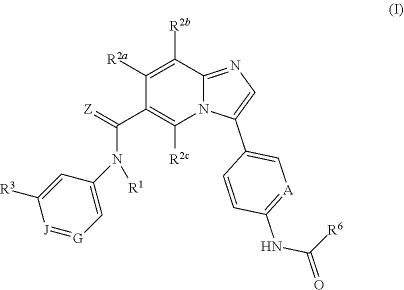

wherein R¹ is selected from C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, halogen, CN, C₁₋₄alkyl and C₁₋₄alkoxy;

### J is CR⁴ or N;

### G is CR⁵ or N;

wherein when J is N, then G is CR⁵; and when G is N, then J is CR⁴;
R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen;

### A is CH or N; and

R⁶ is selected from C₁₋₄alkyl, C₁₋₄alkoxy, C₃₋₆cycloalkyl and C₃₋₆cycloalkyl-C₁₋₃alkyl, wherein each of said groups is optionally substituted substituted with one to three substituents independently selected from halogen;
or a salt or N-oxide thereof.

In a second aspect the present invention provides an agrochemical composition comprising a compound of formula (I), and more particularly an agrochemical composition comprising a fungicidally effective amount of a compound of formula (I). Said composition can further comprise at least one compound selected among an additional active ingredient, an appropriate formulation inert, a carrier, an adjuvant, and any mixtures thereof.

Compounds of formula (I) may be used to control phytopathogenic microorganisms. Thus, in order to control a phytopathogen a compound of formula (I), or a composition comprising a compound of formula (I) according to the invention, may be applied directly to the phytopathogen, or to the locus of a phytopathogen, in particular to a plant susceptible to attack by phytopathogens, or to a propagation material of a plant.

Thus, in a third aspect the present invention provides the use of a compound of formula (I), or a composition comprising a compound of formula (I), as described herein to combat, prevent or control a phytopathogen.

In a fourth aspect the present invention provides a method of combating, preventing or controlling phytopathogens, comprising applying a compound of formula (I), or a composition comprising a compound of formula (I), as described herein to said phytopathogen, or to the locus of said phytopathogen, in particular to a plant susceptible to attack by a phytopathogen, or to a propagation material of a plant.

Compounds of formula (I) are particularly effective in combating, preventing or controlling phytopathogenic fungi, in particular oomycetes. Thus, in a fifth aspect the present invention provides the use of a compound of formula (I), or a composition comprising a compound of formula (I), as described herein to control phytopathogenic fungi, in particular oomycetes.

In a sixth aspect the present invention provides a method of combating, preventing or controlling phytopathogenic fungi, comprising applying a compound of formula (I), or a composition comprising a compound of formula (I), as described herein to said phytopathogenic fungi, or to the locus of said phytopathogenic fungi, in particular to a plant susceptible to attack by phytopathogenic fungi, in particular oomycetes, or to a propagation material of a plant.

In a seventh aspect the present invention provides compounds of formula (XV), formula (XVI), formula (XXI), formula (XXII), formula (II), and formula (XVII), as respectively described in the invention.

Where a group is indicated as being substituted, e.g. alkyl, this includes those groups that are part of other groups, e.g. the alkyl in alkylthio.

### Definitions

The term “halogen” refers to fluorine (fluoro or F), chlorine (chloro or Cl), bromine (bromo or Br) or iodine (iodo or I), preferably fluorine, chlorine or bromine.
The term “Alkyl” as used herein—in isolation or as part of a chemical group—represents straight-chain or branched hydrocarbons, preferably with 1 to 6 carbon atoms, for example methyl, ethyl, n-propyl, isopropyl, n-butyl, isobutyl, s-butyl, t-butyl, pentyl, 1-methylbutyl, 2-methylbutyl, 3-methylbutyl, 1,2-dimethylpropyl, 1,1-dimethylpropyl, 2,2-dimethylpropyl, 1-ethylpropyl, hexyl, 1-methylpentyl, 2-methylpentyl, 3-methylpentyl, 4-methylpentyl, 1,2-dimethylpropyl, 1,3-dimethylbutyl, 1,4-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl and 2-ethylbutyl. Alkyl groups with 1 to 4 carbon atoms are preferred, for example methyl, ethyl, n-propyl, isopropyl, n-butyl, isobutyl, s-butyl or t-butyl.
The term “Alkenyl”—in isolation or as part of a chemical group—represents straight-chain or branched hydrocarbons, preferably with 2 to 6 carbon atoms and at least one double bond, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1, 1-dimethyl-2-butenyl, 1,1-dimethyl-3-butenyl, 1,2-dimethyl-2-butenyl, 1,2-dimethyl-3-butenyl, 1,3-dimethyl-2-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1, 1,2-trimethyl-2-propenyl, 1-ethyl-1-methyl-2-propenyl und 1-ethyl-2-methyl-2-propenyl. Alkenyl groups with 2 to 4 carbon atoms are preferred, for example 2-propenyl, 2-butenyl or 1-methyl-2-propenyl.
The term “Alkynyl”—in isolation or as part of a chemical group—represents straight-chain or branched hydrocarbons, preferably with 2 to 6 carbon atoms and at least one triple bond, for example 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-3-butynyl, 1,2-dimethyl-3-butynyl, 2,2-dimethyl-3-butynyl, 1-ethyl-3-butynyl, 2-ethyl-3-butynyl, 1-ethyl-1-methyl-2-propynyl and 2,5-hexadiynyl. Alkynyls with 2 to 4 carbon atoms are preferred, for example ethynyl, 2-propynyl or 2-butynyl-2-propenyl.
The term “cycloalkyl”—in isolation or as part of a chemical group—represents saturated or partially unsaturated mono-, bi- or tricyclic hydrocarbons, preferably with 3 to 10 carbon atoms, for example cyclopropyl, cyclobutyl, cyclopentyl, cyclohexyl, cycloheptyl, cyclooctyl, bicyclo[2.2.1]heptyl, bicyclo[2.2.2]octyl or adamantyl. Cycloalkyls with 3, 4, 5, 6 or 7 carbon atoms are preferred, for example cyclopropyl or cyclobutyl.
Hydroxyl or hydroxy stands for a —OH group.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

As embodiment 2, there is provided a compound according to embodiment 1, wherein

### A is N; and

R⁶ is selected from C₁₋₄alkyl and C₁₋₄alkoxy, wherein each of said groups is optionally substituted with one to three substituents independently selected from halogen.

As embodiment 3, there is provided a compound according to embodiment 1 or 2, wherein

### A is N; and

R⁶ is selected from methyl, ethyl and methoxy.

As embodiment 4, there is provided a compound according to any one of embodiments 1 to 3, wherein

### A is N; and

R⁶ is methoxy.

As embodiment 5, there is provided a compound according to embodiment 1, wherein

### A is CH; and

R⁶ is selected from C₁₋₄alkyl and C₁₋₄alkoxy, wherein each of said groups is optionally substituted with one to three substituents independently selected from halogen.

As embodiment 6, there is provided a compound according to embodiment 1 or 5, wherein

### A is CH; and

R⁶ is selected from methyl, ethyl and methoxy.

As embodiment 7, there is provided a compound according to embodiment 1, 5 or 6, wherein

### A is CH; and

R⁶ is methoxy.

As embodiment 8, there is provided a compound according to any one of embodiments 1 to 7, wherein R¹ is selected from C₁₋₄alkyl, C₁₋₄alkoxy-C₁₋₄alkyl, C₃₋₆cycloalkyl, C₃₋₆cycloalkyl-C₁₋₃alkyl and C₂₋₄alkynyl, wherein each of said groups is optionally substituted with one to three substituents independently selected from halogen and CN.

As embodiment 9, there is provided a compound according to any one of embodiments 1 to 8, wherein R¹ is selected from methyl, ethyl, propyl, —CH₂CH₂—O—CH₃, cyclopropyl, —CH₂-cyclopropyl, —CH₂—CF₃, —CH₂—C≡CH, CH₂CH₂CN, and —CH₂—CN.

As embodiment 10, there is provided a compound according to any one of embodiments 1 to 9, wherein J is CR⁴ and G is CR⁵; and

R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 11, there is provided a compound according to any one of embodiments 1 to 10, wherein J is CR⁴ and G is CR⁵; and

R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl C₃₋₆cycloalkyl and C₃₋₆cycloalkyl-C₁₋₃alkyl groups is optionally substituted with one to three substituents independently selected from halogen.

As embodiment 12, there is provided a compound according to any one of embodiments 1 to 10, wherein J is CR⁴ and G is CR⁵;

R³ and R⁵ are H; and
R⁴ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 13, there is provided a compound according to embodiment 12,

wherein J is CR⁴ and G is CR⁵;
R³ and R⁵ are H; and
R⁴ is selected from C₁₋₄alkyl, C₁₋₄alkoxy, halogen, CN, and hydroxy, wherein each of the C₁₋₄alkyl and C₁₋₄alkoxy, groups is optionally substituted with one to three substituents independently selected from halogen.

As embodiment 14, there is provided a compound according to to embodiment 12,

wherein J is CR⁴ and G is CR⁵;
R³ and R⁵ are H; and
R⁴ is selected from methyl, methoxy, chloro, fluoro, bromo, CN, and hydroxy.

As embodiment 15, there is provided a compound according to any one of embodiments 1 to 10, wherein

J is CR⁴ and G is CR⁵;

### R³ is H; and

R⁴ and R⁵ are independently selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 16, there is provided a compound according to embodiment 15,

wherein J is CR⁴ and G is CR⁵;

### R³ is H; and

R⁴ and R⁵ are independently selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl and C₃₋₆cycloalkyl-C₁₋₃alkyl groups is optionally substituted with one to three substituents independently selected from halogen.

As embodiment 17, there is provided a compound according to embodiment 15, wherein J is CR⁴ and G is CR⁵;

### R³ is H;

R⁴ is selected from C₁₋₄alkyl, C₁₋₄alkoxy, halogen, CN, and hydroxy, wherein each of the the C₁₋₄alkyl and C₁₋₄alkoxy groups is optionally substituted with one to three substituents independently selected from halogen; and
R⁵ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl, and C₃₋₆cycloalkyl-C₁₋₃alkyl groups is optionally substituted with one to three substituents independently selected from halogen.

As embodiment 18, there is provided a compound according to embodiment 15, wherein J is CR⁴ and G is CR⁵;

### R³ is H;

R⁴ is selected from methyl, methoxy, chloro, fluoro, bromo, CN, and hydroxy; and
R⁵ is selected from methyl, —O—CH₂—CH₂—O—CH₃, —CH₂—CH₂O—CH₃, —O—CH₃, —CH₂—CF₂H, fluoro, chloro, bromo, iodo, cyclopropyl, —C≡CH, CN, —CF₃, isopropyl and —O—CF₂H.

As embodiment 19, there is provided a compound according to any one of embodiments 1 to 10, wherein J is CR⁴ and G is CR⁵;

R³ and R⁴ are H; and
R⁵ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 20, there is provided a compound according to embodiment 19,

wherein J is CR⁴ and G is CR⁵;
R³ and R⁴ are H; and
R⁵ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl and C₃₋₆cycloalkyl-C₁₋₃alkyl groups is optionally substituted with one to three substituents independently selected from halogen.

As embodiment 21, there is provided a compound according to embodiment 19,

wherein J is CR⁴ and G is CR⁵,
R³ and R⁴ are H; and
R⁵ is selected from methyl, —O—CH₂—CH₂—O—CH₃, —CH₂—CH₂O—CH₃, —O—CH₃, —CH₂—CF₂H, fluoro, chloro, bromo, iodo, cyclopropyl, —C≡CH, CN, —CF₃, isopropyl and —O—CF₂H.

As embodiment 22, there is provided a compound according to any one of embodiments 1 to 10, wherein J is CR⁴ and G is CR⁵;

R⁴ is selected from C₁₋₄alkyl, C₁₋₄alkoxy, halogen, CN, and hydroxy, wherein the alkyl groups are unsubstituted or substituted with one to three substituents independently selected from halogen; and
R³ and R⁵ are independently selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl and C₃₋₆cycloalkyl-C₁₋₃alkyl groups is optionally substituted with one to three substituents independently selected from halogen.

As embodiment 23, there is provided a compound according to embodiment 22, wherein J is CR⁴ and G is CR⁵;

R⁴ is selected from methyl, methoxy, chloro, fluoro, bromo, CN and hydroxy; and
R³ and R⁵ are independently selected from methyl, —O—CH₂—CH₂—O—CH₃, —CH₂—CH₂O—CH₃, —O—CH₃, —CH₂—CF₂H, fluoro, chloro, bromo, iodo, cyclopropyl, —C≡CH, CN, —CF₃, isopropyl and —O—CF₂H.

As embodiment 24, there is provided a compound according to any one of embodiments 1 to 10,

wherein J is CR⁴ and G is CR⁵; and
R³, R⁴ and R⁵ are H.

As embodiment 25, there is provided a compound according to any one of embodiments 1 to 10,

wherein J is CR⁴ and G is CR⁵;
R³ and R⁵ are H; and
R⁴ is chloro or fluoro.

As embodiment 26, there is provided a compound according to any one of embodiments 1 to 10,

wherein J is CR⁴ and G is CR⁵;

### R³ is H;

R⁴ is fluoro; and
R⁵ is methyl.

As embodiment 27, there is provided a compound according to any one of embodiments 1 to 10,

wherein J is CR⁴ and G is CR⁵;

### R³ is H;

R⁴ is fluoro; and
R⁵ is —O—CH₂—CH₂—O—CH₃.

As embodiment 28, there is provided a compound according to any one of embodiments 1 to 10,

wherein J is CR⁴ and G is CR⁵;

### R³ is H;

R⁴ is chloro, fluoro, bromo or iodo; and
R⁵ is —O—CH₃.

As embodiment 29, there is provided a compound according to any one of embodiments 1 to 10,

wherein J is CR⁴ and G is CR⁵;

### R³ is H;

### R⁴ is H; and

R⁵ is —CH₂—CF₂H.

As embodiment 30, there is provided a compound according to any one of embodiments 1 to 10,

wherein J is CR⁴ and G is CR⁵;

### R³ is H;

### R⁴ is H; and

R⁵ is chloro, fluoro, bromo or iodo.

As embodiment 31, there is provided a compound according to any one of embodiments 1 to 9,

wherein J is CR⁴ and G is N; and
R³ and R⁴ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 32, there is provided a compound according to embodiment 31,

wherein J is CR⁴ and G is N;

### R³ is H; and

R⁴ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 33, there is provided a compound according to embodiment 31,

wherein J is CR⁴ and G is N; and
R³ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen;

### R⁴ is H.

As embodiment 34, there is provided a compound according to embodiments 31,

wherein J is CR⁴ and G is N; and
R³ and R⁴ are independently selected from H, C₁₋₄alkyl, C₁₋₄alkoxy, halogen, CN and C₂₋₄alkynyl, wherein each of the C₁₋₄alkyl, C₁₋₄alkoxy and C₂₋₄alkynyl groups is optionally substituted with one to three substituents independently selected from halogen.

As embodiment 35, there is provided a compound according to embodiments 34,

wherein J is CR⁴ and G is N; and
R³ and R⁴ are independently selected from H, methyl, —OCH₃, —CF₃, —OCF₂H, halogen, —C≡CH and CN.

As embodiment 36, there is provided a compound according to embodiments 34,

wherein J is CR⁴ and G is N; and
R³ and R⁴ are H.

As embodiment 37, there is provided a compound according to any one of embodiments 1 to 9,

wherein J is N and G is CR⁵; and
R³ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 38, there is provided a compound according to embodiment 37,

wherein J is N and G is CR⁵;

### R³ is H; and

R⁵ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

As embodiment 39, there is provided a compound according to embodiment 37,

wherein J is N and G is CR⁵;
R³ is selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally with one to three substituents independently selected from halogen; and

### R⁵ is H.

As embodiment 40, there is provided a compound according to embodiment 37,

wherein J is N and G is CR⁵; and
R³ and R⁵ are independently selected from H, C₁₋₄alkyl, C₁₋₄alkoxy, halogen, CN and C₂₋₄alkynyl, wherein each of the C₁₋₄alkyl, C₁₋₄alkoxy and C₂₋₄alkynyl groups is optionally substituted with one to three substituents independently selected from halogen.

As embodiment 41, there is provided a compound according to embodiment 37,

wherein J is N and G is CR⁵; and
R³ and R⁵ are independently selected from H, methyl, —OCH₃, —CF₃, —OCF₂H, halogen, —C≡CH and CN.

As embodiment 42, there is provided a compound according to embodiment 37,

wherein J is N and G is CR⁵; and
R³ and R⁵ are H.

As a preferred embodiment 10, there is provided a compound according to any one of embodiments 1 to 9, wherein

### A is N;

### R³ is H;

J is CR⁴ and G is CR⁵;
R⁴ is selected from C₁₋₄alkyl, C₁₋₄alkoxy, halogen, CN, and hydroxy, wherein each of the the C₁₋₄alkyl and C₁₋₄alkoxy groups is optionally substituted with one to three substituents independently selected from halogen;
R⁵ is selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl, and C₃₋₆cycloalkyl-C₁₋₃alkyl groups is optionally substituted with one to three substituents independently selected from halogen; and R⁶ is selected from C₁₋₄alkyl and C₁₋₄alkoxy, wherein each of said groups is optionally substituted with one to three substituents independently selected from halogen.

As a more preferred embodiment 10 wherein Z is O, there is provided a compound according to any one of embodiments 1 to 9, wherein

### A is N;

### R³ is H;

J is CR⁴ and G is CR⁵;
R⁴ is halogen or CN;
R⁵ is C₁₋₄alkoxy, wherein each of the C₁₋₄alkoxy groups is optionally substituted with one to three substituents independently selected from halogen; and
R⁶ is selected from C₁₋₄alkyl and C₁₋₄alkoxy, wherein each of said groups is optionally substituted with one to three substituents independently selected from halogen.

As a more preferred embodiment 10 wherein Z is S, there is provided a compound according to any one of embodiments 1 to 9, wherein

### A is N;

R³ and R⁵ are H;
J is CR⁴ and G is CR⁵;
R⁴ is halogen or CN; and
R⁶ is selected from C₁₋₄alkyl and C₁₋₄alkoxy, wherein each of said groups is optionally substituted with one to three substituents independently selected from halogen.

In a preferred embodiment according to any one of the embodiments 2 to 42, Z is O.

As embodiment 43, there is provided a compound according to any one of embodiments 1 to 42, wherein the compound is selected from:
3-(4-acetamidophenyl)-N-(4-chlorophenyl)-N-methyl-imidazo[1,2-a]pyridine-6-carboxamide,
methyl N-[4-[6-[(4-chlorophenyl)-methyl-carbamoyl]imidazo[1,2-a]pyridin-3-yl]phenyl]carbamate,
3-(6-acetamido-3-pyridyl)-N-(4-chlorophenyl)-N-methyl-imidazo[1,2-a]pyridine-6-carboxamide,
methyl N-[5-[6-[(4-chlorophenyl)-methyl-carbamoyl]imidazo[1,2-a]pyridin-3-yl]-2-pyridyl]carbamate,
methyl N-[4-[6-[(4-hydroxyphenyl)-methyl-carbamoyl]imidazo[1,2-a]pyridin-3-yl]phenyl]carbamate,
methyl N-[4-[6-[(4-cyanophenyl)-methyl-carbamoyl]imidazo[1,2-a]pyridin-3-yl]phenyl]carbamate,
methyl N-[4-[6-[(3-methoxyphenyl)-methyl-carbamoyl]imidazo[1,2-a]pyridin-3-yl]phenyl]carbamate,
methyl N-[4-[6-[(4-methoxyphenyl)-methyl-carbamoyl]imidazo[1,2-a]pyridin-3-yl]phenyl]carbamate,
methyl N-[4-[6-[[3-(difluoromethoxy)phenyl]-methyl-carbamoyl]imidazo[1,2-a]pyridin-3-yl]phenyl]carbamate,
methyl N-[4-[6-[(3-ethylphenyl)-methyl-carbamoyl]imidazo[1,2-a]pyridin-3-yl]phenyl]carbamate,
methyl N-[4-[6-[(4-fluorophenyl)-methyl-carbamoyl]imidazo[1,2-a]pyridin-3-yl]phenyl]carbamate,
methyl N-[4-[6-[methyl-(2-methyl-4-pyridyl)carbamoyl]imidazo[1,2-a]pyridin-3-yl]phenyl]carbamate,
methyl N-[4-[6-[(3,4-dimethylphenyl)-methyl-carbamoyl]imidazo[1,2-a]pyridin-3-yl]phenyl]carbamate,
methyl N-[4-[6-[(4-isopropylphenyl)-methyl-carbamoyl]imidazo[1,2-a]pyridin-3-yl]phenyl]carbamate,
methyl N-[5-[6-[(4-cyanophenyl)-methyl-carbamoyl]imidazo[1,2-a]pyridin-3-yl]-2-pyridyl]carbamate,
methyl N-[4-[6-[methyl(p-tolyl)carbamoyl]imidazo[1,2-a]pyridin-3-yl]phenyl]carbamate,
methyl N-[4-[6-[(4-chloro-3-fluoro-phenyl)-methyl-carbamoyl]imidazo[1,2-a]pyridin-3-yl]phenyl]carbamate,
methyl N-[4-[6-[(4-chlorophenyl)-methyl-carbamoyl]-7-methyl-imidazo[1,2-a]pyridin-3-yl]phenyl]carbamate,
methyl N-[4-[6-[(4-chlorophenyl)-methyl-carbamoyl]-8-methyl-imidazo[1,2-a]pyridin-3-yl]phenyl]carbamate,
methyl N-[4-[6-[(4-chlorophenyl)-methyl-carbamoyl]-5-methyl-imidazo[1,2-a]pyridin-3-yl]phenyl]carbamate,
methyl N-[4-[5-chloro-6-[(4-chlorophenyl)-methyl-carbamoyl]imidazo[1,2-a]pyridin-3-yl]phenyl]carbamate,
methyl N-[4-[8-chloro-6-[(4-chlorophenyl)-methyl-carbamoyl]imidazo[1,2-a]pyridin-3-yl]phenyl]carbamate,
methyl N-[4-[7-chloro-6-[(4-chlorophenyl)-methyl-carbamoyl]imidazo[1,2-a]pyridin-3-yl]phenyl]carbamate,
methyl N-[4-[6-[methyl(4-pyridyl)carbamoyl]imidazo[1,2-a]pyridin-3-yl]phenyl]carbamate,
methyl N-[5-[6-[(4-fluorophenyl)-methyl-carbamoyl]imidazo[1,2-a]pyridin-3-yl]-2-pyridyl]carbamate,
methyl N-[4-[6-[(2-methoxy-4-pyridyl)-methyl-carbamoyl]imidazo[1,2-a]pyridin-3-yl]phenyl]carbamate,
methyl N-[4-[6-[(4-chlorophenyl)-cyclopropyl-carbamoyl]imidazo[1,2-a]pyridin-3-yl]phenyl]carbamate,
methyl N-[4-[6-[(4-chlorophenyl)-(cyclopropylmethyl)carbamoyl]imidazo[1,2-a]pyridin-3-yl]phenyl]carbamate,
methyl N-[4-[6-[(4-chlorophenyl)-(2,2,2-trifluoroethyl)carbamoyl]imidazo[1,2-a]pyridin-3-yl]phenyl]carbamate,
methyl N-[4-[6-[(4-chlorophenyl)-propyl-carbamoyl]imidazo[1,2-a]pyridin-3-yl]phenyl]carbamate,
methyl N-[4-[6-[(4-chlorophenyl)-(cyanomethyl)carbamoyl]imidazo[1,2-a]pyridin-3-yl]phenyl]carbamate,
methyl N-[4-[6-[(4-chlorophenyl)-ethyl-carbamoyl]imidazo[1,2-a]pyridin-3-yl]phenyl]carbamate,
methyl N-[4-[6-[(4-chlorophenyl)-prop-2-ynyl-carbamoyl]imidazo[1,2-a]pyridin-3-yl]phenyl]carbamate,
methyl N-[4-[7-bromo-6-[(4-chlorophenyl)-methyl-carbamoyl]imidazo[1,2-a]pyridin-3-yl]phenyl]carbamate,
methyl N-[4-[8-bromo-6-[(4-chlorophenyl)-methyl-carbamoyl]imidazo[1,2-a]pyridin-3-yl]phenyl]carbamate,
methyl N-[4-[6-[(4-chlorophenyl)-methyl-carbamoyl]-8-fluoro-imidazo[1,2-a]pyridin-3-yl]phenyl]carbamate,
methyl N-[4-[6-[(4-chlorophenyl)-methyl-carbamoyl]-8-methoxy-imidazo[1,2-a]pyridin-3-yl]phenyl]carbamate,
methyl N-[4-[6-[(4-chlorophenyl)-methyl-carbamoyl]-5-cyano-imidazo[1,2-a]pyridin-3-yl]phenyl]carbamate,
methyl N-[4-[6-[(4-chlorophenyl)-methyl-carbamoyl]-7-methoxy-imidazo[1,2-a]pyridin-3-yl]phenyl]carbamate,
methyl N-[4-[6-[(4-chlorophenyl)-methyl-carbamoyl]-8-cyano-imidazo[1,2-a]pyridin-3-yl]phenyl]carbamate,
methyl N-[4-[6-[(4-chlorophenyl)-methyl-carbamoyl]-7-cyano-imidazo[1,2-a]pyridin-3-yl]phenyl]carbamate,
methyl N-[4-[6-[(4-chlorophenyl)-methyl-carbamoyl]-7-fluoro-imidazo[1,2-a]pyridin-3-yl]phenyl]carbamate,
methyl N-[5-[6-[(3-ethynylphenyl)-methyl-carbamoyl]imidazo[1,2-a]pyridin-3-yl]-2-pyridyl]carbamate,
methyl N-[5-[6-[(3-cyanophenyl)-methyl-carbamoyl]imidazo[1,2-a]pyridin-3-yl]-2-pyridyl]carbamate,
methyl N-[5-[6-[(4-fluoro-3-methoxy-phenyl)-methyl-carbamoyl]imidazo[1,2-a]pyridin-3-yl]-2-pyridyl]carbamate,
methyl N-[5-[6-[(4-chloro-3-methyl-phenyl)-methyl-carbamoyl]imidazo[1,2-a]pyridin-3-yl]-2-pyridyl]carbamate,
methyl N-[5-[6-[(3,5-dimethylphenyl)-methyl-carbamoyl]imidazo[1,2-a]pyridin-3-yl]-2-pyridyl]carbamate,
methyl N-[5-[6-[(4-fluoro-3-methyl-phenyl)-methyl-carbamoyl]imidazo[1,2-a]pyridin-3-yl]-2-pyridyl]carbamate,
methyl N-[5-[6-[methyl-[3-(trifluoromethyl)phenyl]carbamoyl]imidazo[1,2-a]pyridin-3-yl]-2-pyridyl]carbamate,
methyl N-[5-[6-[(3,4-difluorophenyl)-methyl-carbamoyl]imidazo[1,2-a]pyridin-3-yl]-2-pyridyl]carbamate,
methyl N-[5-[6-[(4-cyano-3-methoxy-phenyl)-methyl-carbamoyl]imidazo[1,2-a]pyridin-3-yl]-2-pyridyl]carbamate,
methyl N-[5-[6-[(3-fluorophenyl)-methyl-carbamoyl]imidazo[1,2-a]pyridin-3-yl]-2-pyridyl]carbamate,
methyl N-[5-[6-[(4-cyano-3-methyl-phenyl)-methyl-carbamoyl]imidazo[1,2-a]pyridin-3-yl]-2-pyridyl]carbamate,
methyl N-[5-[6-[methyl(m-tolyl)carbamoyl]imidazo[1,2-a]pyridin-3-yl]-2-pyridyl]carbamate,
methyl N-[5-[6-[(5-methoxy-3-pyridyl)-methyl-carbamoyl]imidazo[1,2-a]pyridin-3-yl]-2-pyridyl]carbamate,
methyl N-[5-[6-[methyl-(5-methyl-3-pyridyl)carbamoyl]imidazo[1,2-a]pyridin-3-yl]-2-pyridyl]carbamate,
methyl N-[5-[6-[[3-(difluoromethyl)phenyl]-methyl-carbamoyl]imidazo[1,2-a]pyridin-3-yl]-2-pyridyl]carbamate,
methyl N-[5-[6-[(3,5-dimethoxyphenyl)-methyl-carbamoyl]imidazo[1,2-a]pyridin-3-yl]-2-pyridyl]carbamate,
methyl N-[5-[6-[(3-methoxy-5-methyl-phenyl)-methyl-carbamoyl]imidazo[1,2-a]pyridin-3-yl]-2-pyridyl]carbamate,
methyl N-[5-[6-[(6-fluoro-3-pyridyl)-methyl-carbamoyl]imidazo[1,2-a]pyridin-3-yl]-2-pyridyl]carbamate,
methyl N-[5-[6-[methyl(phenyl)carbamoyl]imidazo[1,2-a]pyridin-3-yl]-2-pyridyl]carbamate,
methyl N-[5-[6-[(3-chlorophenyl)-methyl-carbamoyl]imidazo[1,2-a]pyridin-3-yl]-2-pyridyl]carbamate,
methyl N-[5-[6-[(3-methoxyphenyl)-methyl-carbamoyl]imidazo[1,2-a]pyridin-3-yl]-2-pyridyl]carbamate,
methyl N-[5-[6-[methyl(p-tolyl)carbamoyl]imidazo[1,2-a]pyridin-3-yl]-2-pyridyl]carbamate,
methyl N-[5-[6-[(3,4-dimethylphenyl)-methyl-carbamoyl]imidazo[1,2-a]pyridin-3-yl]-2-pyridyl]carbamate,
methyl N-[5-[6-[[4-(difluoromethoxy)phenyl]-methyl-carbamoyl]imidazo[1,2-a]pyridin-3-yl]-2-pyridyl]carbamate,
methyl N-[5-[6-[(6-methoxy-3-pyridyl)-methyl-carbamoyl]imidazo[1,2-a]pyridin-3-yl]-2-pyridyl]carbamate,
methyl N-[5-[6-[(4-bromophenyl)-methyl-carbamoyl]imidazo[1,2-a]pyridin-3-yl]-2-pyridyl]carbamate,
methyl N-[5-[6-[(3-bromophenyl)-methyl-carbamoyl]imidazo[1,2-a]pyridin-3-yl]-2-pyridyl]carbamate,
methyl N-[5-[6-[(2-methoxy-4-pyridyl)-methyl-carbamoyl]imidazo[1,2-a]pyridin-3-yl]-2-pyridyl]carbamate,
methyl N-[5-[6-[(2-fluoro-4-pyridyl)-methyl-carbamoyl]imidazo[1,2-a]pyridin-3-yl]-2-pyridyl]carbamate,
methyl N-[5-[6-[(3-cyclopropylphenyl)-methyl-carbamoyl]imidazo[1,2-a]pyridin-3-yl]-2-pyridyl]carbamate,
methyl N-[5-[6-[(3-iodophenyl)-methyl-carbamoyl]imidazo[1,2-a]pyridin-3-yl]-2-pyridyl]carbamate,
methyl N-[5-[6-[[3-(2,2-difluoroethyl)phenyl]-methyl-carbamoyl]imidazo[1,2-a]pyridin-3-yl]-2-pyridyl]carbamate,
methyl N-[5-[6-[(4-chloro-3-methoxy-phenyl)-methyl-carbamoyl]imidazo[1,2-a]pyridin-3-yl]-2-pyridyl]carbamate,
methyl N-[5-[6-[(4-fluorophenyl)-(2-methoxyethyl)carbamoyl]imidazo[1,2-a]pyridin-3-yl]-2-pyridyl]carbamate,
methyl N-[5-[6-[[4-fluoro-3-(2-methoxyethoxy)phenyl]-methyl-carbamoyl]imidazo[1,2-a]pyridin-3-yl]-2-pyridyl]carbamate,
   N-(4-fluoro-3-methyl-phenyl)-N-methyl-3-[6-(propanoylamino)-3-pyridyl]imidazo[1,2-a]pyridine-6-carboxamide, and
methyl N-[5-[6-[(4-fluorophenyl)-methyl-carbamothioyl]imidazo[1,2-a]pyridin-3-yl]-2-pyridyl]carbamate.

The compounds in Tables 1.1 to 1.36 below illustrate the compounds of the invention.

Table 1.1 provides compounds of formula (1)

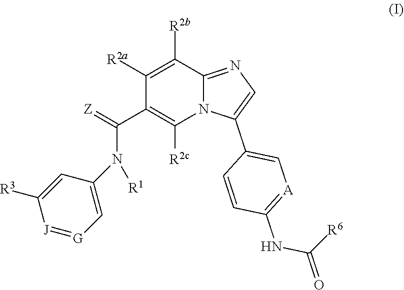

wherein Z is O;
R^{2a} is H, R^{2b} is H, R^{2c} is H and R⁶ is methyl; and
the values of R¹, R³, J, G and Aare as defined in Table Z.1 below.

Each of Tables 1.2 to 1.36 (which follow Table 1.1) discloses individual compounds of the formula (1) in which R^{2a}, R^{2b}, R^{2c} and R⁶ are specifically defined in Tables 1.2 to 1.36, which refer to Table Z.1 wherein R¹, R³, J, G and A are specifically defined.

TABLE Z.1

| Compound | R¹ | R³ | J | G | A |
|---|---|---|---|---|---|
| E1.1 | CH₃ | H | CH | CH | CH |
| E1.2 | CH₃ | CH₃ | CH | CH | CH |
| E1.3 | CH₃ | CH₂CH₃ | CH | CH | CH |
| E1.4 | CH₃ | F | CH | CH | CH |
| E1.5 | CH₃ | Cl | CH | CH | CH |
| E1.6 | CH₃ | Br | CH | CH | CH |
| E1.7 | CH₃ | CN | CH | CH | CH |
| E1.8 | CH₃ | OCH₃ | CH | CH | CH |
| E1.9 | CH₃ | OCH₂CH₃ | CH | CH | CH |
| E1.10 | CH₃ | OCH₂CH₂OCH₃ | CH | CH | CH |
| E1.11 | CH₃ | OH | CH | CH | CH |
| E1.12 | CH₃ | H | N | CH | CH |
| E1.13 | CH₃ | CH₃ | N | CH | CH |
| E1.14 | CH₃ | CH₂CH₃ | N | CH | CH |
| E1.15 | CH₃ | F | N | CH | CH |
| E1.16 | CH₃ | Cl | N | CH | CH |
| E1.17 | CH₃ | Br | N | CH | CH |
| E1.18 | CH₃ | CN | N | CH | CH |
| E1.19 | CH₃ | OCH₃ | N | CH | CH |
| E1.20 | CH₃ | OCH₂CH₃ | N | CH | CH |
| E1.21 | CH₃ | OCH₂CH₂OCH₃ | N | CH | CH |
| E1.22 | CH₃ | OH | N | CH | CH |
| E1.23 | CH₃ | H | CH | N | CH |
| E1.24 | CH₃ | CH₃ | CH | N | CH |
| E1.25 | CH₃ | CH₂CH₃ | CH | N | CH |
| E1.26 | CH₃ | F | CH | N | CH |
| E1.27 | CH₃ | Cl | CH | N | CH |
| E1.28 | CH₃ | Br | CH | N | CH |
| E1.29 | CH₃ | CN | CH | N | CH |
| E1.30 | CH₃ | OCH₃ | CH | N | CH |
| E1.31 | CH₃ | OCH₂CH₃ | CH | N | CH |
| E1.32 | CH₃ | OCH₂CH₂OCH₃ | CH | N | CH |
| E1.33 | CH₃ | OH | CH | N | CH |
| E1.34 | CH₃ | H | C—F | CH | CH |
| E1.35 | CH₃ | CH₃ | C—F | CH | CH |
| E1.36 | CH₃ | CH₂CH₃ | C—F | CH | CH |
| E1.37 | CH₃ | F | C—F | CH | CH |
| E1.38 | CH₃ | Cl | C—F | CH | CH |
| E1.39 | CH₃ | Br | C—F | CH | CH |
| E1.40 | CH₃ | CN | C—F | CH | CH |
| E1.41 | CH₃ | OCH₃ | C—F | CH | CH |
| E1.42 | CH₃ | OCH₂CH₃ | C—F | CH | CH |
| E1.43 | CH₃ | OCH₂CH₂OCH₃ | C—F | CH | CH |
| E1.44 | CH₃ | OH | C—F | CH | CH |
| E1.45 | CH₃ | H | C—Cl | CH | CH |
| E1.46 | CH₃ | CH₃ | C—Cl | CH | CH |
| E1.47 | CH₃ | CH₂CH₃ | C—Cl | CH | CH |
| E1.48 | CH₃ | F | C—Cl | CH | CH |
| E1.49 | CH₃ | Cl | C—Cl | CH | CH |
| E1.50 | CH₃ | Br | C—Cl | CH | CH |
| E1.51 | CH₃ | CN | C—Cl | CH | CH |
| E1.52 | CH₃ | OCH₃ | C—Cl | CH | CH |
| E1.53 | CH₃ | OCH₂CH₃ | C—Cl | CH | CH |
| E1.54 | CH₃ | OCH₂CH₂OCH₃ | C—Cl | CH | CH |
| E1.55 | CH₃ | OH | C—Cl | CH | CH |
| E1.56 | CH₃ | H | C—CH₃ | CH | CH |
| E1.57 | CH₃ | CH₃ | C—CH₃ | CH | CH |
| E1.58 | CH₃ | CH₂CH₃ | C—CH₃ | CH | CH |
| E1.59 | CH₃ | F | C—CH₃ | CH | CH |
| E1.60 | CH₃ | Cl | C—CH₃ | CH | CH |
| E1.61 | CH₃ | Br | C—CH₃ | CH | CH |
| E1.62 | CH₃ | CN | C—CH₃ | CH | CH |
| E1.63 | CH₃ | OCH₃ | C—CH₃ | CH | CH |
| E1.64 | CH₃ | OCH₂CH₃ | C—CH₃ | CH | CH |
| E1.65 | CH₃ | OCH₂CH₂OCH₃ | C—CH₃ | CH | CH |
| E1.66 | CH₃ | OH | C—CH₃ | CH | CH |
| E1.67 | CH₃ | H | C—CH₂CH₃ | CH | CH |
| E1.68 | CH₃ | CH₃ | C—CH₂CH₃ | CH | CH |
| E1.69 | CH₃ | CH₂CH₃ | C—CH₂CH₃ | CH | CH |
| E1.70 | CH₃ | F | C—CH₂CH₃ | CH | CH |
| E1.71 | CH₃ | Cl | C—CH₂CH₃ | CH | CH |
| E1.72 | CH₃ | Br | C—CH₂CH₃ | CH | CH |
| E1.73 | CH₃ | CN | C—CH₂CH₃ | CH | CH |
| E1.74 | CH₃ | OCH₃ | C—CH₂CH₃ | CH | CH |
| E1.75 | CH₃ | OCH₂CH₃ | C—CH₂CH₃ | CH | CH |
| E1.76 | CH₃ | OCH₂CH₂OCH₃ | C—CH₂CH₃ | CH | CH |
| E1.77 | CH₃ | OH | C—CH₂CH₃ | CH | CH |
| E1.78 | CH₃ | H | C—CN | CH | CH |
| E1.79 | CH₃ | CH₃ | C—CN | CH | CH |
| E1.80 | CH₃ | CH₂CH₃ | C—CN | CH | CH |
| E1.81 | CH₃ | F | C—CN | CH | CH |
| E1.82 | CH₃ | Cl | C—CN | CH | CH |
| E1.83 | CH₃ | Br | C—CN | CH | CH |
| E1.84 | CH₃ | CN | C—CN | CH | CH |
| E1.85 | CH₃ | OCH₃ | C—CN | CH | CH |
| E1.86 | CH₃ | OCH₂CH₃ | C—CN | CH | CH |
| E1.87 | CH₃ | OCH₂CH₂OCH₃ | C—CN | CH | CH |
| E1.88 | CH₃ | OH | C—CN | CH | CH |
| E1.89 | CH₃ | H | CH | CH | N |
| E1.90 | CH₃ | CH₃ | CH | CH | N |
| E1.91 | CH₃ | CH₂CH₃ | CH | CH | N |
| E1.92 | CH₃ | F | CH | CH | N |
| E1.93 | CH₃ | Cl | CH | CH | N |
| E1.94 | CH₃ | Br | CH | CH | N |
| E1.95 | CH₃ | CN | CH | CH | N |
| E1.96 | CH₃ | OCH₃ | CH | CH | N |
| E1.97 | CH₃ | OCH₂CH₃ | CH | CH | N |
| E1.98 | CH₃ | OCH₂CH₂OCH₃ | CH | CH | N |
| E1.99 | CH₃ | OH | CH | CH | N |
| E1.100 | CH₃ | H | N | CH | N |
| E1.101 | CH₃ | CH₃ | N | CH | N |
| E1.102 | CH₃ | CH₂CH₃ | N | CH | N |
| E1.103 | CH₃ | F | N | CH | N |
| E1.104 | CH₃ | Cl | N | CH | N |
| E1.105 | CH₃ | Br | N | CH | N |
| E1.106 | CH₃ | CN | N | CH | N |
| E1.107 | CH₃ | OCH₃ | N | CH | N |
| E1.108 | CH₃ | OCH₂CH₃ | N | CH | N |
| E1.109 | CH₃ | OCH₂CH₂OCH₃ | N | CH | N |
| E1.110 | CH₃ | OH | N | CH | N |
| E1.111 | CH₃ | H | CH | N | N |
| E1.112 | CH₃ | CH₃ | CH | N | N |
| E1.113 | CH₃ | CH₂CH₃ | CH | N | N |
| E1.114 | CH₃ | F | CH | N | N |
| E1.115 | CH₃ | Cl | CH | N | N |
| E1.116 | CH₃ | Br | CH | N | N |
| E1.117 | CH₃ | CN | CH | N | N |
| E1.118 | CH₃ | OCH₃ | CH | N | N |
| E1.119 | CH₃ | OCH₂CH₃ | CH | N | N |
| E1.120 | CH₃ | OCH₂CH₂OCH₃ | CH | N | N |
| E1.121 | CH₃ | OH | CH | N | N |
| E1.122 | CH₃ | H | C—F | CH | N |
| E1.123 | CH₃ | CH₃ | C—F | CH | N |
| E1.124 | CH₃ | CH₂CH₃ | C—F | CH | N |
| E1.125 | CH₃ | F | C—F | CH | N |
| E1.126 | CH₃ | Cl | C—F | CH | N |
| E1.127 | CH₃ | Br | C—F | CH | N |
| E1.128 | CH₃ | CN | C—F | CH | N |
| E1.129 | CH₃ | OCH₃ | C—F | CH | N |
| E1.130 | CH₃ | OCH₂CH₃ | C—F | CH | N |
| E1.131 | CH₃ | OCH₂CH₂OCH₃ | C—F | CH | N |
| E1.132 | CH₃ | OH | C—F | CH | N |
| E1.133 | CH₃ | H | C—Cl | CH | N |
| E1.134 | CH₃ | CH₃ | C—Cl | CH | N |
| E1.135 | CH₃ | CH₂CH₃ | C—Cl | CH | N |
| E1.136 | CH₃ | F | C—Cl | CH | N |
| E1.137 | CH₃ | Cl | C—Cl | CH | N |
| E1.138 | CH₃ | Br | C—Cl | CH | N |
| E1.139 | CH₃ | CN | C—Cl | CH | N |
| E1.140 | CH₃ | OCH₃ | C—Cl | CH | N |
| E1.141 | CH₃ | OCH₂CH₃ | C—Cl | CH | N |
| E1.142 | CH₃ | OCH₂CH₂OCH₃ | C—Cl | CH | N |
| E1.143 | CH₃ | OH | C—Cl | CH | N |
| E1.144 | CH₃ | H | C—CH₃ | CH | N |
| E1.145 | CH₃ | CH₃ | C—CH₃ | CH | N |
| E1.146 | CH₃ | CH₂CH₃ | C—CH₃ | CH | N |
| E1.147 | CH₃ | F | C—CH₃ | CH | N |
| E1.148 | CH₃ | Cl | C—CH₃ | CH | N |
| E1.149 | CH₃ | Br | C—CH₃ | CH | N |
| E1.150 | CH₃ | CN | C—CH₃ | CH | N |
| E1.151 | CH₃ | OCH₃ | C—CH₃ | CH | N |
| E1.152 | CH₃ | OCH₂CH₃ | C—CH₃ | CH | N |
| E1.153 | CH₃ | OCH₂CH₂OCH₃ | C—CH₃ | CH | N |
| E1.154 | CH₃ | OH | C—CH₃ | CH | N |
| E1.155 | CH₃ | H | C—CH₂CH₃ | CH | N |
| E1.156 | CH₃ | CH₃ | C—CH₂CH₃ | CH | N |
| E1.157 | CH₃ | CH₂CH₃ | C—CH₂CH₃ | CH | N |
| E1.158 | CH₃ | F | C—CH₂CH₃ | CH | N |
| E1.159 | CH₃ | Cl | C—CH₂CH₃ | CH | N |
| E1.160 | CH₃ | Br | C—CH₂CH₃ | CH | N |
| E1.161 | CH₃ | CN | C—CH₂CH₃ | CH | N |
| E1.162 | CH₃ | OCH₃ | C—CH₂CH₃ | CH | N |
| E1.163 | CH₃ | OCH₂CH₃ | C—CH₂CH₃ | CH | N |
| E1.164 | CH₃ | OCH₂CH₂OCH₃ | C—CH₂CH₃ | CH | N |
| E1.165 | CH₃ | OH | C—CH₂CH₃ | CH | N |
| E1.166 | CH₃ | H | C—CN | CH | N |
| E1.167 | CH₃ | CH₃ | C—CN | CH | N |
| E1.168 | CH₃ | CH₂CH₃ | C—CN | CH | N |
| E1.169 | CH₃ | F | C—CN | CH | N |
| E1.170 | CH | Cl | C—CN | CH | N |
| E1.171 | CH₃ | Br | C—CN | CH | N |
| E1.172 | CH₃ | CN | C—CN | CH | N |
| E1.173 | CH₃ | OCH₃ | C—CN | CH | N |
| E1.174 | CH₃ | OCH₂CH₃ | C—CN | CH | N |
| E1.175 | CH₃ | OCH₂CH₂OCH₃ | C—CN | CH | N |
| E1.176 | CH₃ | OH | C—CN | CH | N |
| E1.177 | CH₂CH₃ | H | CH | CH | CH |
| E1.178 | CH₂CH₃ | CH₃ | CH | CH | CH |
| E1.179 | CH₂CH₃ | CH₂CH₃ | CH | CH | CH |
| E1.180 | CH₂CH₃ | F | CH | CH | CH |
| E1.181 | CH₂CH₃ | Cl | CH | CH | CH |
| E1.182 | CH₂CH₃ | Br | CH | CH | CH |
| E1.183 | CH₂CH₃ | CN | CH | CH | CH |
| E1.184 | CH₂CH₃ | OCH₃ | CH | CH | CH |
| E1.185 | CH₂CH₃ | OCH₂CH₃ | CH | CH | CH |
| E1.186 | CH₂CH₃ | OCH₂CH₂OCH₃ | CH | CH | CH |
| E1.187 | CH₂CH₃ | OH | CH | CH | CH |
| E1.188 | CH₂CH₃ | H | N | CH | CH |
| E1.189 | CH₂CH₃ | CH₃ | N | CH | CH |
| E1.190 | CH₂CH₃ | CH₂CH₃ | N | CH | CH |
| E1.191 | CH₂CH₃ | F | N | CH | CH |
| E1.192 | CH₂CH₃ | Cl | N | CH | CH |
| E1.193 | CH₂CH₃ | Br | N | CH | CH |
| E1.194 | CH₂CH₃ | CN | N | CH | CH |
| E1.195 | CH₂CH₃ | OCH₃ | N | CH | CH |
| E1.196 | CH₂CH₃ | OCH₂CH₃ | N | CH | CH |
| E1.197 | CH₂CH₃ | OCH₂CH₂OCH₃ | N | CH | CH |
| E1.198 | CH₂CH₃ | OH | N | CH | CH |
| E1.199 | CH₂CH₃ | H | CH | N | CH |
| E1.200 | CH₂CH₃ | CH₃ | CH | N | CH |
| E1.201 | CH₂CH₃ | CH₂CH₃ | CH | N | CH |
| E1.202 | CH₂CH₃ | F | CH | N | CH |
| E1.203 | CH₂CH₃ | Cl | CH | N | CH |
| E1.204 | CH₂CH₃ | Br | CH | N | CH |
| E1.205 | CH₂CH₃ | CN | CH | N | CH |
| E1.206 | CH₂CH₃ | OCH₃ | CH | N | CH |
| E1.207 | CH₂CH₃ | OCH₂CH₃ | CH | N | CH |
| E1.208 | CH₂CH₃ | OCH₂CH₂OCH₃ | CH | N | CH |
| E1.209 | CH₂CH₃ | OH | CH | N | CH |
| E1.210 | CH₂CH₃ | H | C—F | CH | CH |
| E1.211 | CH₂CH₃ | CH₃ | C—F | CH | CH |
| E1.212 | CH₂CH₃ | CH₂CH₃ | C—F | CH | CH |
| E1.213 | CH₂CH₃ | F | C—F | CH | CH |
| E1.214 | CH₂CH₃ | Cl | C—F | CH | CH |
| E1.215 | CH₂CH₃ | Br | C—F | CH | CH |
| E1.216 | CH₂CH₃ | CN | C—F | CH | CH |
| E1.217 | CH₂CH₃ | OCH₃ | C—F | CH | CH |
| E1.218 | CH₂CH₃ | OCH₂CH₃ | C—F | CH | CH |
| E1.219 | CH₂CH₃ | OCH₂CH₂OCH₃ | C—F | CH | CH |
| E1.220 | CH₂CH₃ | OH | C—F | CH | CH |
| E1.221 | CH₂CH₃ | H | C—Cl | CH | CH |
| E1.222 | CH₂CH₃ | CH₃ | C—Cl | CH | CH |
| E1.223 | CH₂CH₃ | CH₂CH₃ | C—Cl | CH | CH |
| E1.224 | CH₂CH₃ | F | C—Cl | CH | CH |
| E1.225 | CH₂CH₃ | Cl | C—Cl | CH | CH |
| E1.226 | CH₂CH₃ | Br | C—Cl | CH | CH |
| E1.227 | CH₂CH₃ | CN | C—Cl | CH | CH |
| E1.228 | CH₂CH₃ | OCH₃ | C—Cl | CH | CH |
| E1.229 | CH₂CH₃ | OCH₂CH₃ | C—Cl | CH | CH |
| E1.230 | CH₂CH₃ | OCH₂CH₂OCH₃ | C—Cl | CH | CH |
| E1.231 | CH₂CH₃ | OH | C—Cl | CH | CH |
| E1.232 | CH₂CH₃ | H | C—CH₃ | CH | CH |
| E1.233 | CH₂CH₃ | CH₃ | C—CH₃ | CH | CH |
| E1.234 | CH₂CH₃ | CH₂CH₃ | C—CH₃ | CH | CH |
| E1.235 | CH₂CH₃ | F | C—CH₃ | CH | CH |
| E1.236 | CH₂CH₃ | Cl | C—CH₃ | CH | CH |
| E1.237 | CH₂CH₃ | Br | C—CH₃ | CH | CH |
| E1.238 | CH₂CH₃ | CN | C—CH₃ | CH | CH |
| E1.239 | CH₂CH₃ | OCH₃ | C—CH₃ | CH | CH |
| E1.240 | CH₂CH₃ | OCH₂CH₃ | C—CH₃ | CH | CH |
| E1.241 | CH₂CH₃ | OCH₂CH₂OCH₃ | C—CH₃ | CH | CH |
| E1.242 | CH₂CH₃ | OH | C—CH₃ | CH | CH |
| E1.243 | CH₂CH₃ | H | C—CH₂CH₃ | CH | CH |
| E1.244 | CH₂CH₃ | CH₃ | C—CH₂CH₃ | CH | CH |
| E1.245 | CH₂CH₃ | CH₂CH₃ | C—CH₂CH₃ | CH | CH |
| E1.246 | CH₂CH₃ | F | C—CH₂CH₃ | CH | CH |
| E1.247 | CH₂CH₃ | Cl | C—CH₂CH₃ | CH | CH |
| E1.248 | CH₂CH₃ | Br | C—CH₂CH₃ | CH | CH |
| E1.249 | CH₂CH₃ | CN | C—CH₂CH₃ | CH | CH |
| E1.250 | CH₂CH₃ | OCH₃ | C—CH₂CH₃ | CH | CH |
| E1.251 | CH₂CH₃ | OCH₂CH₃ | C—CH₂CH₃ | CH | CH |
| E1.252 | CH₂CH₃ | OCH₂CH₂OCH₃ | C—CH₂CH₃ | CH | CH |
| E1.253 | CH₂CH₃ | OH | C—CH₂CH₃ | CH | CH |
| E1.254 | CH₂CH₃ | H | C—CN | CH | CH |
| E1.255 | CH₂CH₃ | CH₃ | C—CN | CH | CH |
| E1.256 | CH₂CH₃ | CH₂CH₃ | C—CN | CH | CH |
| E1.257 | CH₂CH₃ | F | C—CN | CH | CH |
| E1.258 | CH₂CH₃ | Cl | C—CN | CH | CH |
| E1.259 | CH₂CH₃ | Br | C—CN | CH | CH |
| E1.260 | CH₂CH₃ | CN | C—CN | CH | CH |
| E1.261 | CH₂CH₃ | OCH₃ | C—CN | CH | CH |
| E1.262 | CH₂CH₃ | OCH₂CH₃ | C—CN | CH | CH |
| E1.263 | CH₂CH₃ | OCH₂CH₂OCH₃ | C—CN | CH | CH |
| E1.264 | CH₂CH₃ | OH | C—CN | CH | CH |
| E1.265 | CH₂CH₃ | H | CH | CH | N |
| E1.266 | CH₂CH₃ | CH₃ | CH | CH | N |
| E1.267 | CH₂CH₃ | CH₂CH₃ | CH | CH | N |
| E1.268 | CH₂CH₃ | F | CH | CH | N |
| E1.269 | CH₂CH₃ | Cl | CH | CH | N |
| E1.270 | CH₂CH₃ | Br | CH | CH | N |
| E1.271 | CH₂CH₃ | CN | CH | CH | N |
| E1.272 | CH₂CH₃ | OCH₃ | CH | CH | N |
| E1.273 | CH₂CH₃ | OCH₂CH₃ | CH | CH | N |
| E1.274 | CH₂CH₃ | OCH₂CH₂OCH₃ | CH | CH | N |
| E1.275 | CH₂CH₃ | OH | CH | CH | N |
| E1.276 | CH₂CH₃ | H | N | CH | N |
| E1.277 | CH₂CH₃ | CH₃ | N | CH | N |
| E1.278 | CH₂CH₃ | CH₂CH₃ | N | CH | N |
| E1.279 | CH₂CH₃ | F | N | CH | N |
| E1.280 | CH₂CH₃ | Cl | N | CH | N |
| E1.281 | CH₂CH₃ | Br | N | CH | N |
| E1.282 | CH₂CH₃ | CN | N | CH | N |
| E1.283 | CH₂CH₃ | OCH₃ | N | CH | N |
| E1.284 | CH₂CH₃ | OCH₂CH₃ | N | CH | N |
| E1.285 | CH₂CH₃ | OCH₂CH₂OCH₃ | N | CH | N |
| E1.286 | CH₂CH₃ | OH | N | CH | N |
| E1.287 | CH₂CH₃ | H | CH | N | N |
| E1.288 | CH₂CH₃ | CH₃ | CH | N | N |
| E1.289 | CH₂CH₃ | CH₂CH₃ | CH | N | N |
| E1.290 | CH₂CH₃ | F | CH | N | N |
| E1.291 | CH₂CH₃ | Cl | CH | N | N |
| E1.292 | CH₂CH₃ | Br | CH | N | N |
| E1.293 | CH₂CH₃ | CN | CH | N | N |
| E1.294 | CH₂CH₃ | OCH₃ | CH | N | N |
| E1.295 | CH₂CH₃ | OCH₂CH₃ | CH | N | N |
| E1.296 | CH₂CH₃ | OCH₂CH₂OCH₃ | CH | N | N |
| E1.297 | CH₂CH₃ | OH | CH | N | N |
| E1.298 | CH₂CH₃ | H | C—F | CH | N |
| E1.299 | CH₂CH₃ | CH₃ | C—F | CH | N |
| E1.300 | CH₂CH₃ | CH₂CH₃ | C—F | CH | N |
| E1.301 | CH₂CH₃ | F | C—F | CH | N |
| E1.302 | CH₂CH₃ | Cl | C—F | CH | N |
| E1.303 | CH₂CH₃ | Br | C—F | CH | N |
| E1.304 | CH₂CH₃ | CN | C—F | CH | N |
| E1.305 | CH₂CH₃ | OCH₃ | C—F | CH | N |
| E1.306 | CH₂CH₃ | OCH₂CH₃ | C—F | CH | N |
| E1.307 | CH₂CH₃ | OCH₂CH₂OCH₃ | C—F | CH | N |
| E1.308 | CH₂CH₃ | OH | C—F | CH | N |
| E1.309 | CH₂CH₃ | H | C—Cl | CH | N |
| E1.310 | CH₂CH₃ | CH₃ | C—Cl | CH | N |
| E1.311 | CH₂CH₃ | CH₂CH₃ | C—Cl | CH | N |
| E1.312 | CH₂CH₃ | F | C—Cl | CH | N |
| E1.313 | CH₂CH₃ | Cl | C—Cl | CH | N |
| E1.314 | CH₂CH₃ | Br | C—Cl | CH | N |
| E1.315 | CH₂CH₃ | CN | C—Cl | CH | N |
| E1.316 | CH₂CH₃ | OCH₃ | C—Cl | CH | N |
| E1.317 | CH₂CH₃ | OCH₂CH₃ | C—Cl | CH | N |
| E1.318 | CH₂CH₃ | OCH₂CH₂OCH₃ | C—Cl | CH | N |
| E1.319 | CH₂CH₃ | OH | C—Cl | CH | N |
| E1.320 | CH₂CH₃ | H | C—CH₃ | CH | N |
| E1.321 | CH₂CH₃ | CH₃ | C—CH₃ | CH | N |
| E1.322 | CH₂CH₃ | CH₂CH₃ | C—CH₃ | CH | N |
| E1.323 | CH₂CH₃ | F | C—CH₃ | CH | N |
| E1.324 | CH₂CH₃ | Cl | C—CH₃ | CH | N |
| E1.325 | CH₂CH₃ | Br | C—CH₃ | CH | N |
| E1.326 | CH₂CH₃ | CN | C—CH₃ | CH | N |
| E1.327 | CH₂CH₃ | OCH₃ | C—CH₃ | CH | N |
| E1.328 | CH₂CH₃ | OCH₂CH₃ | C—CH₃ | CH | N |
| E1.329 | CH₂CH₃ | OCH₂CH₂OCH₃ | C—CH₃ | CH | N |
| E1.330 | CH₂CH₃ | OH | C—CH₃ | CH | N |
| E1.331 | CH₂CH₃ | H | C—CH₂CH₃ | CH | N |
| E1.332 | CH₂CH₃ | CH₃ | C—CH₂CH₃ | CH | N |
| E1.333 | CH₂CH₃ | CH₂CH₃ | C—CH₂CH₃ | CH | N |
| E1.334 | CH₂CH₃ | F | C—CH₂CH₃ | CH | N |
| E1.335 | CH₂CH₃ | Cl | C—CH₂CH₃ | CH | N |
| E1.336 | CH₂CH₃ | Br | C—CH₂CH₃ | CH | N |
| E1.337 | CH₂CH₃ | CN | C—CH₂CH₃ | CH | N |
| E1.338 | CH₂CH₃ | OCH₃ | C—CH₂CH₃ | CH | N |
| E1.339 | CH₂CH₃ | OCH₂CH₃ | C—CH₂CH₃ | CH | N |
| E1.340 | CH₂CH₃ | OCH₂CH₂OCH₃ | C—CH₂CH₃ | CH | N |
| E1.341 | CH₂CH₃ | OH | C—CH₂CH₃ | CH | N |
| E1.342 | CH₂CH₃ | H | C—CN | CH | N |
| E1.343 | CH₂CH₃ | CH₃ | C—CN | CH | N |
| E1.344 | CH₂CH₃ | CH₂CH₃ | C—CN | CH | N |
| E1.345 | CH₂CH₃ | F | C—CN | CH | N |
| E1.346 | CH₂CH₃ | Cl | C—CN | CH | N |
| E1.347 | CH₂CH₃ | Br | C—CN | CH | N |
| E1.348 | CH₂CH₃ | CN | C—CN | CH | N |
| E1.349 | CH₂CH₃ | OCH₃ | C—CN | CH | N |
| E1.350 | CH₂CH₃ | OCH₂CH₃ | C—CN | CH | N |
| E1.351 | CH₂CH₃ | OCH₂CH₂OCH₃ | C—CN | CH | N |
| E1.352 | CH₂CH₃ | OH | C—CN | CH | N |
| E1.353 | CH₂OCH₃ | H | CH | CH | CH |
| E1.354 | CH₂OCH₃ | CH₃ | CH | CH | CH |
| E1.355 | CH₂OCH₃ | CH₂CH₃ | CH | CH | CH |
| E1.356 | CH₂OCH₃ | F | CH | CH | CH |
| E1.357 | CH₂OCH₃ | Cl | CH | CH | CH |
| E1.358 | CH₂OCH₃ | Br | CH | CH | CH |
| E1.359 | CH₂OCH₃ | CN | CH | CH | CH |
| E1.360 | CH₂OCH₃ | OCH₃ | CH | CH | CH |
| E1.361 | CH₂OCH₃ | OCH₂CH₃ | CH | CH | CH |
| E1.362 | CH₂OCH₃ | OCH₂CH₂OCH₃ | CH | CH | CH |
| E1.363 | CH₂OCH₃ | OH | CH | CH | CH |
| E1.364 | CH₂OCH₃ | H | N | CH | CH |
| E1.365 | CH₂OCH₃ | CH₃ | N | CH | CH |
| E1.366 | CH₂OCH₃ | CH₂CH₃ | N | CH | CH |
| E1.367 | CH₂OCH₃ | F | N | CH | CH |
| E1.368 | CH₂OCH₃ | Cl | N | CH | CH |
| E1.369 | CH₂OCH₃ | Br | N | CH | CH |
| E1.370 | CH₂OCH₃ | CN | N | CH | CH |
| E1.371 | CH₂OCH₃ | OCH₃ | N | CH | CH |
| E1.372 | CH₂OCH₃ | OCH₂CH₃ | N | CH | CH |
| E1.373 | CH₂OCH₃ | OCH₂CH₂OCH₃ | N | CH | CH |
| E1.374 | CH₂OCH₃ | OH | N | CH | CH |
| E1.375 | CH₂OCH₃ | H | CH | N | CH |
| E1.376 | CH₂OCH₃ | CH₃ | CH | N | CH |
| E1.377 | CH₂OCH₃ | CH₂CH₃ | CH | N | CH |
| E1.378 | CH₂OCH₃ | F | CH | N | CH |
| E1.379 | CH₂OCH₃ | Cl | CH | N | CH |
| E1.380 | CH₂OCH₃ | Br | CH | N | CH |
| E1.381 | CH₂OCH₃ | CN | CH | N | CH |
| E1.382 | CH₂OCH₃ | OCH₃ | CH | N | CH |
| E1.383 | CH₂OCH₃ | OCH₂CH₃ | CH | N | CH |
| E1.384 | CH₂OCH₃ | OCH₂CH₂OCH₃ | CH | N | CH |
| E1.385 | CH₂OCH₃ | OH | CH | N | CH |
| E1.386 | CH₂OCH₃ | H | C—F | CH | CH |
| E1.387 | CH₂OCH₃ | CH₃ | C—F | CH | CH |
| E1.388 | CH₂OCH₃ | CH₂CH₃ | C—F | CH | CH |
| E1.389 | CH₂OCH₃ | F | C—F | CH | CH |
| E1.390 | CH₂OCH₃ | Cl | C—F | CH | CH |
| E1.391 | CH₂OCH₃ | Br | C—F | CH | CH |
| E1.392 | CH₂OCH₃ | CN | C—F | CH | CH |
| E1.393 | CH₂OCH₃ | OCH₃ | C—F | CH | CH |
| E1.394 | CH₂OCH₃ | OCH₂CH₃ | C—F | CH | CH |
| E1.395 | CH₂OCH₃ | OCH₂CH₂OCH₃ | C—F | CH | CH |
| E1.396 | CH₂OCH₃ | OH | C—F | CH | CH |
| E1.397 | CH₂OCH₃ | H | C—F | CH | CH |
| E1.398 | CH₂OCH₃ | CH₃ | C—Cl | CH | CH |
| E1.399 | CH₂OCH₃ | CH₂CH₃ | C—Cl | CH | CH |
| E1.400 | CH₂OCH₃ | F | C—Cl | CH | CH |
| E1.401 | CH₂OCH₃ | Cl | C—Cl | CH | CH |
| E1.402 | CH₂OCH₃ | Br | C—Cl | CH | CH |
| E1.403 | CH₂OCH₃ | CN | C—Cl | CH | CH |
| E1.404 | CH₂OCH₃ | OCH₃ | C—Cl | CH | CH |
| E1.405 | CH₂OCH₃ | OCH₂CH₃ | C—Cl | CH | CH |
| E1.406 | CH₂OCH₃ | OCH₂CH₂OCH₃ | C—Cl | CH | CH |
| E1.407 | CH₂OCH₃ | OH | C—Cl | CH | CH |
| E1.408 | CH₂OCH₃ | H | C—Cl | CH | CH |
| E1.409 | CH₂OCH₃ | CH₃ | C—CH₃ | CH | CH |
| E1.410 | CH₂OCH₃ | CH₂CH₃ | C—CH₃ | CH | CH |
| E1.411 | CH₂OCH₃ | F | C—CH₃ | CH | CH |
| E1.412 | CH₂OCH₃ | Cl | C—CH₃ | CH | CH |
| E1.413 | CH₂OCH₃ | Br | C—CH₃ | CH | CH |
| E1.414 | CH₂OCH₃ | CN | C—CH₃ | CH | CH |
| E1.415 | CH₂OCH₃ | OCH₃ | C—CH₃ | CH | CH |
| E1.416 | CH₂OCH₃ | OCH₂CH₃ | C—CH₃ | CH | CH |
| E1.417 | CH₂OCH₃ | OCH₂CH₂OCH₃ | C—CH₃ | CH | CH |
| E1.418 | CH₂OCH₃ | OH | C—CH₃ | CH | CH |
| E1.419 | CH₂OCH₃ | H | C—CH₃ | CH | CH |
| E1.420 | CH₂OCH₃ | CH₃ | C—CH₂CH₃ | CH | CH |
| E1.421 | CH₂OCH₃ | CH₂CH₃ | C—CH₂CH₃ | CH | CH |
| E1.422 | CH₂OCH₃ | F | C—CH₂CH₃ | CH | CH |
| E1.423 | CH₂OCH₃ | Cl | C—CH₂CH₃ | CH | CH |
| E1.424 | CH₂OCH₃ | Br | C—CH₂CH₃ | CH | CH |
| E1.425 | CH₂OCH₃ | CN | C—CH₂CH₃ | CH | CH |
| E1.426 | CH₂OCH₃ | OCH₃ | C—CH₂CH₃ | CH | CH |
| E1.427 | CH₂OCH₃ | OCH₂CH₃ | C—CH₂CH₃ | CH | CH |
| E1.428 | CH₂OCH₃ | OCH₂CH₂OCH₃ | C—CH₂CH₃ | CH | CH |
| E1.429 | CH₂OCH₃ | OH | C—CH₂CH₃ | CH | CH |
| E1.430 | CH₂OCH₃ | H | C—CH₂CH₃ | CH | CH |
| E1.431 | CH₂OCH₃ | CH₃ | C—CN | CH | CH |
| E1.432 | CH₂OCH₃ | CH₂CH₃ | C—CN | CH | CH |
| E1.433 | CH₂OCH₃ | F | C—CN | CH | CH |
| E1.434 | CH₂OCH₃ | Cl | C—CN | CH | CH |
| E1.435 | CH₂OCH₃ | Br | C—CN | CH | CH |
| E1.436 | CH₂OCH₃ | CN | C—CN | CH | CH |
| E1.437 | CH₂OCH₃ | OCH₃ | C—CN | CH | CH |
| E1.438 | CH₂OCH₃ | OCH₂CH₃ | C—CN | CH | CH |
| E1.439 | CH₂OCH₃ | OCH₂CH₂OCH₃ | C—CN | CH | CH |
| E1.440 | CH₂OCH₃ | OH | C—CN | CH | CH |
| E1.441 | CH₂OCH₃ | H | CH | CH | N |
| E1.442 | CH₂OCH₃ | CH₃ | CH | CH | N |
| E1.443 | CH₂OCH₃ | CH₂CH₃ | CH | CH | N |
| E1.444 | CH₂OCH₃ | F | CH | CH | N |
| E1.445 | CH₂OCH₃ | Cl | CH | CH | N |
| E1.446 | CH₂OCH₃ | Br | CH | CH | N |
| E1.447 | CH₂OCH₃ | CN | CH | CH | N |
| E1.448 | CH₂OCH₃ | OCH₃ | CH | CH | N |
| E1.449 | CH₂OCH₃ | OCH₂CH₃ | CH | CH | N |
| E1.450 | CH₂OCH₃ | OCH₂CH₂OCH₃ | CH | CH | N |
| E1.451 | CH₂OCH₃ | OH | CH | CH | N |
| E1.452 | CH₂OCH₃ | H | N | CH | N |
| E1.453 | CH₂OCH₃ | CH₃ | N | CH | N |
| E1.454 | CH₂OCH₃ | CH₂CH₃ | N | CH | N |
| E1.455 | CH₂OCH₃ | F | N | CH | N |
| E1.456 | CH₂OCH₃ | Cl | N | CH | N |
| E1.457 | CH₂OCH₃ | Br | N | CH | N |
| E1.458 | CH₂OCH₃ | CN | N | CH | N |
| E1.459 | CH₂OCH₃ | OCH₃ | N | CH | N |
| E1.460 | CH₂OCH₃ | OCH₂CH₃ | N | CH | N |
| E1.461 | CH₂OCH₃ | OCH₂CH₂OCH₃ | N | CH | N |
| E1.462 | CH₂OCH₃ | OH | N | CH | N |
| E1.463 | CH₂OCH₃ | H | CH | N | N |
| E1.464 | CH₂OCH₃ | CH₃ | CH | N | N |
| E1.465 | CH₂OCH₃ | CH₂CH₃ | CH | N | N |
| E1.466 | CH₂OCH₃ | F | CH | N | N |
| E1.467 | CH₂OCH₃ | Cl | CH | N | N |
| E1.468 | CH₂OCH₃ | Br | CH | N | N |
| E1.469 | CH₂OCH₃ | CN | CH | N | N |
| E1.470 | CH₂OCH₃ | OCH₃ | CH | N | N |
| E1.471 | CH₂OCH₃ | OCH₂CH₃ | CH | N | N |
| E1.472 | CH₂OCH₃ | OCH₂CH₂OCH₃ | CH | N | N |
| E1.473 | CH₂OCH₃ | OH | CH | N | N |
| E1.474 | CH₂OCH₃ | H | C—F | CH | N |
| E1.475 | CH₂OCH₃ | CH₃ | C—F | CH | N |
| E1.476 | CH₂OCH₃ | CH₂CH₃ | C—F | CH | N |
| E1.477 | CH₂OCH₃ | F | C—F | CH | N |
| E1.478 | CH₂OCH₃ | Cl | C—F | CH | N |
| E1.479 | CH₂OCH₃ | Br | C—F | CH | N |
| E1.480 | CH₂OCH₃ | CN | C—F | CH | N |
| E1.481 | CH₂OCH₃ | OCH₃ | C—F | CH | N |
| E1.482 | CH₂OCH₃ | OCH₂CH₃ | C—F | CH | N |
| E1.483 | CH₂OCH₃ | OCH₂CH₂OCH₃ | C—F | CH | N |
| E1.484 | CH₂OCH₃ | OH | C—F | CH | N |
| E1.485 | CH₂OCH₃ | H | C—Cl | CH | N |
| E1.486 | CH₂OCH₃ | CH₃ | C—Cl | CH | N |
| E1.487 | CH₂OCH₃ | CH₂CH₃ | C—Cl | CH | N |
| E1.488 | CH₂OCH₃ | F | C—Cl | CH | N |
| E1.489 | CH₂OCH₃ | Cl | C—Cl | CH | N |
| E1.490 | CH₂OCH₃ | Br | C—Cl | CH | N |
| E1.491 | CH₂OCH₃ | CN | C—Cl | CH | N |
| E1.492 | CH₂OCH₃ | OCH₃ | C—Cl | CH | N |
| E1.493 | CH₂OCH₃ | OCH₂CH₃ | C—Cl | CH | N |
| E1.494 | CH₂OCH₃ | OCH₂CH₂OCH₃ | C—Cl | CH | N |
| E1.495 | CH₂OCH₃ | OH | C—Cl | CH | N |
| E1.496 | CH₂OCH₃ | H | C—CH₃ | CH | N |
| E1.497 | CH₂OCH₃ | CH₃ | C—CH₃ | CH | N |
| E1.498 | CH₂OCH₃ | CH₂CH₃ | C—CH₃ | CH | N |
| E1.499 | CH₂OCH₃ | F | C—CH₃ | CH | N |
| E1.500 | CH₂OCH₃ | Cl | C—CH₃ | CH | N |
| E1.501 | CH₂OCH₃ | Br | C—CH₃ | CH | N |
| E1.502 | CH₂OCH₃ | CN | C—CH₃ | CH | N |
| E1.503 | CH₂OCH₃ | OCH₃ | C—CH₃ | CH | N |
| E1.504 | CH₂OCH₃ | OCH₂CH₃ | C—CH₃ | CH | N |
| E1.505 | CH₂OCH₃ | OCH₂CH₂OCH₃ | C—CH₃ | CH | N |
| E1.506 | CH₂OCH₃ | OH | C—CH₃ | CH | N |
| E1.507 | CH₂OCH₃ | H | C—CH₂CH₃ | CH | N |
| E1.508 | CH₂OCH₃ | CH₃ | C—CH₂CH₃ | CH | N |
| E1.509 | CH₂OCH₃ | CH₂CH₃ | C—CH₂CH₃ | CH | N |
| E1.510 | CH₂OCH₃ | F | C—CH₂CH₃ | CH | N |
| E1.511 | CH₂OCH₃ | Cl | C—CH₂CH₃ | CH | N |
| E1.512 | CH₂OCH₃ | Br | C—CH₂CH₃ | CH | N |
| E1.513 | CH₂OCH₃ | CN | C—CH₂CH₃ | CH | N |
| E1.514 | CH₂OCH₃ | OCH₃ | C—CH₂CH₃ | CH | N |
| E1.515 | CH₂OCH₃ | OCH₂CH₃ | C—CH₂CH₃ | CH | N |
| E1.516 | CH₂OCH₃ | OCH₂CH₂OCH₃ | C—CH₂CH₃ | CH | N |
| E1.517 | CH₂OCH₃ | OH | C—CH₂CH₃ | CH | N |
| E1.518 | CH₂OCH₃ | H | C—CN | CH | N |
| E1.519 | CH₂OCH₃ | CH₃ | C—CN | CH | N |
| E1.520 | CH₂OCH₃ | CH₂CH₃ | C—CN | CH | N |
| E1.521 | CH₂OCH₃ | F | C—CN | CH | N |
| E1.522 | CH₂OCH₃ | Cl | C—CN | CH | N |
| E1.523 | CH₂OCH₃ | Br | C—CN | CH | N |
| E1.524 | CH₂OCH₃ | CN | C—CN | CH | N |
| E1.525 | CH₂OCH₃ | OCH₃ | C—CN | CH | N |
| E1.526 | CH₂OCH₃ | OCH₂CH₃ | C—CN | CH | N |
| E1.527 | CH₂OCH₃ | OCH₂CH₂OCH₃ | C—CN | CH | N |
| E1.528 | CH₂OCH₃ | OH | C—CN | CH | N |
| E1.529 | CH₂CH₂OCH₃ | H | CH | CH | CH |
| E1.530 | CH₂CH₂OCH₃ | CH₃ | CH | CH | CH |
| E1.531 | CH₂CH₂OCH₃ | CH₂CH₃ | CH | CH | CH |
| E1.532 | CH₂CH₂OCH₃ | F | CH | CH | CH |
| E1.533 | CH₂CH₂OCH₃ | Cl | CH | CH | CH |
| E1.534 | CH₂CH₂OCH₃ | Br | CH | CH | CH |
| E1.535 | CH₂CH₂OCH₃ | CN | CH | CH | CH |
| E1.536 | CH₂CH₂OCH₃ | OCH₃ | CH | CH | CH |
| E1.537 | CH₂CH₂OCH₃ | OCH₂CH₃ | CH | CH | CH |
| E1.538 | CH₂CH₂OCH₃ | OCH₂CH₂OCH₃ | CH | CH | CH |
| E1.539 | CH₂CH₂OCH₃ | OH | CH | CH | CH |
| E1.540 | CH₂CH₂OCH₃ | H | N | CH | CH |
| E1.541 | CH₂CH₂OCH₃ | CH₃ | N | CH | CH |
| E1.542 | CH₂CH₂OCH₃ | CH₂CH₃ | N | CH | CH |
| E1.543 | CH₂CH₂OCH₃ | F | N | CH | CH |
| E1.544 | CH₂CH₂OCH₃ | Cl | N | CH | CH |
| E1.545 | CH₂CH₂OCH₃ | Br | N | CH | CH |
| E1.546 | CH₂CH₂OCH₃ | CN | N | CH | CH |
| E1.547 | CH₂CH₂OCH₃ | OCH₃ | N | CH | CH |
| E1.548 | CH₂CH₂OCH₃ | OCH₂CH₃ | N | CH | CH |
| E1.549 | CH₂CH₂OCH₃ | OCH₂CH₂OCH₃ | N | CH | CH |
| E1.550 | CH₂CH₂OCH₃ | OH | N | CH | CH |
| E1.551 | CH₂CH₂OCH₃ | H | CH | N | CH |
| E1.552 | CH₂CH₂OCH₃ | CH₃ | CH | N | CH |
| E1.553 | CH₂CH₂OCH₃ | CH₂CH₃ | CH | N | CH |
| E1.554 | CH₂CH₂OCH₃ | F | CH | N | CH |
| E1.555 | CH₂CH₂OCH₃ | Cl | CH | N | CH |
| E1.556 | CH₂CH₂OCH₃ | Br | CH | N | CH |
| E1.557 | CH₂CH₂OCH₃ | CN | CH | N | CH |
| E1.558 | CH₂CH₂OCH₃ | OCH₃ | CH | N | CH |
| E1.559 | CH₂CH₂OCH₃ | OCH₂CH₃ | CH | N | CH |
| E1.560 | CH₂CH₂OCH₃ | OCH₂CH₂OCH₃ | CH | N | CH |
| E1.561 | CH₂CH₂OCH₃ | OH | CH | N | CH |
| E1.562 | CH₂CH₂OCH₃ | H | C—F | CH | CH |
| E1.563 | CH₂CH₂OCH₃ | CH₃ | C—F | CH | CH |
| E1.564 | CH₂CH₂OCH₃ | CH₂CH₃ | C—F | CH | CH |
| E1.565 | CH₂CH₂OCH₃ | F | C—F | CH | CH |
| E1.566 | CH₂CH₂OCH₃ | Cl | C—F | CH | CH |
| E1.567 | CH₂CH₂OCH₃ | Br | C—F | CH | CH |
| E1.568 | CH₂CH₂OCH₃ | CN | C—F | CH | CH |
| E1.569 | CH₂CH₂OCH₃ | OCH₃ | C—F | CH | CH |
| E1.570 | CH₂CH₂OCH₃ | OCH₂CH₃ | C—F | CH | CH |
| E1.571 | CH₂CH₂OCH₃ | OCH₂CH₂OCH₃ | C—F | CH | CH |
| E1.572 | CH₂CH₂OCH₃ | OH | C—F | CH | CH |
| E1.573 | CH₂CH₂OCH₃ | H | C—Cl | CH | CH |
| E1.574 | CH₂CH₂OCH₃ | CH₃ | C—Cl | CH | CH |
| E1.575 | CH₂CH₂OCH₃ | CH₂CH₃ | C—Cl | CH | CH |
| E1.576 | CH₂CH₂OCH₃ | F | C—Cl | CH | CH |
| E1.577 | CH₂CH₂OCH₃ | Cl | C—Cl | CH | CH |
| E1.578 | CH₂CH₂OCH₃ | Br | C—Cl | CH | CH |
| E1.579 | CH₂CH₂OCH₃ | CN | C—Cl | CH | CH |
| E1.580 | CH₂CH₂OCH₃ | OCH₃ | C—Cl | CH | CH |
| E1.581 | CH₂CH₂OCH₃ | OCH₂CH₃ | C—Cl | CH | CH |
| E1.582 | CH₂CH₂OCH₃ | OCH₂CH₂OCH₃ | C—Cl | CH | CH |
| E1.583 | CH₂CH₂OCH₃ | OH | C—Cl | CH | CH |
| E1.584 | CH₂CH₂OCH₃ | H | C—CH₃ | CH | CH |
| E1.585 | CH₂CH₂OCH₃ | CH₃ | C—CH₃ | CH | CH |
| E1.586 | CH₂CH₂OCH₃ | CH₂CH₃ | C—CH₃ | CH | CH |
| E1.587 | CH₂CH₂OCH₃ | F | C—CH₃ | CH | CH |
| E1.588 | CH₂CH₂OCH₃ | Cl | C—CH₃ | CH | CH |
| E1.589 | CH₂CH₂OCH₃ | Br | C—CH₃ | CH | CH |
| E1.590 | CH₂CH₂OCH₃ | CN | C—CH₃ | CH | CH |
| E1.591 | CH₂CH₂OCH₃ | OCH₃ | C—CH₃ | CH | CH |
| E1.592 | CH₂CH₂OCH₃ | OCH₂CH₃ | C—CH₃ | CH | CH |
| E1.593 | CH₂CH₂OCH₃ | OCH₂CH₂OCH₃ | C—CH₃ | CH | CH |
| E1.594 | CH₂CH₂OCH₃ | OH | C—CH₃ | CH | CH |
| E1.595 | CH₂CH₂OCH₃ | H | C—CH₂CH₃ | CH | CH |
| E1.596 | CH₂CH₂OCH₃ | CH₃ | C—CH₂CH₃ | CH | CH |
| E1.597 | CH₂CH₂OCH₃ | CH₂CH₃ | C—CH₂CH₃ | CH | CH |
| E1.598 | CH₂CH₂OCH₃ | F | C—CH₂CH₃ | CH | CH |
| E1.599 | CH₂CH₂OCH₃ | Cl | C—CH₂CH₃ | CH | CH |
| E1.600 | CH₂CH₂OCH₃ | Br | C—CH₂CH₃ | CH | CH |
| E1.601 | CH₂CH₂OCH₃ | CN | C—CH₂CH₃ | CH | CH |
| E1.602 | CH₂CH₂OCH₃ | OCH₃ | C—CH₂CH₃ | CH | CH |
| E1.603 | CH₂CH₂OCH₃ | OCH₂CH₃ | C—CH₂CH₃ | CH | CH |
| E1.604 | CH₂CH₂OCH₃ | OCH₂CH₂OCH₃ | C—CH₂CH₃ | CH | CH |
| E1.605 | CH₂CH₂OCH₃ | OH | C—CH₂CH₃ | CH | CH |
| E1.606 | CH₂CH₂OCH₃ | H | C—CN | CH | CH |
| E1.607 | CH₂CH₂OCH₃ | CH₃ | C—CN | CH | CH |
| E1.608 | CH₂CH₂OCH₃ | CH₂CH₃ | C—CN | CH | CH |
| E1.609 | CH₂CH₂OCH₃ | F | C—CN | CH | CH |
| E1.610 | CH₂CH₂OCH₃ | Cl | C—CN | CH | CH |
| E1.611 | CH₂CH₂OCH₃ | Br | C—CN | CH | CH |
| E1.612 | CH₂CH₂OCH₃ | CN | C—CN | CH | CH |
| E1.613 | CH₂CH₂OCH₃ | OCH₃ | C—CN | CH | CH |
| E1.614 | CH₂CH₂OCH₃ | OCH₂CH₃ | C—CN | CH | CH |
| E1.615 | CH₂CH₂OCH₃ | OCH₂CH₂OCH₃ | C—CN | CH | CH |
| E1.616 | CH₂CH₂OCH₃ | OH | C—CN | CH | CH |
| E1.617 | CH₂CH₂OCH₃ | H | CH | CH | N |
| E1.618 | CH₂CH₂OCH₃ | CH₃ | CH | CH | N |
| E1.619 | CH₂CH₂OCH₃ | CH₂CH₃ | CH | CH | N |
| E1.620 | CH₂CH₂OCH₃ | F | CH | CH | N |
| E1.621 | CH₂CH₂OCH₃ | Cl | CH | CH | N |
| E1.622 | CH₂CH₂OCH₃ | Br | CH | CH | N |
| E1.623 | CH₂CH₂OCH₃ | CN | CH | CH | N |
| E1.624 | CH₂CH₂OCH₃ | OCH₃ | CH | CH | N |
| E1.625 | CH₂CH₂OCH₃ | OCH₂CH₃ | CH | CH | N |
| E1.626 | CH₂CH₂OCH₃ | OCH₂CH₂OCH₃ | CH | CH | N |
| E1.627 | CH₂CH₂OCH₃ | OH | CH | CH | N |
| E1.628 | CH₂CH₂OCH₃ | H | N | CH | N |
| E1.629 | CH₂CH₂OCH₃ | CH₃ | N | CH | N |
| E1.630 | CH₂CH₂OCH₃ | CH₂CH₃ | N | CH | N |
| E1.631 | CH₂CH₂OCH₃ | F | N | CH | N |
| E1.632 | CH₂CH₂OCH₃ | Cl | N | CH | N |
| E1.633 | CH₂CH₂OCH₃ | Br | N | CH | N |
| E1.634 | CH₂CH₂OCH₃ | CN | N | CH | N |
| E1.635 | CH₂CH₂OCH₃ | OCH₃ | N | CH | N |
| E1.636 | CH₂CH₂OCH₃ | OCH₂CH₃ | N | CH | N |
| E1.637 | CH₂CH₂OCH₃ | OCH₂CH₂OCH₃ | N | CH | N |
| E1.638 | CH₂CH₂OCH₃ | OH | N | CH | N |
| E1.639 | CH₂CH₂OCH₃ | H | CH | N | N |
| E1.640 | CH₂CH₂OCH₃ | CH₃ | CH | N | N |
| E1.641 | CH₂CH₂OCH₃ | CH₂CH₃ | CH | N | N |
| E1.642 | CH₂CH₂OCH₃ | F | CH | N | N |
| E1.643 | CH₂CH₂OCH₃ | Cl | CH | N | N |
| E1.644 | CH₂CH₂OCH₃ | Br | CH | N | N |
| E1.645 | CH₂CH₂OCH₃ | CN | CH | N | N |
| E1.646 | CH₂CH₂OCH₃ | OCH₃ | CH | N | N |
| E1.647 | CH₂CH₂OCH₃ | OCH₂CH₃ | CH | N | N |
| E1.648 | CH₂CH₂OCH₃ | OCH₂CH₂OCH₃ | CH | N | N |
| E1.649 | CH₂CH₂OCH₃ | OH | CH | N | N |
| E1.650 | CH₂CH₂OCH₃ | H | C—F | CH | N |
| E1.651 | CH₂CH₂OCH₃ | CH₃ | C—F | CH | N |
| E1.652 | CH₂CH₂OCH₃ | CH₂CH₃ | C—F | CH | N |
| E1.653 | CH₂CH₂OCH₃ | F | C—F | CH | N |
| E1.654 | CH₂CH₂OCH₃ | Cl | C—F | CH | N |
| E1.655 | CH₂CH₂OCH₃ | Br | C—F | CH | N |
| E1.656 | CH₂CH₂OCH₃ | CN | C—F | CH | N |
| E1.657 | CH₂CH₂OCH₃ | OCH₃ | C—F | CH | N |
| E1.658 | CH₂CH₂OCH₃ | OCH₂CH₃ | C—F | CH | N |
| E1.659 | CH₂CH₂OCH₃ | OCH₂CH₂OCH₃ | C—F | CH | N |
| E1.660 | CH₂CH₂OCH₃ | OH | C—F | CH | N |
| E1.661 | CH₂CH₂OCH₃ | H | C—Cl | CH | N |
| E1.662 | CH₂CH₂OCH₃ | CH | C—Cl | CH | N |
| E1.663 | CH₂CH₂OCH₃ | CH₂CH₃ | C—Cl | CH | N |
| E1.664 | CH₂CH₂OCH₃ | F | C—Cl | CH | N |
| E1.665 | CH₂CH₂OCH₃ | Cl | C—Cl | CH | N |
| E1.666 | CH₂CH₂OCH₃ | Br | C—Cl | CH | N |
| E1.667 | CH₂CH₂OCH₃ | CN | C—Cl | CH | N |
| E1.668 | CH₂CH₂OCH₃ | OCH₃ | C—Cl | CH | N |
| E1.669 | CH₂CH₂OCH₃ | OCH₂CH₃ | C—Cl | CH | N |
| E1.670 | CH₂CH₂OCH₃ | OCH₂CH₂OCH₃ | C—Cl | CH | N |
| E1.671 | CH₂CH₂OCH₃ | OH | C—Cl | CH | N |
| E1.672 | CH₂CH₂OCH₃ | H | C—CH₃ | CH | N |
| E1.673 | CH₂CH₂OCH₃ | CH₃ | C—CH₃ | CH | N |
| E1.674 | CH₂CH₂OCH₃ | CH₂CH₃ | C—CH₃ | CH | N |
| E1.675 | CH₂CH₂OCH₃ | F | C—CH₃ | CH | N |
| E1.676 | CH₂CH₂OCH₃ | Cl | C—CH₃ | CH | N |
| E1.677 | CH₂CH₂OCH₃ | Br | C—CH₃ | CH | N |
| E1.678 | CH₂CH₂OCH₃ | CN | C—CH₃ | CH | N |
| E1.679 | CH₂CH₂OCH₃ | OCH₃ | C—CH₃ | CH | N |
| E1.680 | CH₂CH₂OCH₃ | OCH₂CH₃ | C—CH₃ | CH | N |
| E1.681 | CH₂CH₂OCH₃ | OCH₂CH₂OCH₃ | C—CH₃ | CH | N |
| E1.682 | CH₂CH₂OCH₃ | OH | C—CH₃ | CH | N |
| E1.683 | CH₂CH₂OCH₃ | H | C—CH₂CH₃ | CH | N |
| E1.684 | CH₂CH₂OCH₃ | CH₃ | C—CH₂CH₃ | CH | N |
| E1.685 | CH₂CH₂OCH₃ | CH₂CH₃ | C—CH₂CH₃ | CH | N |
| E1.686 | CH₂CH₂OCH₃ | F | C—CH₂CH₃ | CH | N |
| E1.687 | CH₂CH₂OCH₃ | Cl | C—CH₂CH₃ | CH | N |
| E1.688 | CH₂CH₂OCH₃ | Br | C—CH₂CH₃ | CH | N |
| E1.689 | CH₂CH₂OCH₃ | CN | C—CH₂CH₃ | CH | N |
| E1.690 | CH₂CH₂OCH₃ | OCH₃ | C—CH₂CH₃ | CH | N |
| E1.691 | CH₂CH₂OCH₃ | OCH₂CH₃ | C—CH₂CH₃ | CH | N |
| E1.692 | CH₂CH₂OCH₃ | OCH₂CH₂OCH₃ | C—CH₂CH₃ | CH | N |
| E1.693 | CH₂CH₂OCH₃ | OH | C—CH₂CH₃ | CH | N |
| E1.694 | CH₂CH₂OCH₃ | H | C—CN | CH | N |
| E1.695 | CH₂CH₂OCH₃ | CH₃ | C—CN | CH | N |
| E1.696 | CH₂CH₂OCH₃ | CH₂CH₃ | C—CN | CH | N |
| E1.697 | CH₂CH₂OCH₃ | F | C—CN | CH | N |
| E1.698 | CH₂CH₂OCH₃ | Cl | C—CN | CH | N |
| E1.699 | CH₂CH₂OCH₃ | Br | C—CN | CH | N |
| E1.700 | CH₂CH₂OCH₃ | CN | C—CN | CH | N |
| E1.701 | CH₂CH₂OCH₃ | OCH₃ | C—CN | CH | N |
| E1.702 | CH₂CH₂OCH₃ | OCH₂CH₃ | C—CN | CH | N |
| E1.703 | CH₂CH₂OCH₃ | OCH₂CH₂OCH₃ | C—CN | CH | N |
| E1.704 | CH₂CH₂OCH₃ | OH | C—CN | CH | N |
| E1.705 | CH₂CN | H | CH | CH | CH |
| E1.706 | CH₂CN | CH₃ | CH | CH | CH |
| E1.707 | CH₂CN | CH₂CH₃ | CH | CH | CH |
| E1.708 | CH₂CN | F | CH | CH | CH |
| E1.709 | CH₂CN | Cl | CH | CH | CH |
| E1.710 | CH₂CN | Br | CH | CH | CH |
| E1.711 | CH₂CN | CN | CH | CH | CH |
| E1.712 | CH₂CN | OCH₃ | CH | CH | CH |
| E1.713 | CH₂CN | OCH₂CH₃ | CH | CH | CH |
| E1.714 | CH₂CN | OCH₂CH₂OCH₃ | CH | CH | CH |
| E1.715 | CH₂CN | OH | CH | CH | CH |
| E1.716 | CH₂CN | H | N | CH | CH |
| E1.717 | CH₂CN | CH₃ | N | CH | CH |
| E1.718 | CH₂CN | CH₂CH₃ | N | CH | CH |
| E1.719 | CH₂CN | F | N | CH | CH |
| E1.720 | CH₂CN | Cl | N | CH | CH |
| E1.721 | CH₂CN | Br | N | CH | CH |
| E1.722 | CH₂CN | CN | N | CH | CH |
| E1.723 | CH₂CN | OCH₃ | N | CH | CH |
| E1.724 | CH₂CN | OCH₂CH₃ | N | CH | CH |
| E1.725 | CH₂CN | OCH₂CH₂OCH₃ | N | CH | CH |
| E1.726 | CH₂CN | OH | N | CH | CH |
| E1.727 | CH₂CN | H | CH | N | CH |
| E1.728 | CH₂CN | CH₃ | CH | N | CH |
| E1.729 | CH₂CN | CH₂CH₃ | CH | N | CH |
| E1.730 | CH₂CN | F | CH | N | CH |
| E1.731 | CH₂CN | Cl | CH | N | CH |
| E1.732 | CH₂CN | Br | CH | N | CH |
| E1.733 | CH₂CN | CN | CH | N | CH |
| E1.734 | CH₂CN | OCH₃ | CH | N | CH |
| E1.735 | CH₂CN | OCH₂CH₃ | CH | N | CH |
| E1.736 | CH₂CN | OCH₂CH₂OCH₃ | CH | N | CH |
| E1.737 | CH₂CN | OH | CH | N | CH |
| E1.738 | CH₂CN | H | C—F | CH | CH |
| E1.739 | CH₂CN | CH₃ | C—F | CH | CH |
| E1.740 | CH₂CN | CH₂CH₃ | C—F | CH | CH |
| E1.741 | CH₂CN | F | C—F | CH | CH |
| E1.742 | CH₂CN | Cl | C—F | CH | CH |
| E1.743 | CH₂CN | Br | C—F | CH | CH |
| E1.744 | CH₂CN | CN | C—F | CH | CH |
| E1.745 | CH₂CN | OCH₃ | C—F | CH | CH |
| E1.746 | CH₂CN | OCH₂CH₃ | C—F | CH | CH |
| E1.747 | CH₂CN | OCH₂CH₂OCH₃ | C—F | CH | CH |
| E1.748 | CH₂CN | OH | C—F | CH | CH |
| E1.749 | CH₂CN | H | C—Cl | CH | CH |
| E1.750 | CH₂CN | CH₃ | C—Cl | CH | CH |
| E1.751 | CH₂CN | CH₂CH₃ | C—Cl | CH | CH |
| E1.752 | CH₂CN | F | C—Cl | CH | CH |
| E1.753 | CH₂CN | Cl | C—Cl | CH | CH |
| E1.754 | CH₂CN | Br | C—Cl | CH | CH |
| E1.755 | CH₂CN | CN | C—Cl | CH | CH |
| E1.756 | CH₂CN | OCH₃ | C—Cl | CH | CH |
| E1.757 | CH₂CN | OCH₂CH₃ | C—Cl | CH | CH |
| E1.758 | CH₂CN | OCH₂CH₂OCH₃ | C—Cl | CH | CH |
| E1.759 | CH₂CN | OH | C—Cl | CH | CH |
| E1.760 | CH₂CN | H | C—CH₃ | CH | CH |
| E1.761 | CH₂CN | CH₃ | C—CH₃ | CH | CH |
| E1.762 | CH₂CN | CH₂CH₃ | C—CH₃ | CH | CH |
| E1.763 | CH₂CN | F | C—CH₃ | CH | CH |
| E1.764 | CH₂CN | Cl | C—CH₃ | CH | CH |
| E1.765 | CH₂CN | Br | C—CH₃ | CH | CH |
| E1.766 | CH₂CN | CN | C—CH₃ | CH | CH |
| E1.767 | CH₂CN | OCH₃ | C—CH₃ | CH | CH |
| E1.768 | CH₂CN | OCH₂CH₃ | C—CH₃ | CH | CH |
| E1.769 | CH₂CN | OCH₂CH₂OCH₃ | C—CH₃ | CH | CH |
| E1.770 | CH₂CN | OH | C—CH₃ | CH | CH |
| E1.771 | CH₂CN | H | C—CH₂CH₃ | CH | CH |
| E1.772 | CH₂CN | CH₃ | C—CH₂CH₃ | CH | CH |
| E1.773 | CH₂CN | CH₂CH₃ | C—CH₂CH₃ | CH | CH |
| E1.774 | CH₂CN | F | C—CH₂CH₃ | CH | CH |
| E1.775 | CH₂CN | Cl | C—CH₂CH₃ | CH | CH |
| E1.776 | CH₂CN | Br | C—CH₂CH₃ | CH | CH |
| E1.777 | CH₂CN | CN | C—CH₂CH₃ | CH | CH |
| E1.778 | CH₂CN | OCH₃ | C—CH₂CH₃ | CH | CH |
| E1.779 | CH₂CN | OCH₂CH₃ | C—CH₂CH₃ | CH | CH |
| E1.780 | CH₂CN | OCH₂CH₂OCH₃ | C—CH₂CH₃ | CH | CH |
| E1.781 | CH₂CN | OH | C—CH₂CH₃ | CH | CH |
| E1.782 | CH₂CN | H | C—CN | CH | CH |
| E1.783 | CH₂CN | CH₃ | C—CN | CH | CH |
| E1.784 | CH₂CN | CH₂CH₃ | C—CN | CH | CH |
| E1.785 | CH₂CN | F | C—CN | CH | CH |
| E1.786 | CH₂CN | Cl | C—CN | CH | CH |
| E1.787 | CH₂CN | Br | C—CN | CH | CH |
| E1.788 | CH₂CN | CN | C—CN | CH | CH |
| E1.789 | CH₂CN | OCH₃ | C—CN | CH | CH |
| E1.790 | CH₂CN | OCH₂CH₃ | C—CN | CH | CH |
| E1.791 | CH₂CN | OCH₂CH₂OCH₃ | C—CN | CH | CH |
| E1.792 | CH₂CN | OH | C—CN | CH | CH |
| E1.793 | CH₂CN | H | CH | CH | N |
| E1.794 | CH₂CN | CH₃ | CH | CH | N |
| E1.795 | CH₂CN | CH₂CH₃ | CH | CH | N |
| E1.796 | CH₂CN | F | CH | CH | N |
| E1.797 | CH₂CN | Cl | CH | CH | N |
| E1.798 | CH₂CN | Br | CH | CH | N |
| E1.799 | CH₂CN | CN | CH | CH | N |
| E1.800 | CH₂CN | OCH₃ | CH | CH | N |
| E1.801 | CH₂CN | OCH₂CH₃ | CH | CH | N |
| E1.802 | CH₂CN | OCH₂CH₂OCH₃ | CH | CH | N |
| E1.803 | CH₂CN | OH | CH | CH | N |
| E1.804 | CH₂CN | H | N | CH | N |
| E1.805 | CH₂CN | CH₃ | N | CH | N |
| E1.806 | CH₂CN | CH₂CH₃ | N | CH | N |
| E1.807 | CH₂CN | F | N | CH | N |
| E1.808 | CH₂CN | Cl | N | CH | N |
| E1.809 | CH₂CN | Br | N | CH | N |
| E1.810 | CH₂CN | CN | N | CH | N |
| E1.811 | CH₂CN | OCH₃ | N | CH | N |
| E1.812 | CH₂CN | OCH₂CH₃ | N | CH | N |
| E1.813 | CH₂CN | OCH₂CH₂OCH₃ | N | CH | N |
| E1.814 | CH₂CN | OH | N | CH | N |
| E1.815 | CH₂CN | H | CH | N | N |
| E1.816 | CH₂CN | CH₃ | CH | N | N |
| E1.817 | CH₂CN | CH₂CH₃ | CH | N | N |
| E1.818 | CH₂CN | F | CH | N | N |
| E1.819 | CH₂CN | Cl | CH | N | N |
| E1.820 | CH₂CN | Br | CH | N | N |
| E1.821 | CH₂CN | CN | CH | N | N |
| E1.822 | CH₂CN | OCH₃ | CH | N | N |
| E1.823 | CH₂CN | OCH₂CH₃ | CH | N | N |
| E1.824 | CH₂CN | OCH₂CH₂OCH₃ | CH | N | N |
| E1.825 | CH₂CN | OH | CH | N | N |
| E1.826 | CH₂CN | H | C—F | CH | N |
| E1.827 | CH₂CN | CH₃ | C—F | CH | N |
| E1.828 | CH₂CN | CH₂CH₃ | C—F | CH | N |
| E1.829 | CH₂CN | F | C—F | CH | N |
| E1.830 | CH₂CN | Cl | C—F | CH | N |
| E1.831 | CH₂CN | Br | C—F | CH | N |
| E1.832 | CH₂CN | CN | C—F | CH | N |
| E1.833 | CH₂CN | OCH₃ | C—F | CH | N |
| E1.834 | CH₂CN | OCH₂CH₃ | C—F | CH | N |
| E1.835 | CH₂CN | OCH₂CH₂OCH₃ | C—F | CH | N |
| E1.836 | CH₂CN | OH | C—F | CH | N |
| E1.837 | CH₂CN | H | C—Cl | CH | N |
| E1.838 | CH₂CN | CH₃ | C—Cl | CH | N |
| E1.839 | CH₂CN | CH₂CH₃ | C—Cl | CH | N |
| E1.840 | CH₂CN | F | C—Cl | CH | N |
| E1.841 | CH₂CN | Cl | C—Cl | CH | N |
| E1.842 | CH₂CN | Br | C—Cl | CH | N |
| E1.843 | CH₂CN | CN | C—Cl | CH | N |
| E1.844 | CH₂CN | OCH₃ | C—Cl | CH | N |
| E1.845 | CH₂CN | OCH₂CH₃ | C—Cl | CH | N |
| E1.846 | CH₂CN | OCH₂CH₂OCH₃ | C—Cl | CH | N |
| E1.847 | CH₂CN | OH | C—Cl | CH | N |
| E1.848 | CH₂CN | H | C—CH₃ | CH | N |
| E1.849 | CH₂CN | CH₃ | C—CH₃ | CH | N |
| E1.850 | CH₂CN | CH₂CH₃ | C—CH₃ | CH | N |
| E1.851 | CH₂CN | F | C—CH₃ | CH | N |
| E1.852 | CH₂CN | Cl | C—CH₃ | CH | N |
| E1.853 | CH₂CN | Br | C—CH₃ | CH | N |
| E1.854 | CH₂CN | CN | C—CH₃ | CH | N |
| E1.855 | CH₂CN | OCH₃ | C—CH₃ | CH | N |
| E1.856 | CH₂CN | OCH₂CH₃ | C—CH₃ | CH | N |
| E1.857 | CH₂CN | OCH₂CH₂OCH₃ | C—CH₃ | CH | N |
| E1.858 | CH₂CN | OH | C—CH₃ | CH | N |
| E1.859 | CH₂CN | H | C—CH₂CH₃ | CH | N |
| E1.860 | CH₂CN | CH₃ | C—CH₂CH₃ | CH | N |
| E1.861 | CH₂CN | CH₂CH₃ | C—CH₂CH₃ | CH | N |
| E1.862 | CH₂CN | F | C—CH₂CH₃ | CH | N |
| E1.863 | CH₂CN | Cl | C—CH₂CH₃ | CH | N |
| E1.864 | CH₂CN | Br | C—CH₂CH₃ | CH | N |
| E1.865 | CH₂CN | CN | C—CH₂CH₃ | CH | N |
| E1.866 | CH₂CN | OCH₃ | C—CH₂CH₃ | CH | N |
| E1.867 | CH₂CN | OCH₂CH₃ | C—CH₂CH₃ | CH | N |
| E1.868 | CH₂CN | OCH₂CH₂OCH₃ | C—CH₂CH₃ | CH | N |
| E1.869 | CH₂CN | OH | C—CH₂CH₃ | CH | N |
| E1.870 | CH₂CN | H | C—CN | CH | N |
| E1.871 | CH₂CN | CH₃ | C—CN | CH | N |
| E1.872 | CH₂CN | CH₂CH₃ | C—CN | CH | N |
| E1.873 | CH₂CN | F | C—CN | CH | N |
| E1.874 | CH₂CN | Cl | C—CN | CH | N |
| E1.875 | CH₂CN | Br | C—CN | CH | N |
| E1.876 | CH₂CN | CN | C—CN | CH | N |
| E1.877 | CH₂CN | OCH₃ | C—CN | CH | N |
| E1.878 | CH₂CN | OCH₂CH₃ | C—CN | CH | N |
| E1.879 | CH₂CN | OCH₂CH₂OCH₃ | C—CN | CH | N |
| E1.880 | CH₂CN | OH | C—CN | CH | N |

Table 1.2: This table discloses specific compounds of formula (1) wherein R^{2a} is H, R^{2b} is H, R^{2c} is H and R⁶ is methoxy and wherein the values of R¹, R³, J, G and Aare as defined in Table Z.1

Table 1.3: This table discloses specific compounds of formula (1) wherein R^{2a} is methyl, R^{2b} is H, R^{2c} is H and R⁶ is methyl and wherein the values of R¹, R³, J, G and Aare as defined in Table Z.1

Table 1.4: This table discloses specific compounds of formula (1) wherein R^{2a} is methyl, R^{2b} is H, R^{2c} is H and R⁶ is methoxy and wherein the values of R¹, R³, J, G and Aare as defined in Table Z.1

Table 1.5: This table discloses specific compounds of formula (I) wherein R^{2a} is fluoro, R^{2b} is H, R^{2c} is H and R⁶ is methyl and wherein the values of R¹, R³, J, G and Aare as defined in Table Z.1.

Table 1.6: This table discloses specific compounds of formula (I) wherein R^{2a} is fluoro, R^{2b} is H, R^{2c} is H and R⁶ is methoxy and wherein the values of R¹, R³, J, G and Aare as defined in Table Z.1.

Table 1.7: This table discloses specific compounds of formula (I) wherein R^{2a} is chloro, R^{2b} is H, R^{2c} is H and R⁶ is methyl and wherein the values of R¹, R³, J, G and Aare as defined in Table Z.1.

Table 1.8: This table discloses specific compounds of formula (I) wherein R^{2a} is chloro, R^{2b} is H, R^{2c} is H and R⁶ is methoxy and wherein the values of R¹, R³, J, G and Aare as defined in Table Z.1.

Table 1.9: This table discloses specific compounds of formula (I) wherein R^{2a} is bromo, R^{2b} is H, R^{2c} is H and R⁶ is methyl and wherein the values of R¹, R³, J, G and Aare as defined in Table Z.1.

Table 1.10: This table discloses specific compounds of formula (I) wherein R^{2a} is bromo, R^{2b} is H, R^{2c} is H and R⁶ is methoxy and wherein the values of R¹, R³, J, G and Aare as defined in Table Z.1.

Table 1.11: This table discloses specific compounds of formula (I) wherein R^{2a} is cyano, R^{2b} is H, R^{2c} is H and R⁶ is methyl and wherein the values of R¹, R³, J, G and Aare as defined in Table Z.1.

Table 1.12: This table discloses specific compounds of formula (I) wherein R^{2a} is cyano, R^{2b} is H, R^{2c} is H and R⁶ is methoxy and wherein the values of R¹, R³, J, G and Aare as defined in Table Z.1.

Table 1.13: This table discloses specific compounds of formula (I) wherein R^{2a} is methoxy, R^{2b} is H, R^{2c} is H and R⁶ is methyl and wherein the values of R¹, R³, J, G and Aare as defined in Table Z.1.

Table 1.14: This table discloses specific compounds of formula (I) wherein R^{2a} is methoxy, R^{2b} is H, R^{2c} is H and R⁶ is methoxy and wherein the values of R¹, R³, J, G and Aare as defined in Table Z.1.

Table 1.15: This table discloses specific compounds of formula (I) wherein R^{2a} is H, R^{2b} is methyl, R^{2c} is H and R⁶ is methyl and wherein the values of R¹, R³, J, G and Aare as defined in Table Z.1. Table 1.16: This table discloses specific compounds of formula (I) wherein R^{2a} is H, R^{2b} is methyl, R^{2c} is H and R⁶ is methoxy and wherein the values of R¹, R³, J, G and Aare as defined in Table Z.1.

Table 1.17: This table discloses specific compounds of formula (I) wherein R^{2a} is H, R^{2b} is methoxy, R^{2c} is H and R⁶ is methyl and wherein the values of R¹, R³, J, G and A are as defined in Table Z.1.

Table 1.18: This table discloses specific compounds of formula (I) wherein R^{2a} is H, R^{2b} is methoxy, R^{2c} is H and R⁶ is methoxy and wherein the values of R¹, R³, J, G and Aare as defined in Table Z.1.

Table 1.19: This table discloses specific compounds of formula (I) wherein R^{2a} is H, R^{2b} is fluoro, R^{2c} is H and R⁶ is methyl and wherein the values of R¹, R³, J, G and Aare as defined in Table Z.1.

Table 1.20: This table discloses specific compounds of formula (I) wherein R^{2a} is H, R^{2b} is fluoro, R^{2c} is H and R⁶ is methoxy and wherein the values of R¹, R³, J, G and Aare as defined in Table Z.1.

Table 1.21: This table discloses specific compounds of formula (I) wherein R^{2a} is H, R^{2b} is chloro, R^{2c} is H and R⁶ is methyl and wherein the values of R¹, R³, J, G and Aare as defined in Table Z.1.

Table 1.22: This table discloses specific compounds of formula (I) wherein R^{2a} is H, R^{2b} is chloro, R^{2c} is H and R⁶ is methoxy and wherein the values of R¹, R³, J, G and Aare as defined in Table Z.1.

Table 1.23: This table discloses specific compounds of formula (I) wherein R^{2a} is H, R^{2b} is bromo, R^{2c} is H and R⁶ is methyl and wherein the values of R¹, R³, J, G and Aare as defined in Table Z.1.

Table 1.24: This table discloses specific compounds of formula (I) wherein R^{2a} is H, R^{2b} is bromo, R^{2c} is H and R⁶ is methoxy and wherein the values of R¹, R³, J, G and Aare as defined in Table Z.1.

Table 1.25: This table discloses specific compounds of formula (I) wherein R^{2a} is H, R^{2b} is cyano, R^{2c} is H and R⁶ is methyl and wherein the values of R¹, R³, J, G and Aare as defined in Table Z.1.

Table 1.26: This table discloses specific compounds of formula (I) wherein R^{2a} is H, R^{2b} is cyano, R^{2c} is H and R⁶ is methoxy and wherein the values of R¹, R³, J, G and Aare as defined in Table Z.1.

Table 1.27: This table discloses specific compounds of formula (I) wherein R^{2a} is H, R^{2b} is H, R^{2c} is methyl and R⁶ is methyl and wherein the values of R¹, R³, J, G and Aare as defined in Table Z.1.

Table 1.28: This table discloses specific compounds of formula (I) wherein R^{2a} is H, R^{2b} is H, R^{2c} is methyl and R⁶ is methoxy and wherein the values of R¹, R³, J, G and A are as defined in Table Z.1.

Table 1.29: This table discloses specific compounds of formula (I) wherein R^{2a} is H, R^{2b} is H, R^{2c} is fluoro and R⁶ is methyl and wherein the values of R¹, R³, J, G and Aare as defined in Table Z.1. Table 1.30: This table discloses specific compounds of formula (I) wherein R^{2a} is H, R^{2b} is H, R^{2c} is fluoro and R⁶ is methoxy and wherein the values of R¹, R³, J, G and Aare as defined in Table Z.1.

Table 1.31: This table discloses specific compounds of formula (I) wherein R^{2a} is H, R^{2b} is H, R^{2c} is chloro and R⁶ is methyl and wherein the values of R¹, R³, J, G and Aare as defined in Table Z.1.

Table 1.32: This table discloses specific compounds of formula (I) wherein R^{2a} is H, R^{2b} is H, R^{2c} is chloro and R⁶ is methoxy and wherein the values of R¹, R³, J, G and Aare as defined in Table Z.1.

Table 1.33: This table discloses specific compounds of formula (I) wherein R^{2a} is H, R^{2b} is H, R^{2c} is bromo and R⁶ is methyl and wherein the values of R¹, R³, J, G and A are as defined in Table Z.1.

Table 1.34: This table discloses specific compounds of formula (I) wherein R^{2a} is H, R^{2b} is H, R^{2c} is bromo and R⁶ is methoxy and wherein the values of R¹, R³, J, G and Aare as defined in Table Z.1. Table 1.35: This table discloses specific compounds of formula (I) wherein R^{2a} is H, R^{2b} is H, R^{2c} is cyano and R⁶ is methyl and wherein the values of R¹, R³, J, G and Aare as defined in Table Z.1.

Table 1.36: This table discloses specific compounds of formula (I) wherein R^{2a} is H, R^{2b} is H, R^{2c} is cyano and R⁶ is methoxy and wherein the values of R¹, R³, J, G and Aare as defined in Table Z.1.

Compounds according to the invention may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile, improved physico-chemical properties, or increased biodegradability). Compounds according to the invention have particularly advantageous levels of biological activity for protecting plants against oomycetes such as Phytophthora, Plasmopara and Pythium.

Compounds of the present invention can be made as shown in the following schemes, in which, unless otherwise stated, the definition of each variable is as defined above for a compound of formula (1) as defined in embodiment 1.

Compounds of formula (I) wherein Z is O, can be made as shown in the following schemes 1 to 14, in which, unless otherwise stated, the definition of each variable is as defined in the present invention.

Compounds of formula (I) can be prepared via Suzuki cross coupling of compounds of formula (II), wherein X is chloro (Cl), bromo (Br) or iodo (I), and a compound of formula (III), wherein either R⁷ is independently from each other hydrogen, C₁-C₆ alkyl or wherein two R⁷ together can form a C₃-C₈ cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. This transformation is depicted in Scheme 1.

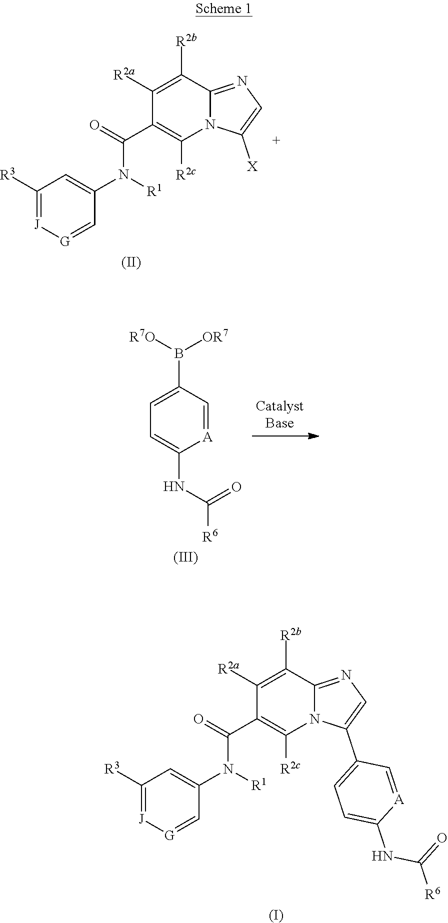

Compounds of formula (II), wherein X is Cl, Br or I, can be prepared by the reaction of a compound of formula (IV), wherein X is Cl, Br or I, with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such, as triethylamine, ethyldiisopropylamine or N-methylmorpholine in a suitable solvent such ethyl acetate, dimethylformamide, tetrahydrofuran or dichloromethane. This transformation is depicted in Scheme 2.

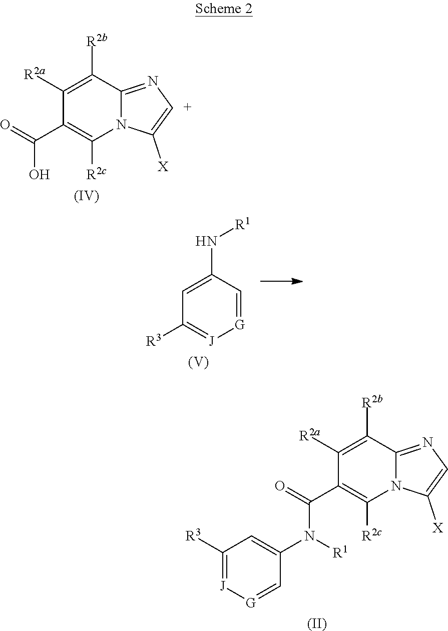

Compounds of formula (IV), wherein X is Cl, Br or I, are commercially available or, alternatively can be prepared by the saponification of compounds of formula (VI), wherein X is Cl, Br or I and R⁸ is a C₁-C₆ alkyl, using a base, such as NaOH or LiOH, in a suitable solvent such as methanol, ethanol or water at temperature between retention time (RT) and reflux. This transformation is depicted in Scheme 3.

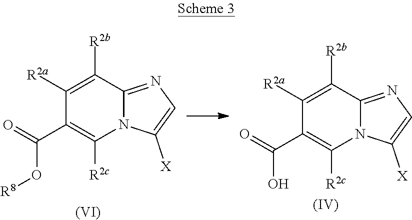

Alternatively, compounds of formula (II), wherein X is Cl, Br or I, can be prepared directly by the reaction of a compounds of formula (VI), wherein X is Cl, Br or I and R⁸ is a C₁-C₆ alkyl, and a compounds of formula (V) in the presence of (CH₃)₃Al or Bis(trimethylaluminum)-1,4-diazabicyclo[2.2.2]octane adduct in a suitable solvent, such as tetrahydrofuran or toluene. These transformations have been described in the literature (see for examples: Weinreb, S. M. et al. Tetrahedron Lett. 1977, 48, 4171; Woodward, S. et al. in Tetrahedron Letters 2006, 47, 5767; Woodward S. et al. in Org. Process Res. Dev. 2015, 19, 831). This transformation is depicted in scheme 4.

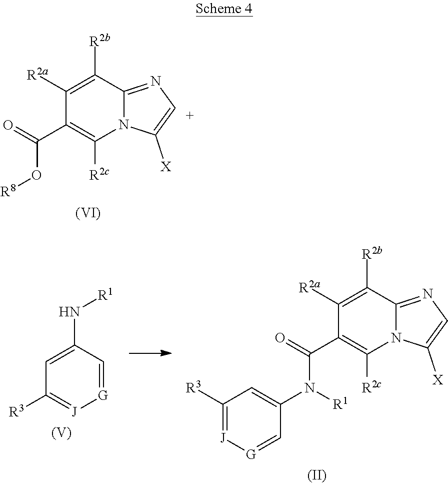

Compounds of formula (VI), wherein X is Cl, Br or I and R⁸ is a C₁-C₆ alkyl, are commercially available or, alternatively, can be prepared from the reaction of a compound of formula (VII), wherein R⁸ is a C₁-C₆ alkyl, and a halogenating agent, such as N-chlorosuccinimide, N-bromosuccinimide, N-iodosuccinimide or bromine in a suitable solvent, such as dichloromethane, chloroform, tetrahydrofuran or acetonitrile. This transformation is depicted in scheme 5.

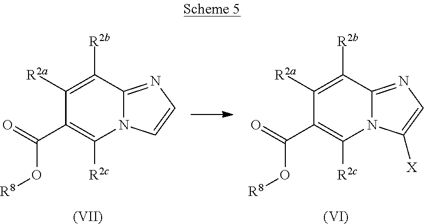

Compounds of formula (VII), wherein R⁸ is a C₁-C₆ alkyl, are commercially available or, alternatively, can be prepared by the reaction of a compound of formula (VIII), wherein X is Cl, Br or I, with carbon monoxide and an alcohol R⁸OH, wherein R⁸ is a C₁-C₆ alkyl, in the presence of a catalyst, such as PdCl2dppf, and, optionally, a base such as triethylamine. This transformation is depicted in scheme 6.

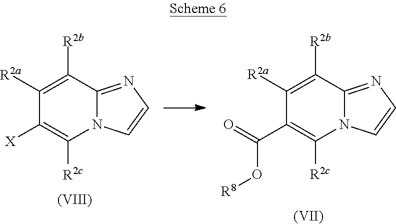

Compounds of formula (VIII) are commercially available or, alternatively, can be prepared by the reaction of a compound of formula (IX), wherein X is Cl, Br or I, and a compound of formula (X), wherein X is Cl, Br or I, or its corresponding acetal of formula (XI), wherein X is Cl, Br or I and either R⁹ is independently from each other C₁-C₆ alkyl or wherein two R⁹ together can form a C₃-C₈ cycloalkyl, in a solvent, such as water, ethanol, acetone or acetonitrile. In some instance, the outcome of the reaction can be improved by using a base, such as sodium bicarbonate or potassium carbonate, or by using an acid, such as p-toluenesulfonic acid or hydrogen bromide. Additionally, this transformation can be utilized to prepare compounds of formula (VII), wherein R⁸ is a C₁-C₆ alkyl, from a compound of formula (XII), wherein R⁸ is a C₁-C₆alkyl, and to prepare compounds of formula (XIII) from a compound of formula (XIV). These transformations are depicted in scheme 7

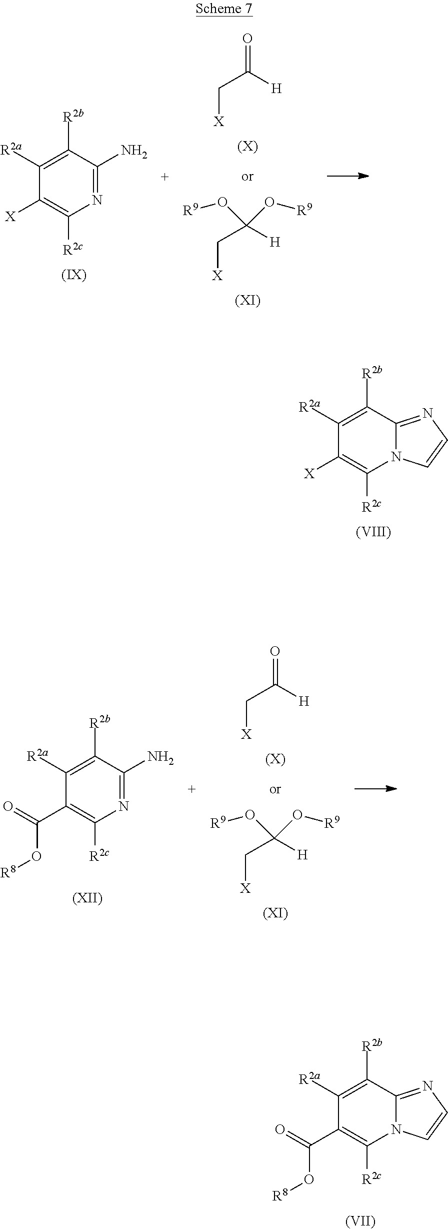

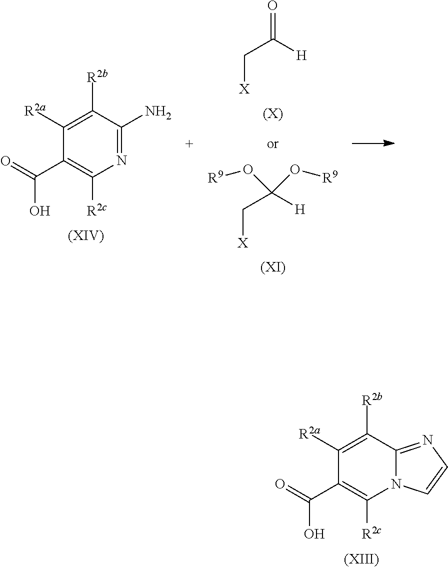

Compounds of formula (IX), wherein X is Cl, Br or I, compounds of formula (XII), wherein R⁸ is a C₁-C₆ alkyl, and compounds of formula (XIV) are prepared by known methods or are commercially available.

Alternatively, compounds of formula (I) can be prepared by the reaction of a compound of formula (XV), with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphophonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. This transformation is depicted in Scheme 8.

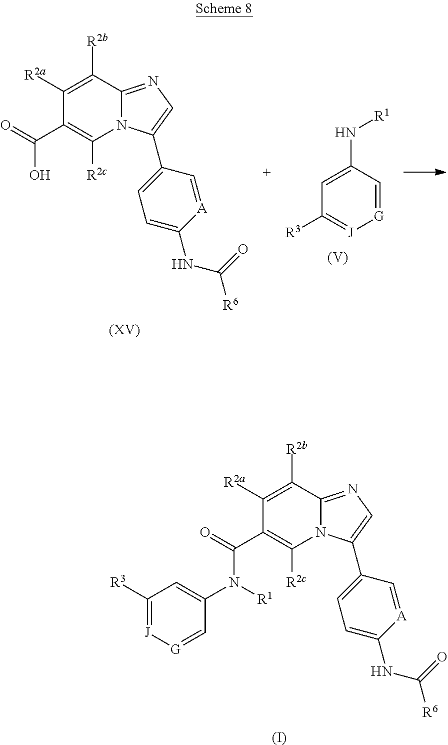

Compound of formula (XV) can be prepared by the saponification of compounds of formula (XVI), wherein R⁸ is a C₁-C₆ alkyl, using a base such as NaOH or LiOH, in a suitable solvent such as methanol, ethanol or water at temperature between RT and reflux. This transformation is depicted in Scheme 9.

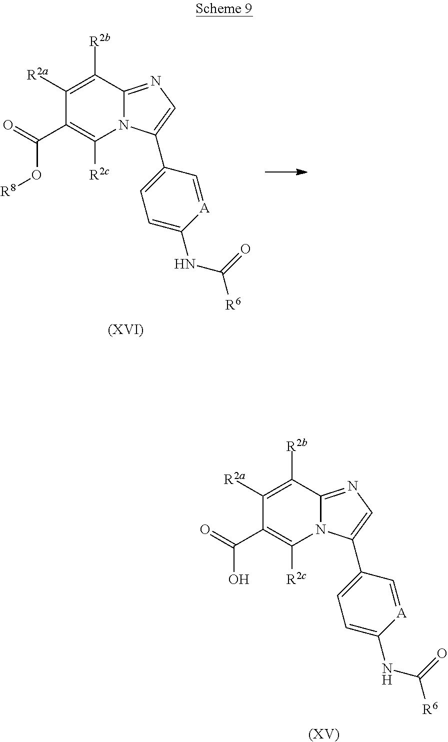

Compounds of formula (XVI), wherein R⁸ is a C₁-C₆ alkyl, can be prepared via Suzuki cross coupling of compounds of formula (VI), wherein X is Cl, Br or I, and R⁸ is C₁-C₆ alkyl, and a compound of formula (III), wherein either R⁷ is independently from each other hydrogen, C₁-C₆ alkyl or wherein two R⁷ together can form a C₃-C₈ cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. Additionally, this transformation can be utilized to prepare a compound of formula (XV) from a compound of formula (IV). These transformations are depicted in Scheme 10.

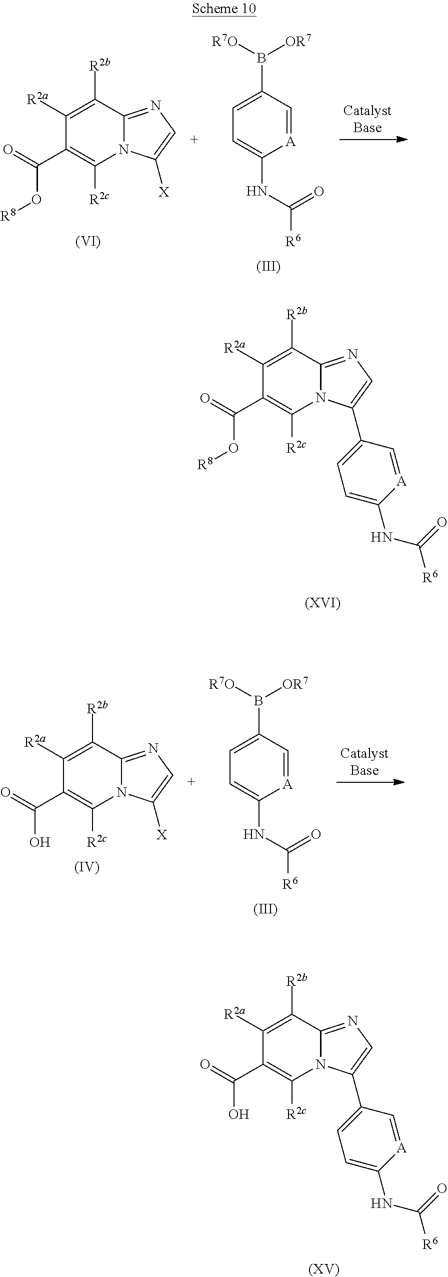

Alternatively, compounds of formula (II), wherein X is Cl, Br or I, can be prepared by the reaction of a compound of formula (XVII) and a halogenating agent, such as N-chlorosuccinimide, N-bromosuccinimide, N-iodosuccinimide or bromine in a suitable solvent, such as dichloromethane, chloroform, tetrahydrofuran or acetonitrile. This transformation is depicted in scheme 11.

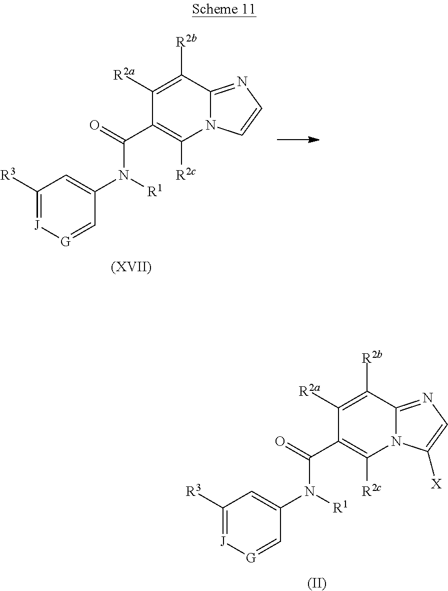

Compounds of formula (XVII) can be prepared by the reaction of a compound of formula (XIII), with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphophonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. This transformation is depicted in Scheme 12.

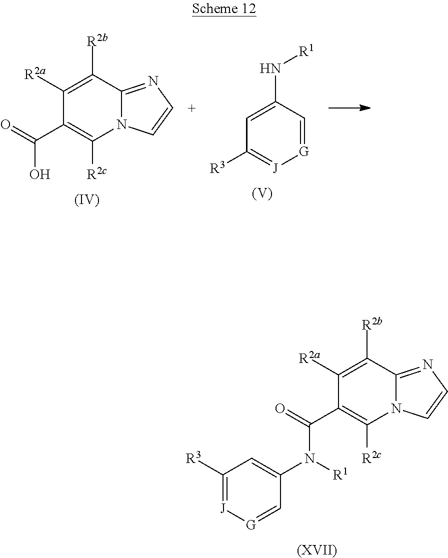

Alternatively, compounds of formula (II) can be prepared by the reaction of a compound of formula (XVIII), with a compounds of formula (XIX), wherein Y is Cl, Br, I, OSO₂CF₃, OSO₂C₆H₄CH₃ or OSO₂CH₃, in the presence of a base, such as Cs₂CO₃, K₂CO₃ or NaOtBu. This transformation is depicted in scheme 13.

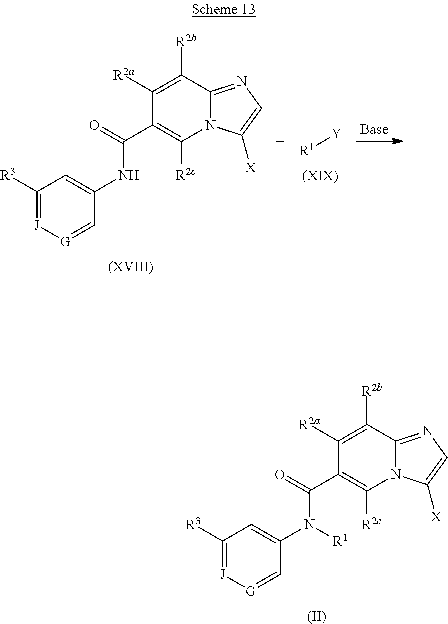

Compounds of formula (XVIII), wherein X is Cl, Br or I, can be prepared by the reaction of a compound of formula (IV), wherein X is Cl, Br or I, with a compounds of formula (XX) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such, as triethylamine, ethyldiisopropylamine or N-methylmorpholine in a suitable solvent such ethyl acetate, dimethylformamide, tetrahydrofuran or dichloromethane. This transformation is depicted in Scheme 14.

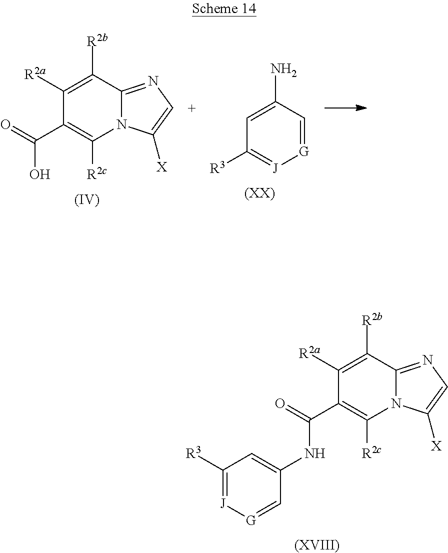

Alternatively, compounds of formula (I) can be prepared by the reaction of a compound of formula (XXI) with a compound of formula (XXII), wherein Y is OH, and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. The transformation can also be accomplished by the reaction of a compound of formula (XXI) with a compound of formula (XXII), wherein Y is Cl, and, optionally, a base such as, triethylamine, ethyldiisopropylamine or pyridine in a suitable solvent such as ethyl acetate, pyridine or tetrahydrofuran. This transformation is depicted in Scheme 15.

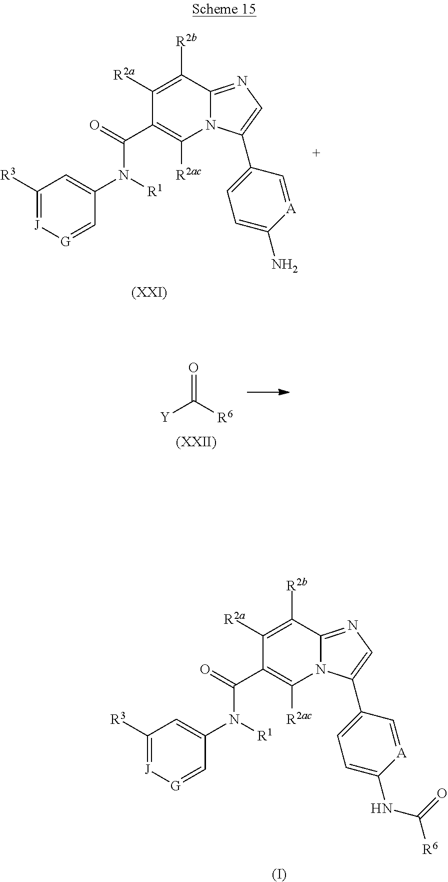

Compounds of formula (XXI) can be prepared via Suzuki cross coupling of compounds of formula (II), wherein X is Cl, Br or I, and a compound of formula (XXIII), wherein either R⁷ is independently from each other hydrogen, C₁-C₆ alkyl or wherein two R⁷ together can form a C₃-C₈ cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. This transformation is depicted in Scheme 16.

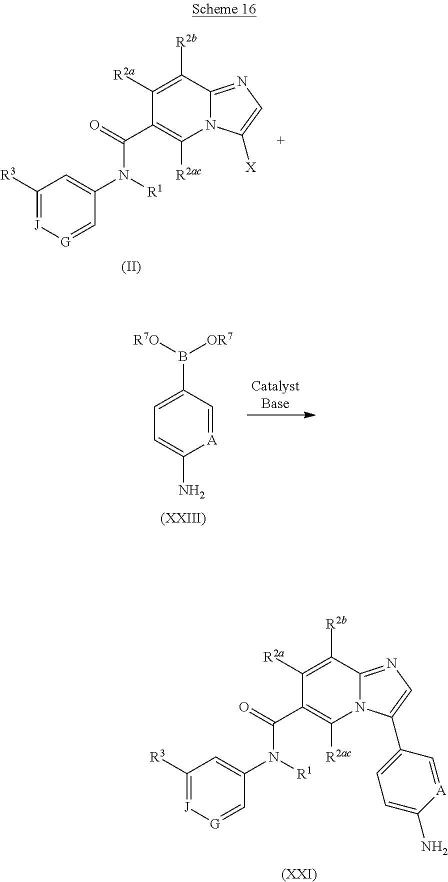

Compounds of formula (V) and of formula (XX) are prepared by known methods or are commercially available.

Compounds of formula (III), wherein either R⁷ is independently from each other hydrogen, C₁-C₆ alkyl or wherein two R⁷ together can form a C₃-C₈ cycloalkyl are prepared by known methods or are commercially available.

It is understood that a person skilled in the art would recognize that the amide coupling reactions described above between an acid, an amine and a coupling agent could also be performed using the corresponding acid chloride and amine. The transformation of an acid into its corresponding acid chloride is well known for the person skilled in the art.

Compounds of formula (Ib), can be prepared by the reaction of a compound of formula (I) wherein Z is O, with phosphorus pentasulfide or Lawesson's reagent (CAS: 19172-47-5) in a suitable solvent such as toluene, xylene or dichloromethane. This transformation is depicted in Scheme 17.

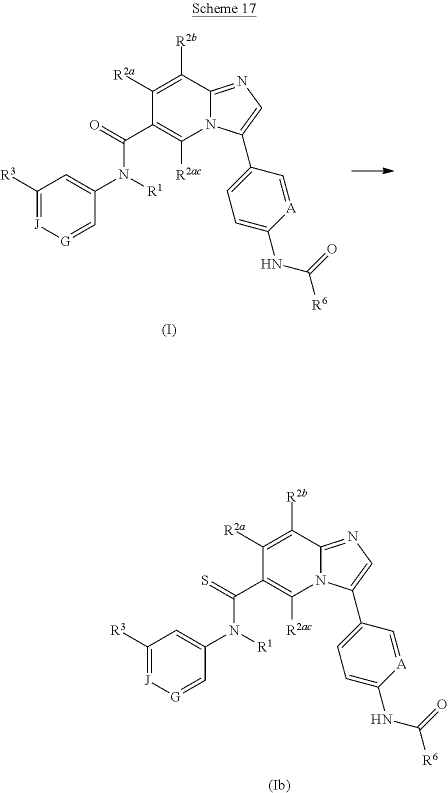

Alternatively, compounds of formula (Ib), wherein Z is S, can be prepared by the reaction of a compound of formula (XXIV) with a compound of formula (XXII), wherein Y is OH, and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. The transformation can also be accomplished by the reaction of a compound of formula (XXI) with a compound of formula (XXII), wherein Y is Cl, and, optionally, a base such as, triethylamine, ethyldiisopropylamine or pyridine in a suitable solvent such as ethyl acetate, pyridine or tetrahydrofuran. These transformations are depicted in Scheme 18.

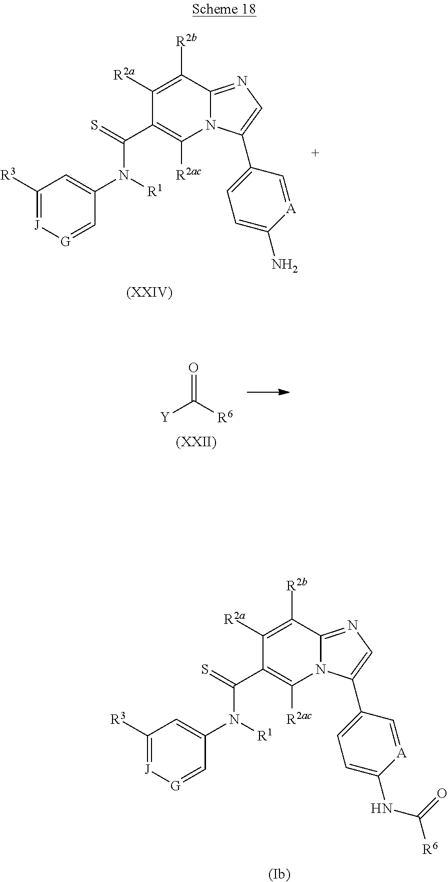

Compounds of formula (XXIV) can be prepared by the reaction of a compound of formula (XXI), with phosphorus pentasulfide or Lawesson's reagent (CAS: 19172-47-5) in a suitable solvent such as toluene, xylene or dichloromethane. This transformation is depicted in Scheme 19.

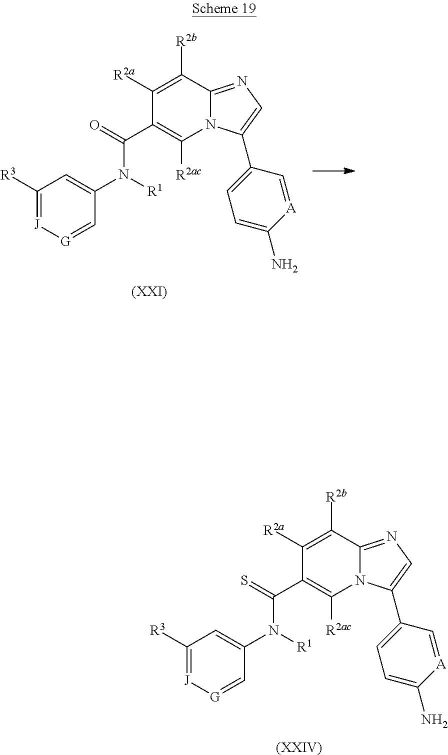

When the term “compound/compounds according to the invention” is used, then this refers to compounds according to any one of embodiments 1 to 43 as defined above.

Alternatively, the compounds according to any one of embodiments 1 to 43, can be obtained by using standard synthesis techniques known to the person skilled in the art. Non-exhaustive examples include oxidation reactions, reduction reactions, hydrolysis reactions, coupling reactions, aromatic nucleophilic or electrophilic substitution reactions, nucleophilic substitution reactions, nucleophilic addition reactions, olefination reactions, oxime formation, alkylation and halogenation reactions.

A compound according to any one of embodiments 1 to 43 can be converted in a manner known per se into another compound according to any one of embodiments 1 to 43 by replacing one or more substituents of the starting compound according to any one of embodiments 1 to 43 in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of the compounds according to any one of embodiments 1 to 43 can be prepared in a manner known per se. Thus, for example, acid addition salts of the compounds according to any one of embodiments 1 to 43 are obtained by treatment with a suitable acid or a suitable ion exchanger reagent and salts with bases are obtained by treatment with a suitable base or with a suitable ion exchanger reagent.

Salts of compounds the compounds according to any one of embodiments 1 to 43 can be converted in the customary manner into the free compounds, acid addition salts, for example, by treatment with a suitable basic compound or with a suitable ion exchanger reagent and salts with bases, for example, by treatment with a suitable acid or with a suitable ion exchanger reagent.

Salts of the compounds according to any one of embodiments 1 to 43 can be converted in a manner known per se into other salts of the compounds according to any one of embodiments 1 to 43, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds according to any one of embodiments 1 to 43, which have salt-forming properties can be obtained in free form or in the form of salts.

The compounds according to any one of embodiments 1 to 43 and, where appropriate, the tautomers thereof, in each case in free form or in salt form, can be present in the form of one of the stereoisomers which are possible or as a mixture of these, for example in the form of pure stereoisomers, such as antipodes and/or diastereomers, or as stereoisomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure stereoisomers and also to all stereoisomer mixtures which are possible and is to be understood in each case in this sense hereinabove and hereinbelow, even when stereochemical details are not mentioned specifically in each case.

Diastereomer mixtures or racemate mixtures of the compounds according to any one of embodiments 1 to 43, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable stereoisomer mixtures, but also by generally known methods of diastereoselective or enantioselective synthesis, for example by carrying out the process according to the invention with starting materials of a suitable stereochemistry.

N-oxides can be prepared by reacting a compound according to any one of embodiments 1 to 5 with a suitable oxidizing agent, for example the H2O2/urea adduct in the presence of an acid anhydride, e.g. trifluoroacetic anhydride. Such oxidations are known from the literature, for example from J. Med. Chem., 32 (12), 2561-73, 1989 or WO 00/15615.

It is advantageous to isolate or synthesize in each case the biologically more effective stereoisomer, for example enantiomer or diastereomer, or stereoisomer mixture, for example enantiomer mixture or diastereomer mixture, if the individual components have a different biological activity.

The compounds according to any one of embodiments 1 to 5 and, where appropriate, the tautomers thereof, in each case in free form or in salt form, can, if appropriate, also be obtained in the form of hydrates and/or include other solvents, for example those which may have been used for the crystallization of compounds which are present in solid form.

The following Examples illustrate, but do not limit, the invention.

The present invention also provides intermediates useful for the preparation of compounds according to any one of embodiments 1 to 43.

The below intermediates are novel and as such form a further aspect of the invention.

A compound of formula (XV)

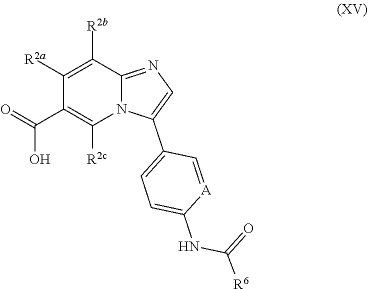

wherein A is CH or N;
R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN, and preferably R⁶ is selected from C₁₋₄alkyl, C₁₋₄alkoxy, C₃₋₆cycloalkyl and C₃₋₆cycloalkyl-C₁₋₃alkyl, wherein each of said groups is optionally substituted with one to three substituents independently selected from halogen; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy.

The preferences for A, R^{2a}, R^{2b}, R^{2c}, and R⁶ are the same as the preferences set out for the corresponding substituents of the compound of formula (I).

A compound of formula (XVI)

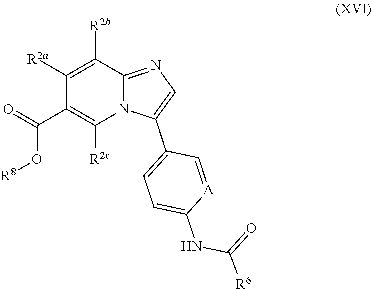

wherein A is CH or N;
R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN; and preferably R⁶ is selected from C₁₋₄alkyl, C₁₋₄alkoxy, C₃₋₆cycloalkyl and C₃₋₆cycloalkyl-C₁₋₃alkyl, wherein each of said groups is optionally substituted with one to three substituents independently selected from halogen;
R⁸ is C₁-C₆ alkyl, and preferably R⁸ is selected from methyl and ethyl; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy.

The preferences for A, R^{2a}, R^{2b}, R^{2c}, and R⁶ are the same as the preferences set out for the corresponding substituents of the compound of formula (I).

A compound of formula (XXI)

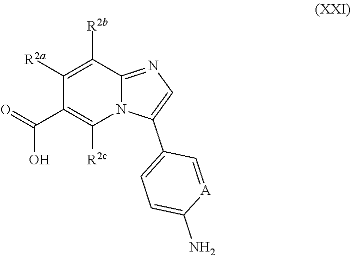

wherein A is CH or N; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy.

The preferences for R^{2a}, R^{2b} and R^{2c} are the same as the preferences set out for the corresponding substituents of the compound of formula (I).

A compound of formula (XXII)

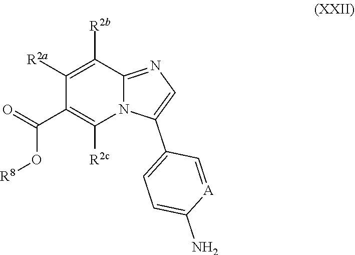

wherein A is CH or N;
R⁸ is C₁-C₆ alkyl, and preferably R^{a} is selected from methyl and ethyl; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy.

The preferences for R^{2a}, R^{2b} and R^{2c} are the same as the preferences set out for the corresponding substituents of the compound of formula (I).

A compound of formula (II)

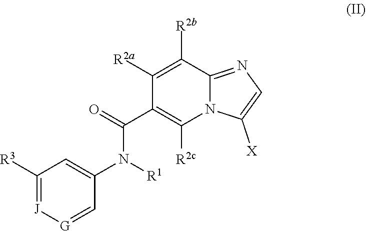

wherein R¹ is selected from C₁₋₄alkyl, C₁₋₄alkoxy-C₁₋₄alkyl, C₃-cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, hydroxy, halogen, CN, C₁₋₆alkyl, C₃₋₆ycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy;

### J is CR⁴ or N;

### G is CR⁵ or N;

wherein when J is N, then G is CR⁵; and when G is N, then J is CR⁴;
R³, R⁴ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN, and preferably R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and
X is Cl, Br or I, and more preferably X is Cl or I.

In a particular embodiment, R³ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN, and preferably R³ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and

R⁴ is selected from H, C₁₋₆alkyl, C₁₋₆alkoxy, C₁₋₆alkoxy-C₁₋₆alkyl, C₁₋₆alkoxy-C₁₋₆alkoxy, halogen, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN, and preferably R⁴ is selected from H, C₁₋₄alkyl, C₁₋₄alkoxy, C₁₋₄alkoxy-C₁₋₄alkyl, C₁₋₄alkoxy-C₁₋₄alkoxy, halogen, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

The preferences for R¹, R^{2a}, R^{2b}, R^{2c}, R³, J, G, R³, R⁴ and R⁵ are the same as the preferences set out for the corresponding substituents of the compound of formula (I).

In a more preferred embodiment according to the compound of formula (II), R⁴ is independently selected from H, C₁₋₄alkyl, C₁₋₄alkoxy, C₁₋₄alkoxy-C₁₋₄alkyl, C₁₋₄alkoxy-C₁₋₄alkoxy, halogen, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and/or X is Cl or I.

A compound of formula (XVII)

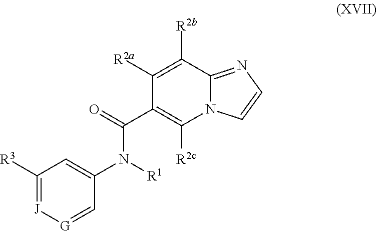

wherein R¹ is selected from C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl, and preferably R^{2a}, R^{2b} and R^{2c} are independently selected from H, halogen, CN, C₁₋₄alkyl and C₁₋₄alkoxy;

### J is CR⁴ or N;

### G is CR⁵ or N;

wherein when J is N, then G is CR⁵; and when G is N, then J is CR⁴; and
R³, R⁴ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN, and preferably R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

The preferences for R¹, R^{2a}, R^{2b}, R^{2c}, R³, J, G, R³, R⁴ and R⁵ are the same as the preferences set out for the corresponding substituents of the compound of formula (I).

In a more preferred embodiment according to the compound of formula (XVII), at least one of R³, R⁴ and R⁵ is not H, and more particularly:
R³ and R⁴ are independently selected from C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and
R⁵ is selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen.

In another more preferred embodiment according to the compound of formula (XVII):
R³ is selected from H, C₁₋₄alkyl, C₁₋₄alkoxy, C₁₋₄alkoxy-C₁₋₄alkyl, C₁₋₄alkoxy-C₁₋₄alkoxy, F, Br, I, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and
R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen. In said another more preferred embodiment, R³ and R⁵ are more particularly not Cl.

In another more preferred embodiment according to the compound of formula (XVII):
R³ is selected from C₁₋₄alkyl, C₁₋₄alkoxy, C₁₋₄alkoxy-C₁₋₄alkyl, C₁₋₄alkoxy-C₁₋₄alkoxy, F, Br, I, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen; and
R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkenyl, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkenyl, C₂₋₄alkynyl, C₃₋₆cycloalkyl and C₃₋₆cycloalkyl-C₁₋₃alkyl groups is optionally substituted with one to three substituents independently selected from halogen. In said another more preferred embodiment, R³ and R⁵ are more particularly not Cl.

The compounds of formula (I) as defined in any one of embodiments 1 to 43 can be used in the agricultural sector and related fields of use e.g. as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula (I) as defined in any one of embodiments 1 to 43 can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula (I) as defined in any one of embodiments 1 to 43 as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It is also possible to use compounds of formula (I) as defined in any one of embodiments 1 to 43 as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula (I) as defined in any one of embodiments 1 to 43 before planting: seed, for example, can be dressed before being sown. The compounds of formula (I) as defined in any one of embodiments 1 to 43 can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds of formula (I) as defined in any one of embodiments 1 to 43 can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

Compounds of formula (I) as defined in any one of embodiments 1 to 43 and fungicidal compositions containing them may be used to control plant diseases caused by a broad spectrum of fungal plant pathogens. They are effective in controlling a broad spectrum of plant diseases, such as foliar pathogens of ornamental, turf, vegetable, field, cereal, and fruit crops.

These fungi and fungal vectors of disease, as well as phytopathogenic bacteria and viruses, which may be controlled are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea,
Coccidioides immitis, Cochliobolus spp, Colletotrichum spp. including C. musae,
Cryptococcus neoformans, Diaporthe spp, Didymella spp, Drechslera spp, Elsinoe spp,
Epidermophyton spp, Erwinia amylovora, Erysiphe spp. including E. cichoracearum,
Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi,
Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp. including T. harzianum, T. pseudokoningii, T. viride,
Trichophyton spp, Typhula spp, Uncinula necator, Urocystis spp, Ustilago spp, Venturia spp. including V. inaequalis, Verticillium spp, and Xanthomonas spp.

In particular, compounds of formula (I) as defined in any one of embodiments 1 to 43 and fungicidal compositions containing them may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes. More particularly, the compounds of formula (I) as defined in any one of embodiments 1 to 43 may be used to control oomycetes.

These pathogens may include:

Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare, Pythium sylvaticum and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola.
Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f. sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae.
Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f. sp. Hordei, Puccinia striiformis f. sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries.
Blastocladiomycetes, such as Physoderma maydis.
Mucoromycetes, such as Choanephora cucurbitarum; Mucor spp.; Rhizopus arrhizus.
As well as diseases caused by other species and genera closely related to those listed above.

In addition to their fungicidal activity, the compounds and compositions comprising compounds of formula (I) as defined in any one of embodiments 1 to 43 may also have activity against bacteria such as Erwinia amylovora, Erwinia caratovora, Xanthomonas campestris, Pseudomonas syringae, Streptomyces scabies and other related species as well as certain protozoa.

Within the scope of the present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 6-endotoxins, vegetative insecticidal proteins (Vip), insecticidal proteins of bacteria colonising nematodes, and toxins produced by scorpions, arachnids, wasps and fungi. An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Toxins that can be expressed by transgenic plants include, for example, insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO03/018810).

More examples of such toxins or transgenic plants capable of synthesising such toxins are disclosed, for example, in EP-A-0 374 753, WO93/07278, WO95/34656, EP-A-0 427 529, EP-A-451 878 and WO03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. B11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 19th Ed., British Crop Protection Council 2021. The compounds of formula (I) as defined in any one of embodiments 1 to 43 may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and/or adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and/or carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides, plant growth regulators, and/or biologicals.

The following mixtures of the compounds of formula I with active ingredients are preferred (where the abbreviation “TX” means “one compound selected from the compounds defined in the Tables 1.1 to 1.36” or “one compound selected from the compounds defined in the Table A”):

an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: RNA (Leptinotarsa decemlineata-specific recombinant double-stranded interfering GS2)+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirobudifen+TX; spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tiorantraniliprole+TX; tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX; a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, Gossyplure® (alternative name; 1:1 mixture of the (Z,E and (Z,Z) isomers of hexadeca-7,11-dien-1-yl-acetate) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B, (alternative name) (839)+TX, trimedlure B₂ (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX; a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chlorophenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B, +TX, trimedlure B₂+TX, trimedlure C+TX, trunc-call+TX, 2-(octylthio)ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imibenconazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl+TX, R-metalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1, 1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1, 1-dimethylethyl)-α-[4′-(trifluoromethoxy) [1,1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (2)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifluoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipactermichiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®®)+TX, Streptomyces lydicus (Actinovate®®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect killerf)+TX, Glycinebetaine (Greenstim®+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®+TX, Reynoutria sachalinensis (Regalia®+TX, Sakalia®)+TX, rotenone (Eco Rotenf)+TX, Rutaceae plant extract (Soleo®)+TX, soybean oil (Ortho ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®);
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable pheromone®)+TX, Peachtree Borer Pheromone (Isomate-P®)+TX, Tomato Pinworm Pheromone (3M Sprayable pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®®)+TX, Aphidius ervi (Ervipar®®)+TX, Aphidius gifuensis+TX, Aphidius matricariae (Aphipar-M®)+TX, Aphidoletes aphidimyza (Aphidend®®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-rid®+TX, Entonem®+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator;
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®+TX, potassium salts of fatty acids (Sanova®)+TX, potassium silicate solution (Sil-Matrix®+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;
(1) antibacterial agents selected from the group of:
(1.1) bacteria, examples of which are Bacillus mojavensis strain R3B (Accession No. NCAIM (P) B001389) (WO 2013/034938) from Certis USA LLC, a subsidiary of Mitsui & Co.+TX; Bacillus pumilus, in particular strain BU F-33, having NRRL Accession No. 50185 (available as part of the CARTISSA® product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661, U.S. Pat. No. 6,060,051)+TX; Bacillus subtilis strain BU1814, (available as VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC, a subsidiary of Mitsui & Co.+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX+TX; Bacillus licheniformis FMCH001 and Bacillus subtilis FMCH002 (QUARTZO® (WG) and PRESENCE® (WP) from FMC Corporation)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC, a subsidiary of Mitsui & Co.+TX; Bacillus mycoides, isolate, having Accession No. B-30890 (available as BMJ TGAI® or WG and LifeGard™ from Certis USA LLC, a subsidiary of Mitsui & Co.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BS03 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botectorm by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321 U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), or strain IK726 (Jensen D F, et al. Development of a biocontrol agent for plant disease control with special emphasis on the near commercial fungal antagonist Clonostachys rosea strain ‘IK726’, Australas Plant Pathol. 2007, 36:95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (having Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICC080 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO® 7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1 (e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. and BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB—SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens μm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BP01 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (having Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (having Accession Nos. NRRL B-50421 and described in U.S. patent application Ser. No. 13/330,576)+TX; Bacillus subtilis, in particular strain AQ30004 (and NRRL B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCH002 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and (3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel from Agrimm Technologies Limited)+TX; Trichoderma atroviride strain SC1 described in International Application No. PCT/IT2008/000196)+TX; Trichoderma asperellum strain kd (e.g. T-Gro from Andermatt Biocontrol)+TX; Trichoderma asperellum strain Eco-T (Plant Health Products, ZA), Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. comprised in Myco-Sol from Helena Chemical Company)+TX; Rhizopogon fulvigleba (e.g. comprised in Myco-Sol from Helena Chemical Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC, a subsidiary of Mitsui & Co.)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 from Becker Microbial Products, IL+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11, (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX from Certis, US)+TX; Bacillus thuringiensis subsp. kurstaki strain EG 7841 (CRYMAX from Certis, US)+TX; Bacillus thuringiensis subsp. tenebrionis strain NB 176 (SD-5428, e.g. NOVODOR® FC from BioFa DE)+TX; Brevibacillus laterosporus (LATERAL from Ecolibrium Biologicals)+TX; Burkholderia spp., in particular Burkholderia rinojensis strain A396 (also known as Burkholderia rinojensis strain MBI 305) (Accession No. NRRL B-50319+TX; WO 2011/106491 and WO 2013/032693+TX; e.g. MB1206 TGAI and ZELTO® from Marrone Bio Innovations)+TX; Chromobacterium subtsugae, in particular strain PRAA4-1T (MBI-203+TX; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ and MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam International Corporation)+TX; Beauveria bassiana strain ATPO2 (Accession No. DSM 24665)+TX; Isaria fumosorosea (previously known as Paecilomyces fumosoroseus) strain Apopka 97) PREFERAL from SePRO+TX; Metarhizium anisopliae 3213-1 (deposited under NRRL accession number 67074) (WO 2017/066094+TX; Pioneer Hi-Bred International)+TX; Metarhizium robertsii 15013-1 (deposited under NRRL accession number 67073)+TX; Metarhizium robertsii23013-3 (deposited under NRRL accession number 67075)+TX; Paecilomyces lilacinus strain 251 (MELOCON from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray+TX; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Heads Up (Chenopodium quinoa saponin extract)+TX; PROBLAD (naturally occurring Blad polypeptide from Lupin seeds), Certis EU+TX; FRACTURE (naturally occurring Blad polypeptide from Lupin seeds), FMC+TX; Pyrethrum/Pyrethrins+TX; Quassia amara+TX; Quercus+TX; Quillaja extract (QL AGRI 35 from BASF)+TX; Reynoutria sachalinensis extract (REGALLIA/REGALIA MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) as defined in any one of embodiments 1 to 43 are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) as defined in any one of embodiments 1 to 43 may be used in the form of (fungicidal) compositions for controlling or protecting against phytopathogenic microorganisms, comprising as active ingredient at least one compound of formula (I) as defined in any one of embodiments 1 to 43 or of at least one preferred individual compound as above-defined, in free form or in agrochemically usable salt form, and at least one of the above-mentioned adjuvants.

The invention therefore provides a composition, preferably a fungicidal composition, comprising at least one compound of formula (I) as defined in any one of embodiments 1 to 43, an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally active compounds, for example an additional fungicidal active ingredient in addition to the compound of formula (I) as defined in any one of embodiments 1 to 43.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula (I) as defined in any one of embodiments 1 to 43 or of a preferred individual compound as above-defined as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula (I) as defined in any one of embodiments 1 to 43, or an agrochemical composition which contains at least one of said compounds, is foliar application. The frequency of application and the rate of application will depend on the risk of infestation by the corresponding pathogen or insect. However, the compounds of formula (I) as defined in any one of embodiments 1 to 43 can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) as defined in any one of embodiments 1 to 43 may also be applied to seeds (coating) by impregnating the seeds or tubers either with a liquid formulation of the fungicide or coating them with a solid formulation.

A formulation, e.g. a composition containing the compound of formula (I) as defined in any one of embodiments 1 to 43, and, if desired, a solid or liquid adjuvant or monomers for encapsulating the compound of formula (I) as defined in any one of embodiments 1 to 43, may be prepared in a known manner, typically by intimately mixing and/or grinding the compound with extenders, for example solvents, solid carriers and, optionally, surface active compounds (surfactants).

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is preferably 1 g to 2000 g of active ingredient per hectare, more preferably 10 to 1000 g/ha, most preferably 10 to 600 g/ha. When used as seed drenching agent, convenient dosages are from 10 mg to 1 g of active substance per kg of seeds.

When the combinations of the present invention are used for treating seed, rates of 0.001 to 50 g of a compound of formula (1) per kg of seed, preferably from 0.01 to 10 g per kg of seed are generally sufficient.

Suitably, a composition comprising a compound of formula (I) as defined in any one of embodiments 1 to 43 according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) as defined in any one of embodiments 1 to 43 together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

### EXAMPLES

The Examples which follow serve to illustrate the invention. Certain compounds of the invention can be distinguished from known compounds by virtue of greater efficacy at low application rates, which can be verified by the person skilled in the art using the experimental procedures outlined in the Examples.

Throughout this description, temperatures are given in degrees Celsius (° C.) and “mp.” means melting point. LC/MS means Liquid Chromatography Mass Spectrometry and the description of the apparatus and the method is as follows:

The LC/MS apparatuses and methods are as follow:

### Method A:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

### Method B:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: O-100% B in 2.5 min; Flow (ml/min) 0.75.
Method 1 and Method 2: Spectra were recorded on one of the instruments described below and using the method 1 or 2 following it:
   Agilent 1100 Series LC/MSD system with DAD\ELSD Alltech 2000ES and Agilent LC\MSD VL (G1956B), SL (G1956B) mass-spectrometer.
   Agilent 1200 Series LC/MSD system with DAD\ELSD Alltech 3300 and Agilent LC\MSD G6130A, G6120B mass-spectrometer.
   Agilent Technologies 1260 Infinity LC/MSD system with DAD\ELSD Alltech 3300 and Agilent LC\MSDG6120B mass-spectrometer.
   Agilent Technologies 1260 Infinity II LC/MSD system with DAD\ELSD G7102A 1290 Infinity II and Agilent LC\MSD G6120B mass-spectrometer.
   Agilent 1260 Series LC/MSD system with DAD\ELSD and Agilent LC\MSD (G6120B) mass-spectrometer.
   UHPLC Agilent 1290 Series LC/MSD system with DAD\ELSD and Agilent LC\MSD (G6125B) mass-spectrometer.

### Details of Method 1:

Column: Agilent Poroshell 120 SB—C18 4.6×30 mm 2.7 μm
Column Temperature: 60° C.
Mobile phase: A—water (0.1% formic acid), B—acetonitrile (0.1% formic acid)
Flow rate: 3 ml/min
Gradient: 0.01 min—1% B, 1.5 min—100% B, 1.73 min—100% B
MS Ionization mode: Electrospray ionization (ESI)
MS Scan range: 83-600 m/z
UV detection: 215 nm, 254 nm, 280 nm

### Details of Method 2:

Column: Agilent Poroshell 120 SB—C18 4.6×30 mm 2.7
Temperature: 60° C.
Mobile phase: A—water (0.1% formic acid), B—acetonitrile (0.1% formic acid)
Flow rate: 3 ml/min
Gradient: 0.01 min—1% B, 1.5 min—100% B, 2.2 min—100% B.
MS Ionization mode: Electrospray ionization (ESI)
MS Scan range: 83-1000 m/z
UV detection: 215 nm, 254 nm, 280 nm

¹H NMR spectra were recorded on a Bruker 400 MHz or Varian Unity 400 spectrometer at 400 MHz using CDCl₃ or DMSO-d6 as solvent and tetramethylsilane as internal standard. Chemical shifts are reported in ppm downfield from the standard (tetramethylsilane=0.00 ppm), coupling constants are in Hz.

The below Table A gathers for compounds of formula (I):
LC/MS data, such as retention time (RT), [M+H]⁺, and the type of method (Method A, Method B, Method 1, or Method 2), and/or
melting point (mp).

TABLE A

| | | | | [M + H] | | |
|---|---|---|---|---|---|---|
| En- | | | RT | (meas- | Meth- | mp |
| try | IUPAC name | STRUCTURE | (min) | ured) | od | (° C.) |
| 1 | 3-(4-acetamidophenyl)- N-(4-chlorophenyl)- N-methyl- imidazo[1,2-a]pyridine- 6-carboxamide | 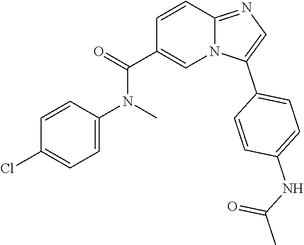 [SMILES: CC(=O)Nc1ccc(-c2cnc3ccc(C(=O)N(C)c4ccc(Cl)cc4)cn23)cc1] | 0.72 | 419 | A | |
| 2 | methyl N-[4-[6-[(4- chlorophenyl)-methyl- carbamoyl]imidazo[1,2- a]pyridin-3- yl]phenyl]carbamate | 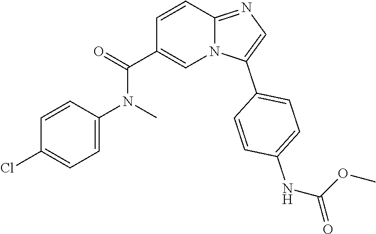 [SMILES: COC(=O)Nc1ccc(-c2cnc3ccc(C(=O)N(C)c4ccc(Cl)cc4)cn23)cc1] | 0.76 | 435 | A | |
| 3 | 3-(6-acetamido-3- pyridyl)-N-(4- chlorophenyl)-N- methyl-imidazo [1,2-a]pyridine-6- carboxamide | 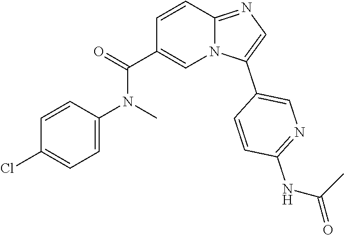 [SMILES: CC(=O)Nc1ccc(-c2cnc3ccc(C(=O)N(C)c4ccc(Cl)cc4)cn23)cn1] | 0.70 | 420 | A | 70-75 |
| 4 | methyl N-[5-[6-[(4- chlorophenyl)-methyl- carbamoyl]imidazo[1,2- a]pyridin-3-yl]-2- pyridyl]carbamate | 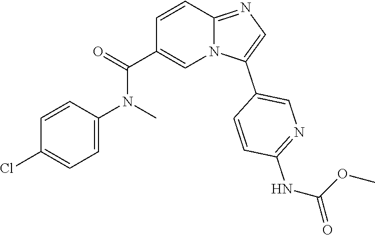 [SMILES: COC(=O)Nc1ccc(-c2cnc3ccc(C(=O)N(C)c4ccc(Cl)cc4)cn23)cn1] | 0.75 | 436 | A | 85-90 |
| 5 | methyl N-[4-[6-[(4- hydroxyphenyl)-methyl- carbamoyl]imidazo[1,2- a]pyridin-3- yl]phenyl]carbamate | 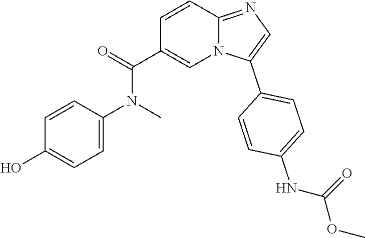 [SMILES: COC(=O)Nc1ccc(-c2cnc3ccc(C(=O)N(C)c4ccc(O)cc4)cn23)cc1] | 0.62 | 417 | A | |
| 6 | methyl N-[4-[6-[(4- cyanophenyl)-methyl- carbamoyl]imidazo[1,2- a]pyridin-3- yl]phenyl]carbamate | 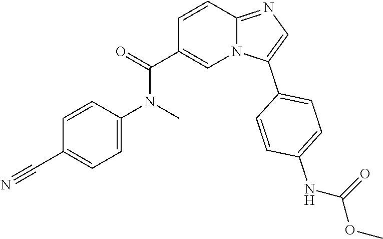 [SMILES: COC(=O)Nc1ccc(-c2cnc3ccc(C(=O)N(C)c4ccc(C#N)cc4)cn23)cc1] | 0.69 | 426 | A | 95-98 |
| 7 | methyl N-[4-[6-[(3- methoxyphenyl)-methyl- carbamoyl]imidazo[1,2- a]pyridin-3- yl]phenyl]carbamate | 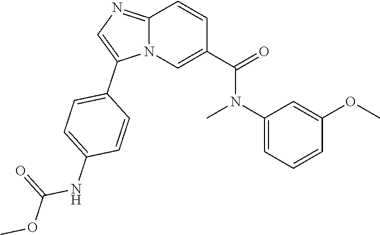 | 0.99 | 431.5 | B | |
| 8 | methyl N-[4-[6-[(4- methoxyphenyl)-methyl- carbamoyl]imidazo[1,2- a]pyridin-3- yl]phenyl]carbamate | 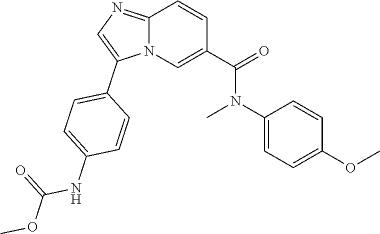 | 0.95 | 431.5 | B | |
| 9 | methyl N-[4-[6-[[3- (difluoromethoxy) phenyl]-methyl- carbamoyl]imidazo[1,2- a]pyridin-3- yl]phenyl]carbamate | 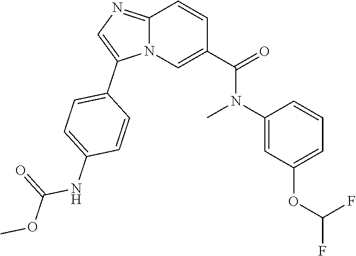 [SMILES: COC(=O)Nc1ccc(-c2cnc3ccc(C(=O)N(C)c4cccc(OC(F)F)c4)cn23)cc1] | 1.03 | 467.5 | B | |
| 10 | methyl N-[4-[6-[(3- ethylphenyl)-methyl- carbamoyl]imidazo[1,2- a]pyridin-3- yl]phenyl]carbamate | 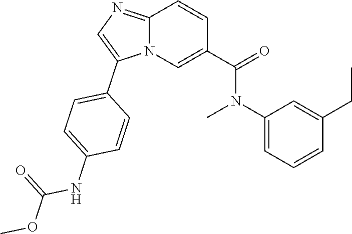 [SMILES: CCc1cccc(N(C)C(=O)c2ccc3ncc(-c4ccc(NC(=O)OC)cc4)n3c2)c1] | 1.12 | 429.6 | B | |
| 11 | methyl N-[4-[6-[(4- fluorophenyl)-methyl- carbamoyl]imidazo[1,2- a]pyridin-3- yl]phenyl]carbamate | 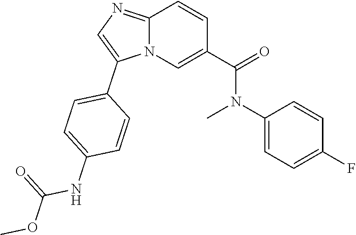 [SMILES: COC(=O)Nc1ccc(-c2cnc3ccc(C(=O)N(C)c4ccc(F)cc4)cn23)cc1] | 0.95 | 419.5 | B | |
| 12 | methyl N-[4-[6-[methyl- (2-methyl-4-pyridyl) carbamoyl]imidazo [1,2-a]pyridin-3- yl]phenyl]carbamate | 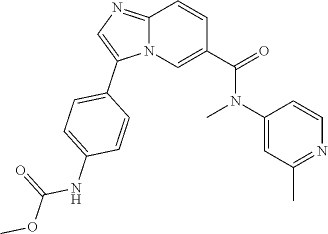 [SMILES: COC(=O)Nc1ccc(-c2cnc3ccc(C(=O)N(C)c4ccnc(C)c4)cn23)cc1] | 0.63 | 416.2 | B | |
| 13 | methyl N-[4-[6-[(3,4- dimethylphenyl)-methyl- carbamoyl]imidazo[1,2- a]pyridin-3- yl]phenyl]carbamate | 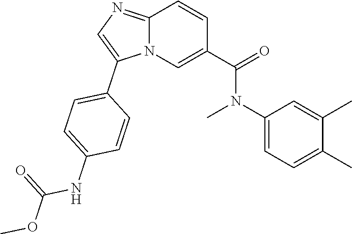 | 1.10 | 429.6 | B | |
| 14 | methyl N-[4-[6-[(4- isopropylphenyl)-methyl- carbamoyl]imidazo[1,2- a]pyridin-3- yl]phenyl]carbamate | 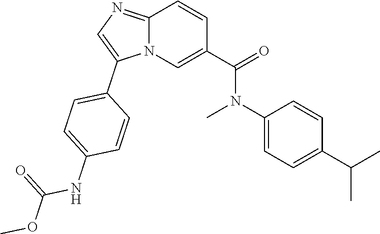 [SMILES: COC(=O)Nc1ccc(-c2cnc3ccc(C(=O)N(C)c4ccc(C(C)C)cc4)cn23)cc1] | 1.22 | 443.6 | B | |
| 15 | methyl N-[5-[6-[(4- cyanophenyl)-methyl- carbamoyl]imidazo[1,2- a]pyridin-3-yl]-2- pyridyl]carbamate | 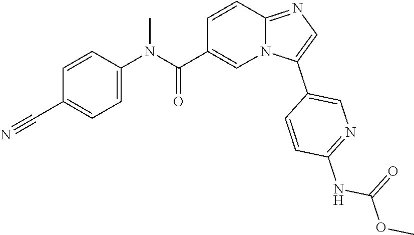 [SMILES: COC(=O)Nc1ccc(-c2cnc3ccc(C(=O)N(C)c4ccc(C#N)cc4)cn23)cn1] | 0.67 | 427 | A | |
| 16 | methyl N-[4-[6-[methyl (p-tolyl)carbamoyl] imidazo [1,2-a]pyridin-3- yl]phenyl]carbamate | 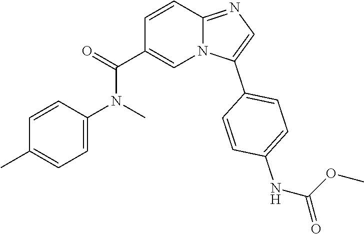 | 0.73 | 415 | A | |
| 17 | methyl N-[4-[6- [(4-chloro-3-fluoro- phenyl)-methyl- carbamoyl]imidazo[1,2- a]pyridin-3- yl]phenyl]carbamate | 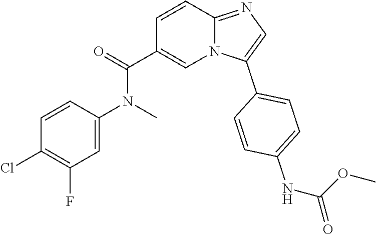 [SMILES: COC(=O)Nc1ccc(-c2cnc3ccc(C(=O)N(C)c4ccc(Cl)c(F)c4)cn23)cc1] | 0.82 | 453 | A | |
| 18 | methyl N-[4-[6-[(4- chlorophenyl)-methyl- carbamoyl]-7-methyl- imidazo[1,2-a]pyridin-3- yl]phenyl]carbamate | 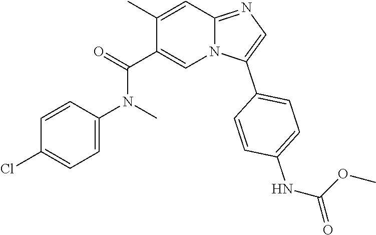 [SMILES: COC(=O)Nc1ccc(-c2cnc3cc(C)c(C(=O)N(C)c4ccc(Cl)cc4)cn23)cc1] | 1.02 | 449.1 | B | 103-108 |
| 19 | methyl N-[4-[6-[(4- chlorophenyl)-methyl- carbamoyl]-8-methyl- imidazo[1,2-a]pyridin-3- yl]phenyl]carbamate | 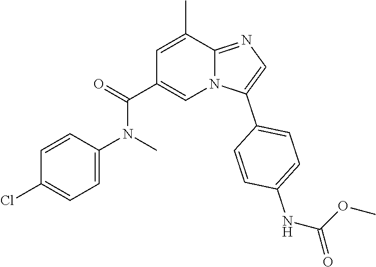 [SMILES: COC(=O)Nc1ccc(-c2cnc3c(C)cc(C(=O)N(C)c4ccc(Cl)cc4)cn23)cc1] | 1.07 | 449.1 | B | 105-110 |
| 20 | methyl N-[4-[6-[(4- chlorophenyl)-methyl- carbamoyl]-5-methyl- imidazo[1,2-a]pyridin-3- yl]phenyl]carbamate | 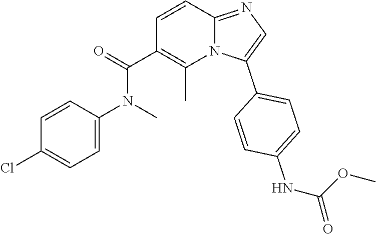 [SMILES: COC(=O)Nc1ccc(-c2cnc3ccc(C(=O)N(C)c4ccc(Cl)cc4)c(C)n23)cc1] | 1.01 | 469.0 | B | 110-115 |
| 21 | methyl N-[4-[5- chloro-6-[(4- chlorophenyl)-methyl- carbamoyl]imidazo[1,2- a]pyridin-3- yl]phenyl]carbamate | 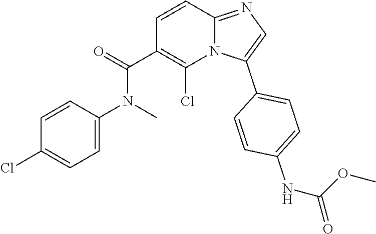 [SMILES: COC(=O)Nc1ccc(-c2cnc3ccc(C(=O)N(C)c4ccc(Cl)cc4)c(Cl)n23)cc1] | 1.27 | 469.0 | B | 125-130 |
| 22 | methyl N-[4-[8- chloro-6-[(4- chlorophenyl)-methyl- carbamoyl]imidazo[1,2- a]pyridin-3- yl]phenyl]carbamate | 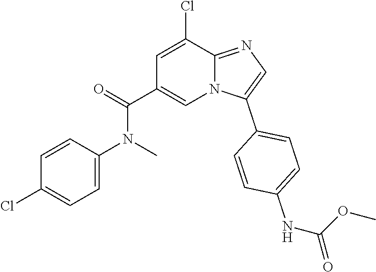 [SMILES: COC(=O)Nc1ccc(-c2cnc3c(Cl)cc(C(=O)N(C)c4ccc(Cl)cc4)cn23)cc1] | 1.42 | 469.0 | B | 135-140 |
| 23 | methyl N-[4-[7- chloro-6-[(4- chlorophenyl)-methyl- carbamoyl]imidazo[1,2- a]pyridin-3- yl]phenyl]carbamate | 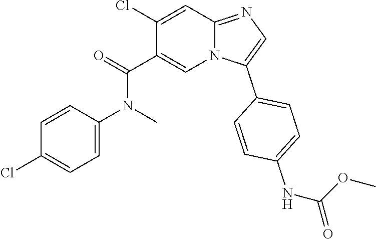 [SMILES: COC(=O)Nc1ccc(-c2cnc3cc(Cl)c(C(=O)N(C)c4ccc(Cl)cc4)cn23)cc1] | 1.26 | 449.1 | B | 125-130 |
| 24 | methyl N-[4-[6- [methyl(4-pyridyl) carbamoyl]imidazo [1,2-a]pyridin-3- yl]phenyl]carbamate | 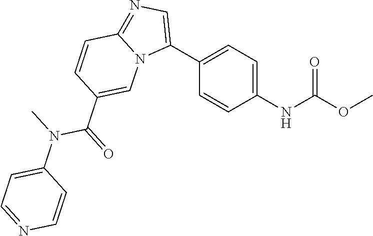 [SMILES: COC(=O)Nc1ccc(-c2cnc3ccc(C(=O)N(C)c4ccncc4)cn23)cc1] | 0.52 | 402 | A | |
| 25 | methyl N-[5-[6-[(4- fluorophenyl)-methyl- carbamoyl]imidazo[1,2- a]pyridin-3-yl]-2- pyridyl]carbamate | 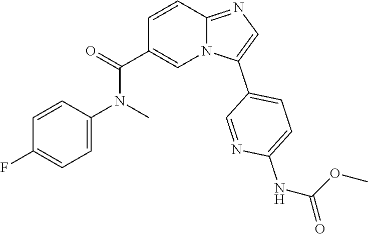 [SMILES: COC(=O)Nc1ccc(-c2cnc3ccc(C(=O)N(C)c4ccc(F)cc4)cn23)cn1] | 0.84 | 420.2 | B | 124-132 |
| 26 | methyl N-[4-[6- [(2-methoxy- 4-pyridyl)-methyl- carbamoyl]imidazo[1,2- a]pyridin-3- yl]phenyl]carbamate | 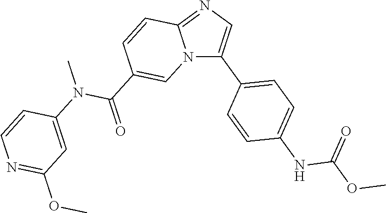 [SMILES: COC(=O)Nc1ccc(-c2cnc3ccc(C(=O)N(C)c4ccnc(OC)c4)cn23)cc1] | 0.67 | 433 | A | |
| 27 | methyl N-[4-[6-[(4- chlorophenyl)- cyclopropyl- carbamoyl]imidazo[1,2- a]pyridin-3- yl]phenyl]carbamate | 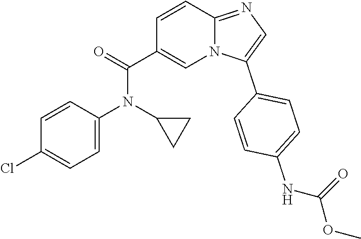 [SMILES: COC(=O)Nc1ccc(-c2cnc3ccc(C(=O)N(c4ccc(Cl)cc4)C4CC4)cn23)cc1] | 0.81 | 462 | A | |
| 28 | methyl N-[4-[6-[(4- chlorophenyl)- (cyclopropylmethyl) carbamoyl] imidazo[1,2-a]pyridin-3- yl]phenyl]carbamate | 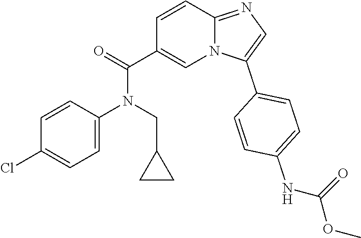 [SMILES: COC(=O)Nc1ccc(-c2cnc3ccc(C(=O)N(CC4CC4)c4ccc(Cl)cc4)cn23)cc1] | 0.87 | 476 | A | |
| 29 | methyl N-[4-[6-[(4- chlorophenyl)-(2,2,2- trifluoroethyl) carbamoyl]imidazo [1,2-a]pyridin-3- yl]phenyl]carbamate | 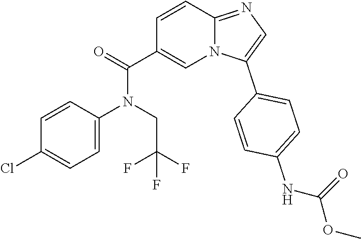 [SMILES: COC(=O)Nc1ccc(-c2cnc3ccc(C(=O)N(CC(F)(F)F)c4ccc(Cl)cc4)cn23)cc1] | 0.86 | 503 | A | |
| 30 | methyl N-[4-[6-[(4- chlorophenyl)-propyl- carbamoyl]imidazo[1,2- a]pyridin-3- yl]phenyl]carbamate | 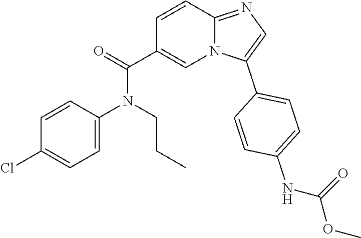 [SMILES: CCCN(C(=O)c1ccc2ncc(-c3ccc(NC(=O)OC)cc3)n2c1)c1ccc(Cl)cc1] | 0.76 | 430 | A | |
| 31 | methyl N-[4-[6-[(4- chlorophenyl)- (cyanomethyl) carbamoyl]imidazo [1,2-a]pyridin-3- yl]phenyl]carbamate | 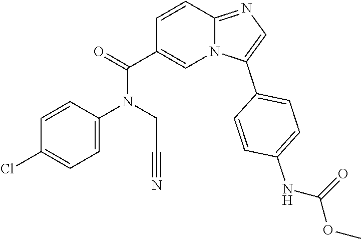 [SMILES: COC(=O)Nc1ccc(-c2cnc3ccc(C(=O)N(CC#N)c4ccc(Cl)cc4)cn23)cc1] | 0.77 | 460 | A | |
| 32 | methyl N-[4-[6-[(4- chlorophenyl)-ethyl- carbamoyl]imidazo[1,2- a]pyridin-3- yl]phenyl]carbamate | 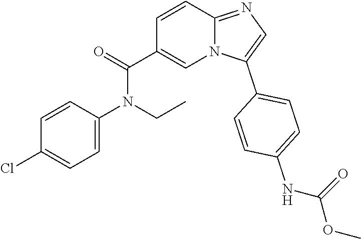 [SMILES: CCN(C(=O)c1ccc2ncc(-c3ccc(NC(=O)OC)cc3)n2c1)c1ccc(Cl)cc1] | 0.80 | 449 | A | |
| 33 | methyl N-[4-[6-[(4- chlorophenyl)- prop-2-ynyl- carbamoyl]imidazo[1,2- a]pyridin-3- yl]phenyl]carbamate | 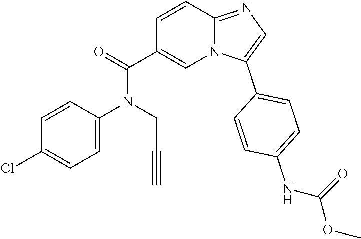 [SMILES: C#CCN(C(=O)c1ccc2ncc(-c3ccc(NC(=O)OC)cc3)n2c1)c1ccc(Cl)cc1] | 0.80 | 459 | A | |
| 34 | methyl N-[4-[7- bromo-6-[(4- chlorophenyl)-methyl- carbamoyl]imidazo[1,2- a]pyridin-3- yl]phenyl]carbamate | 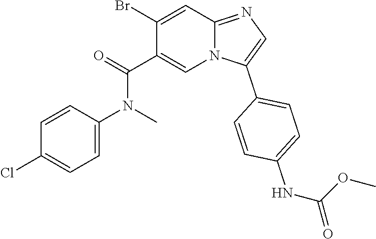 [SMILES: COC(=O)Nc1ccc(-c2cnc3cc(Br)c(C(=O)N(C)c4ccc(Cl)cc4)cn23)cc1] | 1.28 | 512.9 | B | 125-130 |
| 35 | methyl N-[4-[8- bromo-6-[(4- chlorophenyl)-methyl- carbamoyl]imidazo[1,2- a]pyridin-3- yl]phenyl]carbamate | 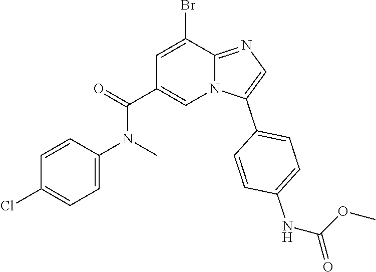 [SMILES: COC(=O)Nc1ccc(-c2cnc3c(Br)cc(C(=O)N(C)c4ccc(Cl)cc4)cn23)cc1] | 1.44 | 513.0 | B | 150-155 |
| 36 | methyl N-[4-[6-[(4- chlorophenyl)-methyl- carbamoyl]-8-fluoro- imidazo[1,2-a]pyridin-3- yl]phenyl]carbamate | 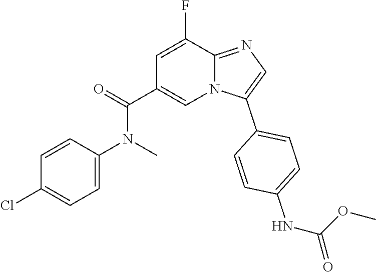 [SMILES: COC(=O)Nc1ccc(-c2cnc3c(F)cc(C(=O)N(C)c4ccc(Cl)cc4)cn23)cc1] | 1.39 | 453.1 | B | 140-145 |
| 37 | methyl N-[4-[6-[(4- chlorophenyl)-methyl- carbamoyl]-8-methoxy- imidazo[1,2-a]pyridin-3- yl]phenyl]carbamate | 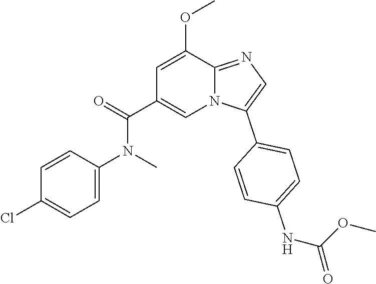 [SMILES: COC(=O)Nc1ccc(-c2cnc3c(OC)cc(C(=O)N(C)c4ccc(Cl)cc4)cn23)cc1] | 1.09 | 465.1 | B | 150-155 |
| 38 | methyl N-[4-[6-[(4- chlorophenyl)-methyl- carbamoyl]-5-cyano- imidazo[1,2-a]pyridin-3- yl]phenyl]carbamate | 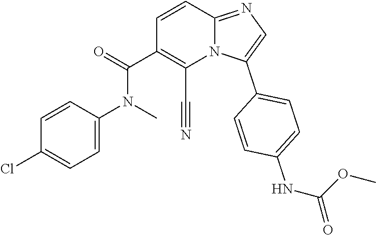 [SMILES: COC(=O)Nc1ccc(-c2cnc3ccc(C(=O)N(C)c4ccc(Cl)cc4)c(C#N)n23)cc1] | 1.34 | 460.1 | B | 130-135 |
| 39 | methyl N-[4-[6-[(4- chlorophenyl)-methyl- carbamoyl]-7-methoxy- imidazo[1,2-a]pyridin-3- yl]phenyl]carbamate | 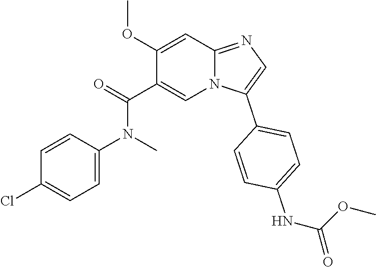 [SMILES: COC(=O)Nc1ccc(-c2cnc3cc(OC)c(C(=O)N(C)c4ccc(Cl)cc4)cn23)cc1] | 1.01 | 465.1 | B | 125-130 |
| 40 | methyl N-[4-[6-[(4- chlorophenyl)-methyl- carbamoyl]-8-cyano- imidazo[1,2-a]pyridin-3- yl]phenyl]carbamate | 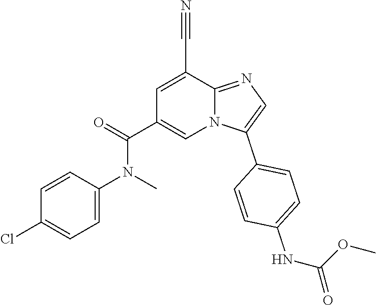 [SMILES: COC(=O)Nc1ccc(-c2cnc3c(C#N)cc(C(=O)N(C)c4ccc(Cl)cc4)cn23)cc1] | 1.42 | 460.1 | B | 150-155 |
| 41 | methyl N-[4-[6-[(4- chlorophenyl)-methyl- carbamoyl]-7-cyano- imidazo[1,2-a]pyridin-3- yl]phenyl]carbamate | 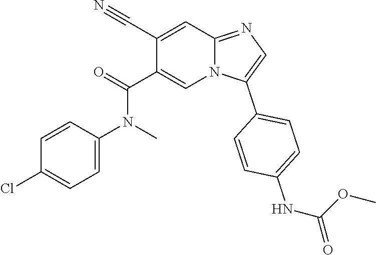 [SMILES: COC(=O)Nc1ccc(-c2cnc3cc(C#N)c(C(=O)N(C)c4ccc(Cl)cc4)cn23)cc1] | 1.39 | 460.1 | B | 175-180 |
| 42 | methyl N-[4-[6-[(4- chlorophenyl)-methyl- carbamoyl]-7-fluoro- imidazo[1,2-a]pyridin-3- yl]phenyl]carbamate | 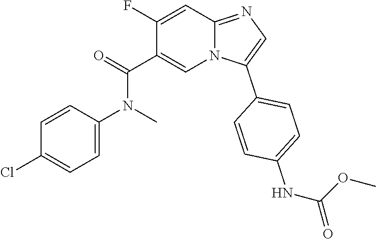 [SMILES: COC(=O)Nc1ccc(-c2cnc3cc(F)c(C(=O)N(C)c4ccc(Cl)cc4)cn23)cc1] | 1.15 | 453.1 | B | 90-95 |
| 43 | methyl N-[5-[6-[(3- ethynylphenyl)-methyl- carbamoyl]imidazo[1,2- a]pyridin-3-yl]-2- pyridyl]carbamate | 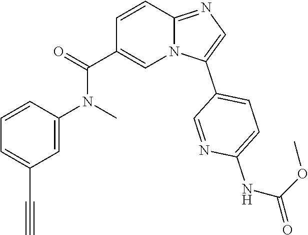 [SMILES: C#Cc1cccc(N(C)C(=O)c2ccc3ncc(-c4ccc(NC(=O)OC)nc4)n3c2)c1] | 0.90 | 426.2 | B | |
| 44 | methyl N-[5-[6-[(3- cyanophenyl)-methyl- carbamoyl]imidazo[1,2- a]pyridin-3-yl]-2- pyridyl]carbamate | 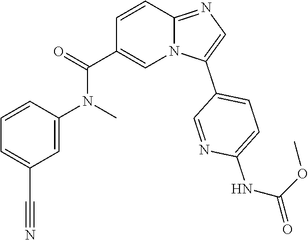 [SMILES: COC(=O)Nc1ccc(-c2cnc3ccc(C(=O)N(C)c4cccc(C#N)c4)cn23)cn1] | 0.76 | 427.2 | B | |
| 45 | methyl N-[5-[6- [(4-fluoro-3- methoxy-phenyl)- methyl-carbamoyl] imidazo[1,2- a]pyridin-3-yl]-2- pyridyl]carbamate | 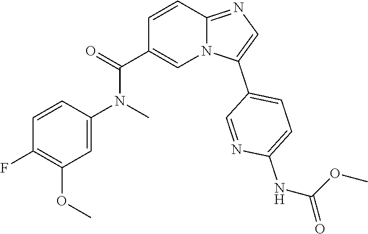 [SMILES: COC(=O)Nc1ccc(-c2cnc3ccc(C(=O)N(C)c4ccc(F)c(OC)c4)cn23)cn1] | 0.87 | 450.2 | B | 208-210 |
| 46 | methyl N-[5-[6- [(4-chloro-3- methyl-phenyl)-methyl- carbamoyl]imidazo[1,2- a]pyridin-3-yl]-2- pyridyl]carbamate | 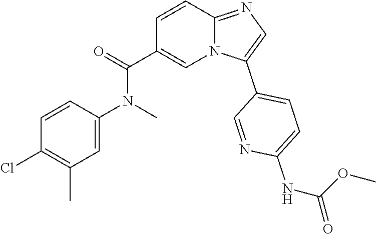 [SMILES: COC(=O)Nc1ccc(-c2cnc3ccc(C(=O)N(C)c4ccc(Cl)c(C)c4)cn23)cn1] | 1.04 | 450.2 | B | |
| 47 | methyl N-[5-[6-[(3,5- dimethylphenyl)-methyl- carbamoyl]imidazo[1,2- a]pyridin-3-yl]-2- pyridyl]carbamate | 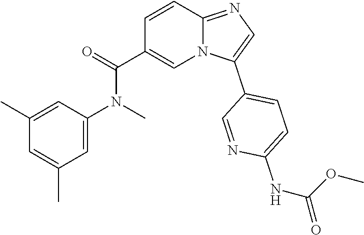 | 1.01 | 430.2 | B | |
| 48 | methyl N-[5-[6- [(4-fluoro-3- methyl-phenyl)-methyl- carbamoyl]imidazo[1,2- a]pyridin-3-yl]-2- pyridyl]carbamate | 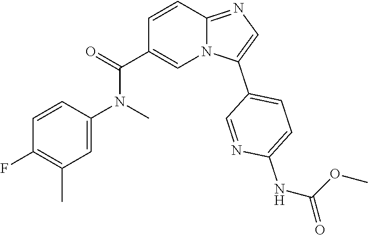 [SMILES: COC(=O)Nc1ccc(-c2cnc3ccc(C(=O)N(C)c4ccc(F)c(C)c4)cn23)cn1] | 0.94 | 434.2 | B | |
| 49 | methyl N-[5-[6- [methyl-[3- (trifluoromethyl)phenyl] carbamoyl]imidazo[1,2- a]pyridin-3-yl]- 2-pyridyl]carbamate | 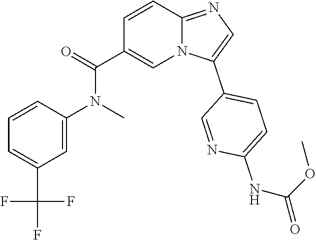 [SMILES: COC(=O)Nc1ccc(-c2cnc3ccc(C(=O)N(C)c4cccc(C(F)(F)F)c4)cn23)cn1] | 1.01 | 470.2 | B | |
| 50 | methyl N-[5-[6-[(3,4- difluorophenyl)-methyl- carbamoyl]imidazo[1,2- a]pyridin-3-yl]-2- pyridyl]carbamate | 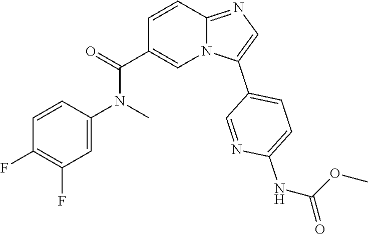 [SMILES: COC(=O)Nc1ccc(-c2cnc3ccc(C(=O)N(C)c4ccc(F)c(F)c4)cn23)cn1] | 0.89 | 438.2 | B | 210-214 |
| 51 | methyl N-[5-[6- [(4-cyano-3- methoxy-phenyl)- methyl-carbamoyl] imidazo[1,2- a]pyridin-3-yl]-2- pyridyl]carbamate | 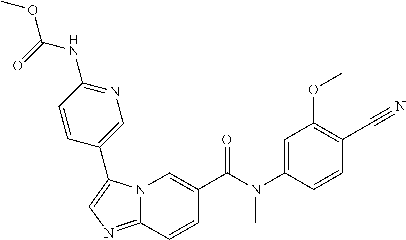 [SMILES: COC(=O)Nc1ccc(-c2cnc3ccc(C(=O)N(C)c4ccc(C#N)c(OC)c4)cn23)cn1] | 0.82 | 457.2 | B | |
| 52 | methyl N-[5-[6-[(3- fluorophenyl)-methyl- carbamoyl]imidazo[1,2- a]pyridin-3-yl]-2- pyridyl]carbamate | 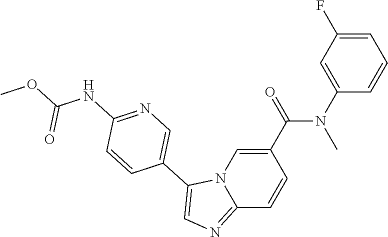 [SMILES: COC(=O)Nc1ccc(-c2cnc3ccc(C(=O)N(C)c4cccc(F)c4)cn23)cn1] | 0.85 | 420.2 | B | |
| 53 | methyl N-[5-[6- [(4-cyano-3- methyl-phenyl)-methyl- carbamoyl]imidazo[1,2- a]pyridin-3-yl]-2- pyridyl]carbamate | 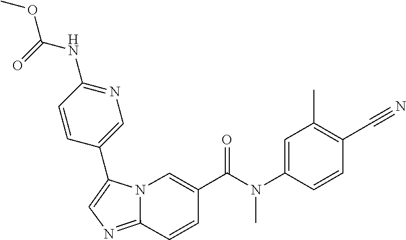 [SMILES: COC(=O)Nc1ccc(-c2cnc3ccc(C(=O)N(C)c4ccc(C#N)c(C)c4)cn23)cn1] | 0.86 | 441.2 | B | |
| 54 | methyl N-[5-[6- [methyl(m- tolyl)carbamoyl]imidazo [1,2-a]pyridin-3-yl]-2- pyridyl]carbamate | 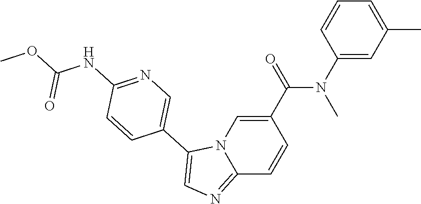 | 0.92 | 416.2 | B | |
| 55 | methyl N-[5-[6- [(5-methoxy- 3-pyridyl)-methyl- carbamoyl]imidazo[1,2- a]pyridin-3-yl]-2- pyridyl]carbamate | 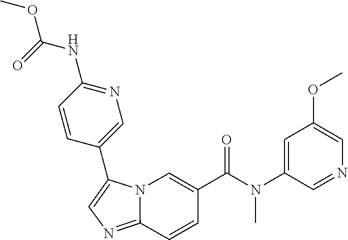 | 0.67 | 433.2 | B | 206-209 |
| 56 | methyl N-[5-[6- [methyl-(5- methyl-3-pyridyl) carbamoyl]imidazo [1,2-a]pyridin-3-yl]-2- pyridyl]carbamate | 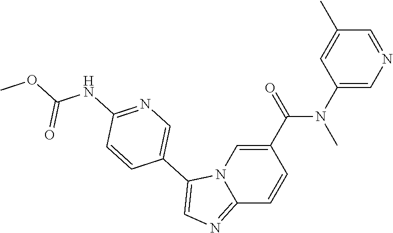 [SMILES: COC(=O)Nc1ccc(-c2cnc3ccc(C(=O)N(C)c4cncc(C)c4)cn23)cn1] | 0.65 | 417.2 | B | |
| 57 | methyl N-[5-[6-[[3- (difluoromethyl)phenyl]- methyl- carbamoyl]imidazo[1,2- a]pyridin-3-yl]-2- pyridyl]carbamate | 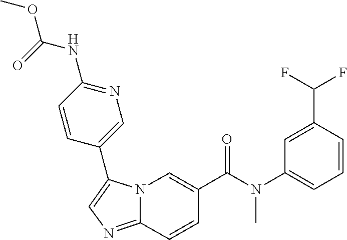 [SMILES: COC(=O)Nc1ccc(-c2cnc3ccc(C(=O)N(C)c4cccc(C(F)F)c4)cn23)cn1] | 0.89 | 452.2 | B | 180-183 |
| 58 | methyl N-[5-[6-[(3,5- dimethoxyphenyl)- methyl-carbamoyl] imidazo[1,2- a]pyridin-3-yl]-2- pyridyl]carbamate | 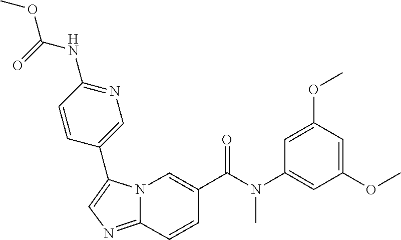 | 0.90 | 462.2 | B | |
| 59 | methyl N-[5-[6- [(3-methoxy- 5-methyl-phenyl)- methyl-carbamoyl] imidazo[1,2- a]pyridin-3-yl]-2- pyridyl]carbamate | 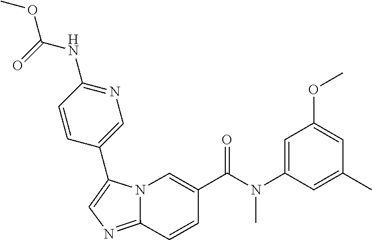 | 0.95 | 446.2 | B | |
| 60 | methyl N-[5-[6- [(6-fluoro-3- pyridyl)-methyl- carbamoyl]imidazo[1,2- a]pyridin-3-yl]-2- pyridyl]carbamate | 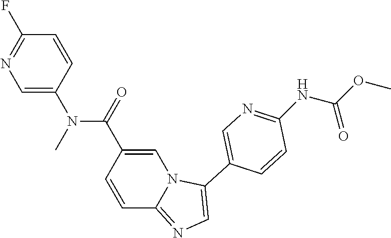 [SMILES: COC(=O)Nc1ccc(-c2cnc3ccc(C(=O)N(C)c4ccc(F)nc4)cn23)cn1] | 0.69 | 421.2 | B | |
| 61 | methyl N-[5-[6- [methyl(phenyl) carbamoyl] imidazo[1,2-a]pyridin- 3-yl]-2- pyridyl]carbamate | 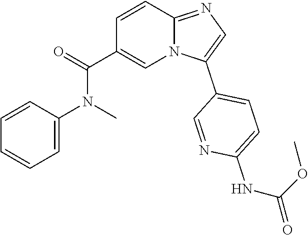 [SMILES: COC(=O)Nc1ccc(-c2cnc3ccc(C(=O)N(C)c4ccccc4)cn23)cn1] | 0.88 | 402.5 | B | |
| 62 | methyl N-[5-[6-[(3- chlorophenyl)-methyl- carbamoyl]imidazo[1,2- a]pyridin-3-yl]-2- pyridyl]carbamate | 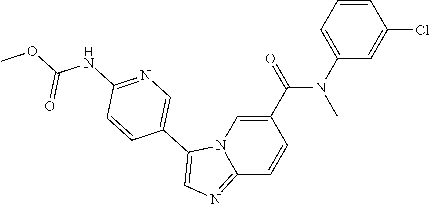 [SMILES: COC(=O)Nc1ccc(-c2cnc3ccc(C(=O)N(C)c4cccc(Cl)c4)cn23)cn1] | 1.00 | 436.1 | B | |
| 63 | methyl N-[5-[6-[(3- methoxyphenyl)-methyl- carbamoyl]imidazo[1,2- a]pyridin-3-yl]-2- pyridyl]carbamate | 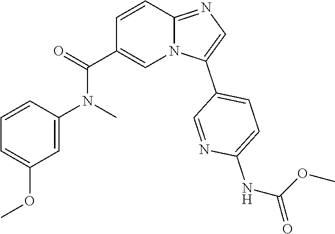 [SMILES: COC(=O)Nc1ccc(-c2cnc3ccc(C(=O)N(C)c4cccc(OC)c4)cn23)cn1] | 0.92 | 432.4 | B | |
| 64 | methyl N-[5-[6-[methyl (p-tolyl)carbamoyl] imidazo [1,2-a]pyridin-3-yl]-2- pyridyl]carbamate | 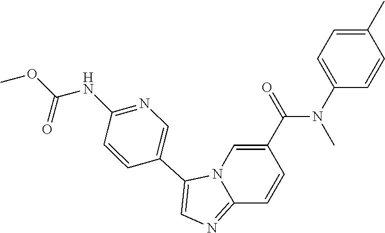 | 0.98 | 416.4 | B | |
| 65 | methyl N-[5-[6-[(3,4- dimethylphenyl)-methyl- carbamoyl]imidazo[1,2- a]pyridin-3-yl]-2- pyridyl]carbamate | 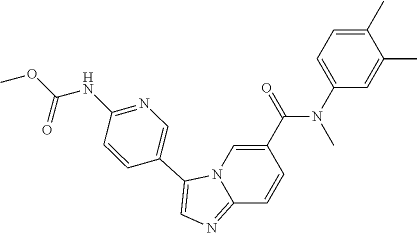 | 1.06 | 430.5 | B | |
| 66 | methyl N-[5-[6-[[4- (difluoromethoxy) phenyl]-methyl- carbamoyl]imidazo[1,2- a]pyridin-3-yl]-2- pyridyl]carbamate | 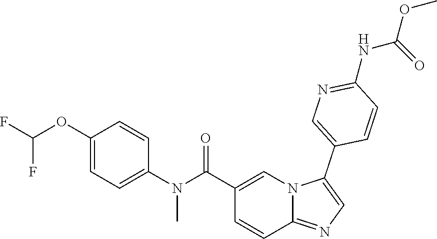 [SMILES: COC(=O)Nc1ccc(-c2cnc3ccc(C(=O)N(C)c4ccc(OC(F)F)cc4)cn23)cn1] | 0.98 | 468.4 | B | |
| 67 | methyl N-[5-[6- [(6-methoxy- 3-pyridyl)-methyl- carbamoyl]imidazo[1,2- a]pyridin-3-yl]-2- pyridyl]carbamate | 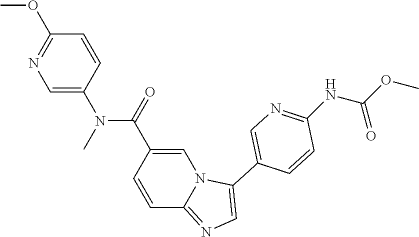 | 0.82 | 433.3 | B | |
| 68 | methyl N-[5-[6-[(4- bromophenyl)-methyl- carbamoyl]imidazo[1,2- a]pyridin-3-yl]-2- pyridyl]carbamate | 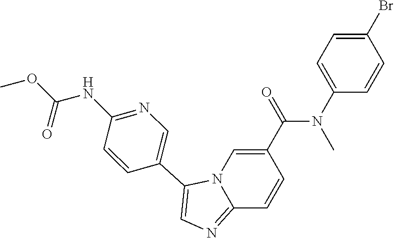 [SMILES: COC(=O)Nc1ccc(-c2cnc3ccc(C(=O)N(C)c4ccc(Br)cc4)cn23)cn1] | 1.03 | 480.1 | B | |
| 69 | methyl N-[5-[6-[(3- bromophenyl)-methyl- carbamoyl]imidazo[1,2- a]pyridin-3-yl]-2- pyridyl]carbamate | 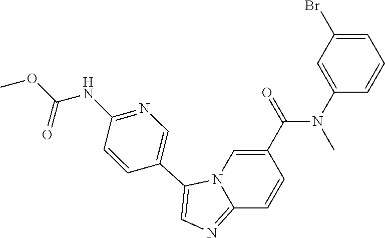 [SMILES: COC(=O)Nc1ccc(-c2cnc3ccc(C(=O)N(C)c4cccc(Br)c4)cn23)cn1] | 1.02 | 480.1 | B | |
| 70 | methyl N-[5-[6- [(2-methoxy- 4-pyridyl)-methyl- carbamoyl]imidazo[1,2- a]pyridin-3-yl]-2- pyridyl]carbamate | 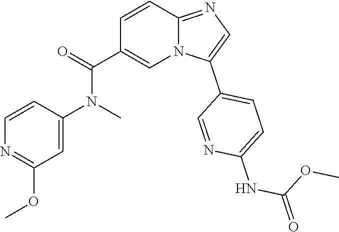 [SMILES: COC(=O)Nc1ccc(-c2cnc3ccc(C(=O)N(C)c4ccnc(OC)c4)cn23)cn1] | 0.84 | 433.2 | B | |
| 71 | methyl N-[5-[6- [(2-fluoro-4- pyridyl)-methyl- carbamoyl]imidazo[1,2- a]pyridin-3-yl]-2- pyridyl]carbamate | 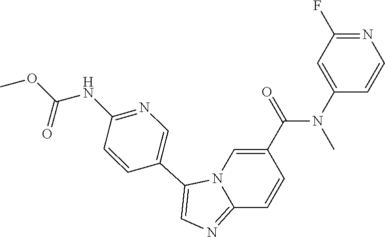 [SMILES: COC(=O)Nc1ccc(-c2cnc3ccc(C(=O)N(C)c4ccnc(F)c4)cn23)cn1] | 0.77 | 421.1 | B | |
| 72 | methyl N-[5-[6-[(3- cyclopropylphenyl)- methyl-carbamoyl] imidazo[1,2- a]pyridin-3-yl]-2- pyridyl]carbamate | 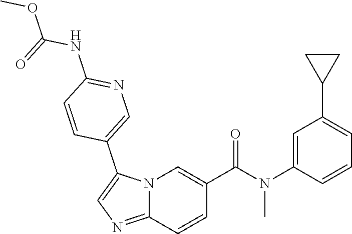 [SMILES: COC(=O)Nc1ccc(-c2cnc3ccc(C(=O)N(C)c4cccc(C5CC5)c4)cn23)cn1] | 1.08 | 442.5 | B | |
| 73 | methyl N-[5-[6-[(3- iodophenyl)-methyl- carbamoyl]imidazo[1,2- a]pyridin-3-yl]-2- pyridyl]carbamate | 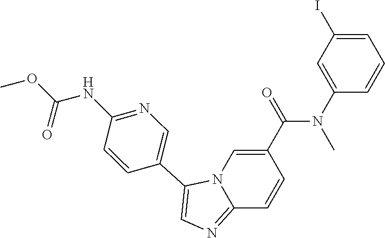 [SMILES: COC(=O)Nc1ccc(-c2cnc3ccc(C(=O)N(C)c4cccc(I)c4)cn23)cn1] | 1.06 | 528.3 | B | |
| 74 | methyl N-[5-[6-[[3-(2,2- difluoroethyl)phenyl]- methyl-carbamoyl] imidazo [1,2-a]pyridin-3-yl]-2- pyridyl]carbamate | 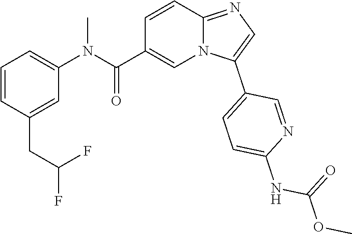 [SMILES: COC(=O)Nc1ccc(-c2cnc3ccc(C(=O)N(C)c4cccc(CC(F)F)c4)cn23)cn1] | 0.74 | 466 | A | |
| 75 | methyl N-[5-[6- [(4-chloro-3- methoxy-phenyl)- methyl-carbamoyl] imidazo[1,2- a]pyridin-3-yl]-2- pyridyl]carbamate | 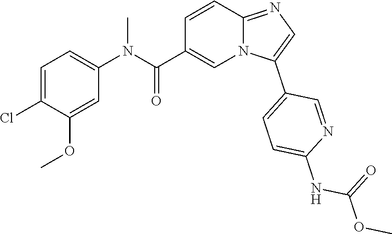 [SMILES: COC(=O)Nc1ccc(-c2cnc3ccc(C(=O)N(C)c4ccc(Cl)c(OC)c4)cn23)cn1] | 0.77 | 466 | A | |
| 76 | methyl N-[5-[6-[(4- fluorophenyl)-(2- methoxyethyl) carbamoyl] imidazo[1,2-a]pyridin- 3-yl]-2- pyridyl]carbamate | 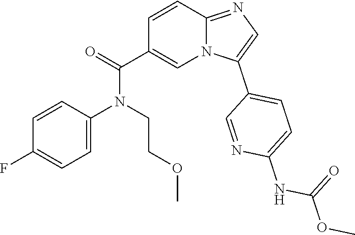 [SMILES: COCCN(C(=O)c1ccc2ncc(-c3ccc(NC(=O)OC)nc3)n2c1)c1ccc(F)cc1] | 0.71 | 464 | A | |
| 77 | methyl N-[5-[6- [[4-fluoro-3- (2-methoxyethoxy) phenyl]-methyl- carbamoyl]imidazo[1,2- a]pyridin-3-yl]-2- pyridyl]carbamate | 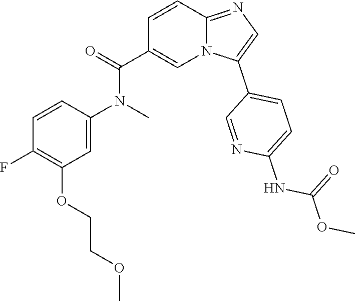 [SMILES: COCCOc1cc(N(C)C(=O)c2ccc3ncc(-c4ccc(NC(=O)OC)nc4)n3c2)ccc1F] | 0.72 | 494 | A | |
| 78 | N-(4-fluoro-3-methyl- phenyl)-N-methyl-3-[6- (propanoylamino)-3- pyridyl]imidazo[1,2- a]pyridine-6- carboxamide | 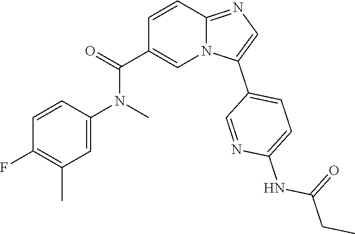 [SMILES: CCC(=O)Nc1ccc(-c2cnc3ccc(C(=O)N(C)c4ccc(F)c(C)c4)cn23)cn1] | 0.76 | 432 | A | |
| 79 | N-(4-fluoro-3-methoxy- phenyl)-3-[6-[[methoxy (methyl)carbamoyl] amino]-3-pyridyl]-N- methyl-imidazo[1,2- a]pyridine-6- carboxamide | 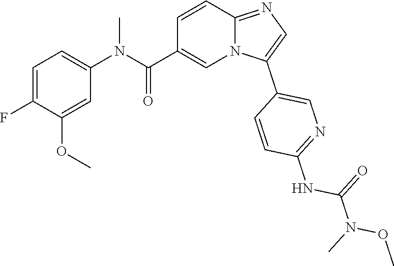 [SMILES: COc1cc(N(C)C(=O)c2ccc3ncc(-c4ccc(NC(=O)N(C)OC)nc4)n3c2)ccc1F] | 0.73 | 479 | A | |
| 80 | N-(4-fluoro-3-methoxy- phenyl)-N-methyl-3-[4- (methylcarbamoyl- amino) phenyl]imidazo[1,2- a]pyridine-6- carboxamide | 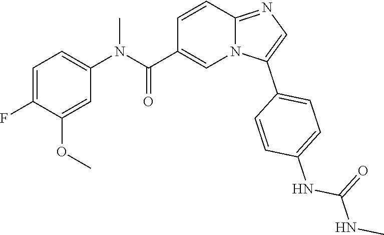 [SMILES: CNC(=O)Nc1ccc(-c2cnc3ccc(C(=O)N(C)c4ccc(F)c(OC)c4)cn23)cc1] | 0.64 | 448 | A | |
| 81 | N-(4-fluoro-3-methoxy- phenyl)-N-methyl-3-[6- (methylcarbamoyl- amino)- 3-pyridyl]imidazo[1,2- a]pyridine-6- carboxamide | 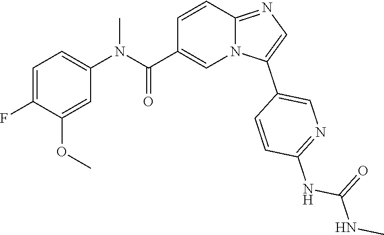 [SMILES: CNC(=O)Nc1ccc(-c2cnc3ccc(C(=O)N(C)c4ccc(F)c(OC)c4)cn23)cn1] | 0.65 | 449 | A | |
| 82 | methyl N-[5-[6-[(4- fluorophenyl)-methyl- carbamoyl]-8-methyl- imidazo[1,2-a]pyridin- 3-yl]-2- pyridyl]carbamate | 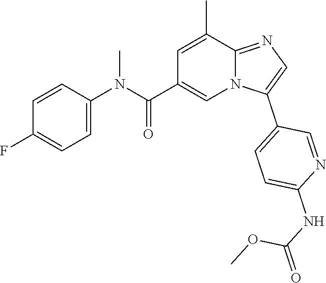 [SMILES: COC(=O)Nc1ccc(-c2cnc3c(C)cc(C(=O)N(C)c4ccc(F)cc4)cn23)cn1] | 0.90 | 434.5 | B | 140-145 |
| 83 | methyl N-[5-[8- cyclopropyl-6-[(4- fluorophenyl)-methyl- carbamoyl]imidazo[1,2- a]pyridin-3-yl]-2- pyridyl]carbamate | 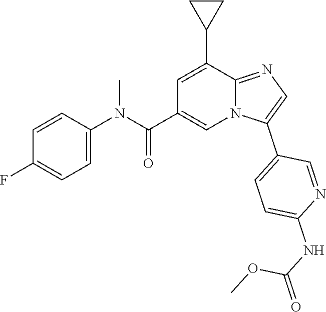 [SMILES: COC(=O)Nc1ccc(-c2cnc3c(C4CC4)cc(C(=O)N(C)c4ccc(F)cc4)cn23)cn1] | 1.01 | 460.6 | B | 210-215 |
| 84 | methyl N-[5-[6-[(4- fluorophenyl)-methyl- carbamoyl]-8-methoxy- imidazo[1,2-a]pyridin- 3-yl]-2- pyridyl]carbamate | 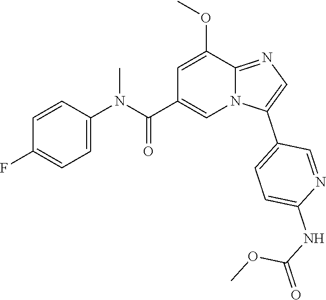 [SMILES: COC(=O)Nc1ccc(-c2cnc3c(OC)cc(C(=O)N(C)c4ccc(F)cc4)cn23)cn1] | 0.93 | 450.5 | B | 200-205 |
| 85 | methyl N-[5-[8- chloro-6-[(4- fluorophenyl)-methyl- carbamoyl]imidazo[1,2- a]pyridin-3-yl]-2- pyridyl]carbamate | 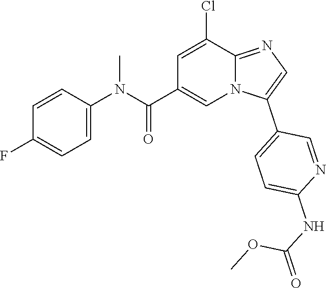 [SMILES: COC(=O)Nc1ccc(-c2cnc3c(Cl)cc(C(=O)N(C)c4ccc(F)cc4)cn23)cn1] | 1.20 | 454.5 | B | 120-125 |
| 86 | methyl N-[5-[6-[(4- fluorophenyl)-methyl- carbamoyl]-7-methyl- imidazo[1,2-a]pyridin- 3-yl]-2- pyridyl]carbamate | 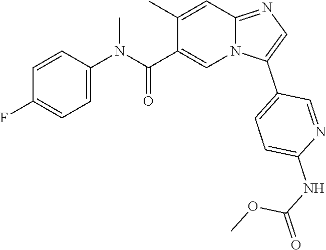 [SMILES: COC(=O)Nc1ccc(-c2cnc3cc(C)c(C(=O)N(C)c4ccc(F)cc4)cn23)cn1] | 0.83 | 434.5 | B | 180-185 |
| 87 | methyl N-[5-[7- cyano-6-[(4- fluorophenyl)-methyl- carbamoyl]imidazo[1,2- a]pyridin-3-yl]-2- pyridyl]carbamate | 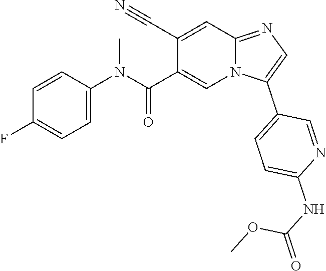 [SMILES: COC(=O)Nc1ccc(-c2cnc3cc(C#N)c(C(=O)N(C)c4ccc(F)cc4)cn23)cn1] | 1.15 | 445.5 | B | 215-220 |
| 88 | 3-[6-(dimethyl- carbamoylamino)- 3-pyridyl]-N-(4-fluoro- 3-methoxy-phenyl)-N- methyl-imidazo[1,2- a]pyridine-6- carboxamide | 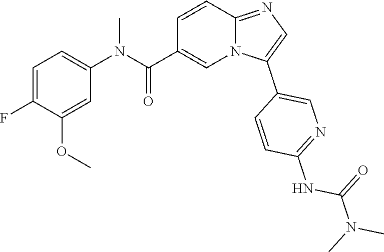 [SMILES: COc1cc(N(C)C(=O)c2ccc3ncc(-c4ccc(NC(=O)N(C)C)nc4)n3c2)ccc1F] | 0.66 | 463 | A | 102-105 |
| 89 | methyl N-[5-[6- [cyanomethyl-(4-fluoro- 3-methoxy-phenyl) carbamoyl]imidazo [1,2-a]pyridin-3-yl]-2- pyridyl]carbamate | 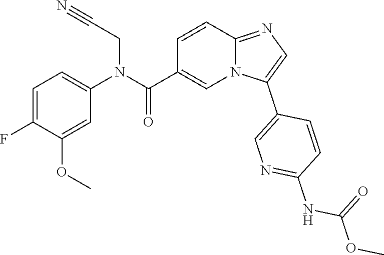 [SMILES: COC(=O)Nc1ccc(-c2cnc3ccc(C(=O)N(CC#N)c4ccc(F)c(OC)c4)cn23)cn1] | 0.71 | 475 | A | |
| 90 | methyl N-[5-[6- [(4-chloro-3- methoxy-phenyl)- (cyanomethyl) carbamoyl] imidazo[1,2-a]pyridin- 3-yl]-2- pyridyl]carbamate | 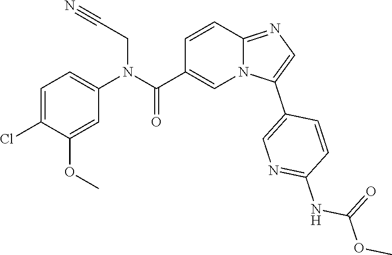 [SMILES: COC(=O)Nc1ccc(-c2cnc3ccc(C(=O)N(CC#N)c4ccc(Cl)c(OC)c4)cn23)cn1] | 0.75 | 491 | A | |
| 91 | methyl 2-(4-fluoro-3- methoxy-N-[3-[6- (methoxycarbonyl- amino)-3- pyridyl]imidazo[1,2- a]pyridine-6- carbonyl]anilino)acetate | 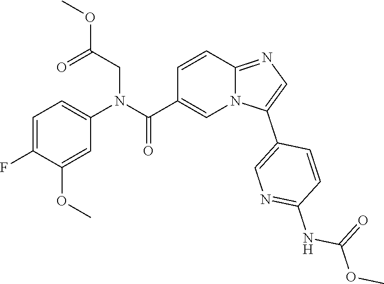 [SMILES: COC(=O)CN(C(=O)c1ccc2ncc(-c3ccc(NC(=O)OC)nc3)n2c1)c1ccc(F)c(OC)c1] | 0.73 | 508 | A | |
| 92 | methyl N-[5-[6- [(4-fluoro-3- methoxy-phenyl)- (methylsulfonylmethyl) carbamoyl] imidazo[1,2-a]pyridin- 3-yl]-2-pyridyl] carbamate | 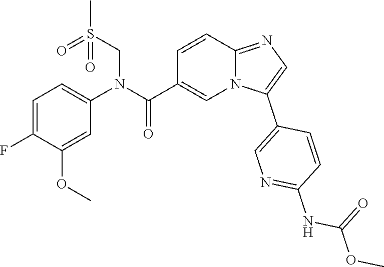 [SMILES: COC(=O)Nc1ccc(-c2cnc3ccc(C(=O)N(CS(C)(=O)=O)c4ccc(F)c(OC)c4)cn23)cn1] | 0.67 | 528 | A | |
| 93 | methyl N-[5-[6- [(4-fluoro-3- methoxy-phenyl)- (methoxymethyl) carbamoyl] imidazo[1,2-a]pyridin- 3-yl]-2- pyridyl]carbamate | 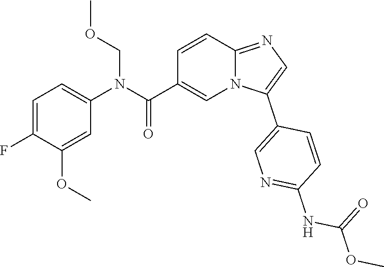 [SMILES: COCN(C(=O)c1ccc2ncc(-c3ccc(NC(=O)OC)nc3)n2c1)c1ccc(F)c(OC)c1] | 0.74 | 480 | A | 65-72 |
| 94 | methyl N-[5-[6- [(4-fluoro-3- methoxy-phenyl)- (methoxymethyl) carbamoyl]- 8-methyl-imidazo[1,2- a]pyridin-3-yl]-2- pyridyl]carbamate | 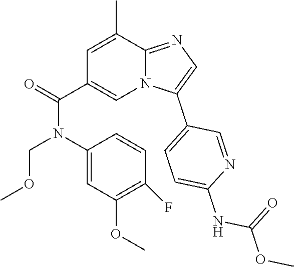 | 0.96 | 494.6 | B | 90-95 |
| 95 | methyl N-[5-[6- [(4-fluoro-3- methoxy-phenyl)- methyl-carbamoyl]- 8-methyl- imidazo[1,2-a]pyridin- 3-yl]-2- pyridyl]carbamate | 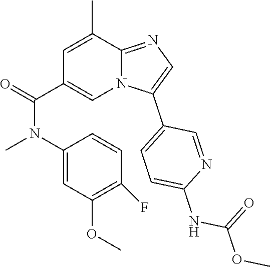 [SMILES: COC(=O)Nc1ccc(-c2cnc3c(C)cc(C(=O)N(C)c4ccc(F)c(OC)c4)cn23)cn1] | 0.92 | 464.5 | B | 160-165 |
| 96 | methyl N-[5-[6- [(4-fluoro-3- hydroxy-phenyl)- methyl-carbamoyl] imidazo[1,2- a]pyridin-3-yl]-2- pyridyl]carbamate | 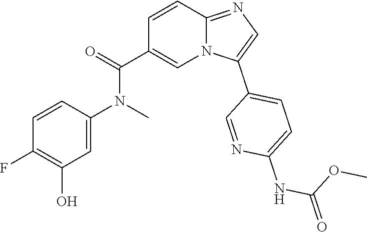 [SMILES: COC(=O)Nc1ccc(-c2cnc3ccc(C(=O)N(C)c4ccc(F)c(O)c4)cn23)cn1] | 0.62 | 436 | A | 185-200 |
| 97 | methyl N-[5-[6- [(4-fluoro-3- methoxy-phenyl)-[2- (methylamino)-2-oxo- ethyl]carbamoyl] imidazo[1,2- a]pyridin-3-yl]-2- pyridyl]carbamate | 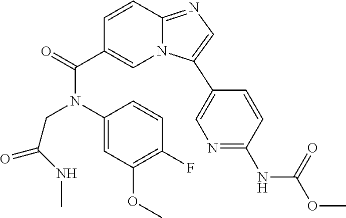 [SMILES: CNC(=O)CN(C(=O)c1ccc2ncc(-c3ccc(NC(=O)OC)nc3)n2c1)c1ccc(F)c(OC)c1] | 0.61 | 507 | A | |
| 98 | methyl N-[5-[8- bromo-6-[(4- fluorophenyl)-methyl- carbamoyl]imidazo[1,2- a]pyridin-3-yl]-2- pyridyl]carbamate | 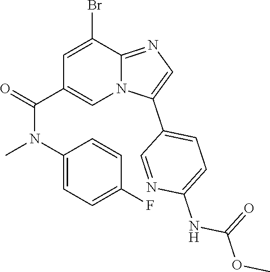 [SMILES: COC(=O)Nc1ccc(-c2cnc3c(Br)cc(C(=O)N(C)c4ccc(F)cc4)cn23)cn1] | 0.84 | 498 | A | |
| 99 | N-(4-fluoro-3-methoxy- phenyl)-3-[6-(methoxy- carbamoylamino)- 3-pyridyl]-N-methyl- imidazo[1,2-a]pyridine- 6-carboxamide | 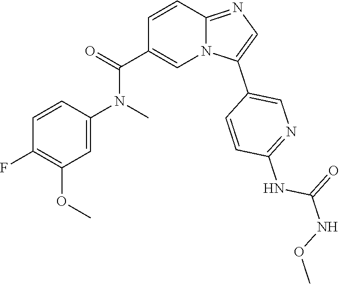 [SMILES: CONC(=O)Nc1ccc(-c2cnc3ccc(C(=O)N(C)c4ccc(F)c(OC)c4)cn23)cn1] | 0.63 | 465 | A | |
| 100 | methyl N-[5-[8- acetamido-6- [(4-fluorophenyl)- methyl-carbamoyl] imidazo[1,2- a]pyridin-3-yl]-2- pyridyl]carbamate | 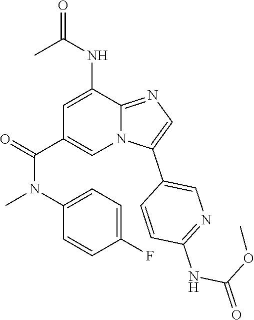 [SMILES: COC(=O)Nc1ccc(-c2cnc3c(NC(C)=O)cc(C(=O)N(C)c4ccc(F)cc4)cn23)cn1] | 0.78 | 477 | A | |
| 101 | methyl N-[5-[6-[(3,5- difluorophenyl)-isobutyl- carbamoyl]imidazo[1,2- a]pyridin-3-yl]-2- pyridyl]carbamate | 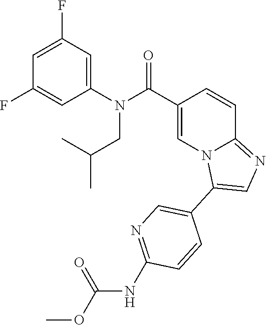 [SMILES: COC(=O)Nc1ccc(-c2cnc3ccc(C(=O)N(CC(C)C)c4cc(F)cc(F)c4)cn23)cn1] | 0.98 | 480.2 | 2 | |
| 102 | ethyl 4-[cyclopropyl- methyl-[3-[6- (methoxycarbonyl- amino)- 3-pyridyl]imidazo[1,2- a]pyridine-6- carbonyl]amino] benzoate | 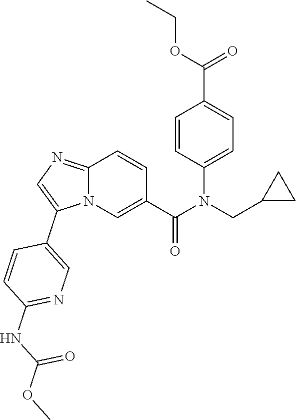 [SMILES: CCOC(=O)c1ccc(N(CC2CC2)C(=O)c2ccc3ncc(-c4ccc(NC(=O)OC)nc4)n3c2)cc1] | 1.24 | 514.0 | 1 | |
| 103 | methyl 4-[[3-[6- (methoxycarbonyl- amino)- 3-pyridyl]imidazo[1,2- a]pyridine-6-carbonyl]- methyl-amino]benzoate | 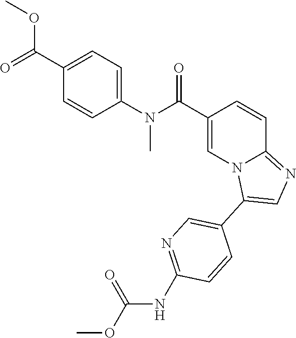 [SMILES: COC(=O)Nc1ccc(-c2cnc3ccc(C(=O)N(C)c4ccc(C(=O)OC)cc4)cn23)cn1] | 0.71 | 460.2 | 1 | |
| 104 | methyl N-[5-[6-[(4- acetylphenyl)-methyl- carbamoyl]imidazo[1,2- a]pyridin-3-yl]-2- pyridyl]carbamate | 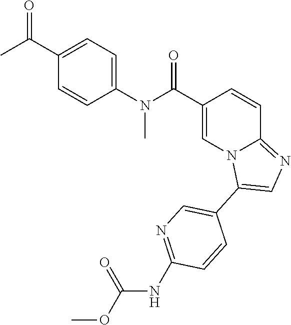 [SMILES: COC(=O)Nc1ccc(-c2cnc3ccc(C(=O)N(C)c4ccc(C(C)=O)cc4)cn23)cn1] | 0.66 | 444.2 | 1 | |
| 105 | methyl N-[5-[6-[(3,4- difluorophenyl)-ethyl- carbamoyl]imidazo[1,2- a]pyridin-3-yl]-2- pyridyl]carbamate | 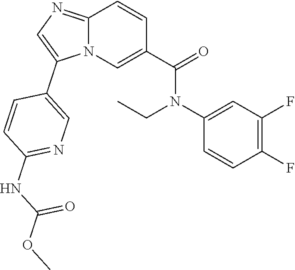 [SMILES: CCN(C(=O)c1ccc2ncc(-c3ccc(NC(=O)OC)nc3)n2c1)c1ccc(F)c(F)c1] | 1.11 | 452.0 | 1 | |
| 106 | methyl N-[5-[6-[(3- methoxyphenyl)-[2- (methylamino)-2-oxo- ethyl]carbamoyl] imidazo[1,2- a]pyridin-3-yl]-2- pyridyl]carbamate | 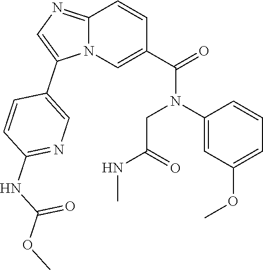 [SMILES: CNC(=O)CN(C(=O)c1ccc2ncc(-c3ccc(NC(=O)OC)nc3)n2c1)c1cccc(OC)c1] | 0.62 | 489.2 | 1 | |
| 107 | methyl N-[5-[6- [(3-chloro-4- cyano-phenyl)-methyl- carbamoyl]imidazo[1,2- a]pyridin-3-yl]-2- pyridyl]carbamate | 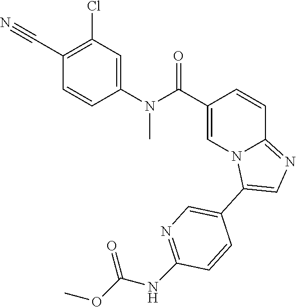 [SMILES: COC(=O)Nc1ccc(-c2cnc3ccc(C(=O)N(C)c4ccc(C#N)c(Cl)c4)cn23)cn1] | 0.73 | 461.2 | 1 | |
| 108 | methyl N-[5-[6- [cyanomethyl(phenyl) carbamoyl] imidazo[1,2-a]pyridin- 3-yl]-2-pyridyl] carbamate | 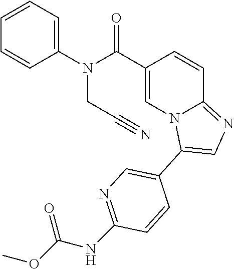 [SMILES: COC(=O)Nc1ccc(-c2cnc3ccc(C(=O)N(CC#N)c4ccccc4)cn23)cn1] | 0.69 | 427.2 | 1 | |
| 109 | methyl N-[5-[6- [cyclobutyl- (4-fluorophenyl) carbamoyl]imidazo [1,2-a]pyridin-3-yl]-2- pyridyl]carbamate | 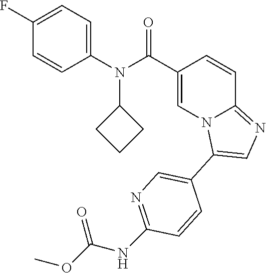 [SMILES: COC(=O)Nc1ccc(-c2cnc3ccc(C(=O)N(c4ccc(F)cc4)C4CCC4)cn23)cn1] | 0.84 | 460.2 | 1 | |
| 110 | methyl N-[5-[6- [cyclopropyl- (4-fluorophenyl) carbamoyl]imidazo [1,2-a]pyridin-3-yl]-2- pyridyl]carbamate | 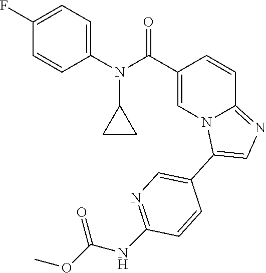 [SMILES: COC(=O)Nc1ccc(-c2cnc3ccc(C(=O)N(c4ccc(F)cc4)C4CC4)cn23)cn1] | 0.76 | 446.2 | 1 | |
| 111 | methyl 2-(3-fluoro- N-[3-[6-(methoxy- carbonylamino)- 3-pyridyl]imidazo[1,2- a]pyridine-6- carbonyl]anilino) acetate | 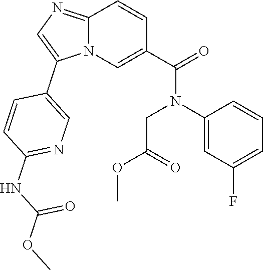 [SMILES: COC(=O)CN(C(=O)c1ccc2ncc(-c3ccc(NC(=O)OC)nc3)n2c1)c1cccc(F)c1] | 0.85 | 478.2 | 1 | |
| 112 | methyl N-[5-[6-[2- hydroxyethyl(phenyl) carbamoyl]imidazo [1,2-a]pyridin-3- yl]-2-pyridyl]carbamate | 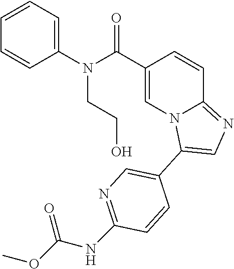 [SMILES: COC(=O)Nc1ccc(-c2cnc3ccc(C(=O)N(CCO)c4ccccc4)cn23)cn1] | 0.88 | 432.2 | 1 | |
| 113 | methyl N-[5-[6- [(3-chloro-4- fluoro-phenyl)-methyl- carbamoyl]imidazo[1,2- a]pyridin-3-yl]-2- pyridyl]carbamate | 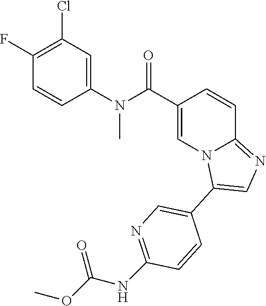 [SMILES: COC(=O)Nc1ccc(-c2cnc3ccc(C(=O)N(C)c4ccc(F)c(Cl)c4)cn23)cn1] | 1.09 | 454.2 | 1 | |
| 114 | methyl N-[5-[6-[(4- ethoxyphenyl)-methyl- carbamoyl]imidazo[1,2- a]pyridin-3-yl]-2- pyridyl]carbamate | 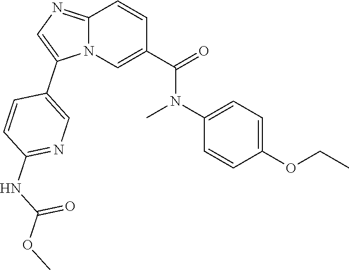 [SMILES: CCOc1ccc(N(C)C(=O)c2ccc3ncc(-c4ccc(NC(=O)OC)nc4)n3c2)cc1] | 1.09 | 446.2 | 1 | |
| 115 | methyl N-[5-[6- [methyl-[3- (methylcarbamoyl) phenyl]carbamoyl] imidazo[1,2- a]pyridin-3-yl]-2- pyridyl]carbamate | 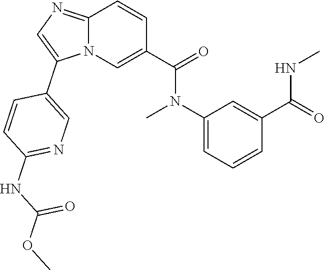 [SMILES: CNC(=O)c1cccc(N(C)C(=O)c2ccc3ncc(-c4ccc(NC(=O)OC)nc4)n3c2)c1] | 0.88 | 459.2 | 1 | |
| 116 | methyl N-[5-[6- [ethyl-(4- fluoro-3-methyl- phenyl)carbamoyl] imidazo [1,2-a]pyridin-3-yl]-2- pyridyl]carbamate | 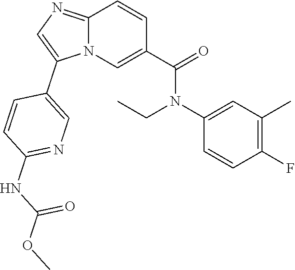 [SMILES: CCN(C(=O)c1ccc2ncc(-c3ccc(NC(=O)OC)nc3)n2c1)c1ccc(F)c(C)c1] | 1.16 | 448.2 | 1 | |
| 117 | methyl N-[5-[6- [(3-chloro-4- methyl-phenyl)-propyl- carbamoyl]imidazo[1,2- a]pyridin-3-yl]-2- pyridyl]carbamate | 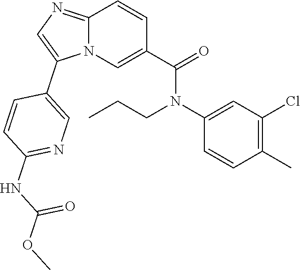 [SMILES: CCCN(C(=O)c1ccc2ncc(-c3ccc(NC(=O)OC)nc3)n2c1)c1ccc(C)c(Cl)c1] | 1.28 | 478.2 | 1 | |
| 118 | methyl N-[5-[6-[(3,4- dimethoxyphenyl)-(2- methoxyethyl) carbamoyl] imidazo[1,2-a]pyridin- 3-yl]-2- pyridyl]carbamate | 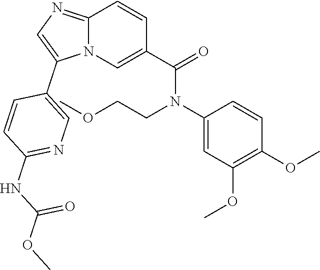 [SMILES: COCCN(C(=O)c1ccc2ncc(-c3ccc(NC(=O)OC)nc3)n2c1)c1ccc(OC)c(OC)c1] | 1.01 | 506.2 | 1 | |
| 119 | methyl N-[5-[6-[(3,4- dimethoxyphenyl)- ethyl-carbamoyl] imidazo[1,2- a]pyridin-3-yl]-2- pyridyl]carbamate | 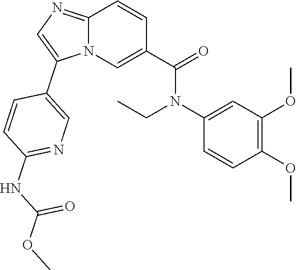 [SMILES: CCN(C(=O)c1ccc2ncc(-c3ccc(NC(=O)OC)nc3)n2c1)c1ccc(OC)c(OC)c1] | 1.01 | 476.2 | 1 | |
| 120 | methyl N-[5-[6- [cyclopropylmethyl- (3-fluoro-4-methyl- phenyl)carbamoyl] imidazo [1,2-a]pyridin-3-yl]-2- pyridyl]carbamate | 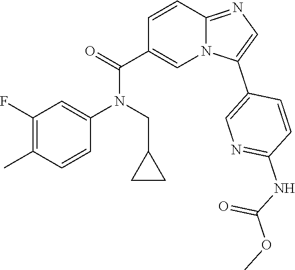 [SMILES: COC(=O)Nc1ccc(-c2cnc3ccc(C(=O)N(CC4CC4)c4ccc(C)c(F)c4)cn23)cn1] | 1.24 | 474.2 | 1 | |
| 121 | methyl N-[5-[6- [[4-chloro-3- (trifluoromethyl) phenyl]-methyl- carbamoyl]imidazo[1,2- a]pyridin-3-yl]-2- pyridyl]carbamate | 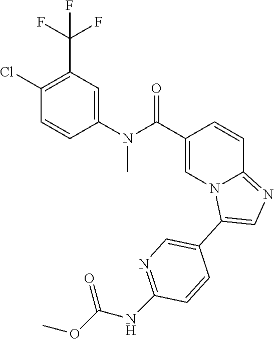 [SMILES: COC(=O)Nc1ccc(-c2cnc3ccc(C(=O)N(C)c4ccc(Cl)c(C(F)(F)F)c4)cn23)cn1] | 0.99 | 504.2 | 1 | |
| 122 | ethyl 3-[ethyl-[3-[6- (methoxycarbonyl- amino)-3- pyridyl]imidazo[1,2- a]pyridine-6-carbonyl] amino]benzoate | 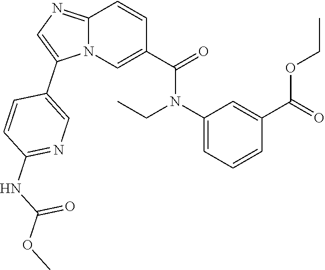 [SMILES: CCOC(=O)c1cccc(N(CC)C(=O)c2ccc3ncc(-c4ccc(NC(=O)OC)nc4)n3c2)c1] | 0.82 | 488.2 | 1 | |
| 123 | methyl N-[5-[6-[(4- chlorophenyl)-(2- ethoxyethyl)carbamoyl] imidazo[1,2-a]pyridin- 3-yl]-2- pyridyl]carbamate | 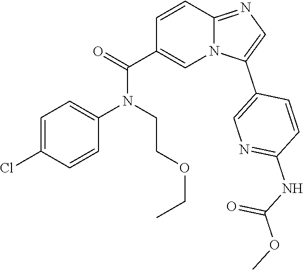 [SMILES: CCOCCN(C(=O)c1ccc2ncc(-c3ccc(NC(=O)OC)nc3)n2c1)c1ccc(Cl)cc1] | 1.16 | 494.0 | 1 | |
| 124 | methyl N-[5-[6- [ethyl-(3- methoxyphenyl) carbamoyl] imidazo[1,2-a]pyridin- 3-yl]-2- pyridyl]carbamate | 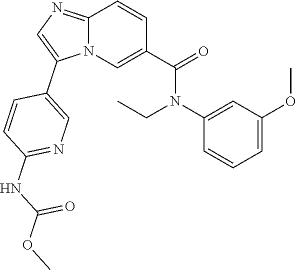 [SMILES: CCN(C(=O)c1ccc2ncc(-c3ccc(NC(=O)OC)nc3)n2c1)c1cccc(OC)c1] | 1.10 | 446.2 | 1 | |
| 125 | methyl N-[5-[6-[2- cyanoethyl(p-tolyl) carbamoyl]imidazo[1,2- a]pyridin-3-yl]-2- pyridyl]carbamate | 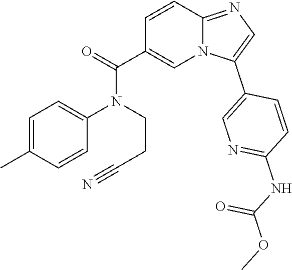 [SMILES: COC(=O)Nc1ccc(-c2cnc3ccc(C(=O)N(CCC#N)c4ccc(C)cc4)cn23)cn1] | 1.08 | 455.2 | 1 | |
| 126 | methyl N-[5-[6- [(4-chloro-3- methyl-phenyl)-propyl- carbamoyl]imidazo[1,2- a]pyridin-3-yl]-2- pyridyl]carbamate | 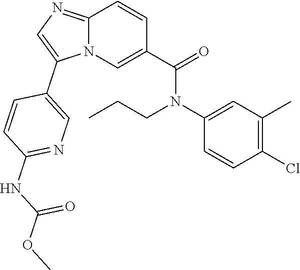 [SMILES: CCCN(C(=O)c1ccc2ncc(-c3ccc(NC(=O)OC)nc3)n2c1)c1ccc(Cl)c(C)c1] | 0.93 | 478.0 | 1 | |
| 127 | methyl N-[5-[6- [ethyl-(3-fluoro- 4-methyl-phenyl) carbamoyl]imidazo [1,2-a]pyridin-3-yl]-2- pyridyl]carbamate | 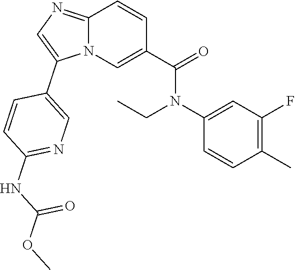 [SMILES: CCN(C(=O)c1ccc2ncc(-c3ccc(NC(=O)OC)nc3)n2c1)c1ccc(C)c(F)c1] | 0.82 | 448.2 | 1 | |
| 128 | methyl N-[5-[6-[(4- chlorophenyl)-(2- cyanoethyl)carbamoyl] imidazo[1,2- a]pyridin-3-yl]-2- pyridyl]carbamate | 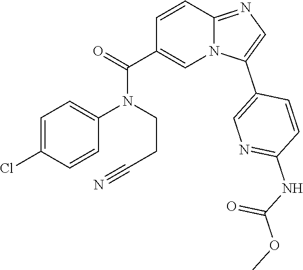 [SMILES: COC(=O)Nc1ccc(-c2cnc3ccc(C(=O)N(CCC#N)c4ccc(Cl)cc4)cn23)cn1] | 0.83 | 475.2 | 2 | |
| 129 | methyl N-[5-[6-[(4- chlorophenyl)-ethyl- carbamoyl]imidazo[1,2- a]pyridin-3-yl]-2- pyridyl]carbamate | 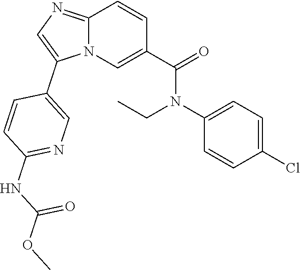 [SMILES: CCN(C(=O)c1ccc2ncc(-c3ccc(NC(=O)OC)nc3)n2c1)c1ccc(Cl)cc1] | | | | |
| 130 | methyl N-[5-[6-[2- cyanoethyl(m-tolyl) carbamoyl]imidazo [1,2-a]pyridin-3-yl]-2- pyridyl]carbamate | 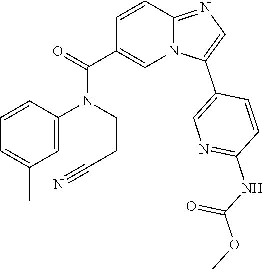 [SMILES: COC(=O)Nc1ccc(-c2cnc3ccc(C(=O)N(CCC#N)c4cccc(C)c4)cn23)cn1] | 1.01 | 455.3 | 1 | |
| 131 | methyl N-[5-[6-[(3,4- dimethoxyphenyl)- methyl-carbamoyl] imidazo[1,2- a]pyridin-3-yl]-2- pyridyl]carbamate | 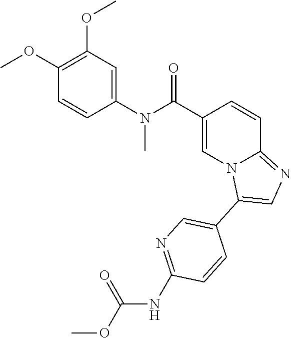 [SMILES: COC(=O)Nc1ccc(-c2cnc3ccc(C(=O)N(C)c4ccc(OC)c(OC)c4)cn23)cn1] | 0.94 | 462.1 | 1 | |
| 132 | methyl N-[5-[6-[2- cyanoethyl-(4- fluorophenyl) carbamoyl]imidazo [1,2-a]pyridin-3-yl]-2- pyridyl]carbamate | 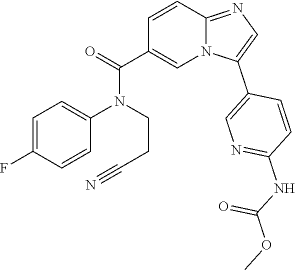 [SMILES: COC(=O)Nc1ccc(-c2cnc3ccc(C(=O)N(CCC#N)c4ccc(F)cc4)cn23)cn1] | 0.88 | 459.2 | 2 | |
| 133 | methyl N-[5-[6- [cyclopropylmethyl-(4- methoxyphenyl) carbamoyl] imidazo[1,2-a]pyridin- 3-yl]-2- pyridyl]carbamate | 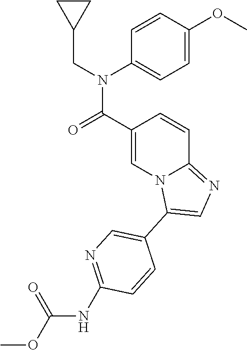 | 0.90 | 472.2 | 2 | |
| 134 | methyl N-[5-[6- [isobutyl(phenyl) carbamoyl] imidazo[1,2-a]pyridin- 3-yl]-2- pyridyl]carbamate | 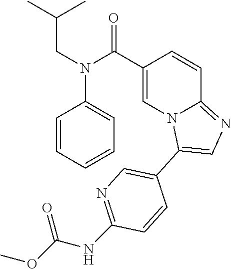 [SMILES: COC(=O)Nc1ccc(-c2cnc3ccc(C(=O)N(CC(C)C)c4ccccc4)cn23)cn1] | 0.93 | 444.2 | 2 | |
| 135 | methyl N-[5-[6-[(3,5- dimethylphenyl)-ethyl- carbamoyl]imidazo[1,2- a]pyridin-3-yl]-2- pyridyl]carbamate | 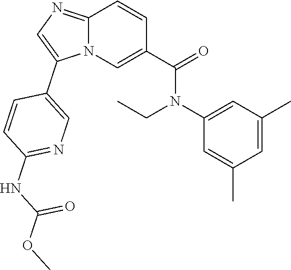 [SMILES: CCN(C(=O)c1ccc2ncc(-c3ccc(NC(=O)OC)nc3)n2c1)c1cc(C)cc(C)c1] | 1.18 | 444.2 | 1 | |
| 136 | methyl N-[5-[6- [ethyl-(4- fluorophenyl) carbamoyl] imidazo[1,2-a]pyridin- 3-yl]-2- pyridyl]carbamate | 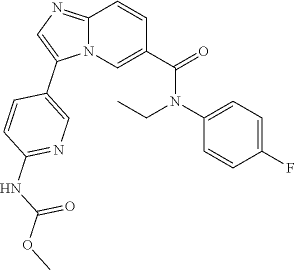 [SMILES: CCN(C(=O)c1ccc2ncc(-c3ccc(NC(=O)OC)nc3)n2c1)c1ccc(F)cc1] | 1.09 | 434.2 | 1 | |
| 137 | methyl N-[5-[6- [ethyl(phenyl) carbamoyl] imidazo[1,2-a]pyridin- 3-yl]-2- pyridyl]carbamate | 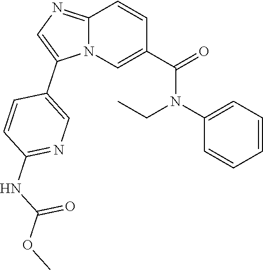 [SMILES: CCN(C(=O)c1ccc2ncc(-c3ccc(NC(=O)OC)nc3)n2c1)c1ccccc1] | 1.04 | 416.2 | 1 | |
| 138 | methyl N-[5-[6-[[2- (dimethylamino)-2-oxo- ethyl]-phenyl- carbamoyl]imidazo[1,2- a]pyridin-3-yl]-2- pyridyl]carbamate | 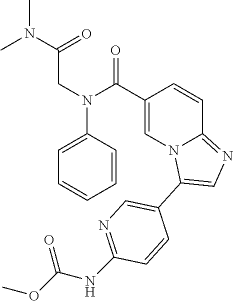 [SMILES: COC(=O)Nc1ccc(-c2cnc3ccc(C(=O)N(CC(=O)N(C)C)c4ccccc4)cn23)cn1] | 0.99 | 473.1 | 1 | |
| 139 | methyl N-[5-[6- [cyclopropylmethyl-(3- fluorophenyl) carbamoyl] imidazo[1,2-a]pyridin- 3-yl]-2- pyridyl]carbamate | 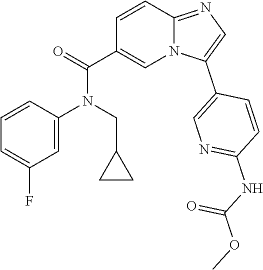 [SMILES: COC(=O)Nc1ccc(-c2cnc3ccc(C(=O)N(CC4CC4)c4cccc(F)c4)cn23)cn1] | 1.18 | 460.2 | 1 | |
| 140 | methyl N-[5-[6-[(4- fluorophenyl)-isopropyl- carbamoyl]imidazo[1,2- a]pyridin-3-yl]-2- pyridyl]carbamate | 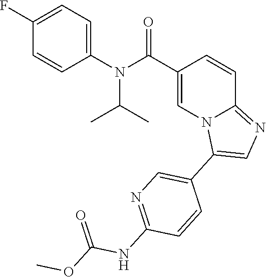 [SMILES: COC(=O)Nc1ccc(-c2cnc3ccc(C(=O)N(c4ccc(F)cc4)C(C)C)cn23)cn1] | 1.10 | 448.2 | 1 | |
| 141 | methyl N-[5-[6-[(3- cyanophenyl)-ethyl- carbamoyl]imidazo[1,2- a]pyridin-3-yl]-2- pyridyl]carbamate | 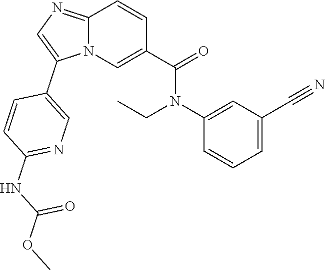 [SMILES: CCN(C(=O)c1ccc2ncc(-c3ccc(NC(=O)OC)nc3)n2c1)c1cccc(C#N)c1] | 1.04 | 441.2 | 1 | |
| 142 | methyl N-[5-[6- [cyanomethyl-(4- methoxyphenyl) carbamoyl] imidazo[1,2-a]pyridin- 3-yl]-2- pyridyl]carbamate | 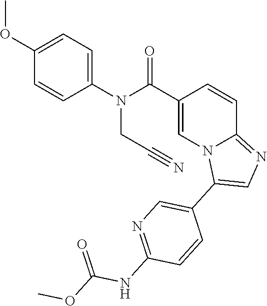 [SMILES: COC(=O)Nc1ccc(-c2cnc3ccc(C(=O)N(CC#N)c4ccc(OC)cc4)cn23)cn1] | 1.04 | 457.2 | 1 | |
| 143 | methyl N-[5-[6- [cyanomethyl(p- tolyl)carbamoyl] imidazo [1,2-a]pyridin-3-yl]-2- pyridyl]carbamate | 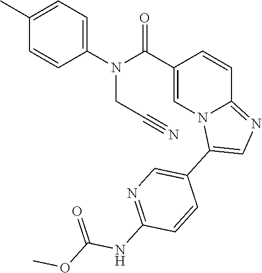 [SMILES: COC(=O)Nc1ccc(-c2cnc3ccc(C(=O)N(CC#N)c4ccc(C)cc4)cn23)cn1] | 1.05 | 441.2 | 1 | |
| 144 | methyl N-[5-[6- [methyl-[3- (trifluoromethoxy) phenyl]carbamoyl] imidazo[1,2- a]pyridin-3-yl]-2- pyridyl]carbamate | 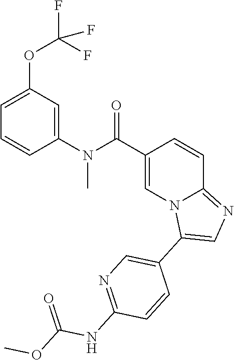 [SMILES: COC(=O)Nc1ccc(-c2cnc3ccc(C(=O)N(C)c4cccc(OC(F)(F)F)c4)cn23)cn1] | 1.16 | 486.0 | 1 | |
| 145 | methyl N-[5-[6- [ethyl-(3-fluorophenyl) carbamoyl] imidazo[1,2-a]pyridin- 3-yl]-2- pyridyl]carbamate | 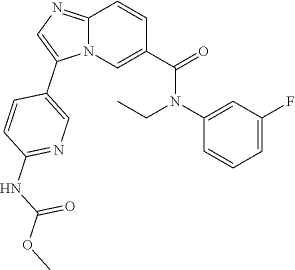 [SMILES: CCN(C(=O)c1ccc2ncc(-c3ccc(NC(=O)OC)nc3)n2c1)c1cccc(F)c1] | 1.06 | 434.2 | 1 | |
| 146 | methyl N-[5-[6-[(3- fluorophenyl)-[2- (methylamino)-2-oxo- ethyl]carbamoyl] imidazo [1,2-a]pyridin-3-yl]-2- pyridyl]carbamate | 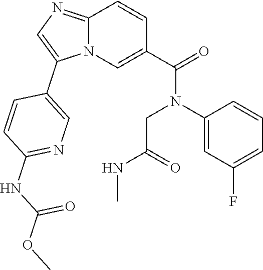 [SMILES: CNC(=O)CN(C(=O)c1ccc2ncc(-c3ccc(NC(=O)OC)nc3)n2c1)c1cccc(F)c1] | 0.95 | 477.2 | 1 | |
| 147 | methyl N-[5-[6- [cyclopropyl(phenyl) carbamoyl]imidazo [1,2-a]pyridin-3- yl]-2-pyridyl]carbamate | 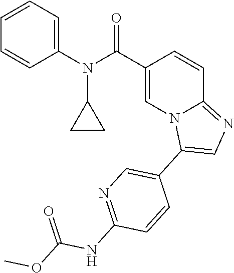 [SMILES: COC(=O)Nc1ccc(-c2cnc3ccc(C(=O)N(c4ccccc4)C4CC4)cn23)cn1] | 0.92 | 428.0 | 1 | |
| 148 | methyl N-[5-[6- [(4-chloro-3- methyl-phenyl)-(2- cyanoethyl)carbamoyl] imidazo[1,2- a]pyridin-3-yl]-2- pyridyl]carbamate | 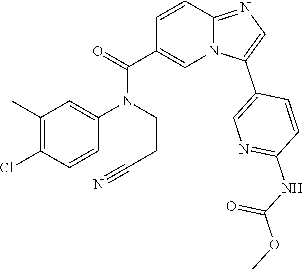 [SMILES: COC(=O)Nc1ccc(-c2cnc3ccc(C(=O)N(CCC#N)c4ccc(Cl)c(C)c4)cn23)cn1] | 1.13 | 489.2 | 1 | |
| 149 | methyl N-[5-[6-[2- cyanoethyl-(3- methoxyphenyl) carbamoyl] imidazo[1,2-a]pyridin- 3-yl]-2- pyridyl]carbamate | 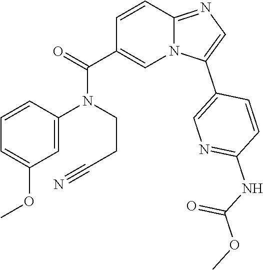 [SMILES: COC(=O)Nc1ccc(-c2cnc3ccc(C(=O)N(CCC#N)c4cccc(OC)c4)cn23)cn1] | 1.03 | 471.2 | 1 | |
| 150 | ethyl 2-(4-fluoro- N-[3-[6-(methoxy- carbonylamino)- 3-pyridyl]imidazo[1,2- a]pyridine-6- carbonyl]anilino)acetate | 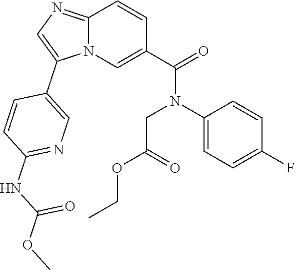 [SMILES: CCOC(=O)CN(C(=O)c1ccc2ncc(-c3ccc(NC(=O)OC)nc3)n2c1)c1ccc(F)cc1] | 0.86 | 492.2 | 2 | |
| 151 | N-(4-fluoro-3-methoxy- phenyl)-N-methyl-3-[6- (propanoylamino)-3- pyridyl]imidazo[1,2- a]pyridine-6- carboxamide | 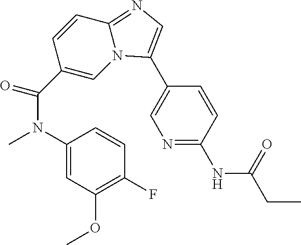 [SMILES: CCC(=O)Nc1ccc(-c2cnc3ccc(C(=O)N(C)c4ccc(F)c(OC)c4)cn23)cn1] | | | | 135-137 |
| 152 | N-(4-fluoro-3-methoxy- phenyl)-3-[6-[(2- methoxyacetyl)amino]- 3-pyridyl]-N-methyl- imidazo[1,2-a]pyridine- 6-carboxamide | 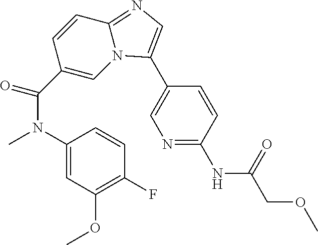 [SMILES: COCC(=O)Nc1ccc(-c2cnc3ccc(C(=O)N(C)c4ccc(F)c(OC)c4)cn23)cn1] | | | | 140-142 |
| 153 | 3-(6-acetamido-3- pyridyl)-N-(4-fluoro- 3-methoxy-phenyl)- N-methyl-imidazo[1,2- a]pyridine-6- carboxamide | 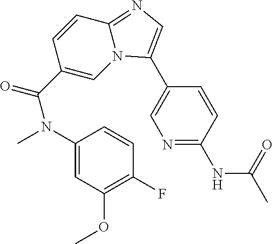 [SMILES: COc1cc(N(C)C(=O)c2ccc3ncc(-c4ccc(NC(C)=O)nc4)n3c2)ccc1F] | | | | 120-122 |
| 154 | 3-[6-(cyclopropane- carbonylamino)-3- pyridyl]-N-(4-fluoro-3- methoxy-phenyl)- N-methyl-imidazo [1,2-a]pyridine-6- carboxamide | 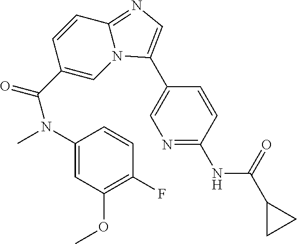 [SMILES: COc1cc(N(C)C(=O)c2ccc3ncc(-c4ccc(NC(=O)C5CC5)nc4)n3c2)ccc1F] | | | | 106-108 |
| 155 | 3-(6-acetamido-3- pyridyl)-N-(4- fluorophenyl)-N-methyl- imidazo[1,2-a]pyridine- 6-carboxamide | 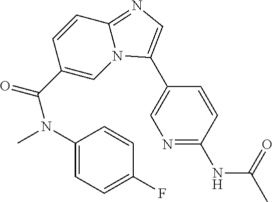 [SMILES: CC(=O)Nc1ccc(-c2cnc3ccc(C(=O)N(C)c4ccc(F)cc4)cn23)cn1] | | | | 196-198 |
| 156 | N-(4-fluorophenyl)- 3-[6-[(2-methoxyacetyl) amino]-3- pyridyl]-N-methyl- imidazo[1,2-a]pyridine- 6-carboxamide | 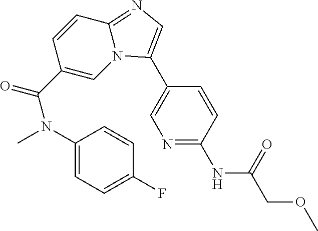 [SMILES: COCC(=O)Nc1ccc(-c2cnc3ccc(C(=O)N(C)c4ccc(F)cc4)cn23)cn1] | | | | 155-157 |
| 157 | methyl 4-[[3-[4- (methoxycarbonylamino) phenyl]imidazo[1,2- a]pyridine-6- carbonyl]-methyl- amino]benzoate | 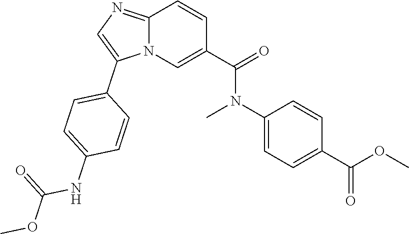 | 0.97 | 459.5 | B | |
| 158 | methyl N-[4-[6- [(4-fluoro-3-methoxy- phenyl)-methyl- carbamoyl]imidazo[1,2- a]pyridin-3- yl]phenyl]carbamate | 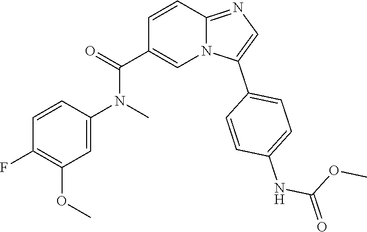 [SMILES: COC(=O)Nc1ccc(-c2cnc3ccc(C(=O)N(C)c4ccc(F)c(OC)c4)cn23)cc1] | 0.70 | 449 | A | 208-210 |
| 159 | methyl N-[5-[6- [methyl-(4- methylsulfanylphenyl) carbamoyl]imidazo [1,2-a]pyridin- 3-yl]-2-pyridyl] carbamate | 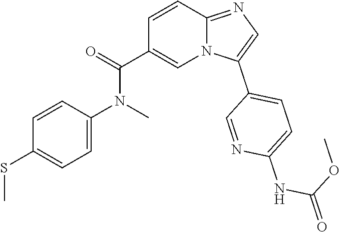 [SMILES: COC(=O)Nc1ccc(-c2cnc3ccc(C(=O)N(C)c4ccc(SC)cc4)cn23)cn1] | 0.95 | 448.2 | B | |
| 160 | methyl N-[5-[6- [methyl-(3- methylsulfanylphenyl) carbamoyl]imidazo [1,2-a]pyridin- 3-yl]-2-pyridyl] carbamate | 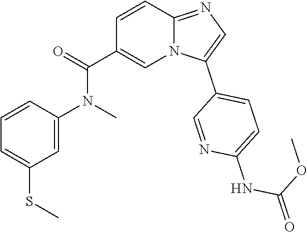 [SMILES: COC(=O)Nc1ccc(-c2cnc3ccc(C(=O)N(C)c4cccc(SC)c4)cn23)cn1] | 0.95 | 448.2 | B | |
| 161 | methyl N-[5-[6- [methyl-(4- methylsulfonylphenyl) carbamoyl]imidazo [1,2-a]pyridin- 3-yl]-2-pyridyl] carbamate | 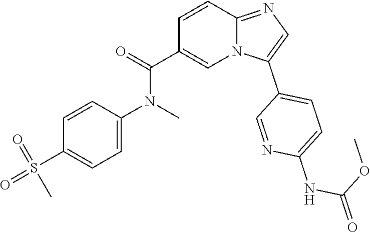 [SMILES: COC(=O)Nc1ccc(-c2cnc3ccc(C(=O)N(C)c4ccc(S(C)(=O)=O)cc4)cn23)cn1] | 0.66 | 480.2 | B | |
| 162 | methyl N-[5-[6- [methyl-(3- methylsulfonylphenyl) carbamoyl]imidazo [1,2-a]pyridin- 3-yl]-2-pyridyl] carbamate | 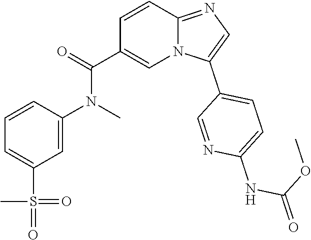 [SMILES: COC(=O)Nc1ccc(-c2cnc3ccc(C(=O)N(C)c4cccc(S(C)(=O)=O)c4)cn23)cn1] | 0.66 | 480.2 | B | |
| 163 | methyl N-[5-[6- [methyl-[4- (methylcarbamoyl) phenyl]carbamoyl] imidazo[1,2- a]pyridin-3-yl]-2- pyridyl]carbamate | 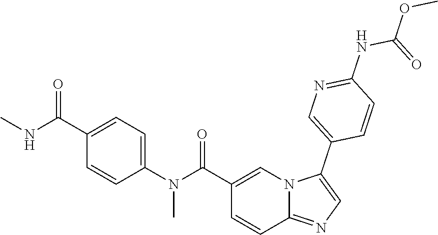 | 0.63 | 459.2 | B | |
| 164 | methyl N-[5-[6-[(4- fluorophenyl)-methyl- carbamothioyl]imidazo [1,2-a]pyridin-3-yl]-2- pyridyl]carbamate | 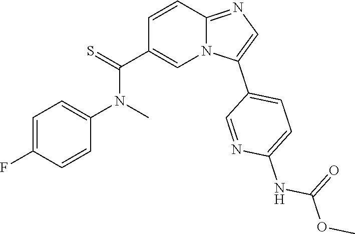 [SMILES: COC(=O)Nc1ccc(-c2cnc3ccc(C(=S)N(C)c4ccc(F)cc4)cn23)cn1] | 0.77 | 435 | A | |
| 165 | 3-[6-(cyclo- propanecarbonylamino)- 3-pyridyl]-N-(4- fluorophenyl)-N- methyl-imidazo[1,2- a]pyridine-6- carboxamide | 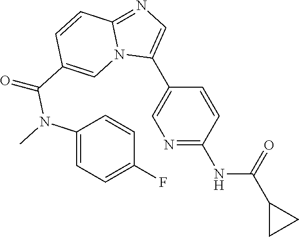 [SMILES: CN(C(=O)c1ccc2ncc(-c3ccc(NC(=O)C4CC4)nc3)n2c1)c1ccc(F)cc1] | | | | 143-145 |
| 166 | N-(4-fluorophenyl)- N-methyl-3-[6- (propanoylamino)-3- pyridyl]imidazo[1,2- a]pyridine-6- carboxamide | 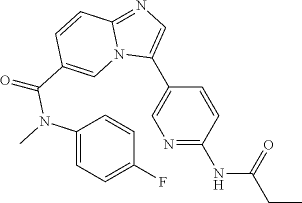 [SMILES: CCC(=O)Nc1ccc(-c2cnc3ccc(C(=O)N(C)c4ccc(F)cc4)cn23)cn1] | | | | |
| 167 | 3-(6-acetamido-3- pyridyl)-N-(4-fluoro- 3-methoxy-phenyl)- N,8-dimethyl-imidazo [1,2-a]pyridine- 6-carboxamide | 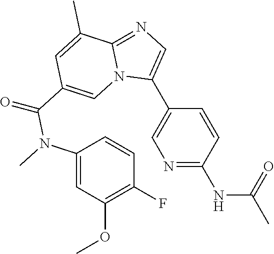 [SMILES: COc1cc(N(C)C(=O)c2cc(C)c3ncc(-c4ccc(NC(C)=O)nc4)n3c2)ccc1F] | | | | 180-182 |
| 168 | 3-(6-acetamido-3- pyridyl)-N-(4- fluorophenyl)-N,8- dimethyl-imidazo[1,2- a]pyridine-6- carboxamide | 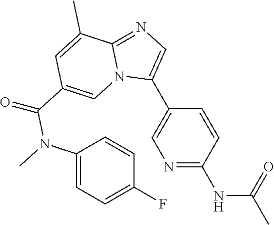 [SMILES: CC(=O)Nc1ccc(-c2cnc3c(C)cc(C(=O)N(C)c4ccc(F)cc4)cn23)cn1] | | | | 178-180 |
| 169 | methyl N-[5-[8- acetamido-6- [(4-fluoro-3-methoxy- phenyl)-methyl- carbamoyl]imidazo[1,2- a]pyridin-3-yl]-2- pyridyl]carbamate | 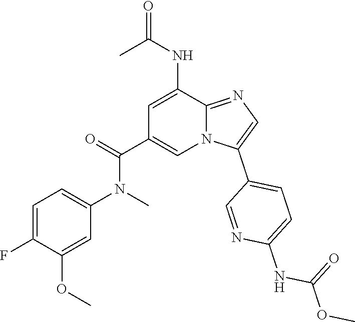 [SMILES: COC(=O)Nc1ccc(-c2cnc3c(NC(C)=O)cc(C(=O)N(C)c4ccc(F)c(OC)c4)cn23)cn1] | | | | 216-218 |
| 170 | methyl N-[5-[8- ethyl-6-[(4- fluorophenyl)-methyl- carbamoyl]imidazo[1,2- a]pyridin-3-yl]-2- pyridyl]carbamate | 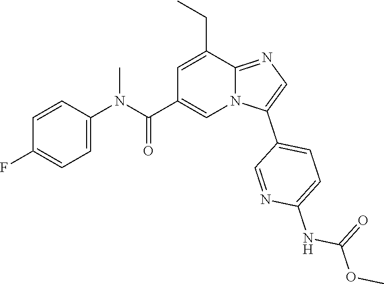 [SMILES: CCc1cc(C(=O)N(C)c2ccc(F)cc2)cn2c(-c3ccc(NC(=O)OC)nc3)cnc12] | | | | 183-185 |
| 171 | methyl N-[5-[6- [(3-chloro-4- fluoro-phenyl)-methyl- carbamoyl]-8-methyl- imidazo[1,2-a]pyridin- 3-yl]-2- pyridyl]carbamate | 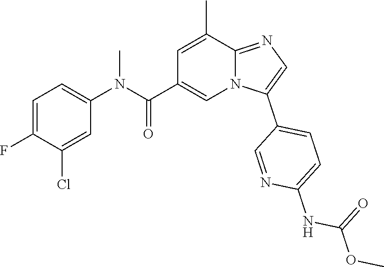 [SMILES: COC(=O)Nc1ccc(-c2cnc3c(C)cc(C(=O)N(C)c4ccc(F)c(Cl)c4)cn23)cn1] | | | | 147-149 |
| 172 | methyl N-[5-[6-[(4- fluorophenyl)-methyl- carbamoyl]-8- (methoxymethyl) imidazo[1,2- a]pyridin-3-yl]-2- pyridyl]carbamate | 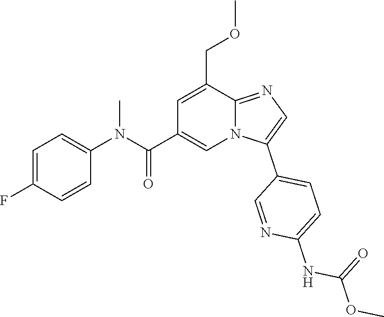 [SMILES: COCc1cc(C(=O)N(C)c2ccc(F)cc2)cn2c(-c3ccc(NC(=O)OC)nc3)cnc12] | | | | 191-193 |
| 173 | methyl N-[5-[6- [(4-fluoro-3- methoxy-phenyl)-(2- methoxyethyl) carbamoyl] imidazo[1,2-a]pyridin- 3-yl]-2- pyridyl]carbamate | 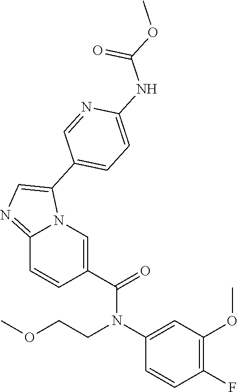 | | | | 172-175 |
| 174 | methyl N-[5-[6-[(3,4- difluorophenyl)-methyl- carbamoyl]-8-methyl- imidazo[1,2-a]pyridin- 3-yl]-2- pyridyl]carbamate | 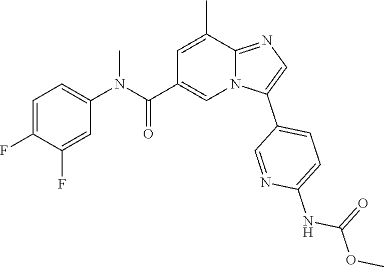 [SMILES: COC(=O)Nc1ccc(-c2cnc3c(C)cc(C(=O)N(C)c4ccc(F)c(F)c4)cn23)cn1] | | | | 138-140 |
| 175 | methyl N-[5-[6- [(3-cyano-4- fluoro-phenyl)-methyl- carbamoyl]imidazo[1,2- a]pyridin-3-yl]-2- pyridyl]carbamate | 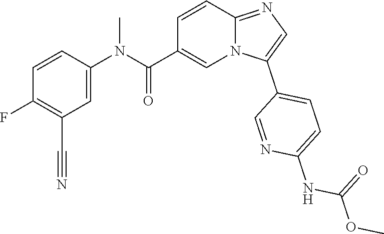 [SMILES: COC(=O)Nc1ccc(-c2cnc3ccc(C(=O)N(C)c4ccc(F)c(C#N)c4)cn23)cn1] | | | | 243-245 |
| 176 | ethyl 2-fluoro-5- [[3-[6-(methoxy- carbonylamino)- 3-pyridyl]imidazo[1,2- a]pyridine-6-carbonyl]- methyl-amino]benzoate | 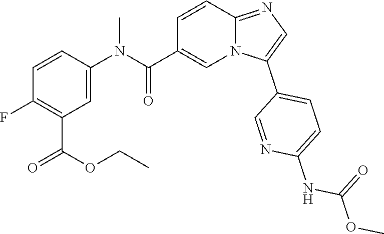 [SMILES: CCOC(=O)c1cc(N(C)C(=O)c2ccc3ncc(-c4ccc(NC(=O)OC)nc4)n3c2)ccc1F] | | | | 136-138 |
| 177 | methyl N-[5-[6- [(4-fluoro-3- methoxy-phenyl)-(2- methoxyethyl) carbamoyl]- 8-methyl-imidazo[1,2- a]pyridin-3-yl]-2- pyridyl]carbamate | 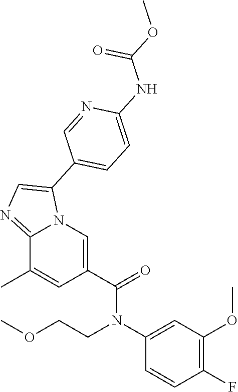 | | | | 159-162 |
| 178 | methyl N-[5-[6- [ethyl-(4- fluoro-3-methoxy- phenyl)carbamoyl] imidazo [1,2-a]pyridin-3-yl]-2- pyridyl]carbamate | 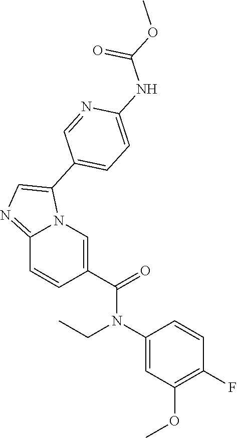 [SMILES: CCN(C(=O)c1ccc2ncc(-c3ccc(NC(=O)OC)nc3)n2c1)c1ccc(F)c(OC)c1] | | | | 145-148 |
| 179 | methyl N-[5-[6-[2- cyanoethyl-(4-fluoro-3- methoxy-phenyl) carbamoyl]- 8-methyl-imidazo[1,2- a]pyridin-3-yl]-2- pyridyl]carbamate | 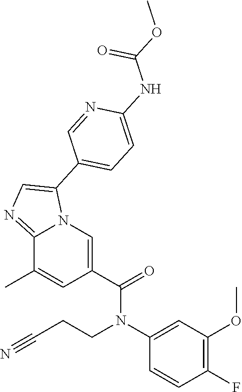 | | | | 152-154 |
| 180 | methyl N-[5-[6-[2- cyanoethyl-(4-fluoro-3- methoxy- phenyl)carbamoyl] imidazo [1,2-a]pyridin-3-yl]-2- pyridyl]carbamate | 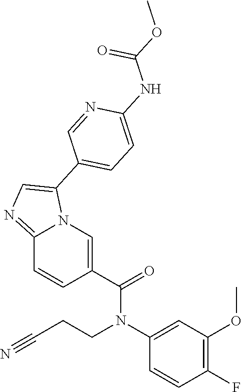 [SMILES: COC(=O)Nc1ccc(-c2cnc3ccc(C(=O)N(CCC#N)c4ccc(F)c(OC)c4)cn23)cn1] | | | | 134-137 |
| 181 | methyl N-[5-[6- [(4-cyano-3- ethoxy-phenyl)-methyl- carbamoyl]imidazo[1,2- a]pyridin-3-yl]-2- pyridyl]carbamate | 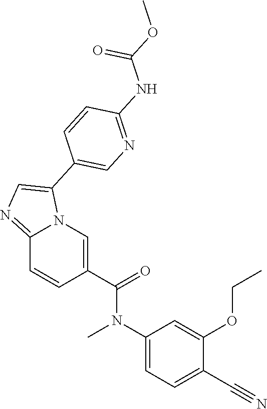 [SMILES: CCOc1cc(N(C)C(=O)c2ccc3ncc(-c4ccc(NC(=O)OC)nc4)n3c2)ccc1C#N] | | | | 212-216 |
| 182 | methyl N-[5-[6- [(4-fluoro-3-methoxy- phenyl)-prop-2- ynyl-carbamoyl] imidazo[1,2- a]pyridin-3-yl]-2- pyridyl]carbamate | 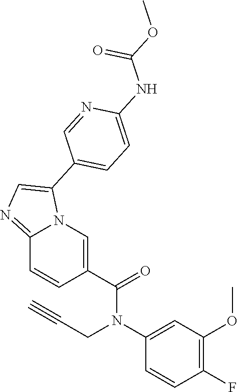 [SMILES: C#CCN(C(=O)c1ccc2ncc(-c3ccc(NC(=O)OC)nc3)n2c1)c1ccc(F)c(OC)c1] | | | | 161-163 |
| 183 | methyl N-[5-[6- [cyclopropyl- (4-fluoro-3-methoxy- phenyl)carbamoyl] imidazo[1,2- a]pyridin-3-yl]-2- pyridyl]carbamate | 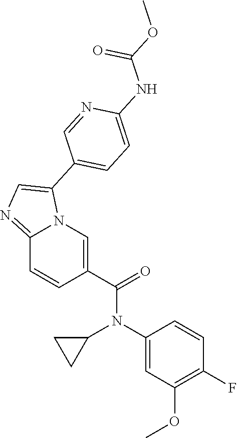 [SMILES: COC(=O)Nc1ccc(-c2cnc3ccc(C(=O)N(c4ccc(F)c(OC)c4)C4CC4)cn23)cn1] | | | | 112-114 |
| 184 | methyl N-[5-[6- [(4-chloro-3- methoxy-phenyl)- methyl-carbamoyl]- 8-methyl-imidazo [1,2-a]pyridin-3- yl]-2-pyridyl]carbamate | 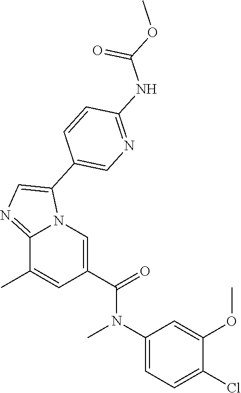 | | | | 196-199 |
| 185 | methyl N-[5-[6- [(4-cyano-3-methoxy- phenyl)-methyl- carbamoyl]-8-methyl- imidazo[1,2-a]pyridin- 3-yl]-2- pyridyl]carbamate | 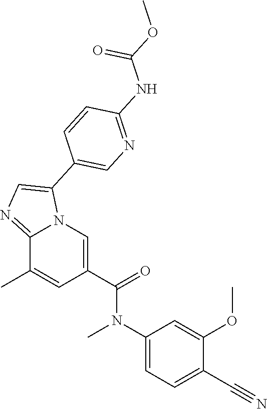 [SMILES: COC(=O)Nc1ccc(-c2cnc3c(C)cc(C(=O)N(C)c4ccc(C#N)c(OC)c4)cn23)cn1] | | | | 197-199 |
| 186 | methyl N-[5-[6- [(4-cyano-3- fluoro-phenyl)-methyl- carbamoyl]-8-methyl- imidazo[1,2-a]pyridin- 3-yl]-2- pyridyl]carbamate | 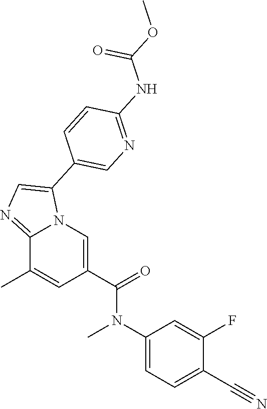 [SMILES: COC(=O)Nc1ccc(-c2cnc3c(C)cc(C(=O)N(C)c4ccc(C#N)c(F)c4)cn23)cn1] | | | | 205-207 |
| 187 | methyl N-[5-[6- [ethyl-(4- fluoro-3-methoxy- phenyl)carbamoyl]- 8-methyl- imidazo[1,2-a]pyridin- 3-yl]-2- pyridyl]carbamate | 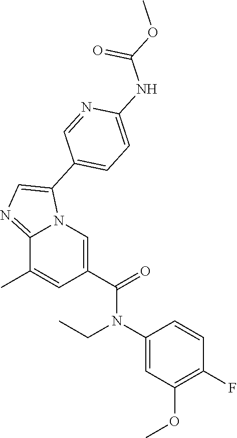 [SMILES: CCN(C(=O)c1cc(C)c2ncc(-c3ccc(NC(=O)OC)nc3)n2c1)c1ccc(F)c(OC)c1] | | | | 119-121 |
| 188 | methyl N-[5-[6- [(3-ethoxy-4- fluoro-phenyl)-methyl- carbamoyl]-8-methyl- imidazo[1,2-a]pyridin- 3-yl]-2- pyridyl]carbamate | 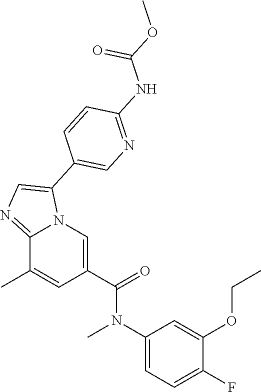 [SMILES: CCOc1cc(N(C)C(=O)c2cc(C)c3ncc(-c4ccc(NC(=O)OC)nc4)n3c2)ccc1F] | | | | 182-185 |
| 189 | methyl N-[5-[6- [(3-ethoxy-4- fluoro-phenyl)-methyl- carbamoyl]imidazo[1,2- a]pyridin-3-yl]-2- pyridyl]carbamate | 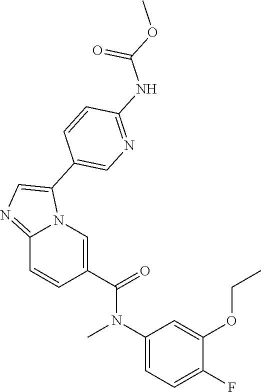 [SMILES: CCOc1cc(N(C)C(=O)c2ccc3ncc(-c4ccc(NC(=O)OC)nc4)n3c2)ccc1F] | | | | 182-184 |
| 190 | methyl N-[5-[6- [(4-cyano-3- ethoxy-phenyl)-methyl- carbamoyl]-8-methyl- imidazo[1,2-a]pyridin- 3-yl]-2- pyridyl]carbamate | 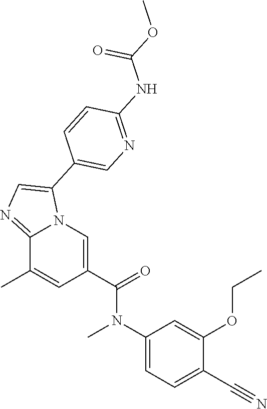 [SMILES: CCOc1cc(N(C)C(=O)c2cc(C)c3ncc(-c4ccc(NC(=O)OC)nc4)n3c2)ccc1C#N] | | | | 219-222 |
| 191 | methyl N-[5-[6- [(4-cyano-3- fluoro-phenyl)-methyl- carbamoyl]imidazo[1,2- a]pyridin-3-yl]-2- pyridyl]carbamate | 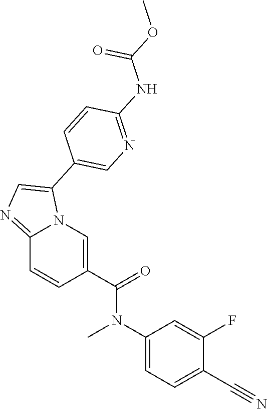 [SMILES: COC(=O)Nc1ccc(-c2cnc3ccc(C(=O)N(C)c4ccc(C#N)c(F)c4)cn23)cn1] | | | | 232-234 |
| 192 | methyl N-[5- [6-[cyclopropyl- (4-fluoro-3-methoxy- phenyl)carbamoyl]- 8-methyl- imidazo[1,2-a]pyridin- 3-yl]-2- pyridyl]carbamate | 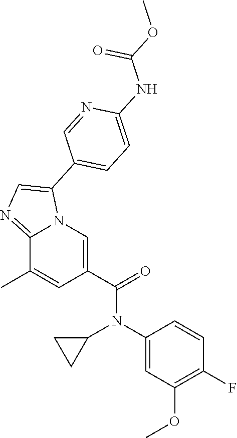 [SMILES: COC(=O)Nc1ccc(-c2cnc3c(C)cc(C(=O)N(c4ccc(F)c(OC)c4)C4CC4)cn23)cn1] | | | | 114-117 |
| 193 | methyl N-[5-[6- [(4-fluoro-3- methoxy-phenyl)- prop-2-ynyl- carbamoyl]-8-methyl- imidazo[1,2-a]pyridin- 3-yl]-2- pyridyl]carbamate | 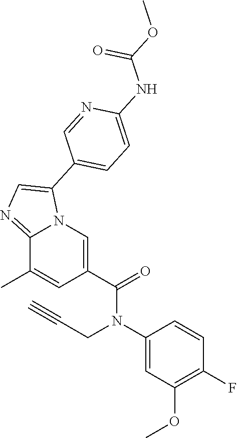 [SMILES: C#CCN(C(=O)c1cc(C)c2ncc(-c3ccc(NC(=O)OC)nc3)n2c1)c1ccc(F)c(OC)c1] | | | | 168-170 |
| 194 | methyl N-[5-[8- ethyl-6-[(4-fluoro- 3-methoxy-phenyl)- methyl- carbamoyl]imidazo[1,2- a]pyridin-3-yl]-2- pyridyl]carbamate | 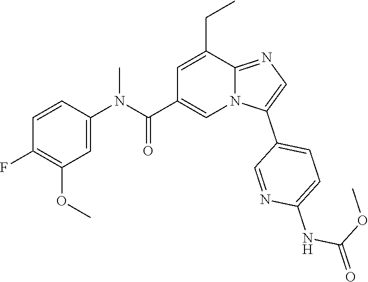 [SMILES: CCc1cc(C(=O)N(C)c2ccc(F)c(OC)c2)cn2c(-c3ccc(NC(=O)OC)nc3)cnc12] | | | | 168-170 |
| 195 | methyl N-[5-[8- (cyclopropylmethyl)-6- [(4-fluorophenyl)- methyl-carbamoyl] imidazo[1,2- a]pyridin-3-yl]-2- pyridyl]carbamate | 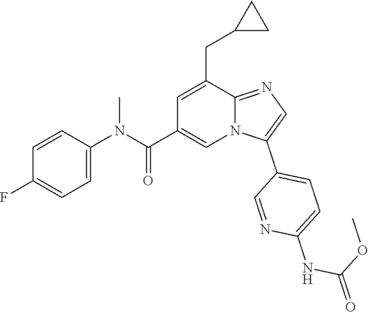 [SMILES: COC(=O)Nc1ccc(-c2cnc3c(CC4CC4)cc(C(=O)N(C)c4ccc(F)cc4)cn23)cn1] | | | | 176-178 |
| 196 | methyl N-[5-[6- [(3-cyano-4- fluoro-phenyl)-methyl- carbamoyl]-8-methyl- imidazo[1,2-a]pyridin- 3-yl]-2- pyridyl]carbamate | 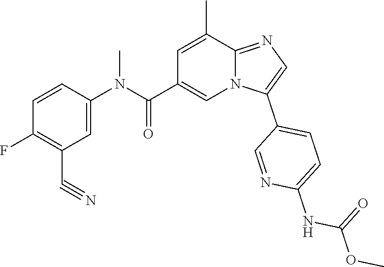 [SMILES: COC(=O)Nc1ccc(-c2cnc3c(C)cc(C(=O)N(C)c4ccc(F)c(C#N)c4)cn23)cn1] | | | | 133-135 |
| 197 | ethyl 2-fluoro-5-[[3-[6- (methoxycarbonyl- amino)- 3-pyridyl]-8-methyl- imidazo[1,2-a]pyridine- 6-carbonyl]-methyl- amino]benzoate | 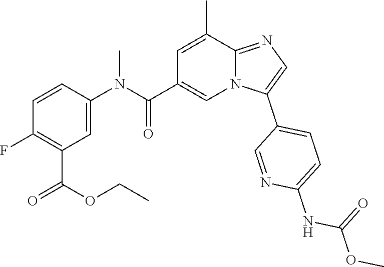 [SMILES: CCOC(=O)c1cc(N(C)C(=O)c2cc(C)c3ncc(-c4ccc(NC(=O)OC)nc4)n3c2)ccc1F] | | | | 124-126 |
| 198 | methyl N-[5-[6- [(4-fluoro- 3-methoxy-phenyl)- methyl-carbamoyl]-8- (methoxymethyl) imidazo[1,2- a]pyridin-3-yl]-2- pyridyl]carbamate | 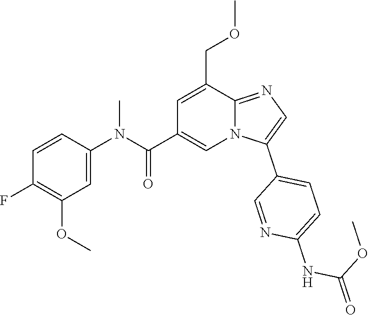 [SMILES: COCc1cc(C(=O)N(C)c2ccc(F)c(OC)c2)cn2c(-c3ccc(NC(=O)OC)nc3)cnc12] | | | | 180-182 |
| 199 | methyl N-[5-[6- [[4-fluoro-3-(methyl- carbamoyl)phenyl]- methyl-carbamoyl]- 8-methyl- imidazo[1,2-a]pyridin- 3-yl]-2- pyridyl]carbamate | 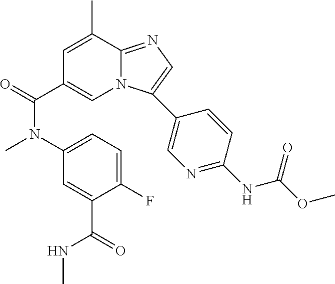 [SMILES: CNC(=O)c1cc(N(C)C(=O)c2cc(C)c3ncc(-c4ccc(NC(=O)OC)nc4)n3c2)ccc1F] | | | | 117-119 |
| 200 | methyl N-[5-[8- cyclopropyl- 6-[(4-fluoro-3-methoxy- phenyl)-methyl- carbamoyl]imidazo[1,2- a]pyridin-3-yl]-2- pyridyl]carbamate | 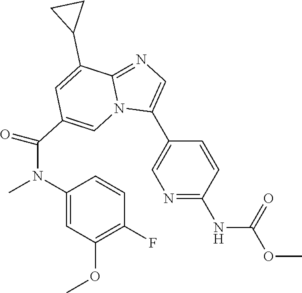 [SMILES: COC(=O)Nc1ccc(-c2cnc3c(C4CC4)cc(C(=O)N(C)c4ccc(F)c(OC)c4)cn23)cn1] | | | | 237-239 |
| 201 | methyl N-[5-[8- (cyclopropylmethyl)- 6-[(4-fluoro-3- methoxy-phenyl)- methyl-carbamoyl] imidazo[1,2- a]pyridin-3-yl]-2- pyridyl]carbamate | 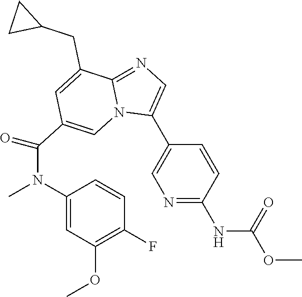 [SMILES: COC(=O)Nc1ccc(-c2cnc3c(CC4CC4)cc(C(=O)N(C)c4ccc(F)c(OC)c4)cn23)cn1] | | | | 218-220 |
| 202 | methyl N-[5-[6- [[4-fluoro-3- (methylcarbamoyl) phenyl]-methyl- carbamoyl]imidazo [1,2-a]pyridin-3-yl]-2- pyridyl]carbamate | 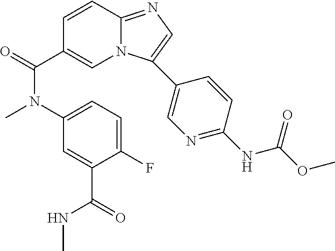 [SMILES: CNC(=O)c1cc(N(C)C(=O)c2ccc3ncc(-c4ccc(NC(=O)OC)nc4)n3c2)ccc1F] | | | | 220-222 |
| 203 | methyl 2-fluoro-5-[[3-[6- (methoxycarbonyl- amino)-3-pyridyl] imidazo[1,2- a]pyridine-6-carbonyl]- methyl-amino]benzoate | 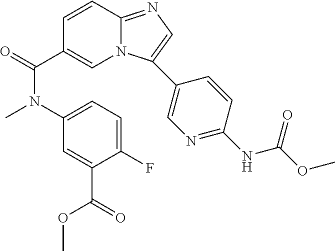 [SMILES: COC(=O)Nc1ccc(-c2cnc3ccc(C(=O)N(C)c4ccc(F)c(C(=O)OC)c4)cn23)cn1] | | | | 138-140 |
| 204 | methyl 2-fluoro-5-[[3-[6- (methoxycarbonyl- amino)-3-pyridyl]- 8-methyl-imidazo [1,2-a]pyridine-6- carbonyl]-methyl- amino]benzoate | 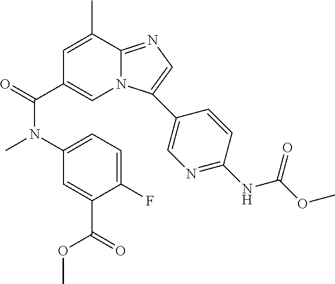 [SMILES: COC(=O)Nc1ccc(-c2cnc3c(C)cc(C(=O)N(C)c4ccc(F)c(C(=O)OC)c4)cn23)cn1] | | | | 151-153 |
| 205 | methyl N-[5-[6- [cyanomethyl-(4-fluoro- 3-methoxy-phenyl) carbamoyl]- 8-methyl-imidazo[1,2- a]pyridin-3-yl]-2- pyridyl]carbamate | 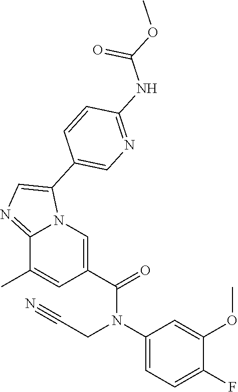 | | | | 159-162 |
| 206 | ethyl N-[5-[6- [(4-fluoro-3-methoxy- phenyl)-methyl- carbamoyl]imidazo[1,2- a]pyridin-3-yl]-2- pyridyl]carbamate | 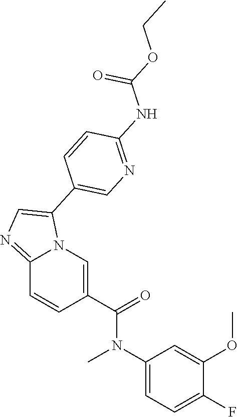 [SMILES: CCOC(=O)Nc1ccc(-c2cnc3ccc(C(=O)N(C)c4ccc(F)c(OC)c4)cn23)cn1] | | | | 191-193 |
| 207 | ethyl N-[5-[6- [(4-fluoro-3-methoxy- phenyl)-methyl- carbamoyl]-8-methyl- imidazo[1,2-a]pyridin- 3-yl]-2- pyridyl]carbamate | 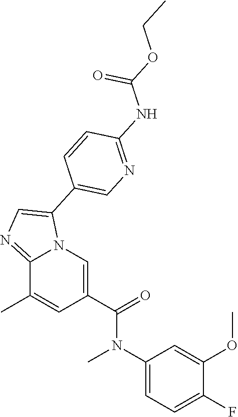 | | | | 181-183 |
| 208 | ethyl N-[5-[6-[(4- fluorophenyl)-methyl- carbamoyl]imidazo[1,2- a]pyridin-3-yl]-2- pyridyl]carbamate | 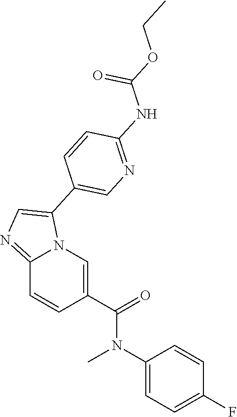 [SMILES: CCOC(=O)Nc1ccc(-c2cnc3ccc(C(=O)N(C)c4ccc(F)cc4)cn23)cn1] | | | | 123-125 |
| 209 | ethyl N-[5-[6-[(4- fluorophenyl)-methyl- carbamoyl]-8-methyl- imidazo[1,2-a]pyridin- 3-yl]-2- pyridyl]carbamate | 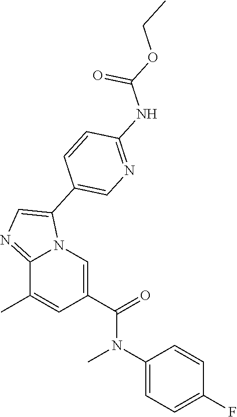 [SMILES: CCOC(=O)Nc1ccc(-c2cnc3c(C)cc(C(=O)N(C)c4ccc(F)cc4)cn23)cn1] | | | | 108-111 |
| 210 | ethyl N-[5-[6-[(2- methoxy-4-pyridyl)- methyl-carbamoyl]- 8-methyl-imidazo[1,2- a]pyridin-3-yl]-2- pyridyl]carbamate | 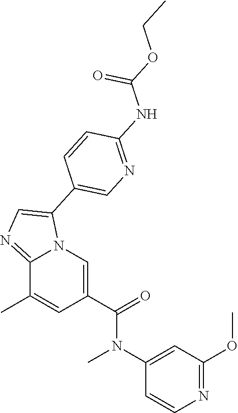 | | | | 231-233 |
| 211 | methyl N-[5-[6- [(2-methoxy- 4-pyridyl)-methyl- carbamoyl]-8-methyl- imidazo[1,2-a]pyridin- 3-yl]-2- pyridyl]carbamate | 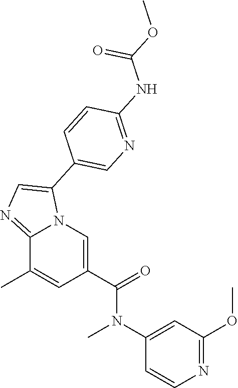 | | | | 161-163 |
| 212 | methyl N-[5-[6- [(4-cyano-3- methyl-phenyl)-methyl- carbamoyl]-8-methyl- imidazo[1,2-a]pyridin- 3-yl]-2- pyridyl]carbamate | 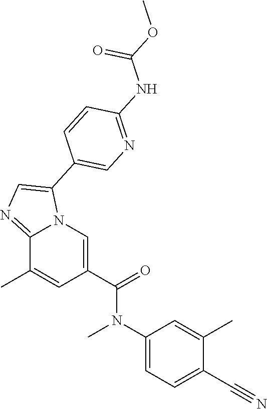 [SMILES: COC(=O)Nc1ccc(-c2cnc3c(C)cc(C(=O)N(C)c4ccc(C#N)c(C)c4)cn23)cn1] | | | | 132-135 |
| 213 | methyl N-[5-[8-amino- 6-[(4-fluoro-3-methoxy- phenyl)-methyl- carbamoyl]imidazo[1,2- a]pyridin-3-yl]-2- pyridyl]carbamate | 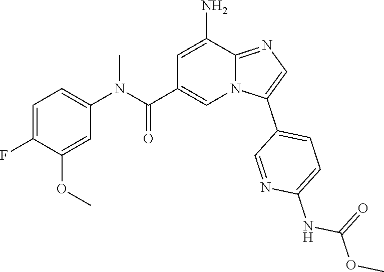 [SMILES: COC(=O)Nc1ccc(-c2cnc3c(N)cc(C(=O)N(C)c4ccc(F)c(OC)c4)cn23)cn1] | | | | |

Compound 129 was specifically characterized by NMR on a Brucker AVANCE III 400 MHz:

¹H NMR (DMSO-d₆, 400 MHz) δ: 10.48 (s, 1H), 8.40 (d, J=2.3 Hz, 1H), 8.34 (s, 1H), 8.02 (d, J=8.6 Hz, 1H), 7.79 (s, 1H), 7.74 (dd, J=8.7, 2.5 Hz, 1H), 7.50 (d, J=9.4 Hz, 1H), 7.39-7.32 (m, 4H), 7.14 (d, J=9.3 Hz, 1H), 3.87 (q, J=7.1, 7.1, 2H), 3.74 (s, 3H), 1.09 (t, J=7.0, 7.0 Hz, 3H).

Compound 166 was specifically characterized by NMR on a Brucker AVANCE III 400 MHz:

¹H NMR (DMSO-d₆, 400 MHz) δ: 1.08-1.33 (t, 3H), 2.43-2.48 (q, 2H), 3.35-3.39 (s, 3H), 7.05-7.22 (m, 3H), 7.35-7.41 (m, 2H), 7.50 (d, J=9.4 Hz, 1H), 7.77-7.82 (m, 2H), 8.26 (d, J=8.5 Hz, 1H), 8.35 (s, 1, H), 8.41 (d, J=2.0 Hz, 1H), 10.66 (s, 1H).

Compound 213 was specifically characterized by NMR on a Brucker AVANCE III 400 MHz:

¹H NMR (DMSO-d₆, 400 MHz) δ: 10.34 (s, 1H), 8.21 (d, 1H), 7.89 (d, 1H), 7.47-7.58 (m, 2H), 7.41 (s, 1H), 7.15-7.20 (m, 1H), 6.93-7.0 (m, 1H), 6.56-6.61 (m, 1H), 6.27 (s, 1H), 5.75 (br s, 2H), 3.64 (app s, 6H), 3.25 (s, 3H).

Example 1: This example illustrates the preparation of methyl N-[4-[6-[(4-fluorophenyl)-methyl-carbamoyl]imidazo[1,2-a]pyridin-3-yl]phenyl]carbamate (compound 11)

### Step 1: Preparation of methyl 3-(4-aminophenyl)imidazo[1,2-a]pyridine-6-carboxylate

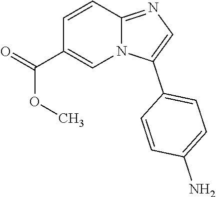 [SMILES: COC(=O)c1ccc2ncc(-c3ccc(N)cc3)n2c1]

Methyl 3-bromoimidazo[1,2-a]pyridine-6-carboxylate (1000 mg, 3.80 mmol, 1.00 eq.) and (4-aminophenyl)boronic acid hydrochloride salt (893 mg, 4.94 mmol, 1.30 eq.) were dissolved in dioxane (57 mL) and water (5.7 mL) to afford a yellow solution. Cesium carbonate (3130 mg, 9.51 mmol, 2.50 eq.) was added and the mixture was purged with a stream of argon for 5 minutes. CATACXIUM® Pd G3 (146 mg, 0.190 mmol, 0.05 eq.) was then added and the reaction mixture was heated over night at 80° C. The mixture was cooled down, diluted with ethyl acetate, and treated with a saturated solution of NaHCO₃. The desired material was extracted with ethyl acetate, the combined organic layers were washed with water, brine, dried over sodium sulfate, filtered, and concentrated under reduced pressure. The crude residue was purified over silica gel cartridge (ethyl acetate/MeOH) to afford methyl 3-(4-aminophenyl)imidazo[1,2-a]pyridine-6-carboxylate as a beige solid.

LC/MS (Method A) retention time=0.40 min; [M+H]⁺=268

¹H NMR (400 MHz, CDCl₃) δ: 9.01 (dd, J=1.1, 1.8 Hz, 1H), 7.75-7.71 (m, 1H), 7.68 (s, 1H), 7.67-7.64 (m, 1H), 7.38-7.33 (m, 2H), 6.89-6.84 (m, 2H), 3.94 (s, 3H), 3.93-3.88 (br s, 2H).

### Step 2: Preparation of methyl 3-[4-(methoxycarbonylamino)phenyl]imidazo[1,2-a]pyridine-6-carboxylate

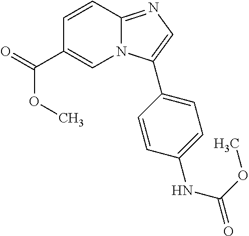 [SMILES: COC(=O)Nc1ccc(-c2cnc3ccc(C(=O)OC)cn23)cc1]

A mixture of methyl 3-(4-aminophenyl)imidazo[1,2-a]pyridine-6-carboxylate (905 mg, 3.22 mmol, 1.00 eq.) and pyridine (1280 mg, 1.31 mL, 16.1 mmol, 5.00 eq.) were dissolved in ethyl acetate (36 mL). Methyl chloroformate (614 mg, 0.502 mL, 6.43 mmol, 3.00 eq.) was added dropwise at 20°−25° C. and the mixture was stirred at room temperature for an additional 4 hours. The reaction mixture was then treated with a saturated solution of NaHCO₃. The precipitate that formed was filtered, and washed with water and ethyl acetate to afford methyl 3-[4-(methoxycarbonylamino)phenyl]imidazo[1,2-a]pyridine-6-carboxylate as an off-white solid.

LC/MS (Method A) retention time=0.66 min; [M+H]⁺=326

¹H NMR (400 MHz, DMSO-d₆) δ: 9.92 (br s, 1H), 8.95-8.91 (m, 1H), 7.82 (s, 1H), 7.76-7.71 (m, 1H), 7.71-7.65 (m, 3H), 7.64-7.59 (m, 2H), 3.88 (s, 3H), 3.72 (s, 3H).

### Step 3: Preparation of 3-[4-(methoxycarbonylamino)phenyl]imidazo[1,2-a]pyridine-6-carboxylic acid

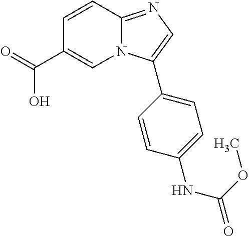 [SMILES: COC(=O)Nc1ccc(-c2cnc3ccc(C(=O)O)cn23)cc1]

Methyl 3-[4-(methoxycarbonylamino)phenyl]imidazo[1,2-a]pyridine-6-carboxylate (818 mg, 2.34 mmol, 1.00 eq.) was dissolved in THE/water 3:1 (40 mL). Lithium hydroxide (150 mg, 3.51 mmol, 1.50 eq.) was then added and the mixture was stirred at room temperature for 4 hours. The mixture was then diluted with water (10 mL) and the THF was evaporated under reduced pressure. The residue was acidified with HCl 2N to obtain a pH of 4. The suspension was filtered, the solid obtained was washed with water and dried in vacuo to 3-[4-(methoxycarbonylamino)phenyl]imidazo[1,2-a]pyridine-6-carboxylic acid as an off-white solid.

LC/MS (Method A) retention time=0.32 min; [M+H]⁺=310

¹H NMR (400 MHz, DMSO-d₆) δ 13.75-12.89 (br s, 1H), 9.89 (br s, 1H), 8.93 (t, J=1.1 Hz, 1H), 7.84 (s, 1H), 7.74-7.67 (m, 3H), 7.64-7.58 (m, 2H), 3.72 (s, 3H).

### Step 4: Preparation of methyl N-[4-[6-[(4-fluorophenyl)-methyl-carbamoyl]imidazo[1,2-a]pyridin-3-yl]phenyl]carbamate

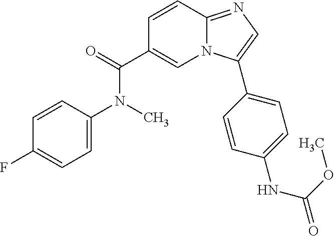 [SMILES: COC(=O)Nc1ccc(-c2cnc3ccc(C(=O)N(C)c4ccc(F)cc4)cn23)cc1]

To a solution of 3-[4-(methoxycarbonylamino)phenyl]imidazo[1,2-a]pyridine-6-carboxylic acid (0.042 mmol, 1 eq.) in dimethylacetamide (0.24 mL) was added a solution N-methyl-4-fluoroaniline (0.084 mmol, 2 eq.) in dimethylacetamide (0.40 mL), diisopropylethylamine (0.252 mmol, 6.0 eq) and a solution of bis(2-oxo-3-oxazolidinyl)phosphinic chloride (BOP—Cl) (0.105 mmol, 2.5 eq.). The vial was sealed and stirred at 70° C. for 16 hours. All the volatiles were evaporated under reduced pressure, and the resulting slurry was dissolved in methanol (1 mL), filtered, and concentrated again under reduced pressure. The crude residue was dissolved in dimethylacetamide (900 uL) and transferred to a preparative HPLC for purification to yield methyl N-[4-[6-[(4-fluorophenyl)-methyl-carbamoyl]imidazo[1,2-a]pyridin-3-yl]phenyl]carbamate as an off-white solid).

LC/MS (Method A) retention time=0.71 min; [M+H]⁺=419

¹H NMR (400 MHz, DMSO-d₆) δ 9.87 (br s, 1H), 8.30 (s, 1H), 7.68 (s, 1H), 7.66 (d, J=8.7 Hz, 2H), 7.51 (dd, J=0.7, 9.4 Hz, 1H), 7.41-7.34 (m, 2H), 7.27 (d, J=8.7 Hz, 2H), 7.22-7.06 (m, 3H), 3.72 (s, 3H), 3.37 (s, 3H).

Example 2: This example illustrates the preparation of methyl N-[4-[6-[(4-chlorophenyl)-methyl-carbamoyl]-8-methyl-imidazo[1,2-a]pyridin-3-yl]phenyl]carbamate (Compound 19)

### Step 1: Preparation of methyl 8-methylimidazo[1,2-a]pyridine-6-carboxylate

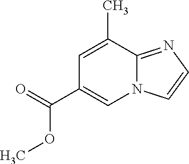

A sealed tube was charged with methyl 8-bromoimidazo[1,2-a]pyridine-6-carboxylate (750 mg, 2.65 mmol, 1 eq), methylboronic acid (323 mg, 5.29 mmol, 2 eq), potassium carbonate (731 mg, 5.29 mmol, 2 eq) and THE (20 mL). The reaction mixture was degassed with argon for 2 minutes, then chloro(2-dicyclohexylphosphino-2′,4′,6′-triisopropyl-1,1′-biphenyl)[2-(2′-amino-1,1′-biphenyl)]palladium(II) (104 mg, 0.132 mmol, 0.05 eq) was added. The reaction mixture was stirred at 80° C. for 6 hours. After cooling down to room temperature, the reaction mixture was filtered through a Celite pad and washed with ethyl acetate. The filtrate was washed with water, then brine, dried over sodium sulfate, filtered, and concentrated under reduced pressure. The crude residue was purified by flash chromatography to give methyl 8-methylimidazo[1,2-a]pyridine-6-carboxylate as an off-white solid.

¹H NMR (400 MHz, CDCl₃) δ: 8.81 (s, 1H), 7.70 (d, J=1.2 Hz, 1H), 7.66 (d, J=1.2 Hz, 1H), 7.54 (s, 1H), 3.95 (s, 3H), 2.65 (s, 3H).

### Step 2: Preparation of methyl 3-iodo-8-methyl-imidazo[1,2-a]pyridine-6-carboxylate

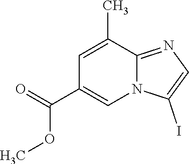

To a stirred solution of methyl 8-methylimidazo[1,2-a]pyridine-6-carboxylate (530 mg, 2.37 mmol, 1 eq) in dimethylformamide (10 mL) was added at room temperature N-iodosuccinimide (1.07 g, 4.74 mmol, 2 eq). The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 3-iodo-8-methyl-imidazo[1,2-a]pyridine-6-carboxylate as a brown solid.

¹H NMR (400 MHz, CDCl₃) δ: 8.76 (s, 1H), 7.79 (s, 1H), 7.67 (s, 1H), 3.99 (s, 3H), 2.76 (s, 3H).

### Step 3: Preparation of methyl 3-[4-(methoxycarbonylamino)phenyl]-8-methyl-imidazo[1,2-a]pyridine-6-carboxylate

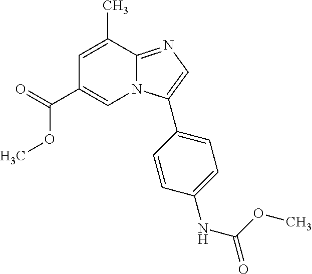 [SMILES: COC(=O)Nc1ccc(-c2cnc3c(C)cc(C(=O)OC)cn23)cc1]

A sealed tube was charged with methyl 3-iodo-8-methyl-imidazo[1,2-a]pyridine-6-carboxylate (50.0 mg, 0.127 mmol, 1 eq), methyl N-[4-(4,4,5,5-tetramethyl-1,3,2-dioxaborolan-2-yl)phenyl]carbamate (commercially available, CAS 844500-75-0, 77.9 mg, 0.253 mmol, 2 eq), potassium carbonate (2 mol/L, 0.127 mL, 0.253 mmol, 2 eq) and 1,4-dioxane (5 mL). The reaction mixture was degassed with argon for 2 minutes, then chloro(2-dicyclohexylphosphino-2′,4′,6′-triisopropyl-1,1′-biphenyl)[2-(2′-amino-1,1′-biphenyl)]palladium(II) (4.98 mg, 0.00633 mmol, 0.05 eq) was added. The reaction mixture was stirred under microwave irradiation at 80° C. for 6 hours. After cooling down to room temperature, the reaction mixture was filtered through a Celite pad which was further flushed with ethyl acetate. The filtrate was washed with water, then brine, dried over sodium sulfate, filtered, and concentrated under reduced pressure. The crude residue was purified by flash chromatography to give methyl 3-[4-(methoxycarbonylamino)phenyl]-8-methyl-imidazo[1,2-a]pyridine-6-carboxylate as an off-white solid.

¹H NMR (400 MHz, DMSO-d₆) δ: 9.91 (s, 1H), 8.79 (s, 1H), 7.6 (s, 1H), 7.67 (d, J=8.5 Hz, 2H) 7.58 (d, J=8.5 Hz, 2H), 7.52 (br s, 1H), 3.86 (s, 3H), 3.71 (s, 3H), 2.57 (s, 3H).

### Step 4: Preparation of 3-[4-(methoxycarbonylamino)phenyl]-8-methyl-imidazo[1,2-a]pyridine-6-carboxylic acid

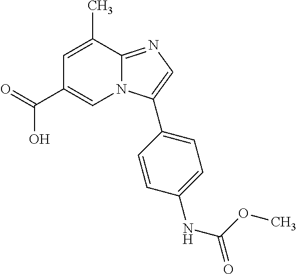 [SMILES: COC(=O)Nc1ccc(-c2cnc3c(C)cc(C(=O)O)cn23)cc1]

To a stirred solution of methyl 3-[4-(methoxycarbonylamino)phenyl]-8-methyl-imidazo[1,2-a]pyridine-6-carboxylate (30.0 mg, 0.0796 mmol, 1 eq) in a 3:1 mixture of THF:water (5 mL) was added at 0° C. lithium hydroxide monohydrate (6.68 mg, 0.159 mmol, 2 eq). The reaction mixture was stirred for 3 hours at room temperature after which it was concentrated under reduced pressure. The resulting residue was diluted with water and acidified with saturated NaHSO₄ aq. The precipitate that appeared, was filtered, and dried under reduced pressure to give 3-[4-(methoxycarbonylamino)phenyl]-8-methyl-imidazo[1,2-a]pyridine-6-carboxylic acid that was used in the next step without further purification.

¹H NMR (400 MHz, DMSO-d₆) δ: 13.24 (br s, 1H), 9.90 (s, 1H) 8.78 (s, 1H), 7.76 (s, 1H), 7.67 (d, J=8.8 Hz, 2H), 7.58 (d, J=8.8 Hz, 2H), 7.49-7.53 (m, 1H), 3.70 (s, 3H), 2.56 (s, 3H).

### Step 5: Preparation of methyl N-[4-[6-[(4-chlorophenyl)-methyl-carbamoyl]-8-methyl-imidazo[1,2-a]pyridin-3-yl]phenyl]carbamate

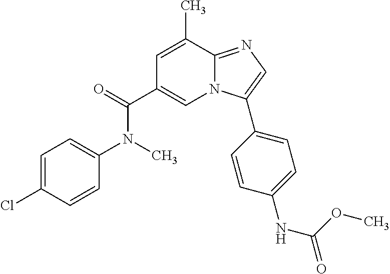 [SMILES: COC(=O)Nc1ccc(-c2cnc3c(C)cc(C(=O)N(C)c4ccc(Cl)cc4)cn23)cc1]

To a stirred solution of 3-[4-(methoxycarbonylamino)phenyl]-8-methyl-imidazo[1,2-a]pyridine-6-carboxylic acid (90.0 mg, 0.241 mmol, 1 eq) in pyridine (4 mL) was added 4-chloro-N-methyl-aniline (40.9 mg, 0.722 mmol, 1.2 eq). Then phosphorus(V) oxychloride (0.0661 mL, 0.722 mmol, 5 eq) was added at 0° C. The reaction mixture was stirred for 3 hours at room temperature. The reaction mixture was quenched with iced water and extracted twice with ethyl acetate. The combined organic layers were washed with saturated NaHSO₄ aq., then brine, dried over sodium sulfate, filtered, and concentrated under reduced pressure. The crude residue was purified by reverse phase chromatography to give methyl N-[4-[6-[(4-chlorophenyl)-methyl-carbamoyl]-8-methyl-imidazo[1,2-a]pyridin-3-yl]phenyl]carbamate as an amorphous white solid.

¹H NMR (400 MHz, CDCl₃) δ: 7.95 (s, 1H), 7.60 (s, 1H), 7.50 (d, J=8.1 Hz, 2H), 7.33 (d, J=8.6 Hz, 2H), 7.12 (s, 1H), 7.01-7.10 (m, 4H), 6.81 (br s, 1H), 3.85 (s, 3H), 3.47 (s, 3H), 2.56 (s, 3H).

Example 3: This example illustrates the preparation of methyl N-[4-[6-[(4-chlorophenyl)-methyl-carbamoyl]-5-methyl-imidazo[1,2-a]pyridin-3-yl]phenyl]carbamate (Compound 20)

### Step 1: Preparation of methyl 5-methylimidazo[1,2-a]pyridine-6-carboxylate

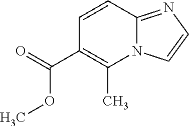

A sealed tube was charged with methyl 6-amino-2-methyl-pyridine-3-carboxylate (50.0 mg, 0.295 mmol, 1 eq), ethanol (3 mL) and 2-chloroacetaldehyde (231 mg, 1.47 mmol, 5 eq). The reaction mixture was stirred at 80° C. for 16 hours. After cooling down to room temperature, the reaction mixture was poured into a pre-stirred cold solution of saturated NaHCO₃ aq. and stirred for 10 minutes. The reaction mixture was then diluted with dichloromethane. The organic layer was separated, washed with brine, dried over sodium sulfate, filtered, and concentrated under reduced pressure. The crude residue was purified by column chromatography to give methyl 5-methylimidazo[1,2-a]pyridine-6-carboxylate as a brown solid.

¹H NMR (400 MHz, DMSO-d₆) δ: 8.49 (s, 1H), 8.30 (s, 1H), 8.17 (d, J=9.5 Hz, 1H), 7.90 (d, J=9.5 Hz, 1H), 3.93 (s, 3H), 3.03 (s, 3H).

### Step 2: Preparation of methyl 3-iodo-5-methyl-imidazo[1,2-a]pyridine-6-carboxylate

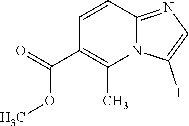

To a stirred solution of methyl 5-methylimidazo[1,2-a]pyridine-6-carboxylate (50.0 mg, 0.197 mmol, 1 eq) in dimethylformamide (0.5 mL) was added at room temperature N-iodosuccinimide (44.4 mg, 0.197 mmol, 1 eq). The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure.

The crude residue was purified by flash chromatography to give methyl 3-iodo-5-methyl-imidazo[1,2-a]pyridine-6-carboxylate as an off-white solid.

¹H NMR (400 MHz, DMSO-d₆) δ: 7.73 (br s, 1H), 7.53-7.56 (m, 2H), 3.86 (m, 3H), 3.38 (s, 3H).

### Step 3: Preparation of methyl 3-[4-(methoxycarbonylamino)phenyl]-5-methyl-imidazo[1,2-a]pyridine-6-carboxylate

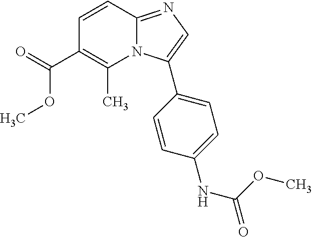 [SMILES: COC(=O)Nc1ccc(-c2cnc3ccc(C(=O)OC)c(C)n23)cc1]

A microwave vial was charged with methyl 3-iodo-5-methyl-imidazo[1,2-a]pyridine-6-carboxylate (450 mg, 1.42 mmol, 1 eq), methyl N-[4-(4,4,5,5-tetramethyl-1,3,2-dioxaborolan-2-yl)phenyl]carbamate (commercially available, CAS 844500-75-0, 592 mg, 2.14 mmol, 1.5 eq), cesium carbonate (928 mg, 2.85 mmol, 2 eq), 1,4-dioxane (10 mL) and water (2 mL). The reaction mixture was degassed with argon for 5 minutes, then Pd(dppf)Cl₂ (104 mg, 0.142 mmol, 0.1 eq) was added and it was degassed for an additional 2 minutes. The reaction mixture was heated to 90° C. under microwave irradiation and stirred for 16 hours. After cooling down to room temperature, the reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 3-[4-(methoxycarbonylamino)phenyl]-5-methyl-imidazo[1,2-a]pyridine-6-carboxylate as an off-white solid.

¹H NMR (400 MHz, DMSO-d₆) δ: 9.86 (s, 1H), 7.57-7.60 (m, 2H), 7.56 (s, 1H), 7.54 (d, J=8.5 Hz, 2H), 7.42 (d, J=8.5 Hz, 2H), 3.84 (s, 3H), 3.70 (s, 3H), 2.45 (s, 3H).

### Step 4: Preparation of 3-[4-(methoxycarbonylamino)phenyl]-5-methyl-imidazo[1,2-a]pyridine-6-carboxylic acid

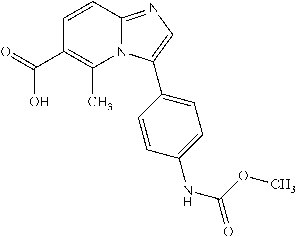 [SMILES: COC(=O)Nc1ccc(-c2cnc3ccc(C(=O)O)c(C)n23)cc1]

To a stirred solution of methyl 3-[4-(methoxycarbonylamino)phenyl]-5-methyl-imidazo[1,2-a]pyridine-6-carboxylate (10.0 mg, 0.00295 mmol, 1 eq) in THE (2 mL) and methanol (0.5 mL) was added at room temperature a solution of lithium hydroxide monohydrate (2.60 mg, 0.00589 mmol, 2 eq) in water (0.5 mL). The reaction mixture was stirred for 16 hours at room temperature. The reaction mixture was concentrated under reduced pressure and the resulting residue was diluted with water, and extracted with tert-butyl methyl ether. The aqueous layer was acidified with saturated NaHSO₄ aq., after which a precipitate appeared. The precipitate was dissolved in ethyl acetate, washed with brine, dried over sodium sulfate, filtered, and concentrated under reduced pressure to give 3-[4-(methoxycarbonylamino)phenyl]-5-methyl-imidazo[1,2-a]pyridine-6-carboxylic acid that was used in the next step without further purification.

¹H NMR (400 MHz, DMSO-d₆) δ: 13.20 (br s, 1H), 9.86 (s, 1H), 7.62 (d, J=7.6 Hz, 1H), 7.51-7.58 (m, 4H), 7.41 (d, J=8.5 Hz, 2H), 3.70 (s, 3H), 2.47 (s, 3H).

### Step 5: Preparation of methyl N-[4-[6-[(4-chlorophenyl)-methyl-carbamoyl]-5-methyl-imidazo[1,2-a]pyridin-3-yl]phenyl]carbamate

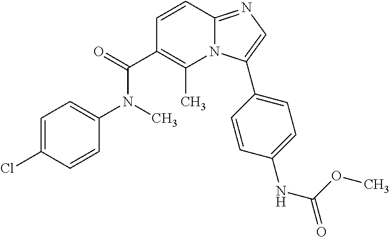 [SMILES: COC(=O)Nc1ccc(-c2cnc3ccc(C(=O)N(C)c4ccc(Cl)cc4)c(C)n23)cc1]

To a stirred solution of 3-[4-(methoxycarbonylamino)phenyl]-5-methyl-imidazo[1,2-a]pyridine-6-carboxylic acid (110 mg, 0.304 mmol, 1 eq) in pyridine (2 mL) was added 4-chloro-N-methyl-aniline (64.6 mg, 0.456 mmol, 1.5 eq). Then phosphorus(V) oxychloride (0.0836 mL, 0.913 mmol, 5 eq) was added at 0° C. The reaction mixture was warmed to room temperature and stirred for an additional hour. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl N-[4-[6-[(4-chlorophenyl)-methyl-carbamoyl]-5-methyl-imidazo[1,2-a]pyridin-3-yl]phenyl]carbamate.

¹H NMR (400 MHz, CDCl₃) δ: 7.46 (m, 4H), 7.15-7.26 (m, 3H), 6.89-7.03 (m, 4H), 6.80 (s, 1H), 3.82 (s, 3H), 3.45 (br s, 3H), 2.10 (br s, 3H).

Example 4: This example illustrates the preparation of methyl N-[4-[7-chloro-6-[(4-chlorophenyl)-methyl-carbamoyl]imidazo[1,2-a]pyridin-3-yl]phenyl]carbamate (Compound 23)

### Step 1: Preparation of methyl 2-amino-4-chloro-1,2-dihydropyridine-5-carboxylate

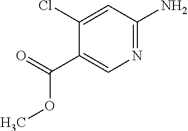

An autoclave was charged with 4-chloro-5-iodo-pyridin-2-amine (100 mg, 0.393 mmol, 1 eq), triethylamine (0.110 mL, 0.786 mmol, 2 eq) and methanol (10 mL). The reaction mixture was degassed with argon for 5 minutes, then 1,1′-bis(diphenylphosphino)ferrocene-palladium(II)dichloride dichloromethane complex (16.0 mg, 0.0196 mmol, 0.05 eq) was added and it was degassed for 5 additional minutes. The reaction mixture was stirred at 100° C. for 16 hours under CO atmosphere (100 PSI). After cooling down to room temperature, the reaction mixture was concentrated under reduced pressure. The resulting residue was diluted with water and extracted twice with ethyl acetate. The combined organic layers were washed with water, then brine, dried over sodium sulfate, filtered, and concentrated under reduced pressure. The crude residue was purified by flash chromatography to give methyl 2-amino-4-chloro-1,2-dihydropyridine-5-carboxylate as an off-white solid.

¹H NMR (400 MHz, DMSO-d₆) δ: 8.49 (s, 1H), 7.06 (s, 2H), 6.52 (s, 1H), 3.76 (s, 3H).

### Step 2: Preparation of methyl 7-chloroimidazo[1,2-a]pyridine-6-carboxylate

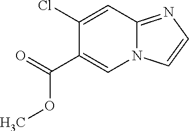

An autoclave was charged with methyl 2-amino-4-chloro-1,2-dihydropyridine-5-carboxylate (1.20 g, 6.04 mmol, 1 eq), ethanol (20 mL) and 2-chloroacetaldehyde (0.474 g, 6.04 mmol, 1 eq). The reaction mixture was stirred at 80° C. for 16 hours. After cooling down to room temperature, the reaction mixture was concentrated under reduced pressure. The residue was diluted with water and extracted twice with ethyl acetate. The combined organic layers were washed with water, then brine, dried over sodium sulfate, filtered, and concentrated under reduced pressure. The crude residue was purified by column chromatography to give methyl 7-chloroimidazo[1,2-a]pyridine-6-carboxylate as an off-white solid.

¹H NMR (400 MHz, DMSO-d₆) δ: 9.34 (d, J=0.8 Hz, 1H), 8.12 (dd, J=0.8, 1.3 Hz, 1H), 7.87 (s, 1H), 7.73 (d, J=1.3 Hz, 1H), 3.88 (s, 3H).

### Step 3: Preparation of methyl 7-chloro-3-iodo-imidazo[1,2-a]pyridine-6-carboxylate

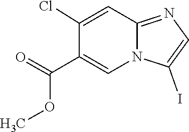

To a stirred solution of methyl 7-chloroimidazo[1,2-a]pyridine-6-carboxylate (900 mg, 3.42 mmol, 1 eq) in dimethylformamide (9 mL) was added at room temperature N-iodosuccinimide (1.15 g, 5.13 mmol, 1.5 eq). The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 7-chloro-3-iodo-imidazo[1,2-a]pyridine-6-carboxylate as a brown solid.

¹H NMR (400 MHz, CDCl₃) δ: 8.77 (s, 1H), 7.77 (s, 1H), 7.73 (s, 1H), 4.01 (s, 3H).

### Step 4: Preparation of methyl 7-chloro-3-[4-(methoxycarbonylamino)phenyl]imidazo[1,2-a]pyridine-6-carboxylate

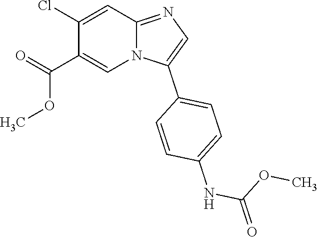

A microwave vial was charged with methyl 7-chloro-3-iodo-imidazo[1,2-a]pyridine-6-carboxylate (800 mg, 2.26 mmol, 1 eq), methyl N-[4-(4,4,5,5-tetramethyl-1,3,2-dioxaborolan-2-yl)phenyl]carbamate (commercially available, CAS 844500-75-0, 939 mg, 3.39 mmol, 1.5 eq), cesium carbonate (1.47 g, 4.52 mmol, 2 eq), 1,4-dioxane (15 mL) and water (2 mL). The reaction mixture was degassed with argon for 5 minutes, then Pd(dppf)Cl₂ (165 mg, 0.226 mmol, 0.1 eq) was added and it was again degassed for 2 additional minutes. The reaction mixture was heated under microwave irradiation at 90° C. and stirred for 16 hours. After cooling down to room temperature, the reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 7-chloro-3-[4-(methoxycarbonylamino)phenyl]imidazo[1,2-a]pyridine-6-carboxylate as an off-white solid.

¹H NMR (400 MHz, DMSO-d₆) δ: 9.91 (s, 1H), 8.91 (s, 1H), 7.93 (s, 1H), 7.83 (s, 1H), 7.67 (d, J=8.8 Hz, 2H), 7.61 (d, J=8.8 Hz, 2H), 3.85 (s, 3H), 3.71 (s, 3H).

### Step 5: Preparation of 7-chloro-3-[4-(methoxycarbonylamino)phenyl]imidazo[1,2-a]pyridine-6-carboxylic acid

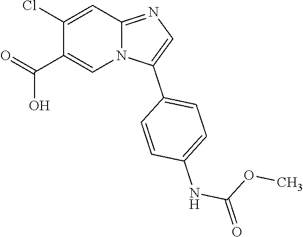 [SMILES: COC(=O)Nc1ccc(-c2cnc3cc(Cl)c(C(=O)O)cn23)cc1]

To a stirred solution of methyl 7-chloro-3-[4-(methoxycarbonylamino)phenyl]imidazo[1,2-a]pyridine-6-carboxylate (550 mg, 1.53 mmol, 1 eq) in THE (10 mL) and methanol (10 mL) was added at room temperature a solution of lithium hydroxide monohydrate (128 mg, 3.06 mmol, 2 eq) in water (2 mL). The reaction mixture was stirred for 16 hours at room temperature. The reaction mixture was concentrated under reduced pressure. The resulting residue was diluted with water and neutralized with 2M HCl. A precipitate appeared, which was filtered and dried under reduced pressure to give 7-chloro-3-[4-(methoxycarbonylamino)phenyl]imidazo[1,2-a]pyridine-6-carboxylic acid as an off-white solid that was used in the next step without further purification.

¹H NMR (400 MHz, DMSO-d₆) δ: 13.54 (br s, 1H), 9.90 (s, 1H), 8.90 (s, 1H), 7.89 (s, 1H), 7.82 (s, 1H), 7.66 (d, J=8.8 Hz, 2H), 7.61 (d, J=8.8 Hz, 2H), 3.70 (s, 3H).

### Step 6: Preparation of methyl N-[4-[7-chloro-6-[(4-chlorophenyl)-methyl-carbamoyl]imidazo[1,2-a]pyridin-3-yl]phenyl]carbamate

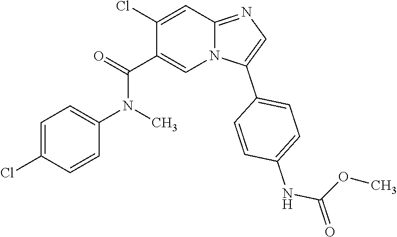 [SMILES: COC(=O)Nc1ccc(-c2cnc3cc(Cl)c(C(=O)N(C)c4ccc(Cl)cc4)cn23)cc1]

To a stirred solution of 7-chloro-3-[4-(methoxycarbonylamino)phenyl]imidazo[1,2-a]pyridine-6-carboxylic acid (200 mg, 0.550 mmol, 1 eq) in pyridine (5 mL) was added 4-chloro-N-methyl-aniline (93.4 mg, 0.659 mmol, 1.2 eq). Then phosphorus(V) oxychloride (0.252 mL, 2.75 mmol, 5 eq) was added at 0° C. The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl N-[4-[7-chloro-6-[(4-chlorophenyl)-methyl-carbamoyl]imidazo[1,2-a]pyridin-3-yl]phenyl]carbamate as an off-white solid.

¹H NMR (400 MHz, DMSO-d₆) δ: 9.90 (s, 1H), 8.60 (br s, 1H), 7.76 (br s, 1H), 7.72 (br s, 1H), 7.65 (d, J=8.3 Hz, 2H), 7.30-7.48 (m, 6H), 3.71 (s, 3H), 3.32 (s, 3H).

Example 5: This example illustrates the preparation of N-[5-[6-[(4-fluorophenyl)-methyl-carbamoyl]imidazo[1,2-a]pyridin-3-yl]-2-pyridyl]carbamate (example 25).

### Step 1: Preparation of methyl N-[5-(4,4,5,5-tetramethyl-1,3,2-dioxaborolan-2-yl)-2-pyridyl]carbamate

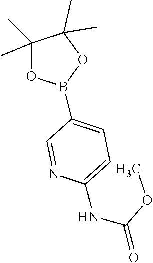 [SMILES: COC(=O)Nc1ccc(B2OC(C)(C)C(C)(C)O2)cn1]

2-Aminopyridine-5-boronic acid pinacol ester (2000 mg, 8.63 mmol, 1.00 eq.) and pyridine (2.79 g, 2.85 mL, 34.5 mmol, 4.00 eq.) were dissolved in ethyl acetate (50 mL) and cooled down to 10° C. Methyl chloroformate (3.30 g, 2.70 mL, 34.5 mmol, 4.00 eq.) was then added dropwise at 10°−15° C. After the addition was completed, the mixture was stirred at room temperature for an additional 2 hours. The mixture was then diluted with ethyl acetate, treated with a saturated solution of NaHCO₃, and extracted with ethyl acetate. The combined organic layers were washed with brine, dried over sodium sulfate, filtered, and concentrated under reduced pressure to afford methyl N-[5-(4,4,5,5-tetramethyl-1,3,2-dioxaborolan-2-yl)-2-pyridyl]carbamate as a white solid and was used for the next step without further purification.

LC/MS (Method A) retention time=0.16 min; [M+H]⁺=279

¹H NMR (400 MHz, CDCl₃) δ: 8.64 (dd, J=0.9, 1.7 Hz, 1H), 8.10-8.05 (m, 1H), 7.99 (dd, J=0.7, 8.4 Hz, 1H), 7.28 (s, 1H), 3.83 (s, 3H), 1.37 (s, 12H)

### Step 2: Preparation of lithium 3-[6-(methoxycarbonylamino)-3-pyridyl]imidazo[1,2-a]pyridine-6-carboxylate

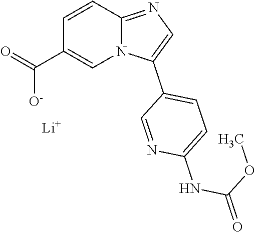 [SMILES: COC(=O)Nc1ccc(-c2cnc3ccc(C(=O)[O-])cn23)cn1 ; [Li+]]

Methyl 3-bromoimidazo[1,2-a]pyridine-6-carboxylate (3000 mg, 11.76 mmol, 1.00 eq.) and methyl N-[5-(4,4,5,5-tetramethyl-1,3,2-dioxaborolan-2-yl)-2-pyridyl]carbamate (5010 mg, 17.1 mmol, 1.50 eq.) were dissolved in dioxane (75 mL) and water (7.5 mL). Cesium carbonate (7510 mg, 22.8 mmol, 2.0 eq.) was added and the mixture was purged with a stream of argon for 5 minutes. Then, CATACXIUM® Pd G3 (437 mg, 0.570 mmol, 0.05 eq.) was added and the reaction mixture was heated to 85° C. for 17 hours. The mixture was cooled down to room temperature and filtered through a pad of Celite. The filtrate was concentrated under reduced pressure and the suspension which formed was filtered. The solid was washed with ethyl acetate and dried under vacuum to afford methyl 3-[6-(methoxycarbonylamino)-3-pyridyl]imidazo[1,2-a]pyridine-6-carboxylate as a yellow solid. This crude residue (2140 mg, 6.23 mmol, 1.00 eq.) was dissolved in a mixture of THE/water 3:1 (152 mL), lithium hydroxide (400 mg, 1.50 equiv., 9.35 mmol) was added and the reaction mixture was stirred at room temperature for 17 hours. The mixture was filtered and washed with THF. The resulting solid was dried in vacuo to afford lithium 3-[6-(methoxycarbonylamino)-3-pyridyl]imidazo[1,2-a]pyridine-6-carboxylate as an off-white solid.

LC/MS (Method A) retention time=0.25 min; [M]⁻=311

¹H NMR (400 MHz, DMSO-d₆) δ: 10.42 (br s, 1H), 8.75 (s, 1H), 8.52 (d, J=1.8 Hz, 1H), 8.14-8.00 (m, 2H), 7.79-7.70 (m, 2H), 7.49 (br d, J=0.7 Hz, 1H), 3.72 (s, 3H).

### Step 3: Preparation of methyl N-[5-[6-[(4-fluorophenyl)-methyl-carbamoyl]imidazo[1,2-a]pyridin-3-yl]-2-pyridyl]carbamate

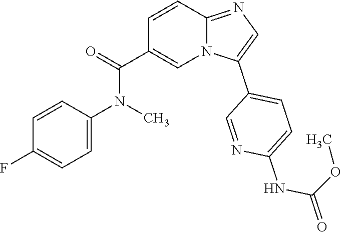 [SMILES: COC(=O)Nc1ccc(-c2cnc3ccc(C(=O)N(C)c4ccc(F)cc4)cn23)cn1]

To a mixture of lithium 3-[6-(methoxycarbonylamino)-3-pyridyl]imidazo[1,2-a]pyridine-6-carboxylate (1 eq., 0.042 mmol) in dimethylacetamide (0.24 mL) was added a solution N-methyl-4-fluoroaniline (2 eq., 0.084 mmol) in dimethylacetamide (0.40 mL), diisopropylethylamine (6.0 eq., 0.252 mmol) and a stock solution of bis(2-oxo-3-oxazolidinyl)phosphinic chloride (BOP—Cl) (2.5 eq., 0.101 mmol). The reaction mixture was stirred at 70° C. for 16 hours and then cooled to room temperature before all the volatiles were evaporated under reduced pressure. The resulting residue was dissolved in methanol (1 mL), filtered and concentrated under reduce pressure. This crude residue was dissolved in dimethylacetamide (900 uL) and purified via HPLC to afford methyl N-[5-[6-[(4-fluorophenyl)-methyl-carbamoyl]imidazo[1,2-a]pyridin-3-yl]-2-pyridyl]carbamate as an off-white solid.

LC/MS (Method A) retention time=0.70 min; [M+H]⁺=420

¹H NMR (400 MHz, CDCl₃) δ: 8.29-8.19 (m, 2H), 8.15 (d, J=8.4 Hz, 1H), 7.93 (br s, 1H), 7.69 (s, 1H), 7.58 (dd, J=2.4, 8.5 Hz, 1H), 7.49 (d, J=9.4 Hz, 1H), 7.16 (dd, J=1.8, 9.4 Hz, 1H), 7.13-7.02 (m, 4H), 3.89 (s, 3H), 3.49 (s, 3H).

Example 6: This example illustrates the preparation of methyl N-[4-[6-[(4-chlorophenyl)-(cyanomethyl)carbamoyl]imidazo[1,2-a]pyridin-3-yl]phenyl]carbamate (Compound 31)

### Step 1: Preparation of 3-bromo-N-(4-chlorophenyl)imidazo[1,2-a]pyridine-6-carboxamide

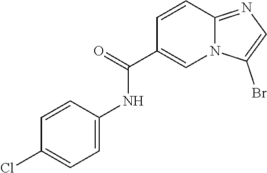 [SMILES: O=C(Nc1ccc(Cl)cc1)c1ccc2ncc(Br)n2c1]

A mixture of 3-bromoimidazo[1,2-a]pyridine-6-carboxylic acid (500 mg, 1.0 eq, 1.97 mmol), 4-chloroaniline (308 mg, 1.2 eq, 2.36 mmol) and diisopropylethylamine (1290 mg, 5.0 eq, 9.85 mmol, 1.73 mL) was dissolved in dimethylacetamide (15 mL). The mixture was stirred at room temperature for 5 minutes before the addition of propanephosphonic acid anhydride (50% wt. in ethyl acetate, 3140 mg, 2.93 mL, 4.93 mmol, 2.5 eq.). The reaction was heated at 40° C. for 2 hours. After the reaction mixture was cooled down to room temperature, it was diluted with ethyl acetate, quenched with a saturated solution of NaHCO₃ and extracted with ethyl acetate. The combined organic layers were washed with water and brine, dried over sodium sulfate, filtered, and concentrated under reduced pressure. The crude residue was purified by flash chromatography (cyclohexane/ethyl acetate) to yield 3-bromo-N-(4-chlorophenyl)imidazo[1,2-a]pyridine-6-carboxamide as a beige solid.

LC/MS (Method A) retention time=0.92 min; [M+H]⁺=352

¹H NMR (400 MHz, DMSO-d₆) δ: 10.69 (s, 1H), 9.01 (s, 1H), 7.96-7.72 (m, 4H), 7.52-7.37 (m, 2H).

### Step 2: Preparation of 3-bromo-N-(4-chlorophenyl)-N-(cyanomethyl)imidazo[1,2-a]pyridine-6-carboxamide

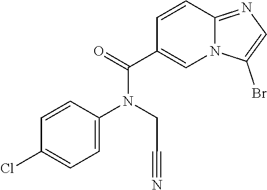 [SMILES: N#CCN(C(=O)c1ccc2ncc(Br)n2c1)c1ccc(Cl)cc1]

To a solution of 3-bromo-N-(4-chlorophenyl)imidazo[1,2-a]pyridine-6-carboxamide (74.0 mg, 1.00 eq, 0.194 mmol) in methyltetrahydrofuran (3 mL) cooled at 0° C. was added sodium hydride (11.8 mg, 1.50 eq, 0.291 mmol), and the resulting mixture was allowed to warm to room temperature an stirred for ab additional 2 hours. The reaction mixture was once again cooled down to 0° C. and 2-bromoacetonitrile (48.7 mg, 2.00 eq, 0.394 mmol, 0.028 mL) was added dropwise at 0-5° C. The mixture was then allowed to stir at 55° C. for 17 hours. The mixture was diluted with ethyl acetate, treated with a saturated solution of NaHCO₃, and extracted with ethyl acetate. The combined organic layers were then washed with brine, dried over sodium sulfate, filtered, and concentrated under reduced pressure. The crude residue was used directly in the next step.

LC/MS (Method A) retention time=0.86 min, [M+H]⁺=391

¹H NMR (400 MHz, CDCl₃) δ: 8.39-8.33 (m, 1H), 7.67-7.60 (m, 2H), 7.42-7.35 (m, 2H), 7.23-7.15 (m, 2H), 7.07-7.01 (m, 1H), 4.76 (s, 2H).

### Step 3: Preparation of methyl N-[4-(4,4,5,5-tetramethyl-1,3,2-dioxaborolan-2-yl)phenyl]carbamate

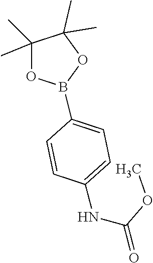 [SMILES: COC(=O)Nc1ccc(B2OC(C)(C)C(C)(C)O2)cc1]

A mixture of 4-(4,4,5,5-Tetramethyl-1,3,2-dioxaborolan-2-yl)aniline (2.00 g, 1.00 eq, 8.95 mmol) and pyridine (2.17 g, 3.00 eq, 26.8 mmol, 2.21 mL) was dissolved in ethyl acetate (100 mL). Methyl chloroformate (1.71 g, 2.00 eq, 17.9 mmol, 1.40 mL) was then added dropwise at 10° C. and the mixture was stirred at room temperature for 2 hours. The mixture was diluted with ethyl acetate, treated with a saturated solution of NaHCO₃, and extracted twice with ethyl acetate. The combined organic layers were washed with water and brine, dried over sodium sulfate, filtered, and concentrated under reduced pressure. The crude residue was purified by flash chromatography (cyclohexane/ethyl acetate) to yield methyl N-[4-(4,4,5,5-tetramethyl-1,3,2-dioxaborolan-2-yl)phenyl]carbamate as a colorless oil.

LC/MS (Method A) retention time=1.00 min; [M+H]⁺=278

¹H NMR (400 MHz, CDCl₃) δ: 7.70-7.83 (m, 2H), 7.33-7.47 (m, 2H), 6.57-6.77 (m, 1H), 3.79 (s, 3H), 1.34 (s, 12H).

### Step 4: Preparation of methyl N-[5-[6-[(4-chlorophenyl)-methyl-carbamoyl]imidazo[1,2-a]pyridin-3-yl]-2-pyridyl]carbamate

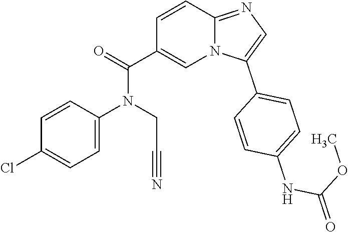 [SMILES: COC(=O)Nc1ccc(-c2cnc3ccc(C(=O)N(CC#N)c4ccc(Cl)cc4)cn23)cc1]

A mixture of 3-bromo-N-(4-chlorophenyl)-N-(cyanomethyl)imidazo[1,2-a]pyridine-6-carboxamide (50.0 mg, 1.00 eq, 0.116 mmol) and methyl N-[4-(4,4,5,5-tetramethyl-1,3,2-dioxaborolan-2-yl)phenyl]carbamate (43.8 mg, 1.30 eq, 0.150 mmol) was dissolved in dioxane (0.8 mL) and water (0.24 mL). Cesium carbonate (76.0 mg, 2.00 eq, 0.231 mmol) was added and the reaction mixture was flushed with argon for 5 minutes. Then CATACXIUM® Pd G3 (4.43 mg, 0.05 eq, 0.00578 mmol) was added and the mixture was irradiated under microwave at 95° C. for 45 minutes. The mixture was diluted with ethyl acetate, treated with a saturated solution of NaHCO₃ and extracted with ethyl acetate. The combined organic layers were then washed with brine, dried over sodium sulfate, filtered, and concentrated under reduced pressure. The crude residue was purified by reverse phase preparative HPLC (water/acetonitrile) to yield 9 mg of methyl N-[5-[6-[(4-chlorophenyl)-methyl-carbamoyl]imidazo[1,2-a]pyridin-3-yl]-2-pyridyl]carbamate as an off-white solid.

LC/MS (Method A) retention time=0.77 min; [M+H]⁺=460

¹H NMR (400 MHz, CDCl₃) δ: 8.28-8.34 (m, 1H), 7.63-7.68 (m, 1H), 7.47-7.59 (m, 3H), 7.38-7.44 (m, 2H), 7.13-7.23 (m, 5H), 6.91-7.00 (m, 1H), 4.72 (s, 2H), 3.87 (s, 3H).

Example 7: This example illustrates the preparation of methyl N-[4-[6-[(4-chlorophenyl)-prop-2-ynyl-carbamoyl]imidazo[1,2-a]pyridin-3-yl]phenyl]carbamate (Compound 33)

### Step 1: Preparation of 3-bromo-N-(4-chlorophenyl)-N-prop-2-ynyl-imidazo[1,2-a]pyridine-6-carboxamide

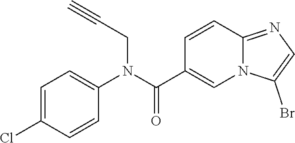 [SMILES: C#CCN(C(=O)c1ccc2ncc(Br)n2c1)c1ccc(Cl)cc1]

To 3-bromoimidazo[1,2-a]pyridine-6-carboxylic acid (112 mg, 0.44 mmol, 1.0 eq.) in methyltetrahydrofuran (4.5 mL) at room temperature was added dimethylformamide (3.3 mg, 0.003 mL, 0.044 mmol, 0.1 eq.) and oxalyl chloride (68.6 mg, 0.047 mL, 0.53 mmol, 1.20 eq). After 3 hours the reaction mixture was evaporated to dryness to afford 3-bromoimidazo[1,2-a]pyridine-6-carbonyl chloride as a crude residue. In another flask, 4-chloro-N-prop-2-yl-aniline (79.0 mg, 0.44 mmol, 1.0 eq.) and pyridine (106 mg, 0.11 mL, 1.32 mmol, 3.0 eq.) were dissolved in methyltetrahydrofuran (4 mL). This mixture was added to the 3-bromoimidazo[1,2-a]pyridine-6-carbonyl chloride prepared above and the resulting mixture was stirred at 70° C. for 16 hours. The reaction mixture was then cooled down to room temperature, diluted with ethyl acetate, treated with a saturated solution of NaHCO₃, extracted with ethyl acetate. The combined organic layers were washed with water and brine, dried over sodium sulfate, filtered, and concentrated under reduced pressure. The crude residue was purified by flash chromatography (cyclohexane/ethyl acetate) to afford 3-bromo-N-(4-chlorophenyl)-N-prop-2-ynyl-imidazo[1,2-a]pyridine-6-carboxamide as a yellow resin.

LC/MS (Method A) retention time=0.91 min; [M+H]⁺=388

¹H NMR (400 MHz, CDCl₃) δ: 8.36 (dd, J=0.9, 1.7 Hz, 1H), 7.64 (s, 1H), 7.38 (dd, J=0.9, 9.4 Hz, 1H), 7.36-7.32 (m, 2H), 7.22-7.17 (m, 2H), 7.08 (dd, J=1.7, 9.4 Hz, 1H), 4.70 (d, J=2.6 Hz, 2H), 2.33 (t, J=2.4 Hz, 1H).

### Step 2: Preparation of methyl N-[4-[6-[(4-chlorophenyl)-prop-2-ynyl-carbamoyl]imidazo[1,2-a]pyridin-3-yl]phenyl]carbamate

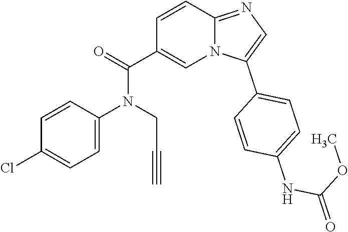

In a microwave vial, 3-bromo-N-(4-chlorophenyl)-N-prop-2-ynyl-imidazo[1,2-a]pyridine-6-carboxamide (80.0 mg, 0.189 mmol, 1.0 eq.) and methyl N-[4-(4,4,5,5-tetramethyl-1,3,2-dioxaborolan-2-yl)phenyl]carbamate (71.8 mg, 0.246 mmol, 1.3 eq.) were dissolved in dioxane (2.8 mL) and water (0.8 mL). Cesium carbonate (125.0 mg, 0.379 mmol, 2.0 eq.) was added and the reaction mixture was purged with argon. Finally, tetrakis(triphenylphosphine)palladium(0) (11.1 mg, 0.05 eq, 0.01 mmol) was added, and the reaction mixture was heated under microwave irradiation at 100° C. for 80 minutes. The mixture mixture was diluted with ethyl acetate, treated with a saturated solution of NaHCO₃ and extracted with ethyl acetate. The combined organic layers were then washed with brine, dried over sodium sulfate, filtered, and concentrated under reduced pressure. The crude residue was purified by flash chromatography (cyclohexane/ethyl acetate) to afford methyl N-[4-[6-[(4-chlorophenyl)-prop-2-ynyl-carbamoyl]imidazo[1,2-a]pyridin-3-yl]phenyl]carbamate as a yellow solid.

LC/MS (Method A) retention time=0.86 min. [M+H]⁺=391

¹H NMR (400 MHz, CDCl₃) δ: 8.31-8.28 (m, 1H), 7.64 (s, 1H), 7.56 (d, J=8.4 Hz, 2H), 7.50 (dd, J=0.7, 9.5 Hz, 1H), 7.39-7.33 (m, 2H), 7.23 (dd, J=1.8, 9.5 Hz, 1H), 7.21-7.13 (m, 4H), 7.09 (br s, 1H), 4.66 (d, J=2.6 Hz, 2H), 3.85 (s, 3H), 2.29 (t, J=2.4 Hz, 1H).

Example 8: This example illustrates the preparation of methyl N-[4-[7-bromo-6-[(4-chlorophenyl)-methyl-carbamoyl]imidazo[1,2-a]pyridin-3-yl]phenyl]carbamate (Compound 34)

### Step 1: Preparation of 4-bromo-5-iodo-pyridin-2-amine

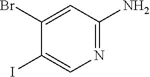

To a stirred solution of 4-bromopyridin-2-amine (500 mg, 2.75 mmol, 1 eq) in acetonitrile (10 mL) was added at room temperature N-iodosuccinimide (1.23 g, 5.49 mmol, 1.5 eq). The reaction mixture was stirred for 24 hours at 50° C. After cooling down to room temperature, the reaction mixture was diluted with water and extracted twice with ethyl acetate. The combined organic layers were washed with brine, dried over sodium sulfate, filtered, and concentrated under reduced pressure. The crude residue was purified by flash chromatography to give 4-bromo-5-iodo-pyridin-2-amine as a brown solid.

¹H NMR (400 MHz, DMSO-d₆) δ: 8.19 (s, 1H), 6.87 (s, 1H), 6.37 (br s, 2H).

### Step 2: Preparation of 7-bromo-6-iodo-imidazo[1,2-a]pyridine

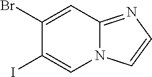 [SMILES: Brc1cc2nccn2cc1I]

A sealed tube was charged with 4-bromo-5-iodo-pyridin-2-amine (10.5 g, 31.6 mmol, 1 eq) 2-chloroacetaldehyde (45%, 22.3 mL, 158 mmol, 5 eq). The reaction mixture was stirred at 80° C. for 16 hours. After cooling down to room temperature, the reaction mixture was poured into a pre-stirred cold solution of saturated NaHCO₃ aq. and it was left stirring for 5 minutes. Then the reaction mixture was diluted with ethyl acetate. The organic layer was separated, washed with brine, dried over sodium sulfate, filtered, and concentrated under reduced pressure. The crude residue was purified by column chromatography to give 7-bromo-6-iodo-imidazo[1,2-a]pyridine as an off-white solid.

¹H NMR (400 MHz, CDCl₃) δ: 8.57 (s, 1H), 8.02 (s, 1H), 7.61 (s, 1H), 7.54 (s, 1H).

### Step 3: Preparation of methyl 7-bromoimidazo[1,2-a]pyridine-6-carboxylate

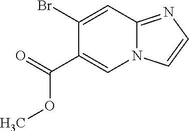

An autoclave was charged with 7-bromo-6-iodo-imidazo[1,2-a]pyridine (6.40 g, 17.8 mmol, 1 eq), sodium acetate (3.66 g, 44.6 mmol, 2.5 eq), 1,4-bis(diphenylphosphino)butane (0.761 g, 1.78 mmol, 0.1 eq) and methanol (100 mL). The reaction mixture was degassed with nitrogen for 5 minutes, then bis(triphenylphosphine)palladium(II) chloride (0.626 g, 0.892 mmol, 0.05 eq) was added. The reaction mixture was stirred at 80° C. for 16 hours under CO atmosphere (200 PSI). After cooling down to room temperature, the reaction mixture was concentrated under reduced pressure. The crude residue was purified by flash chromatography to give methyl 7-bromoimidazo[1,2-a]pyridine-6-carboxylate as a brown solid.

¹H NMR (400 MHz, CDCl₃) δ: 8.83 (s, 1H), 7.97 (s, 1H), 7.70-7.72 (m, 1H), 7.65 (br s, 1H), 3.97 (s, 3H).

### Step 4: Preparation of 7-bromoimidazo[1,2-a]pyridine-6-carboxylic acid

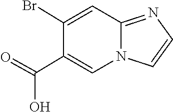 [SMILES: O=C(O)c1cn2ccnc2cc1Br]

To a stirred solution of methyl 7-bromoimidazo[1,2-a]pyridine-6-carboxylate (3.30 g, 11.6 mmol, 1 eq) in a 4:4:2 mixture of THF:methanol:water (40 mL) was added at room temperature lithium hydroxide monohydrate (0.977 g, 23.3 mmol, 2 eq). The reaction mixture was stirred for 1 hour at room temperature and then concentrated under reduced pressure. The resulting residue was diluted with water and acidified with saturated NaHSO₄ aq. A precipitate appeared, which was filtered and dried under reduced pressure to give 7-bromoimidazo[1,2-a]pyridine-6-carboxylic acid as a brown solid that was used in the next step without further purification.

¹H NMR (400 MHz, DMSO-d₆) δ: 13.21-13.75 (br s, 1H), 9.27 (s, 1H), 8.08 (s, 1H), 7.99 (s, 1H) 7.68 (d, J=1.0 Hz, 1H).

### Step 5: Preparation of 7-bromo-N-(4-chlorophenyl)-N-methyl-imidazo[1,2-a]pyridine-6-carboxamide

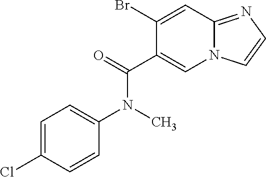 [SMILES: CN(C(=O)c1cn2ccnc2cc1Br)c1ccc(Cl)cc1]

To a stirred solution of 7-bromoimidazo[1,2-a]pyridine-6-carboxylic acid (50.0 mg, 0.176 mmol, 1 eq) in dimethylformamide (2 mL) were added 4-chloro-N-methyl-aniline (30.0 mg, 0.212 mmol, 1.2 eq), triethylamine (0.0737 mL, 0.529 mmol, 3 eq) and propanephosphonic acid anhydride (50% wt. in ethyl acetate, 0.210 mL, 0.353 mmol, 2 eq). The reaction mixture was stirred for 16 hours at room temperature. The reaction mixture was diluted with ethyl acetate, washed with water, then brine, dried over sodium sulfate, filtered, and concentrated under reduced pressure. The crude residue was purified by flash chromatography to give 7-bromo-N-(4-chlorophenyl)-N-methyl-imidazo[1,2-a]pyridine-6-carboxamide as an off-white solid.

¹H NMR (400 MHz, DMSO-d₆) δ: 8.56-8.79 (m, 1H), 7.89 (br s, 2H) 7.59 (br s, 1H) 7.25-7.45 (m, 4H) 3.38 (br s, 3H).

### Step 6: Preparation of 7-bromo-N-(4-chlorophenyl)-3-iodo-N-methyl-imidazo[1,2-a]pyridine-6-carboxamide

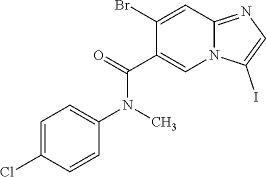 [SMILES: CN(C(=O)c1cn2c(I)cnc2cc1Br)c1ccc(Cl)cc1]

To a stirred solution of 7-bromo-N-(4-chlorophenyl)-N-methyl-imidazo[1,2-a]pyridine-6-carboxamide (800 mg, 2.15 mmol, 1 eq) in acetonitrile (20 mL) at room temperature was added at N-iodosuccinimide (967 mg, 4.30 mmol, 2 eq). The reaction mixture was stirred for 16 hours at room temperature, after which it was diluted with water, and extracted twice with ethyl acetate. The combined organic layers were washed with brine, dried over sodium sulfate, filtered, and concentrated under reduced pressure. The crude residue was purified by flash chromatography to give 7-bromo-N-(4-chlorophenyl)-3-iodo-N-methyl-imidazo[1,2-a]pyridine-6-carboxamide as a brown solid.

¹H NMR (400 MHz, DMSO-d₆) δ: 8.46 (br s, 1H), 7.92 (br s, 1H), 7.70 (br s, 1H), 7.26-7.47 (m, 4H), 3.40 (br s, 3H).

### Step 7: Preparation of methyl N-[4-[7-bromo-6-[(4-chlorophenyl)-methyl-carbamoyl]imidazo[1,2-a]pyridin-3-yl]phenyl]carbamate

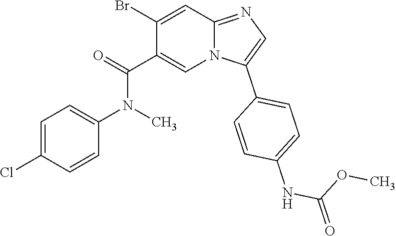 [SMILES: COC(=O)Nc1ccc(-c2cnc3cc(Br)c(C(=O)N(C)c4ccc(Cl)cc4)cn23)cc1]

A microwave vial was charged with 7-bromo-N-(4-chlorophenyl)-3-iodo-N-methyl-imidazo[1,2-a]pyridine-6-carboxamide (50.0 mg, 0.0917 mmol, 1 eq), methyl N-[4-(4,4,5,5-tetramethyl-1,3,2-dioxaborolan-2-yl)phenyl]carbamate (commercially available, CAS 844500-75-0, 42.4 mg, 0.138 mmol, 1.5 eq), cesium carbonate (59.8 mg, 0.183 mmol, 2 eq), 1,4-dioxane (1.5 mL) and water (0.5 mL). The reaction mixture was degassed with argon for 2 minutes, then cataCXium® A Pd G3 (3.34 mg, 0.00459 mmol, 0.05 eq) was added. The reaction mixture was then stirred under microwave irradiation at 90° C. for 1 hour. After cooling down to room temperature, the reaction mixture was filtered through a Celite pad and washed with ethyl acetate. The filtrate was washed with water, then brine, dried over sodium sulfate, filtered, and concentrated under reduced pressure. The crude residue was purified by flash chromatography to give methyl N-[4-[7-bromo-6-[(4-chlorophenyl)-methyl-carbamoyl]imidazo[1,2-a]pyridin-3-yl]phenyl]carbamate as an off-white solid.

¹H NMR (400 MHz, DMSO-d₆) δ: 9.90 (s, 1H), 8.56 (br s, 1H) 7.92 (br s, 1H), 7.70 (br s, 1H), 7.65 (d, J=8.6 Hz, 2H), 7.29-7.55 (m, 6H), 3.71 (s, 3H), 3.36 (br s, 3H).

Example 9: This example illustrates the preparation of methyl N-[4-[8-bromo-6-[(4-chlorophenyl)-methyl-carbamoyl]imidazo[1,2-a]pyridin-3-yl]phenyl]carbamate (Compound 35)

### Step 1: Preparation of 8-bromoimidazo[1,2-a]pyridine-6-carboxylic acid

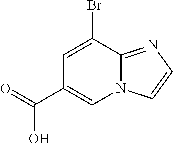 [SMILES: O=C(O)c1cc(Br)c2nccn2c1]

To a stirred solution of methyl 8-bromoimidazo[1,2-a]pyridine-6-carboxylate (2.00 g, 7.84 mmol, 1 eq) in THE (10 mL) and methanol (5 mL) was added at room temperature a solution of lithium hydroxide monohydrate (0.658 g, 15.7 mmol, 2 eq) in water (5 mL). The reaction mixture was stirred for 16 hours at room temperature. The reaction mixture was concentrated under reduced pressure. The resulting residue was diluted with water and neutralized with 2M HCl. A precipitate appeared which was filtered and dried under reduced pressure to give 8-bromoimidazo[1,2-a]pyridine-6-carboxylic acid as an off-white solid that was used in the next step without further purification.

¹H NMR (400 MHz, DMSO-d₆) δ: 9.40 (d, J=1.3 Hz, 1H), 8.33 (d, J=1.3 Hz, 1H), 8.03 (d, J=1.3 Hz, 1H), 7.89 (d, J=1.3 Hz, 1H).

### Step 2: Preparation of 8-bromo-N-(4-chlorophenyl)-N-methyl-imidazo[1,2-a]pyridine-6-carboxamide

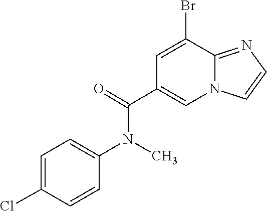 [SMILES: CN(C(=O)c1cc(Br)c2nccn2c1)c1ccc(Cl)cc1]

To a stirred solution of 8-bromoimidazo[1,2-a]pyridine-6-carboxylic acid (1.40 g, 5.81 mmol, 1 eq) in pyridine (20 mL) was added 4-chloro-N-methyl-aniline (0.822 g, 5.81 mmol, 1 eq). Then phosphorus(V) oxychloride (2.66 mL, 29.0 mmol, 5 eq) was added at 0° C. The reaction mixture was then warmed to room temperature and stirred for an additional hour. The reaction mixture was quenched with saturated NaHCO₃ aq., diluted with water, and extracted twice with ethyl acetate. The combined organic layers were washed with water, then brine, dried over sodium sulfate, filtered, and concentrated under reduced pressure. The crude residue was purified by flash chromatography to give 8-bromo-N-(4-chlorophenyl)-N-methyl-imidazo[1,2-a]pyridine-6-carboxamide as an off-white solid.

¹H NMR (400 MHz, CDCl₃) δ: 8.29 (d, J=1.0 Hz, 1H), 7.77 (s, 1H), 7.61 (s, 1H), 7.30-7.34 (m, 2H), 7.29 (d, J=1.2 Hz, 1H), 7.04-7.09 (m, 2H), 3.49 (s, 3H).

### Step 3: Preparation of 8-bromo-N-(4-chlorophenyl)-3-iodo-N-methyl-imidazo[1,2-a]pyridine-6-carboxamide

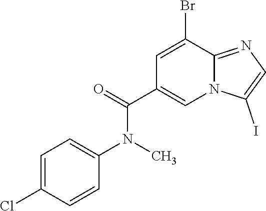 [SMILES: CN(C(=O)c1cc(Br)c2ncc(I)n2c1)c1ccc(Cl)cc1]

To a stirred solution of 8-bromo-N-(4-chlorophenyl)-N-methyl-imidazo[1,2-a]pyridine-6-carboxamide (500 mg, 1.30 mmol, 1 eq) in dimethylformamide (3 mL) was added at room temperature N-iodosuccinimide (440 mg, 1.95 mmol, 1.5 eq). The reaction mixture was stirred for 16 hours at room temperature. The reaction mixture was then diluted with water and extracted twice with ethyl acetate. The combined organic layers were washed with brine, dried over sodium sulfate, filtered, and concentrated under reduced pressure. The crude residue was purified by flash chromatography to give 8-bromo-N-(4-chlorophenyl)-3-iodo-N-methyl-imidazo[1,2-a]pyridine-6-carboxamide as a brown solid.

¹H NMR (400 MHz, DMSO-d₆) δ: 8.17 (d, J=1.2 Hz, 1H), 7.78 (s, 1H), 7.51 (d, J=1.2 Hz, 1H), 7.39 (s, 4H), 3.40 (s, 3H).

### Step 4: Preparation of methyl N-[4-[8-bromo-6-[(4-chlorophenyl)-methyl-carbamoyl]imidazo[1,2-a]pyridin-3-yl]phenyl]carbamate

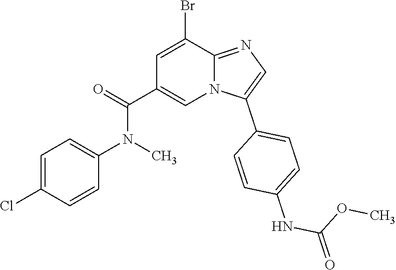 [SMILES: COC(=O)Nc1ccc(-c2cnc3c(Br)cc(C(=O)N(C)c4ccc(Cl)cc4)cn23)cc1]

A microwave vial was charged with 8-bromo-N-(4-chlorophenyl)-3-iodo-N-methyl-imidazo[1,2-a]pyridine-6-carboxamide (200 mg, 0.408 mmol, 1 eq), methyl N-[4-(4,4,5,5-tetramethyl-1,3,2-dioxaborolan-2-yl)phenyl]carbamate (commercially available, CAS 844500-75-0, 113 mg, 0.408 mmol, 1 eq), cesium carbonate (265 mg, 0.816 mmol, 2 eq), 1,4-dioxane (10 mL) and water (2 mL). The reaction mixture was degassed with argon for 5 minutes, then cataCXium (7.30 mg, 0.0204 mmol, 0.05 eq) and Pd(OAc)₂ (2.7 mg, 0.012 mmol, 0.03 eq.) were added. The reaction mixture was stirred at 80° C. for 30 minutes under microwave irradiation. After cooling down to room temperature, the reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl N-[4-[8-bromo-6-[(4-chlorophenyl)-methyl-carbamoyl]imidazo[1,2-a]pyridin-3-yl]phenyl]carbamate as an off-white solid.

¹H NMR (400 MHz, DMSO-d₆) δ: 9.91 (s, 1H), 8.19 (s, 1H), 7.74 (s, 1H), 7.63 (d, J=8.6 Hz, 2H), 7.53 (d, J=1.2 Hz, 1H), 7.34-7.44 (m, 4H), 7.17 (d, J=8.6 Hz, 2H), 3.72 (s, 3H), 3.37 (s, 3H).

Example 10: This example illustrates the preparation of methyl N-[4-[6-[(4-chlorophenyl)-methyl-carbamoyl]-7-methoxy-imidazo[1,2-a]pyridin-3-yl]phenyl]carbamate (Compound 39)

### Step 1: Preparation of 5-iodo-4-methoxy-pyridin-2-amine

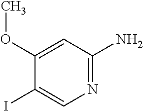

To a stirred solution of 4-methoxypyridin-2-amine (1.00 g, 7.65 mmol, 1 eq) in dimethylformamide at room temperature was added N-iodosuccinimide (2.58 g, 11.5 mmol, 1.5 eq). The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give 5-iodo-4-methoxy-pyridin-2-amine as an off-white solid.

¹H NMR (400 MHz, CDCl₃) δ: 8.16 (s, 1H), 5.98 (s, 1H), 3.87 (s, 3H).

### Step 2: Preparation of methyl 6-amino-4-methoxy-pyridine-3-carboxylate

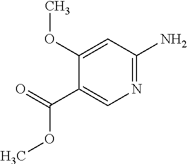

An autoclave was charged with 5-iodo-4-methoxy-pyridin-2-amine (1.00 g, 4.00 mmol, 1 eq), triethylamine (1.11 mL, 8.00 mmol, 2 eq) and methanol (20 mL). The reaction mixture was degassed with argon for 5 minutes. The 1,1′-bis(diphenylphosphino)ferrocene-palladium(II)dichloride dichloromethane complex (163 mg, 0.200 mmol, 0.05 eq) was then added and the reaction mixture was degassed for 5 additional minutes. The reaction mixture was then stirred at 100° C. for 16 hours under CO gas (100 PSI). After cooling down to room temperature, the reaction mixture was concentrated under reduced pressure. The resulting residue was diluted with water and extracted twice with ethyl acetate. The combined organic layers were washed with water, then brine, dried over sodium sulfate, filtered, and concentrated under reduced pressure. The crude residue was purified by flash chromatography to give methyl 6-amino-4-methoxy-pyridine-3-carboxylate as an off-white solid.

¹H NMR (400 MHz, DMSO-d₆) δ: 8.33 (s, 1H), 7.07 (br s, 2H), 6.12 (s, 1H), 3.81 (s, 3H), 3.71 (s, 3H).

### Step 3: Preparation of methyl 7-methoxyimidazo[1,2-a]pyridine-6-carboxylate

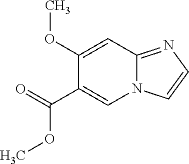

A sealed tube was charged with methyl 6-amino-4-methoxy-pyridine-3-carboxylate (100 mg, 0.521 mmol, 1 eq), water (5 mL) and bromoacetaldehyde dimethyl acetal (0.186 g, 1.04 mmol, 2 eq). The reaction mixture was stirred at room temperature for 16 hours. The reaction mixture was poured into a pre-stirred cold solution of saturated NaHCO₃ aq. and was left stirring for 10 minutes. Then the reaction mixture was diluted with dichloromethane. The organic layer was separated, washed with brine, dried over sodium sulfate, filtered, and concentrated under reduced pressure. The crude residue was purified by column chromatography to give methyl 7-methoxyimidazo[1,2-a]pyridine-6-carboxylate as a brown solid.

¹H NMR (400 MHz, DMSO-d₆) δ: 9.12 (s, 1H), 7.92-7.95 (m, 1H), 7.61 (d, J=1.2 Hz, 1H), 7.10 (s, 1H), 3.90 (s, 3H), 3.83 (s, 3H).

### Step 4: Preparation of methyl 3-iodo-7-methoxy-imidazo[1,2-a]pyridine-6-carboxylate

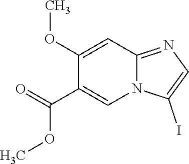 [SMILES: COC(=O)c1cn2c(I)cnc2cc1OC]

To a stirred solution of methyl 7-methoxyimidazo[1,2-a]pyridine-6-carboxylate (100 mg, 0.364 mmol, 1 eq) in dimethylformamide (0.5 mL) was added at room temperature N-iodosuccinimide (123 mg, 0.546 mmol, 1.2 eq). The reaction mixture was stirred for 16 hours at room temperature. The reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 3-iodo-7-methoxy-imidazo[1,2-a]pyridine-6-carboxylate.

¹H NMR (400 MHz, DMSO-d₆) δ: 8.56 (s, 1H), 7.64 (s, 1H), 7.15 (s, 1H), 3.89 (s, 3H), 3.85 (s, 3H).

### Step 5: Preparation of methyl 7-methoxy-3-[4-(methoxycarbonylamino)phenyl]imidazo[1,2-a]pyridine-6-carboxylic acid

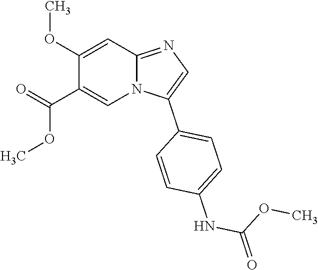

A microwave vial was charged with methyl 3-iodo-7-methoxy-imidazo[1,2-a]pyridine-6-carboxylate (400 mg, 1.14 mmol, 1 eq), methyl N-[4-(4,4,5,5-tetramethyl-1,3,2-dioxaborolan-2-yl)phenyl]carbamate (commercially available, CAS 844500-75-0, 349 mg, 1.26 mmol, 1.1 eq), cesium carbonate (746 mg, 2.29 mmol, 2 eq), 1,4-dioxane (3 mL) and water (0.5 mL). The reaction mixture was degassed with argon for 5 minutes, then cataCXium® A Pd G3 (43.9 mg, 0.0572 mmol, 0.05 eq) was added and it was again degassed for 2 additional minutes. The reaction mixture was stirred under microwave irradiation at 95° C. for 1 hour. After cooling down to room temperature, the reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl 7-methoxy-3-[4-(methoxycarbonylamino)phenyl]imidazo[1,2-a]pyridine-6-carboxylate as an off-white solid.

¹H NMR (400 MHz, DMSO-d₆) δ: 9.86 (s, 1H), 8.71 (s, 1H), 7.59-7.62 (m, 2H), 7.61 (s, 1H), 7.53-7.58 (m, 2H), 7.13 (s, 1H), 3.90 (s, 3H), 3.79 (s, 3H), 3.70 (s, 3H).

### Step 6: Preparation of 7-methoxy-3-[4-(methoxycarbonylamino)phenyl]imidazo[1,2-a]pyridine-6-carboxylic acid

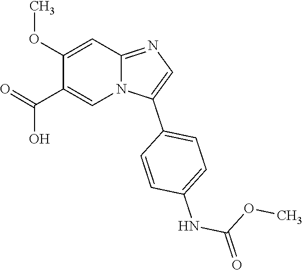 [SMILES: COC(=O)Nc1ccc(-c2cnc3cc(OC)c(C(=O)O)cn23)cc1]

To a stirred solution of methyl 7-methoxy-3-[4-(methoxycarbonylamino)phenyl]imidazo[1,2-a]pyridine-6-carboxylate (150 mg, 0.401 mmol, 1 eq) in THE (10 mL) and methanol (5 mL) was added at room temperature a solution of lithium hydroxide monohydrate (33.7 mg, 0.802 mmol, 2 eq) in water (5 mL). The reaction mixture was stirred for 1 hour at room temperature. The reaction mixture was concentrated under reduced pressure. The resulting residue was diluted with water, neutralized with 2 M HCl. The precipitate that appeared was filtered and dried under reduced pressure to give 7-methoxy-3-[4-(methoxycarbonylamino)phenyl]imidazo[1,2-a]pyridine-6-carboxylic acid as an off-white solid that was used in the next step without further purification.

¹H NMR (400 MHz, DMSO-d₆) δ: 9.86 (s, 1H), 8.71 (s, 1H), 7.61-7.66 (m, 2H), 7.60 (s, 1H), 7.53-7.58 (m, 2H), 7.09 (s, 1H), 3.89 (s, 3H), 3.70 (s, 3H).

### Step 7: Preparation of methyl N-[4-[6-[(4-chlorophenyl)-methyl-carbamoyl]-7-methoxy-imidazo[1,2-a]pyridin-3-yl]phenyl]carbamate

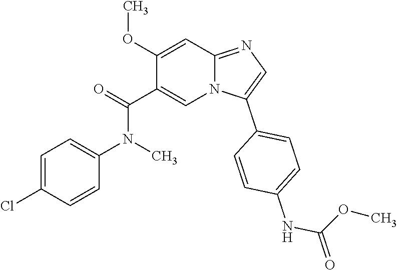 [SMILES: COC(=O)Nc1ccc(-c2cnc3cc(OC)c(C(=O)N(C)c4ccc(Cl)cc4)cn23)cc1]

To a stirred solution of 7-methoxy-3-[4-(methoxycarbonylamino)phenyl]imidazo[1,2-a]pyridine-6-carboxylic acid (20.0 mg, 0.059 mmol, 1 eq) in pyridine (1 mL) was added 4-chloro-N-methyl-aniline (12.4 mg, 0.0879 mmol, 1.5 eq). Then phosphorus(V) oxychloride (0.0268 mL, 0.293 mmol, 5 eq) was added at 0° C. The reaction mixture was stirred for 1 hour at room temperature. The reaction mixture was concentrated under reduced pressure. The resulting residue was diluted with water, neutralized with 2M HCl. A precipitate appeared, it was filtered and dried under reduced pressure. The crude residue was purified by flash chromatography to give methyl N-[4-[6-[(4-chlorophenyl)-methyl-carbamoyl]-7-methoxy-imidazo[1,2-a]pyridin-3-yl]phenyl]carbamate.

¹H NMR (400 MHz, DMSO-d₆) δ: 9.85 (s, 1H), 8.41 (s, 1H), 7.61-7.65 (d, J=8.6 Hz, 2H), 7.40-7.55 (m, 3H), 7.22-7.41 (m, 3H), 6.80-6.87 (br s, 1H), 3.70 (s, 3H), 3.67 (br s, 3H), 3.33 (br s, 3H).

Example 11: This example illustrates the preparation of methyl N-[4-[6-[(4-chlorophenyl)-methyl-carbamoyl]-8-cyano-imidazo[1,2-a]pyridin-3-yl]phenyl]carbamate (Compound 40)

### Step 1: Preparation of N-(4-chlorophenyl)-8-cyano-N-methyl-imidazo[1,2-a]pyridine-6-carboxamide

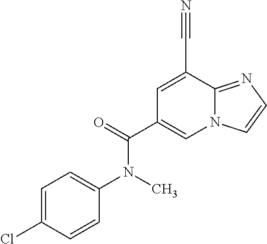 [SMILES: CN(C(=O)c1cc(C#N)c2nccn2c1)c1ccc(Cl)cc1]

A sealed tube was charged with 8-bromo-N-(4-chlorophenyl)-N-methyl-imidazo[1,2-a]pyridine-6-carboxamide (previously described in example 9, step 2, 300 mg, 0.740 mmol, 1 eq), Zn(CN)₂ (87.0 mg, 0.740 mmol, 1 eq) and dimethylformamide (2 mL). The reaction mixture was degassed with nitrogen for 15 minutes, then Xantphos (41.1 mg, 0.0740 mmol, 0.1 eq) and tris(dibenzylideneacetone)dipalladium(0) (67.8 mg, 0.0740 mmol, 0.1 eq) were added. The reaction mixture was heated to at 140° C. and stirred for 16 hours. After cooling down to room temperature, the reaction mixture was poured into iced water and extracted twice with ethyl acetate. The combined organic layers were washed with brine, dried over sodium sulfate, filtered, and concentrated under reduced pressure. The crude residue was purified by column chromatography to give N-(4-chlorophenyl)-8-cyano-N-methyl-imidazo[1,2-a]pyridine-6-carboxamide.

¹H NMR (400 MHz, DMSO-d₆) δ: 8.99 (d, J=1.5 Hz, 1H), 8.13 (d, J=1.3 Hz, 1H), 7.73 (d, J=1.5 Hz, 1H), 7.70 (d, J=1.3 Hz, 1H), 7.38-7.41 (m, 2H), 7.32-7.36 (m, 2H), 3.40 (s, 3H).

### Step 2: Preparation of N-(4-chlorophenyl)-8-cyano-3-iodo-N-methyl-imidazo[1,2-a]pyridine-6-carboxamide

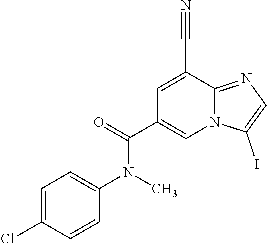 [SMILES: CN(C(=O)c1cc(C#N)c2ncc(I)n2c1)c1ccc(Cl)cc1]

To a stirred solution of N-(4-chlorophenyl)-8-cyano-N-methyl-imidazo[1,2-a]pyridine-6-carboxamide (250 mg, 0.603 mmol, 1 eq) in dimethylformamide (0.5 mL) was added at room temperature N-iodosuccinimide (136 mg, 0.603 mmol, 1 eq). The reaction mixture was stirred for 16 hours at room temperature. The reaction mixture was diluted with water and extracted twice with ethyl acetate. The combined organic layers were washed with brine, dried over sodium sulfate, filtered, and concentrated under reduced pressure. The crude residue was purified by flash chromatography to give N-(4-chlorophenyl)-8-cyano-3-iodo-N-methyl-imidazo[1,2-a]pyridine-6-carboxamide as an off-white solid.

¹H NMR (400 MHz, DMSO-d₆) δ: 8.44 (s, 1H) 7.89 (br s, 1H), 7.88 (s, 1H), 7.40 (s, 4H), 3.41 (s, 3H).

### Step 3: Preparation of methyl N-[4-[6-[(4-chlorophenyl)-methyl-carbamoyl]-8-cyano-imidazo[1,2-a]pyridin-3-yl]phenyl]carbamate

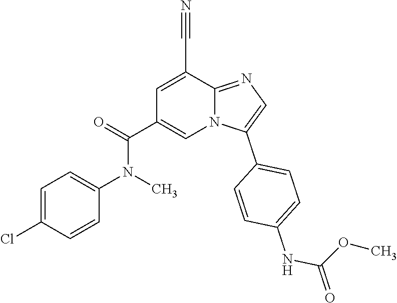 [SMILES: COC(=O)Nc1ccc(-c2cnc3c(C#N)cc(C(=O)N(C)c4ccc(Cl)cc4)cn23)cc1]

A microwave vial was charged with N-(4-chlorophenyl)-8-cyano-3-iodo-N-methyl-imidazo[1,2-a]pyridine-6-carboxamide (100 mg, 0.218 mmol, 1 eq), methyl N-[4-(4,4,5,5-tetramethyl-1,3,2-dioxaborolan-2-yl)phenyl]carbamate (commercially available, CAS 844500-75-0, 66.3 mg, 0.239 mmol, 1.1 eq), cesium carbonate (142 mg, 0.435 mmol, 2 eq), 1,4-dioxane (3 mL) and water (0.5 mL). The reaction mixture was degassed with argon for 5 minutes, then cataCXium® A Pd G3 (8.34 mg, 0.0109 mmol, 0.05 eq) was added. The reaction mixture was stirred at 95° C. for 1 hour under microwave irradiation. After cooling down to room temperature, the reaction mixture was diluted with water and extracted twice with ethyl acetate. The combined organic layers were washed with water, then brine, dried over sodium sulfate, filtered, and concentrated under reduced pressure. The crude residue was purified by flash chromatography to give methyl N-[4-[6-[(4-chlorophenyl)-methyl-carbamoyl]-8-cyano-imidazo[1,2-a]pyridin-3-yl]phenyl]carbamate as a yellow solid.

¹H NMR (400 MHz, DMSO-d₆) δ: 9.93 (s, 1H), 8.46 (s, 1H), 7.90 (d, J=1.3 Hz, 1H), 7.84 (s, 1H), 7.64 (d, J=8.8 Hz, 2H), 7.35-7.43 (m, 4H), 7.21 (d, J=8.5 Hz, 2H), 3.72 (s, 3H), 3.38 (s, 3H).

Example 12: This example illustrates the preparation of methyl N-[4-[6-[(4-chlorophenyl)-methyl-carbamoyl]-7-cyano-imidazo[1,2-a]pyridin-3-yl]phenyl]carbamate (Compound 41)

### Step 1: Preparation of N-(4-chlorophenyl)-7-cyano-N-methyl-imidazo[1,2-a]pyridine-6-carboxamide

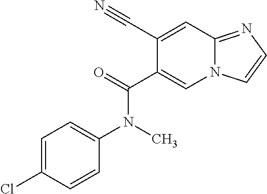 [SMILES: CN(C(=O)c1cn2ccnc2cc1C#N)c1ccc(Cl)cc1]

A sealed tube was charged with 7-bromo-N-(4-chlorophenyl)-N-methyl-imidazo[1,2-a]pyridine-6-carboxamide (previously described in example 8, step 5, 1.00 g, 2.47 mmol, 1 eq), Zn(CN)₂ (0.580 g, 4.94 mmol, 2 eq) and dimethylformamide (15 mL). The reaction mixture was degassed with nitrogen for 5 minutes, then Xantphos (0.137 g, 0.247 mmol, 0.1 eq) and tris(dibenzylideneacetone)dipalladium(0) (0.226 g, 0.247 mmol, 0.1 eq) were added. The reaction mixture was stirred at 140° C. for 16 hours. After cooling down to room temperature, the reaction mixture was poured into iced water and extracted twice with ethyl acetate. The combined organic layers were washed with brine, dried over sodium sulfate, filtered, and concentrated under reduced pressure. The crude residue was purified by column chromatography to give N-(4-chlorophenyl)-7-cyano-N-methyl-imidazo[1,2-a]pyridine-6-carboxamide as an off-white solid.

¹H NMR (400 MHz, DMSO-d₆) δ: 8.78 (s, 1H), 8.36 (s, 1H), 8.10 (s, 1H), 7.85 (d, J=1.10 Hz, 1H), 7.36-7.43 (m, 2H) 7.27-7.36 (m, 2H) 3.42 (s, 3H).

### Step 2: Preparation of N-(4-chlorophenyl)-7-cyano-3-iodo-N-methyl-imidazo[1,2-a]pyridine-6-carboxamide

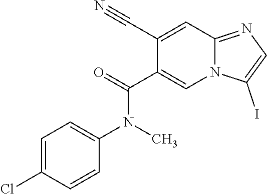 [SMILES: CN(C(=O)c1cn2c(I)cnc2cc1C#N)c1ccc(Cl)cc1]

To a stirred solution of N-(4-chlorophenyl)-7-cyano-N-methyl-imidazo[1,2-a]pyridine-6-carboxamide (450 mg, 1.32 mmol, 1 eq) in acetonitrile (20 mL) was added at room temperature N-iodosuccinimide (593 mg, 2.64 mmol, 2 eq). The reaction mixture was stirred for 16 hours at room temperature. The reaction mixture was diluted with water and extracted twice with ethyl acetate. The combined organic layers were washed with brine, dried over sodium sulfate, filtered, and concentrated under reduced pressure. The crude residue was purified by flash chromatography to give N-(4-chlorophenyl)-7-cyano-3-iodo-N-methyl-imidazo[1,2-a]pyridine-6-carboxamide as a brown solid.

¹H NMR (400 MHz, DMSO-d₆) δ: 8.40 (s, 1H), 7.99 (s, 1H), 7.39 (m, 5H), 3.43 (s, 3H).

### Step 3: Preparation of methyl N-[4-[6-[(4-chlorophenyl)-methyl-carbamoyl]-7-cyano-imidazo[1,2-a]pyridin-3-yl]phenyl]carbamate

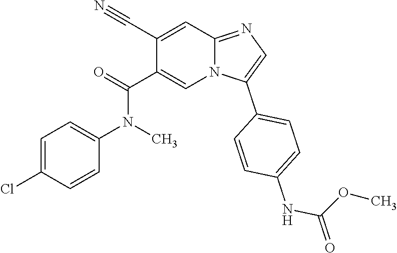 [SMILES: COC(=O)Nc1ccc(-c2cnc3cc(C#N)c(C(=O)N(C)c4ccc(Cl)cc4)cn23)cc1]

A microwave vial was charged with N-(4-chlorophenyl)-7-cyano-3-iodo-N-methyl-imidazo[1,2-a]pyridine-6-carboxamide (300 mg, 0.666 mmol, 1 eq), methyl N-[4-(4,4,5,5-tetramethyl-1,3,2-dioxaborolan-2-yl)phenyl]carbamate (commercially available, CAS 844500-75-0, 308 mg, 1.00 mmol, 1.5 eq), cesium carbonate (434 mg, 1.33 mmol, 2 eq), 1,4-dioxane (7.5 mL) and water (2.5 mL). The reaction mixture was degassed with argon for 2 minutes, then cataCXium® A Pd G3 (24.3 mg, 0.0333 mmol, 0.05 eq) was added. The reaction mixture was stirred at 90° C. for 1 hour under microwave irradiation. After cooling down to room temperature, the reaction mixture was filtered through a Celite pad and washed with ethyl acetate. The filtrate was washed with water, then brine, dried over sodium sulfate, filtered, and concentrated under reduced pressure. The crude residue was purified by flash chromatography to give methyl N-[4-[6-[(4-chlorophenyl)-methyl-carbamoyl]-7-cyano-imidazo[1,2-a]pyridin-3-yl]phenyl]carbamate as a yellow solid.

¹H NMR (400 MHz, DMSO-d₆) δ: 9.94 (s, 1H), 8.47 (br s, 1H) 8.43 (s, 1H), 7.99 (s, 1H), 7.65 (d, J=8.7 Hz, 2H), 7.33-7.43 (m, 4H), 7.25 (br d, J=6.6 Hz, 2H), 3.72 (s, 3H), 3.40 (s, 3H).

Example 13: This example illustrates the preparation of methyl N-[5-[6-[(4-fluoro-3-methoxy-phenyl)-methyl-carbamoyl]imidazo[1,2-a]pyridin-3-yl]-2-pyridyl]carbamate (Compound 45)

### Step 1: Preparation of 3-bromo-N-(4-fluoro-3-methoxy-phenyl)-N-methyl-imidazo[1,2-a]pyridine-6-carboxamide

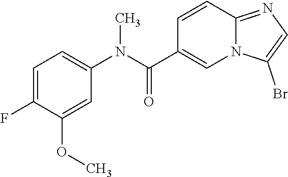 [SMILES: COc1cc(N(C)C(=O)c2ccc3ncc(Br)n3c2)ccc1F]

To a mixture of 3-bromoimidazo[1,2-a]pyridine-6-carboxylic acid (0.700 g, 2.90 mmol) in N,N-dimethylacetamide (17.5 mL) was added 4-fluoro-3-methoxy-N-methyl-aniline (0.712 g, 4.36 mmol) followed by N-ethyl-N-isopropyl-propan-2-amine (1.88 g, 2.49 mL, 14.5 mmol) and propanephosphonic acid anhydride (50% in ethyl acetate, 4.32 mL, 7.26 mmol). The reaction mixture was stirred for 1 h at 50° C. The mixture was then poured into ice water, and then extracted with ethyl acetate. The combined organic layers were washed with saturated NaHCO₃ aq. and brine, dried over MgSO₄, filtered, and concentrated under reduced pressure. The crude residue was treated with diisopropylether and a precipitation occurred. The precipitate was filtered and washed with diisopropylether to give 3-bromo-N-(4-fluoro-3-methoxy-phenyl)-N-methyl-imidazo[1,2-a]pyridine-6-carboxamide as a white solid.

LC/MS (Method A) retention time=0.81 min; [M+H]⁺=378

¹H NMR (400 MHz, CDCl₃) δ: 8.29 (s, 1H), 7.62 (s, 1H), 7.39 (dd, J=9.5, 0.7 Hz, 1H), 7.11 (dd, J=9.5, 1.8 Hz, 1H), 7.00 (dd, J=10.9, 8.7 Hz, 1H), 6.78 (dd, J=7.3, 2.5 Hz, 1H), 6.62-6.70 (m, 1H), 3.81 (s, 3H), 3.52 (s, 3H).

### Step 2: Preparation of N-[5-[6-[(4-fluoro-3-methoxy-phenyl)-methyl-carbamoyl]imidazo[1,2-a]pyridin-3-yl]-2-pyridyl]carbamate

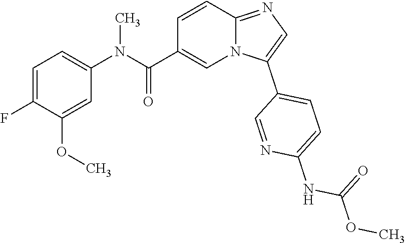 [SMILES: COC(=O)Nc1ccc(-c2cnc3ccc(C(=O)N(C)c4ccc(F)c(OC)c4)cn23)cn1]

A mixture of 3-bromo-N-(4-fluoro-3-methoxy-phenyl)-N-methyl-imidazo[1,2-a]pyridine-6-carboxamide (0.809 g, 2.14 mmol, 1 eq.), methyl N-[5-[6-[(4-fluoro-3-methoxy-phenyl)-methyl-carbamoyl]imidazo[1,2-a]pyridin-3-yl]-2-pyridyl]carbamate (0.877 g, 3.00 mmol, 1.4 eq.) and cesium carbonate (1.045 g, 3.21 mmol, 1.5 eq.) in water (4.3) and 1,4-dioxane (12.8 mL) was flushed with argon for 5 min. Tetrakis(triphenylphosphine)palladium(0) (0.127 g, 0.107 mmol, 0.05 eq.) was then added and the reaction mixture was heated under microwave irradiation at 100° C. and stirred for 1 h. The reaction mixture was cooled down to room temperature and then water was added. The aqueous layer was extracted ethyl acetate and the combined organic layers were washed with brine, dried over Na₂SO₄, filtered, and concentrated under reduced pressure. The crude residue was purified over a silica gel cartridge (cyclohexane/[ethyl acetate/EtOH 3/1]) to afford methyl N-[5-[6-[(4-fluoro-3-methoxy-phenyl)-methyl-carbamoyl]imidazo[1,2-a]pyridin-3-yl]-2-pyridyl]carbamate as a white solid.

LC/MS (Method A) retention time=0.70; [M+H]⁺=450

¹H NMR (400 MHz, CDCl₃) δ: 8.28-8.26 (dd, J=0.7, 2.2 Hz, 1H), 8.20 (s, 2H), 8.15 (d, J=8.4 Hz, 1H), 7.68 (s, 1H), 7.55 (dd, J=2.4, 8.5 Hz, 1H), 7.51 (dd, J=0.7, 9.4 Hz, 1H), 7.23 (dd, J=1.6, 9.3 Hz, 1H), 7.00 (dd, J=8.5, 10.7 Hz, 1H), 6.74 (dd, J=2.5, 7.3 Hz, 1H), 6.64-6.58 (m, 1H), 3.87 (s, 3H), 3.80 (s, 3H), 3.47 (s, 3H)

Example 14: This example illustrates the preparation of methyl N-[5-[6-[(4-cyano-3-methoxy-phenyl)-methyl-carbamoyl]imidazo[1,2-a]pyridin-3-yl]-2-pyridyl]carbamate (Compound 51)

### Step 1: Preparation of 3-bromo-N-(4-cyano-3-methoxy-phenyl)-N-methyl-imidazo[1,2-a]pyridine-6-carboxamide

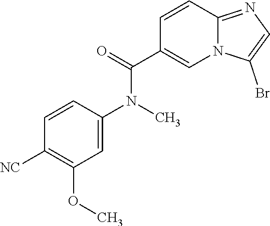 [SMILES: COc1cc(N(C)C(=O)c2ccc3ncc(Br)n3c2)ccc1C#N]

To a solution of 2-methoxy-4-(methylamino)benzonitrile (0.187 g, 1.12 mmol) in toluene (0.75 mL) at room temperature under argon, was slowly added a solution of trimethylaluminium in toluene (0.45 g, 0.56 mL, 1.12 mmol). The reaction mixture was stirred for 30 min at room temperature and then methyl 3-bromoimidazo[1,2-a]pyridine-6-carboxylate (0.100 g, 0.372 mmol) was added. The reaction mixture was then stirred for 1 h at 90° C. The reaction was then cooled to 0° C. and carefully quenched with a 1M HCl solution. The resulting mixture was treated with a saturated NaHCO₃ aq. solution until the aqueous layer reached a basic pH, and then extracted with ethyl acetate. The combined organic layers were washed with brine, dried over Na₂SO₄, filtered, concentrated under reduced pressure, and purified over a silica gel cartridge (cyclohexane/[Ethyl acetate/EtOH]; 3/1) to give 3-bromo-N-(4-cyano-3-methoxy-phenyl)-N-methyl-imidazo[1,2-a]pyridine-6-carboxamide as a yellow solid.

LC/MS (Method A) retention time=0.76 min; [M+H]⁺=385 NMR: ¹H NMR (400 MHz, CDCl₃) δ: ppm 8.43 (s, 1H), 7.73 (s, 1H), 7.67 (d, J=9.5 Hz, 1H), 7.51 (d, J=8.0 Hz, 1H), 7.22 (dd, J=9.5, 1.5 Hz, 1H), 6.74-6.81 (m, 2H), 3.87 (s, 3H), 3.59 (s, 3H).

### Step 2: Preparation of methyl N-[5-[6-[(4-cyano-3-methoxy-phenyl)-methyl-carbamoyl]imidazo[1,2-a]pyridin-3-yl]-2-pyridyl]carbamate

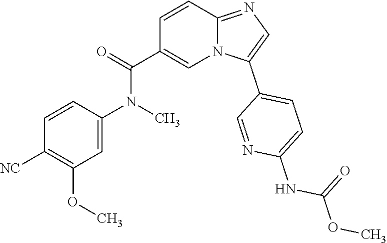 [SMILES: COC(=O)Nc1ccc(-c2cnc3ccc(C(=O)N(C)c4ccc(C#N)c(OC)c4)cn23)cn1]

In a microwave test tube was added 3-bromo-N-(4-cyano-3-methoxy-phenyl)-N-methyl-imidazo[1,2-a]pyridine-6-carboxamide (130 mg, 0.338 mmol), 2-methoxycarbonylaminopyridine-5-boronic acid pinacolester (136 mg, 0.473 mmol), cesium carbonate (167 mg, 0.506 mmol), 1,4-dioxane (10 mL), water (3.4 mL) and tetrakis(triphenylphosphine)palladium(0) (19.7 mg, 0.0168 mmol). The reaction mixture was purged with argon and heated at 100° C. under MW irradiation for 30 min. The reaction mixture was cooled down to room temperature, water was added to it, and the resulting mixture was extracted with ethyl acetate. The combined organic layers were washed with brine, dried over Na₂SO₄, filtered, concentrated under reduced pressure, and purified over a silica gel cartridge (CH₂Cl₂/MeOH) to afford methyl N-[5-[6-[(4-cyano-3-methoxy-phenyl)-methyl-carbamoyl]imidazo[1,2-a]pyridin-3-yl]-2-pyridyl]carbamate as a beige solid.

LC/MS (Method A) retention time=0.68 min; [M+H]⁺=457

NMR: ¹H NMR (400 MHz, DMSO-d₆) δ: 10.45 (s, 1H), 8.40 (dt, J=3.3, 1.6 Hz, 2H), 8.00 (dd, J=8.7, 0.7 Hz, 1H), 7.77-7.85 (m, 1H), 7.71-7.77 (m, 1H), 7.57-7.63 (m, 1H), 7.55 (d, J=9.5 Hz, 1H), 7.34 (d, J=1.8 Hz, 1H), 7.17-7.20 (m, 1H), 6.92 (dd, J=8.4, 1.8 Hz, 1H), 3.80 (s, 3H), 3.74 (s, 3H), 3.44 (s, 3H).

Example 15: This example illustrates the preparation of methyl N-[5-[6-[(4-fluorophenyl)-(2-methoxyethyl)carbamoyl]imidazo[1,2-a]pyridin-3-yl]-2-pyridyl]carbamate (Compound 76)

### Step 1: Preparation of 3-bromo-N-(4-fluorophenyl)imidazo[1,2-a]pyridine-6-carboxamide

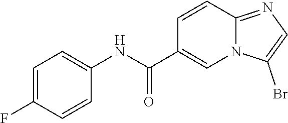 [SMILES: O=C(Nc1ccc(F)cc1)c1ccc2ncc(Br)n2c1]

To a solution of 3-bromoimidazo[1,2-a]pyridine-6-carboxylic acid (1.00 g, 4.00 mmol) in dimethylacetamide (16 mL) was added 4-fluoroaniline (0.47 mL, 4.8 mmol) followed by N,N-diisopropylethylamine (3.5 mL, 20 mmol) and propanephosphonic acid anhydride (50% wt. in ethyl acetate, 4.8 mL, 8.0 mmol) and the reaction mixture was stirred at 50° C. overnight. The reaction mixture was then cooled down to room temperature, water was added, and the mixture was extracted with ethyl acetate. The combined organic layers were washed with brine, dried over Na₂SO₄, filtered, and concentrated under reduced pressure. During the evaporation a precipitate formed which was filtered and washed with ethyl acetate to afford 3-bromo-N-(4-fluorophenyl)imidazo[1,2-a]pyridine-6-carboxamide as a yellow solid.

LC/MS (Method A) retention time=0.82; [M+H]⁺=334

¹H NMR (400 MHz, DMSO-d₆) δ: 10.57 (s, 1H), 8.97 (s, 1H), 7.83-7.89 (m, 2H), 7.76-7.81 (m, 3H), 7.21-7.27 (m, 2H).

### Step 2: Preparation of 3-bromo-N-(4-fluorophenyl)-N-(2-methoxyethyl)imidazo[1,2-a]pyridine-6-carboxamide

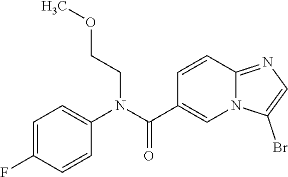 [SMILES: COCCN(C(=O)c1ccc2ncc(Br)n2c1)c1ccc(F)cc1]

To a solution of 3-bromo-N-(4-fluorophenyl)imidazo[1,2-a]pyridine-6-carboxamide (0.100 g, 0.299 mmol) in diethylformamide (3 mL) was added 2-bromoethyl methyl ether (0.064 g, 0.043 mL, 0.45 mmol) and cesium carbonate (0.294 g, 0.898 mmol). The reaction mixture was stirred overnight at 80° C. The reaction mixture was cooled to rt, water was added, and the mixture was extracted with ethyl acetate. The combined organic layers were washed with brine, dried over Na₂SO₄, filtered, concentrated under reduced pressure, and purified over a silica gel cartridge (cyclohexane/ethyl acetate) to afford 3-bromo-N-(4-fluorophenyl)-N-(2-methoxyethyl)imidazo[1,2-a]pyridine-6-carboxamide as a white solid.

LC/MS (Method A) retention time=0.82 min; [M+H]⁺=392

¹H NMR (400 MHz, DMSO-d₆) δ: 8.37 (br s, 1H), 7.69 (s, 1H), 7.55-7.65 (m, 1H), 7.19-7.25 (m, 3H), 7.03 (t, J=8.4 Hz, 2H), 4.12 (t, J=5.4 Hz, 2H), 3.68 (t, J=5.4 Hz, 2H), 3.40 (s, 3H).

### Step 3: Preparation of methyl N-[5-[6-[(4-fluorophenyl)-(2-methoxyethyl)carbamoyl]imidazo[1,2-a]pyridin-3-yl]-2-pyridyl]carbamate

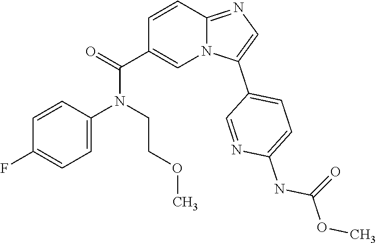

In a microwave test tube was added 3-bromo-N-(4-fluorophenyl)-N-(2-methoxyethyl)imidazo[1,2-a]pyridine-6-carboxamide (0.085 g, 0.22 mmol), 2-methoxycarbonylaminopyridine-5-boronic acid pinacolester (0.087 g, 0.30 mmol), cesium carbonate (0.107 g, 0.325 mmol), 1,4-dioxane (6.5 mL), water (2.2 mL) and tetrakis(triphenylphosphine)palladium(0) (0.013 mg, 0.011 mmol). The reaction mixture was purged with argon and heated at 100° C. under MW irradiation for 30 min. The reaction mixture was cooled to room temperature, water was added, and the mixture was extracted with ethyl acetate. The combined organic layers were washed with brine, dried over Na₂SO₄, filtered, concentrated under reduced pressure. The crude residue was purified over a silica gel cartridge (dichloromethane/MeOH) to afford methyl N-[5-[6-[(4-fluorophenyl)-(2-methoxyethyl)carbamoyl]imidazo[1,2-a]pyridin-3-yl]-2-pyridyl]carbamate as a beige solid.

LC/MS (Method A) retention time=0.72 min; [M+H]⁺=464

¹H NMR (400 MHz, DMSO-d₆) δ: 10.45 (s, 1H), 8.37-8.42 (m, 1H), 8.31-8.37 (m, 1H), 8.02 (d, J=8.7 Hz, 1H), 7.77-7.85 (m, 2H), 7.46-7.53 (m, 1H), 7.32-7.40 (m, 2H), 7.08-7.18 (m, 3H), 3.98 (t, J=5.6 Hz, 2H), 3.74 (s, 3H), 3.51 (br t, J=5.6 Hz, 2H), 3.21 (s, 3H).

Example 16: This example illustrates the preparation of methyl N-[5-[6-[[4-fluoro-3-(2-methoxyethoxy)phenyl]-methyl-carbamoyl]imidazo[1,2-a]pyridin-3-yl]-2-pyridyl]carbamate (Compound 77)

### Step 1: Preparation of 3-bromo-N-(4-fluoro-3-hydroxy-phenyl)-N-methyl-imidazo[1,2-a]pyridine-6-carboxamide

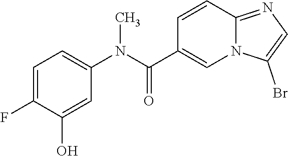 [SMILES: CN(C(=O)c1ccc2ncc(Br)n2c1)c1ccc(F)c(O)c1]

To a suspension of 3-bromo-N-(4-fluoro-3-methoxy-phenyl)-N-methyl-imidazo[1,2-a]pyridine-6-carboxamide (previously described in example 13, step 1, 0.910 g, 2.41 mmol) in dichloromethane (12.0 mL) at room temperature was added dropwise boron tribromide (1 M solution in dichloromethane, 4.8 mL, 4.81 mmol). The reaction mixture was stirred at room temperature for 72 h. The reaction mixture was then very slowly poured into water, basified with saturated NaHCO₃ aq. and extracted with dichloromethane. The combined organic layers were washed with brine, dried over MgSO₄, filtered, and concentrated under reduced pressure. The crude residue was purified over a silica gel cartridge (cyclohexane/[ethyl acetate/EtOH 3/1]) as eluent to give 3-bromo-N-(4-fluoro-3-hydroxy-phenyl)-N-methyl-imidazo[1,2-a]pyridine-6-carboxamide as a brown gum.

LC/MS (Method A) retention time=0.70 min; [M+H]⁺=364

¹H NMR (400 MHz, CDCl₃) δ: 8.39 (s, 1H), 7.57 (s, 1H), 7.19-7.26 (m, 1H), 6.97-7.04 (m, 2H), 6.79 (dd, J=7.6, 2.9 Hz, 1H), 6.59-6.66 (m, 1H), 3.51 (s, 3H).

### Step 2: Preparation of 3-bromo-N-[4-fluoro-3-(2-methoxyethoxy)phenyl]-N-methyl-imidazo[1,2-a]pyridine-6-carboxamide

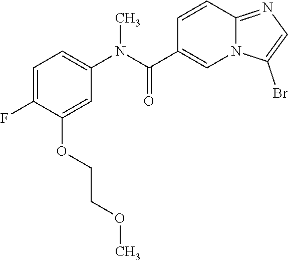 [SMILES: COCCOc1cc(N(C)C(=O)c2ccc3ncc(Br)n3c2)ccc1F]

A mixture of 3-bromo-N-(4-fluoro-3-hydroxy-phenyl)-N-methyl-imidazo[1,2-a]pyridine-6-carboxamide (0.100 g, 0.275 mmol), 1-bromo-2-methoxy-ethane (0.412 mmol, 0.039 mL) and potassium carbonate (0.096 mg, 0.69 mmol) was dissolved in dimethylformamide (4 mL). The reaction mixture was stirred for 3.5 h at rt before it was diluted with ethyl acetate, quenched with water, and extracted with ethyl acetate. The organic layers were washed with water and brine, dried over Na₂SO₄, filtered, and concentrated under reduced pressure. The crude residue was purified over a silica gel cartridge (cyclohexane/[ethyl acetate/EtOH 3/1]) to afford 3-bromo-N-[4-fluoro-3-(2-methoxyethoxy)phenyl]-N-methyl-imidazo[1,2-a]pyridine-6-carboxamide as an orange solid.

LC/MS (Method A) retention time=0.82 min; [M+H]⁺=422

¹H NMR (400 MHz, CDCl₃) δ: 8.31 (dd, J=1.8, 1.1 Hz, 1H), 7.62 (s, 1H), 7.38 (dd, J=9.5, 1.1 Hz, 1H), 7.09 (dd, J=9.5, 1.5 Hz, 1H), 7.00 (dd, J=10.7, 8.5 Hz, 1H), 6.85 (dd, J=7.3, 2.5 Hz, 1H), 6.65-6.72 (m, 1H), 4.08-4.13 (m, 2H), 3.67-3.73 (m, 2H), 3.51 (s, 3H), 3.40 (s, 3H).

### Step 3: Preparation of methyl N-[5-[6-[[4-fluoro-3-(2-methoxyethoxy)phenyl]-methyl-carbamoyl]imidazo[1,2-a]pyridin-3-yl]-2-pyridyl]carbamate

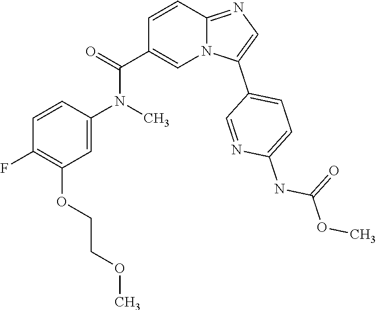

In a microwave test tube containing 3-bromo-N-[4-fluoro-3-(2-methoxyethoxy)phenyl]-N-methyl-imidazo[1,2-a]pyridine-6-carboxamide (0.088 g, 0.21 mmol) was added 2-methoxycarbonylaminopyridine-5-boronic acid pinacolester (0.084 g, 0.29 mmol), cesium carbonate (0.103 g, 0.313 mmol), 1,4-dioxane (6.3 mL), water (2.1 mL) and tetrakis(triphenylphosphine)palladium(0) (0.012 g, 0.010 mmol). The reaction mixture was purged with argon and heated at 100° C. under MW irradiation for 30 min. The reaction was quenched with water and the resulting mixture was extracted with ethyl acetate. The combined organic layers were washed with brine, dried over Na₂SO₄, filtered, and concentrated under reduced pressure. The crude residue was purified over a silica gel cartridge (cyclohexane/[Ethyl acetate/EtOH 3/1]) to afford N-[5-[6-[[4-fluoro-3-(2-methoxyethoxy)phenyl]-methyl-carbamoyl]imidazo[1,2-a]pyridin-3-yl]-2-pyridyl]carbamate as an orange gum.

LC/MS (Method A) retention time=0.72 min; [M+H]⁺=494

¹H NMR (400 MHz, CDCl₃) δ: 8.28 (s, 1H), 8.12-8.16 (m, 2H), 8.02 (d, J=1.5 Hz, 1H), 7.69 (s, 1H), 7.59-7.64 (m, 1H), 7.53-7.58 (m, 1H), 7.35 (dd, J=9.5, 1.8 Hz, 1H), 6.94-7.02 (m, 1H), 6.86 (dd, J=7.3, 2.5 Hz, 1H), 6.54-6.58 (m, 1H), 4.18-4.23 (m, 2H), 3.88 (s, 3H), 3.80-3.86 (m, 2H), 3.50 (s, 3H), 3.47 (s, 3H).

Example 17: This example illustrates the preparation of N-(4-fluoro-3-methoxy-phenyl)-N-methyl-3-[6-(propanoylamino)-3-pyridyl]imidazo[1,2-a]pyridine-6-carboxamide (Compound 151) Step 1: Preparation of N-[5-(4,4,5,5-tetramethyl-1,3,2-dioxaborolan-2-yl)-2-pyridyl]propanamide

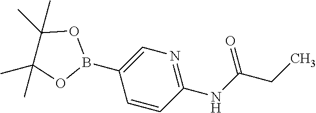 [SMILES: CCC(=O)Nc1ccc(B2OC(C)(C)C(C)(C)O2)cn1]

To a stirred solution of 2-aminopyridine-5-boronic acid pinacolester (1.0 g, 4.3 mmol) in ethyl acetate (50 mL) was added pyridine (1.4 mL, 17.3 mmol) under nitrogen at 0° C. and stirred for 10 min. Propanoyl chloride (1.597 g, 1.508 mL, 17.26 mmol) was then added and the reaction mixture was stirred at ambient temperature for 1 h. The reaction mixture was then diluted with water. The aqueous layer was extracted with ethyl acetate and the combined organic layers were washed with brine, dried over Na₂SO₄, filtered, and concentrated under reduced pressure to afford N-[5-(4,4,5,5-tetramethyl-1,3,2-dioxaborolan-2-yl)-2-pyridyl]propanamide as a gum. The crude material was used for next step without further purification, Step 2: Preparation of N-(4-fluoro-3-methoxy-phenyl)-N-methyl-3-[6-(propanoylamino)-3-pyridyl]imidazo[1,2-a]pyridine-6-carboxamide

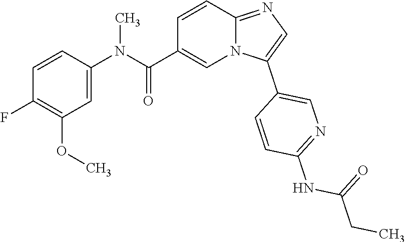 [SMILES: CCC(=O)Nc1ccc(-c2cnc3ccc(C(=O)N(C)c4ccc(F)c(OC)c4)cn23)cn1]

A mixture of 3-bromo-N-(4-fluoro-3-methoxy-phenyl)-N-methyl-imidazo[1,2-a]pyridine-6-carboxamide (0.200 g, 0.513 mmol), N-[5-(4,4,5,5-tetramethyl-1,3,2-dioxaborolan-2-yl)-2-pyridyl]propanamide (0.212 g, 0.769 mmol) and sodium carbonate (0.163 g, 1.54 mmol) in water (2.05 mL) and acetonitrile (4.10 mL) was flushed with nitrogen for 10 min. Chloro(2-dicyclohexylphosphino-2′,4′,6′-triisopropyl-1,1′-biphenyl)[2-(2′-amino-1,1′-biphenyl)]palladium(II) (0.042 g, 0.051 mmol) was then added and the reaction mixture was heated under microwave irradiation at 100° C. and stirred for 3 h. The reaction mixture was cooled down to room temperature and then water was added. The aqueous layer was extracted with ethyl acetate and the combined organic layers were washed with brine, dried over Na₂SO₄, filtered, and concentrated under reduced pressure. The crude residue was purified over a C₁₈ silica cartridge (40% water-acetonitrile as eluent) to afford N-(4-fluoro-3-methoxy-phenyl)-N-methyl-3-[6-(propanoylamino)-3-pyridyl]imidazo[1,2-a]pyridine-6-carboxamide as a white solid.

¹H NMR (400 MHz, DMSO-d₆) δ: 10.66 (s, 1H), 8.43 (d, J=1.7 Hz, 1H), 8.34 (s, 1H), 8.27 (d, J=8.8 Hz, 1H), 7.80 (s, 1H), 7.77 (dd, J=8.6, 2.3 Hz, 1H), 7.53 (d, J=9.4 Hz, 1H), 7.30 (dd, J=7.9, 2.5 Hz, 1H), 7.21 (dd, J=9.4, 1.5 Hz, 1H), 7.07 (dd, J=11.3, 8.6 Hz, 1H), 6.78 (br d, J=1.1 Hz, 1H), 3.72 (s, 3H), 3.38 (s, 3H), 2.44-2.49 (m, 2H), 1.12 (t, J=7.6 Hz, 3H)

Example 18: This example illustrates the preparation of N-(4-fluoro-3-methoxy-phenyl)-3-[6-[[methoxy(methyl)carbamoyl]amino]-3-pyridyl]-N-methyl-imidazo[1,2-a]pyridine-6-carboxamide (Compound 79)

### Step 1: Preparation of 3-(6-amino-3-pyridyl)-N-(4-fluoro-3-methoxy-phenyl)-N-methyl-imidazo[1,2-a]pyridine-6-carboxamide

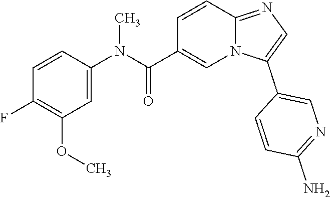 [SMILES: COc1cc(N(C)C(=O)c2ccc3ncc(-c4ccc(N)nc4)n3c2)ccc1F]

A mixture of 3-bromo-N-(4-fluoro-3-methoxy-phenyl)-N-methyl-imidazo[1,2-a]pyridine-6-carboxamide (0.300 g, 0.793 mmol, 1 eq.), 5-(4,4,5,5-tetramethyl-1,3,2-dioxaborolan-2-yl)pyridin-2-amine (0.249 g, 1.11 mmol) and cesium carbonate (0.39 g, 1.19 mmol) in water (3.97 mL) in 1,4-dioxane (11.9 mL) was flushed with argon for 10 min. Tetrakis(triphenylphosphine)palladium(0) (0.046 g, 0.040 mmol) was then added and the reaction mixture was heated under microwave irradiation at 100° C. for 1 h. The reaction mixture was cooled down to room temperature and then water was added. The aqueous layer was extracted with ethyl acetate and the combined organic layers were washed with brine, dried over Na₂SO₄, filtered, and concentrated under reduced pressure to afford crude mass of 3-(6-amino-3-pyridyl)-N-(4-fluoro-3-methoxy-phenyl)-N-methyl-imidazo[1,2-a]pyridine-6-carboxamide as a brown solid, which was taken for next step without further purifications.

LC/MS (Method A) retention time=0.51 min; [M+H]⁺=392

### Step 2: Preparation of N-(4-fluoro-3-methoxy-phenyl)-3-[6-[[methoxy(methyl)carbamoyl]amino]-3-pyridyl]-N-methyl-imidazo[1,2-a]pyridine-6-carboxamide

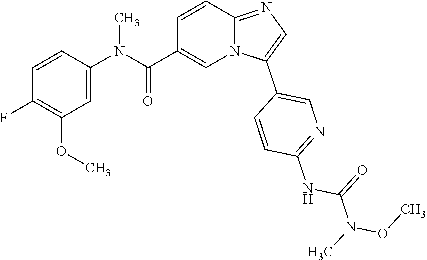 [SMILES: COc1cc(N(C)C(=O)c2ccc3ncc(-c4ccc(NC(=O)N(C)OC)nc4)n3c2)ccc1F]

To a solution of 3-(6-amino-3-pyridyl)-N-(4-fluoro-3-methoxy-phenyl)-N-methyl-imidazo[1,2-a]pyridine-6-carboxamide (0.075 g, 0.19 mmol) in ethyl acetate (0.48 mL), was added pyridine (0.15 mL, 1.9 mmol) under nitrogen at 0° C. The reaction mixture was stirred for 10 min and then N-methoxy-N-methylcarbamoyl chloride (120 mg, 0.96 mmol) was added and the reaction mixture was stirred at ambient temperature for an additional 2 h. The reaction mixture was then diluted with water. The aqueous layer was extracted ethyl acetate and the combined organic layers were washed with brine, dried over Na₂SO₄, filtered, concentrated under reduced pressure and purified over a silica gel cartridge (ethyl acetate/cyclohexane) to afford N-(4-fluoro-3-methoxy-phenyl)-3-[6-[[methoxy(methyl)carbamoyl]amino]-3-pyridyl]-N-methyl-imidazo[1,2-a]pyridine-6-carboxamide as a brown solid.

LC/MS (Method A) retention time=0.73; [M+H]⁺=479

¹H NMR (400 MHz, CDCl₃) δ: 8.55 (s, 1H) 8.26-8.35 (m, 1H), 8.24 (d, J=1.8 Hz, 1H), 8.21 (br s, 1H), 7.65-7.80 (m, 2H), 7.52-7.60 (m, 1H), 7.38 (dd, J=9.5, 1.5 Hz, 1H), 7.02 (dd, J=10.5, 8.7 Hz, 1H), 6.66-6.83 (m, 1H), 6.61 (dt, J=8.4, 3.1 Hz, 1H), 3.85 (s, 3H), 3.81 (s, 3H), 3.48 (s, 3H), 3.28 (s, 3H).

Example 19: This example illustrates the preparation of methyl N-[5-[6-[(4-fluorophenyl)-methyl-carbamothioyl]imidazo[1,2-a]pyridin-3-yl]-2-pyridyl]carbamate (Compound 164)

### Step 1: Preparation of methyl N-[5-[6-[(4-fluorophenyl)-methyl-carbamothioyl]imidazo[1,2-a]pyridin-3-yl]-2-pyridyl]carbamate

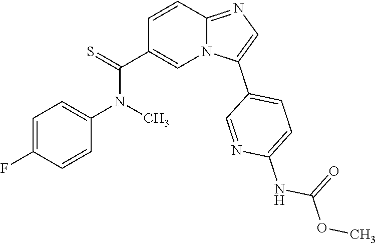 [SMILES: COC(=O)Nc1ccc(-c2cnc3ccc(C(=S)N(C)c4ccc(F)cc4)cn23)cn1]

To a microwave test tube containing a mixture of methyl N-[5-[6-[(4-fluorophenyl)-methyl-carbamoyl]imidazo[1,2-a]pyridin-3-yl]-2-pyridyl]carbamate (0.205 g, 0.489 mmol) in toluene (2 mL) was added Lawesson's reagent (0.112 g, 0.269 mmol). The resulting mixture was heated to 100° C. and stirred for 2 h. The reaction mixture was cooled to rt, and the solvent evaporated under reduced pressure. The crude residue was purified over a silica gel cartridge (cyclohexane/[Ethyl acetate/EtOH 3/1]) to afford methyl N-[5-[6-[(4-fluorophenyl)-methyl-carbamothioyl]imidazo[1,2-a]pyridin-3-yl]-2-pyridyl]carbamate as a yellow solid.

LC/MS (Method A) retention time=0.77 min; [M+H]⁺=435

¹H NMR (400 MHz, CDCl₃) δ: 8.84 (br s, 1H), 8.32 (d, J=2.2 Hz, 1H), 8.20 (d, J=8.7 Hz, 1H), 8.16 (br s, 1H), 7.67 (s, 1H), 7.64 (br dd, J=8.7, 1.8 Hz, 1H), 7.50 (br d, J=9.4 Hz, 1H), 7.21 (dd, J=9.4, 1.8 Hz, 1H), 7.05-7.12 (m, 2H), 6.97-7.05 (m, 2H), 3.89 (br s, 3H), 3.87 (s, 3H).

### BIOLOGICAL EXAMPLES

The fungicidal activity of the compounds of the invention have been tested as follows:

Phytophthora infestans/Tomato/Leaf Disc Preventative (Late Blight):

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

The following compounds in Table A gave at least 80% control of Phytophthora infestans at 200 ppm when compared to untreated control under the same conditions, which showed extensive disease development:

2, 3, 4, 6, 7, 10, 11, 15, 19, 25, 26, 27, 31, 32, 37, 43, 45, 46, 48, 50, 51, 52, 53, 55, 57, 62, 63, 70, 71, 73, 75, 76, 77, 80, 81, 82, 83, 84, 85, 86, 89, 90, 92, 93, 94, 95, 96, 97, 98, 99, 100, 105, 107, 109, 110, 113, 115, 116, 119, 123, 124, 128, 129, 130, 132, 134, 136, 140, 149, 158, 164, 168, 169, 170, 171, 172, 173, 174, 177, 178.

Plasmopara viticola/Grape/Leaf Disc Preventative (Late Blight):

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

The following compounds in Table A gave at least 80% control of Plasmopara viticola at 200 ppm when compared to untreated control under the same conditions, which showed extensive disease development:

1, 2, 3, 4, 6, 7, 9, 11, 15, 19, 25, 26, 27, 31, 32, 37, 45, 46, 48, 50, 51, 53, 57, 58, 61, 63, 64, 70, 73, 75, 76, 78, 82, 84, 85, 86, 89, 90, 93, 94, 95, 96, 98, 100, 105, 107, 110, 113, 116, 124, 126, 136, 137, 149, 158, 164, 169, 170, 171,172, 173, 174, 177, 178.

Pythium ultimum/Liquid Culture (Seedling Damping Off):

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application.

The following compounds in Table A gave at least 80% control of Pythium ultimum at 20 ppm when compared to untreated control under the same conditions, which showed extensive disease development:

1, 2, 3, 4, 6, 7, 9, 10, 11, 12, 13, 15, 16, 18, 19, 24, 25, 26, 27, 28, 29, 30, 31, 32, 33, 34, 37, 39, 41, 42, 43, 45, 46, 48, 50, 51, 52, 53, 54, 55, 57, 58, 59, 60, 61, 62, 63, 64, 65, 66, 68, 69, 70, 71, 72, 73, 75, 76, 77, 78, 80, 81, 82, 83, 84, 85, 86, 89, 90, 91, 92, 93, 94, 95, 96, 97, 98, 99, 100, 104, 105, 107, 108, 109, 110, 112, 113, 115, 116, 121, 122, 123, 124, 125, 126, 127, 128, 129, 130, 132, 136, 137, 140, 141, 143, 145, 147, 148, 149, 150, 158, 164, 165, 166, 168, 169, 170, 171, 172, 173, 174, 176, 178.

1. A compound of formula (I) 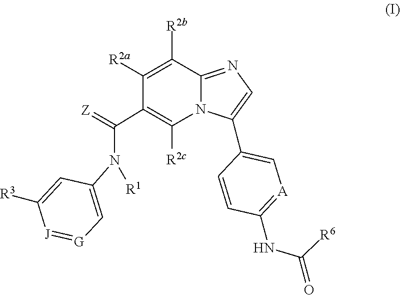
wherein Z is O or S;
R¹ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen, hydroxy and CN;
R^{2a}, R^{2b} and R^{2c} independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
J is CR⁴ or N;
G is CR⁵ or N;
wherein when J is N, then G is CR⁵; and when G is N, then J is CR⁴;
R³, R⁴ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN;
A is CH or N; and
R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN; or a salt or N-oxide thereof.

2. The compound or salt according to claim 1, wherein
A is N; and
R⁶ is selected from C₁₋₄alkyl and C₁₋₄alkoxy, wherein each of said groups is optionally substituted with one to three substituents independently selected from halogen.

3. The compound or salt according to claim 1, wherein
A is CH; and
R⁶ is selected from C₁₋₄alkyl and C₁₋₄alkoxy, wherein each of said groups is optionally substituted with one to three substituents independently selected from halogen.

4. The compound or salt according to claim 1, wherein
R⁶ is selected from methyl, ethyl and methoxy.

5. The compound or salt according to claim 1, wherein
R¹ is selected from C₁₋₄alkyl, C₁₋₄alkoxy-C₁₋₄alkyl, C₃₋₆cycloalkyl, C₃₋₆cycloalkyl-C₁₋₃alkyl and C₂₋₄alkynyl, wherein each of said groups is optionally substituted with one to three substituents independently selected from halogen and CN.

6. The compound or salt according to claim 1,
wherein J is CR⁴ and G is CR⁵; and
R³, R⁴ and R⁵ are independently selected from H, C₁₋₄alkyl, C₁₋₄alkoxy, C₁₋₄alkoxy-C₁₋₄alkyl, C₁₋₄alkoxy-C₁₋₄alkoxy, halogen, CN, C₂₋₄alkynyl, C₃₋₆cycloalkyl, C₃₋₆cycloalkyl-C₁₋₃alkyl and hydroxy, wherein each of the C₁₋₄alkyl, C₁₋₄alkoxy, C₁₋₄alkoxy-C₁₋₄alkyl, C₁₋₄alkoxy-C₁₋₄alkoxy, C₂₋₄alkynyl, C₃₋₆cycloalkyl and C₃₋₆cycloalkyl-C₁₋₃alkyl groups is optionally substituted with one to three substituents independently selected from halogen.

7. The compound or salt according to claim 1,
wherein J is CR⁴ and G is N; and
R³ and R⁴ are independently selected from H, C₁₋₄alkyl, C₁₋₄alkoxy, halogen, CN and C₂₋₄alkynyl, wherein each of said groups is optionally substituted with one to three substituents independently selected from halogen.

8. The compound or salt according to claim 1,
wherein J is N and G is CR⁵; and
R³ and R⁵ are independently selected from H, C₁₋₄alkyl, C₁₋₄alkoxy, halogen, CN and C₂₋₄alkynyl, wherein each of said groups is optionally substituted with one to three substituents independently selected from halogen.

9. A composition comprising a fungicidally effective amount of a compound as defined in claim 1.

10. A composition according to claim 9, wherein the composition further comprises at least one compound selected among an additional active ingredient, an appropriate formulation inert, a carrier, an adjuvant, and any mixtures thereof.

11. A method of combating, preventing or controlling phytopathogenic diseases which comprises applying to a phytopathogen, to the locus of a phytopathogen, or to a plant susceptible to attack by a phytopathogen, or to propagation material thereof, a fungicidally effective amount of a compound according to claim 1 or a composition comprising a compound according to claim 1.

12. A compound of formula (XV) 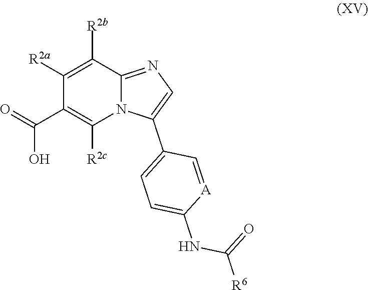
wherein A is CH or N;
R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
or
a compound of formula (XVI) 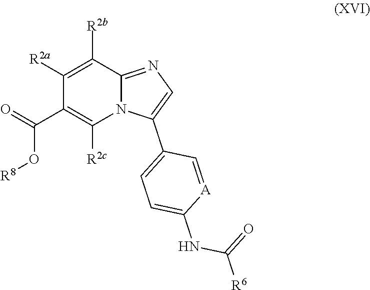
wherein A is CH or N;
R⁶ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN;
R⁸ is C₁-C₆ alkyl; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl.

13. A compound of formula (XXI) 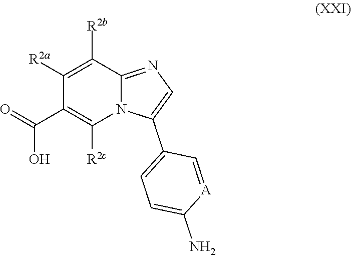
wherein A is CH or N; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
or
a compound of formula (XXII) 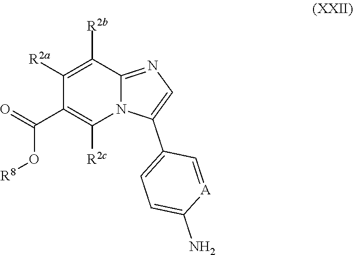
wherein A is CH or N;
R⁸ is C₁-C₆ alkyl; and
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
or
a compound of formula (II) 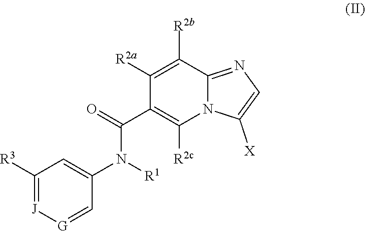
wherein R¹ is selected from C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
J is CR⁴ or N;
G is CR⁵ or N;
wherein when J is N, then G is CR⁵; and when G is N, then J is CR⁴;
R³ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN;
R⁴ is selected from H, C₁₋₆alkyl, C₁₋₆alkoxy, C₁₋₆alkoxy-C₁₋₆alkyl, C₁₋₆alkoxy-C₁₋₆alkoxy, halogen, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN; and
X is Cl, Br or I;
or
a compound of formula (XVII) 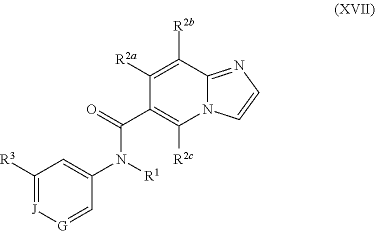
wherein R¹ is selected from C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl, C₁₋₄alkoxy and CN, wherein each of the C₁₋₄alkyl, C₁₋₄alkoxy-C₁₋₄alkyl, C₃₋₆cycloalkyl, C₃₋₆cycloalkyl-C₁₋₃alkyl, C₂₋₄alkenyl, C₂₋₄alkynyl and C₁₋₄alkoxy groups is optionally substituted with one to three substituents independently selected from halogen and CN;
R^{2a}, R^{2b} and R^{2c} are independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
J is CR⁴ or N;
G is CR⁵ or N;
wherein when J is N, then G is CR⁵; and when G is N, then J is CR⁴; and
R³, R⁴ and R⁵ are independently selected from H, C₁₋₆alkyl, C₁₋₆alkoxy, C₁₋₆alkoxy-C₁₋₆alkyl, C₁₋₆alkoxy-C₁₋₆alkoxy, halogen, CN, C₂₋₆alkenyl, C₂₋₆alkynyl, C₃₋₆cycloalkyl, C₃₋₆cycloalkyl-C₁₋₆alky, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, C₁₋₆alkylcarbonyl, and hydroxy, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen and CN.

14. (canceled)

15. (canceled)
